(19) United States
(12) Patent Application Publication
(10) Pub. No.: US 2020/0361987 A1
(43) Pub. Date: Nov. 19, 2020

(54) DEUTERATED TETRAPEPTIDES THAT TARGET MITOCHONDRIA

(71) Applicant: STEALTH BIOTHERAPEUTICS CORP., Grand Cayman (KY)

(72) Inventor: Guozhu Zheng, Lexington, MA (US)

(21) Appl. No.: 16/764,278

(22) PCT Filed: Nov. 14, 2018
(86) PCT No.: PCT/US18/60997

§ 371 Date: May 14, 2020

Related U.S. Application Data
(60) Provisional application No. 62/586,336, filed on Nov. 15, 2017.

(51) Int. Cl. C07K 5/107; A61P 13/12

(52) U.S. Cl. CPC C07K 5/1016; A61K 38/00; A61P 13/12

(57) ABSTRACT

Disclosed are deuterated analogs of SBT-20 and elamipretide (MTP-131). The compounds are useful for the treatment and prevention of ischemia-reperfusion injury (e.g., cardiac ischemia-reperfusion injury) or myocardial infarction.

### RELATED APPLICATION

This application claims the benefit of priority to U.S. Provisional Patent Application Ser. No. 62/586,336, filed Nov. 15, 2017.

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of many drug candidates in clinical trials. While formulation technologies and prodrug strategies can be employed in some cases to improve or alter certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This approach, however, introduces a number of potential treatment problems, such as poor patient compliance with the dosing regimen, side effects that become more acute with higher doses, and increased cost of treatment.

Another ADME limitation that affects many medicines is the formation of toxic or biologically reactive metabolites. As a result, some patients receiving the drug may experience toxicities, or the safe dosing of such drugs may be limited, such that patients receive a suboptimal amount of the active agent. In certain cases, modifying dosing intervals or formulation approaches can help to reduce clinical adverse effects, but often the formation of undesirable metabolites is intrinsic to the metabolism of the compound.

A potentially attractive strategy for improving metabolic properties of a drug is deuterium modification. In this approach, one attempts to either prevent or slow down protease activity that breaks down a peptide analog by using unnatural amino acid building blocks, and/or to slow the cytochrome P450-mediated metabolism of a drug or to reduce the formation of undesirable metabolites by replacing one or more hydrogen atoms with deuterium atoms. In addition, deuterium analogs, although their size and shape are not significantly altered as compared to the non-deuterated analogs, have increased molecular weight. This increased molecular weight may affect their ADME profiles in a surprising and unpredictable manner to alter their efficacy in vivo. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium typically does not affect the biochemical potency or selectivity of the drug as compared to the original chemical entity that contains only hydrogen atoms.

Elamipretide (MTP-131) and SBT-20 are mitochondria-targeting compounds with therapeutic potential for treating diseases associated with mitochondrial dysfunction. Because of the potential therapeutic applications of elamipretide and SBT-20, there exists a need to develop analogs with improved metabolic properties.

### SUMMARY OF THE INVENTION

An aspect of the invention is a deuterated analog of SBT-20.

More specifically, the invention provides a compound of formula (I), or a pharmaceutically acceptable salt thereof:

Aaa¹-Aaa²-Aaa³-Aaa⁴-NH₂ (I);

wherein:
Aaa¹ is an L-phenylalanine residue;
Aaa² is a D-arginine residue;
Aaa³ is an L-phenylalanine residue;
Aaa⁴ is an L-lysine residue; and
at least one hydrogen atom is replaced by a deuterium atom.

In further embodiments, the invention provides a compound of formula (I) in which:

Aaa¹ is an L-phenylalanine residue or an amino acid residue selected from the group consisting of:

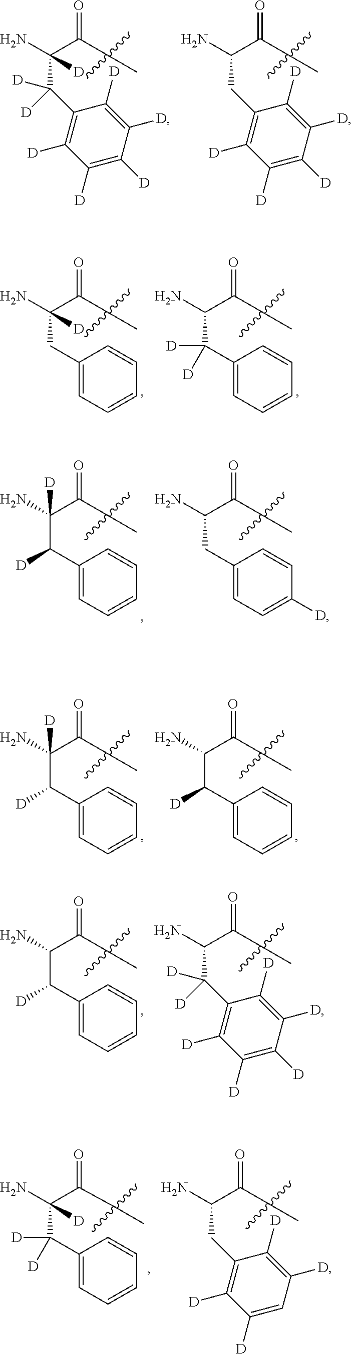

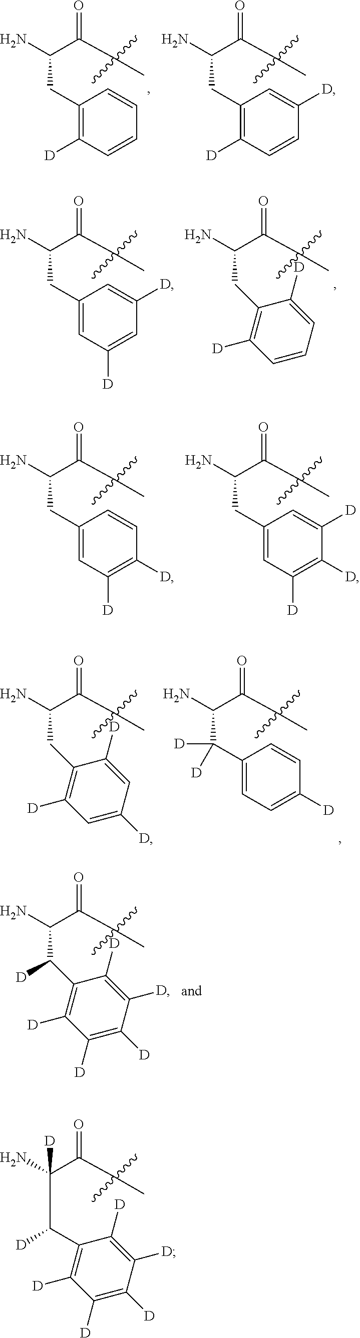

Aaa² is a D-arginine residue or an amino acid residue selected from the group consisting of:

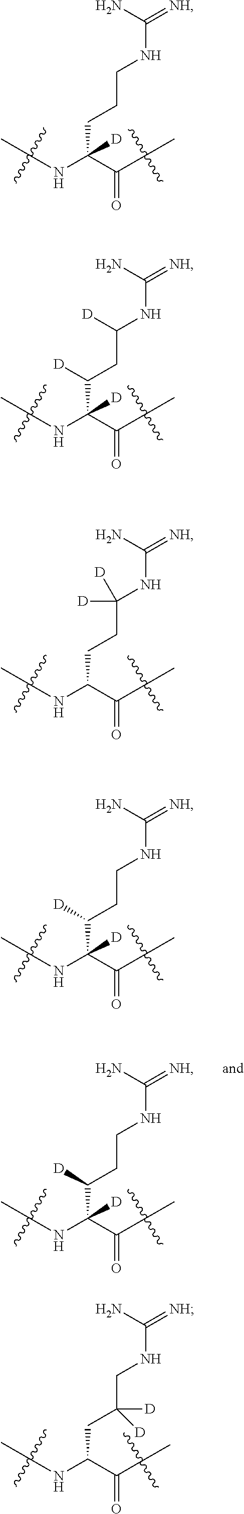

Aaa³ is an L-phenylalanine residue or an amino acid residue selected from the group consisting of:

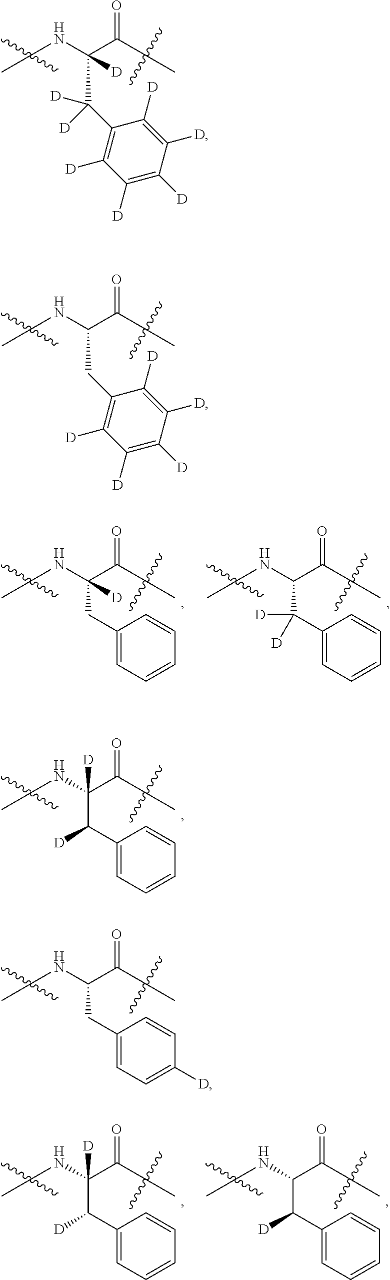

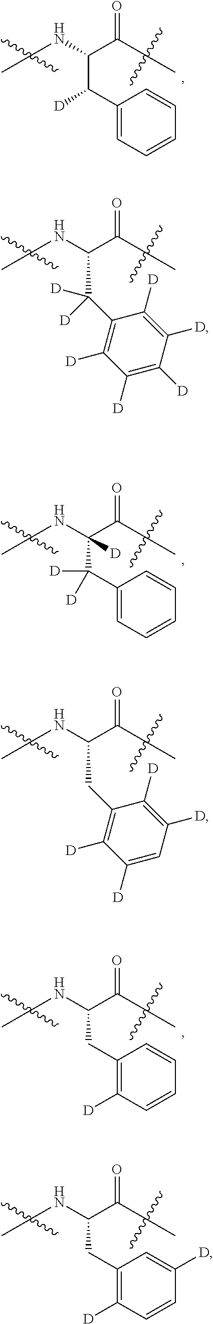

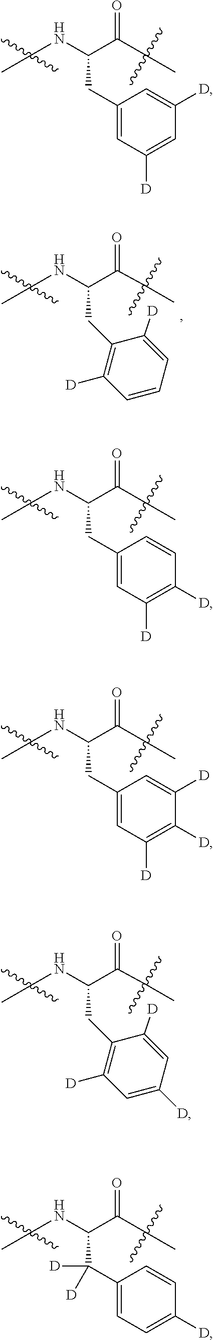

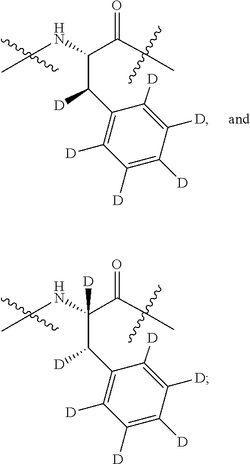

and

Aaa⁴ is an L-lysine residue or an amino acid residue selected from the group consisting of:

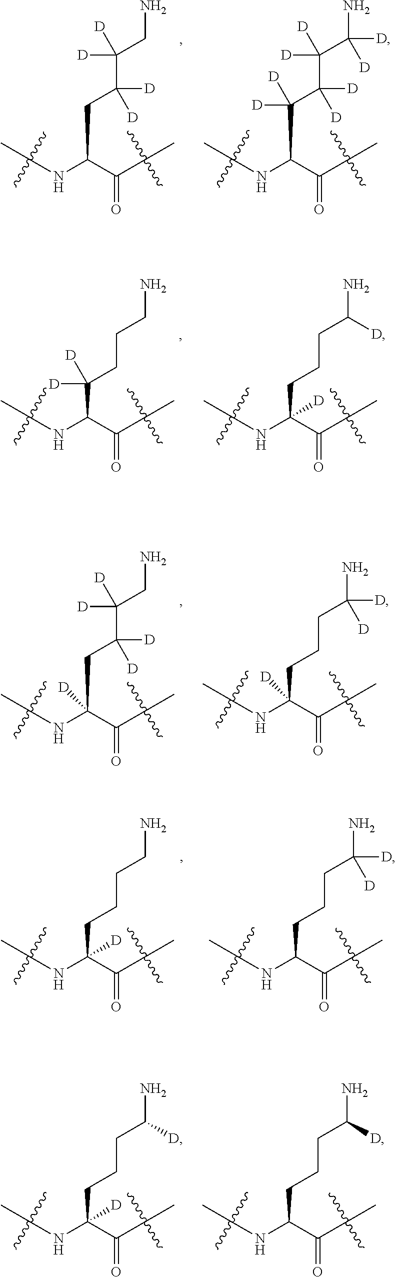

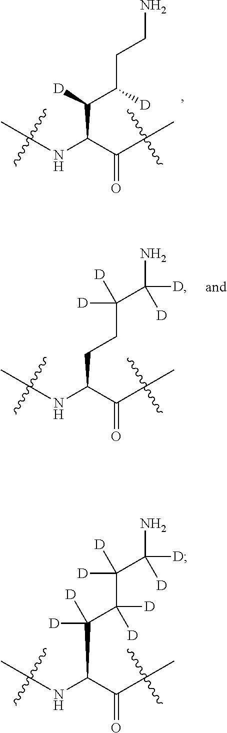

provided that the compound of formula (I) is not Phe-D-Ag-Phe-Lys-NH₂.

Another aspect of the invention is a deuterated analog of elamipretide (MTP-131).

More specifically the invention provides a compound of formula (II), or a pharmaceutically acceptable salt thereof:

Aaa⁵-Aaa⁶-Aaa⁷-Aaa⁸-NH₂ (II);

wherein:
Aaa⁵ is a D-arginine residue;

### Aaa⁶ is:

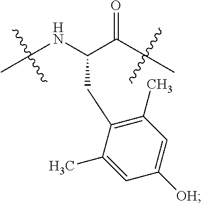 [SMILES: *N[C@@H](Cc1c(C)cc(O)cc1C)C(*)=O]

Aaa⁷ is an L-lysine residue;
Aaa⁸ is an L-phenylalanine residue; and
at least one hydrogen atom is replaced by a deuterium atom.

In further embodiments, the invention provides a compound of formula (II) in which:
Aaa⁵ is a D-arginine residue or an amino acid residue selected from the group consisting of:

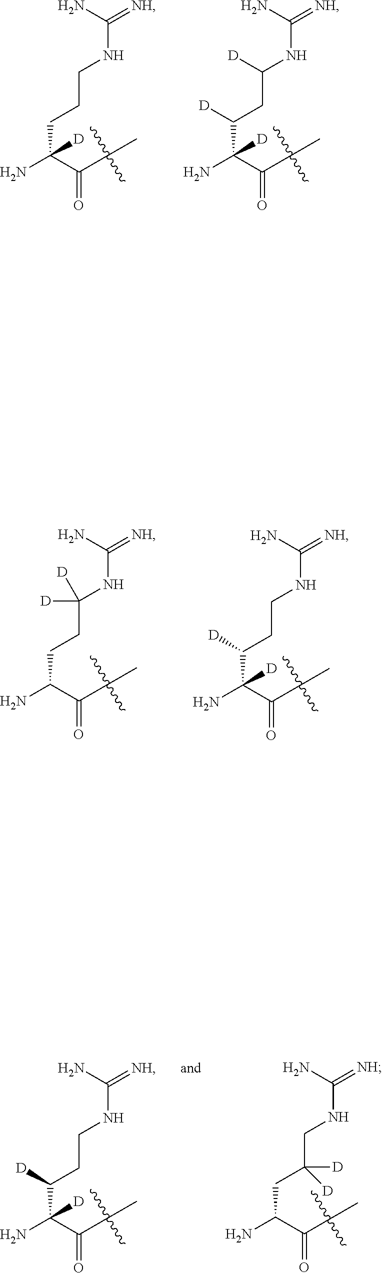

Aaa⁶ is:

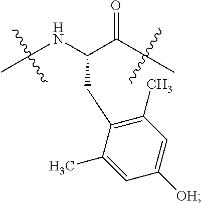 [SMILES: *N[C@@H](Cc1c(C)cc(O)cc1C)C(*)=O]

Aaa⁷ is an L-lysine residue or an amino acid residue selected from the group consisting of:

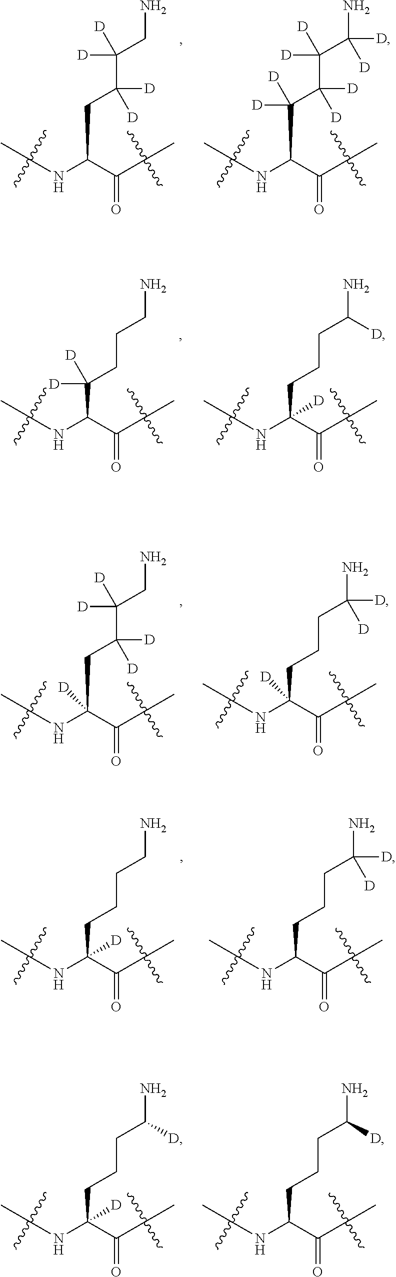

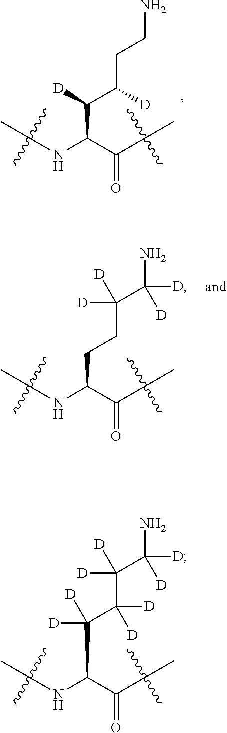

and
Aaa⁸ is an L-phenylalanine residue or an amino acid residue selected from the group consisting of:

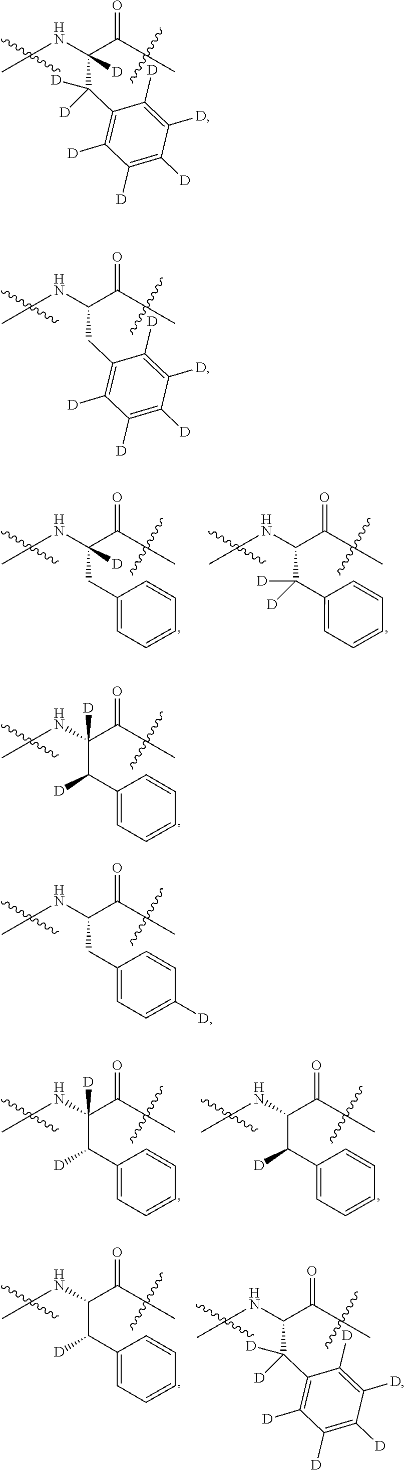

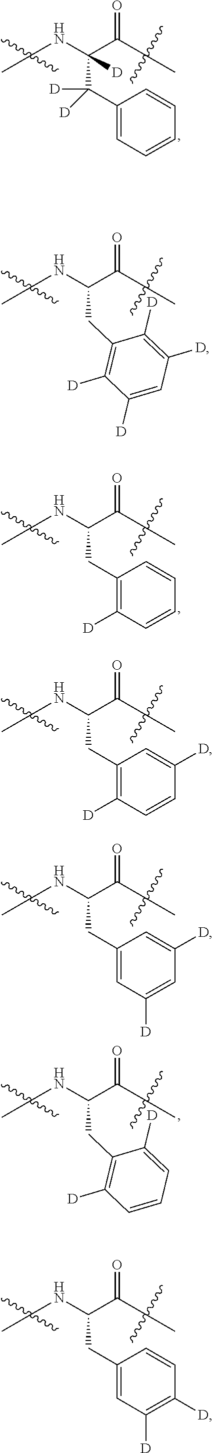

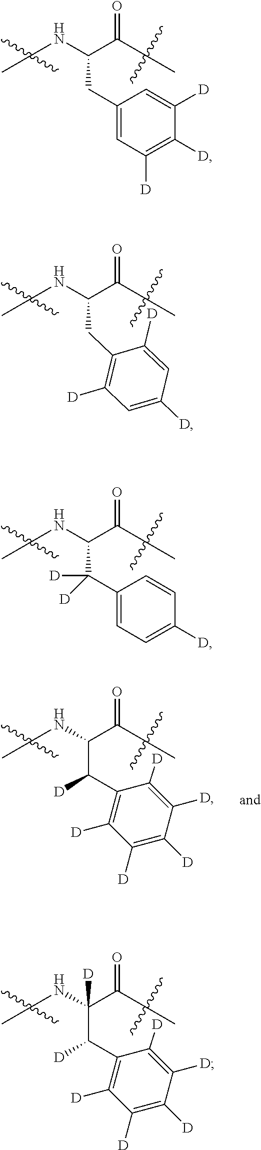

provided that the compound of formula (II) is not D-Arg-DMT-Lys-Phe-NH₂; wherein DMT represents

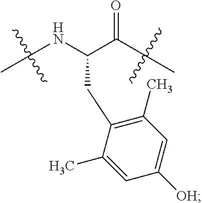 [SMILES: *N[C@@H](Cc1c(C)cc(O)cc1C)C(*)=O]

Another aspect of the invention is a pharmaceutical composition, comprising a compound of the invention, or a pharmaceutically acceptable salt, and a pharmaceutically acceptable carrier.

The invention also provides methods of treating or preventing ischemia-reperfusion injury, comprising administering to a subject in need thereof a therapeutically effective amount of a compound of the invention.

The invention also provides methods of treating or preventing myocardial infarction, comprising administering to a subject in need thereof a therapeutically effective amount of a compound of the invention.

### BRIEF DESCRIPTION OF THE DRAWINGS

FIG. 1 depicts various deuterated amino acid residues useful in the present invention.

FIG. 2 depicts various deuterated amino acid residues useful in the present invention.

FIG. 3 depicts various deuterated amino acid residues useful in the present invention.

FIG. 4 depicts various deuterated amino acid residues useful in the present invention.

FIG. 5 depicts various deuterated amino acid residues useful in the present invention.

FIG. 6 depicts various deuterated amino acid residues useful in the present invention.

FIG. 7 depicts various deuterated amino acid residues useful in the present invention.

FIG. 8 depicts various deuterated amino acid residues useful in the present invention.

FIG. 9 depicts various deuterated amino acid residues useful in the present invention.

FIG. 10 depicts various deuterated amino acid residues useful in the present invention.

FIG. 11 depicts various deuterated amino acid residues useful in the present invention.

FIG. 12 depicts various deuterated amino acid residues useful in the present invention.

FIGS. 13A-13C depict raw ITC data for Example 8 non-deuterated. FIG. 13A replicates Table 3, entry 1. FIG. 13B replicates Table 3, entry 2. FIG. 13C replicates Table 3, entry 3.

FIGS. 14A-14C depict raw ITC data for Example 1 non-deuterated. FIG. 14A replicates Table 3, entry 6. FIG. 14B replicates Table 3, entry 7. FIG. 14C replicates Table 3, entry 8.

FIGS. 15A-15D depict raw ITC data for Example 1. FIG. 15A replicates Table 3, entry 11. FIG. 15B replicates Table 3, entry 12. FIG. 15C replicates Table 13, entry 3. FIG. 15D replicates Table 3, entry 14.

FIG. 16A depicts the binding mode of type A Example 8 non-deuterated. FIG. 16B depicts type B Example 1 non-deuterated.

FIG. 17 depicts that, in Example 1, deuterated and non-deuterated compounds have different binding modes.

FIG. 18 shows a bar graph depicting infarct size reduction (%) compared to non-deuterated control.

FIG. 19A is a bar graph showing NGAL-1 reduction compared to non-deuterated control.

FIG. 19B is a bar graph showing KIM-1 reduction compared to non-deuterated control.

FIG. 19C is a bar graph showing urea reduction compared to non-deuterated control.

FIG. 19D is a bar graph showing creatinine reduction compared to non-deuterated control.

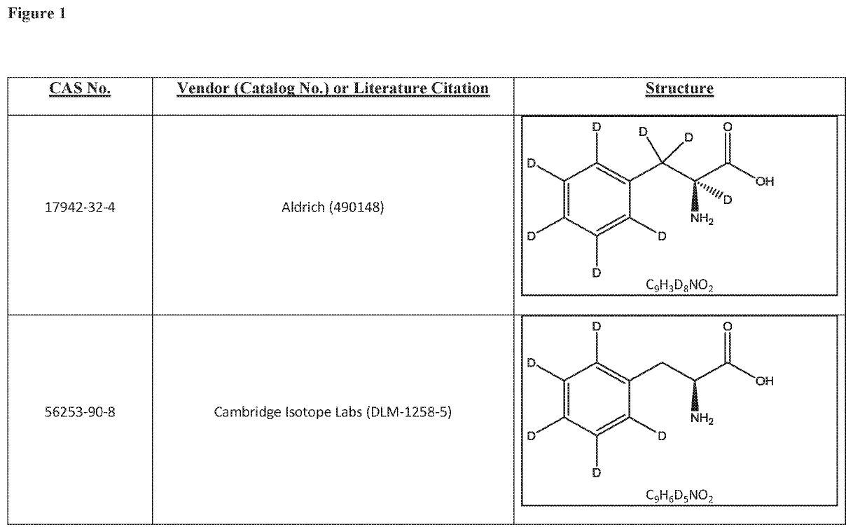

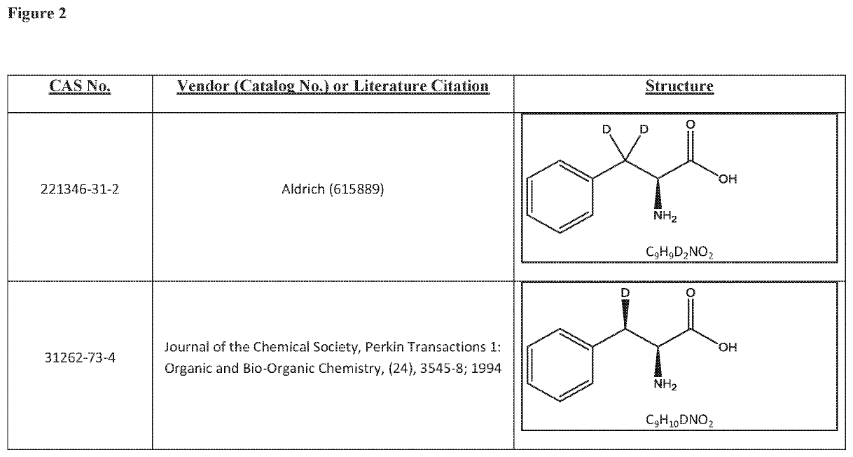

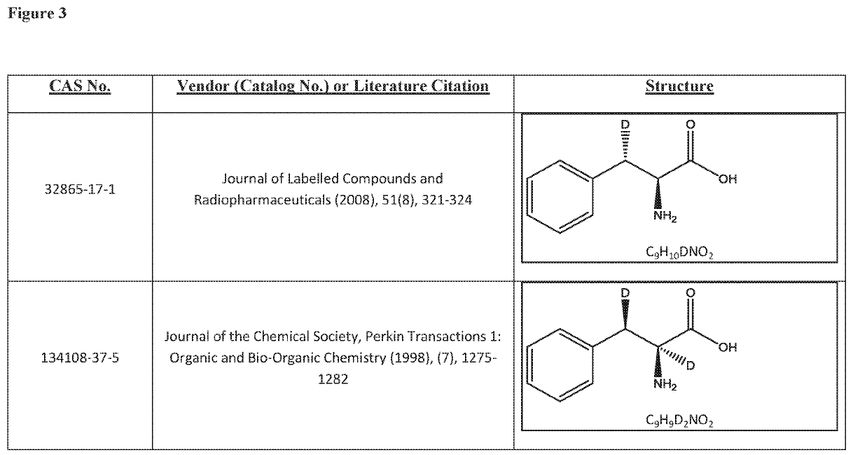

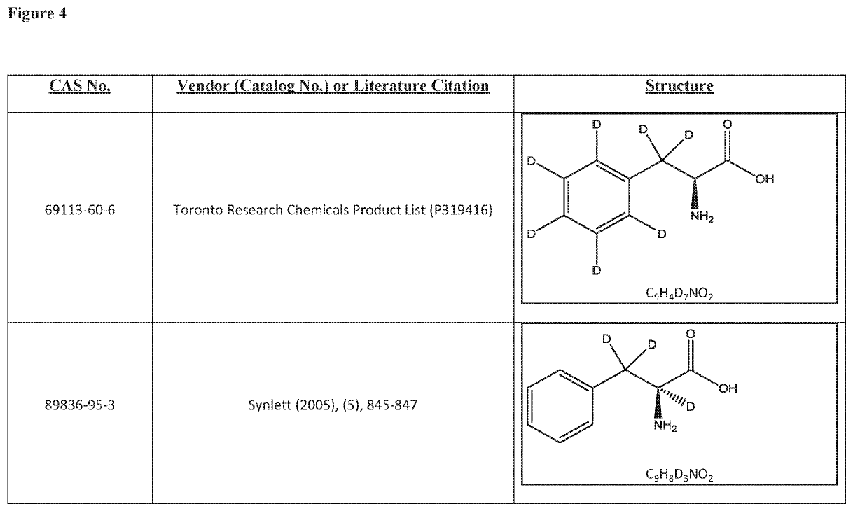

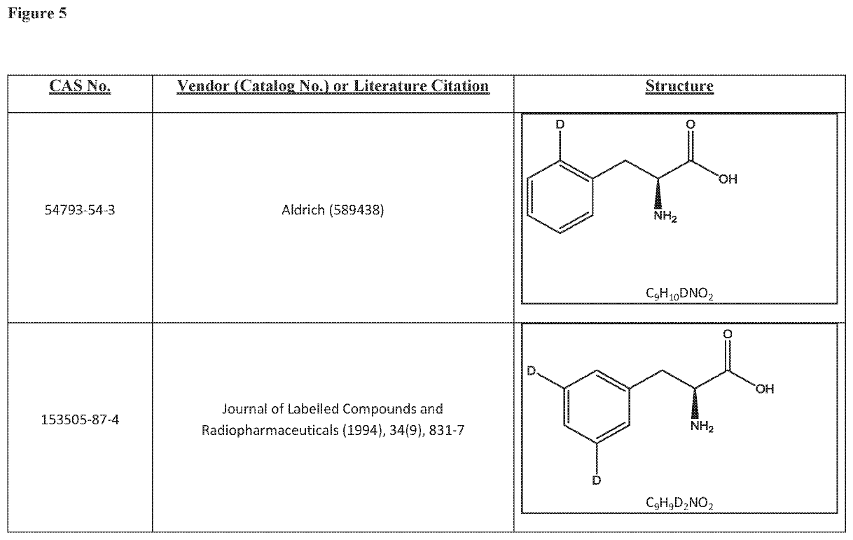

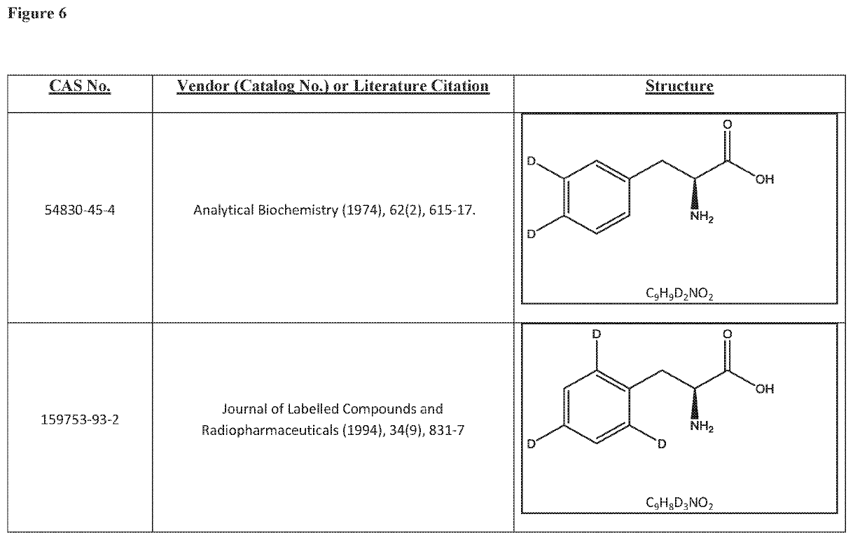

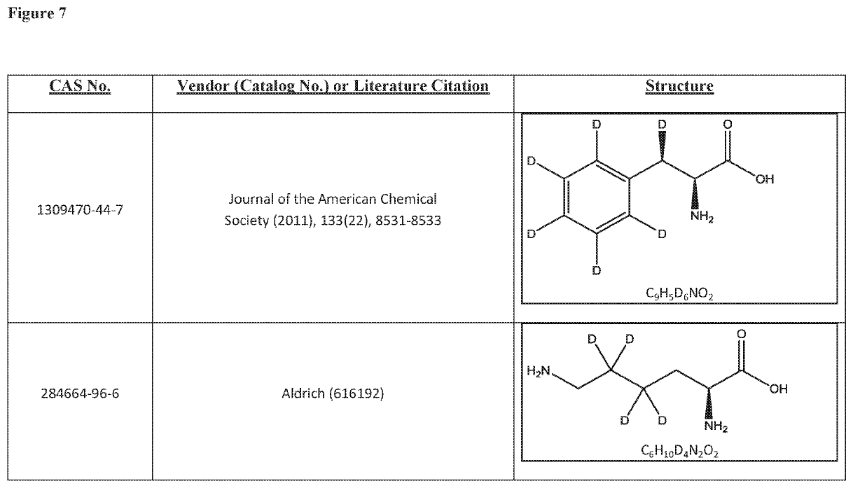

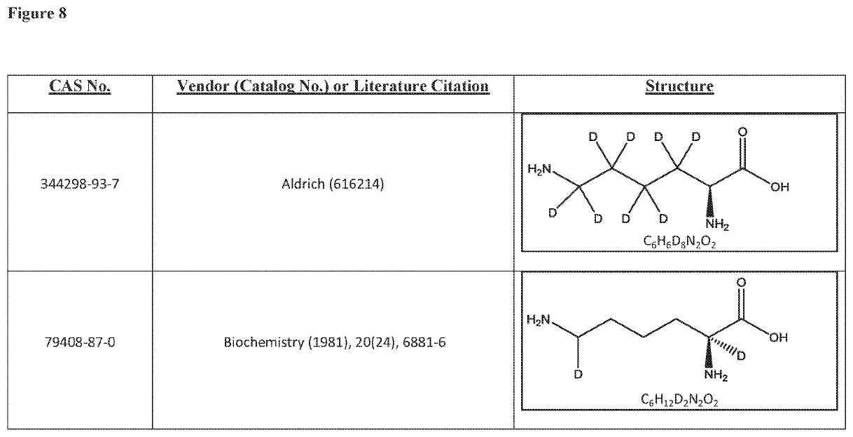

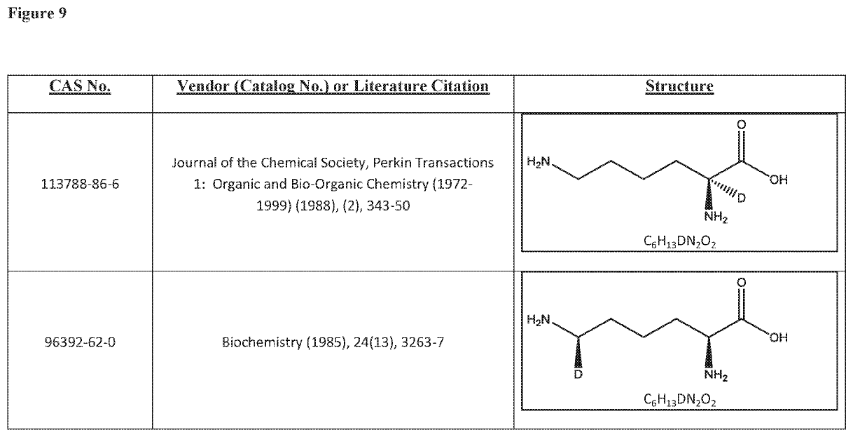

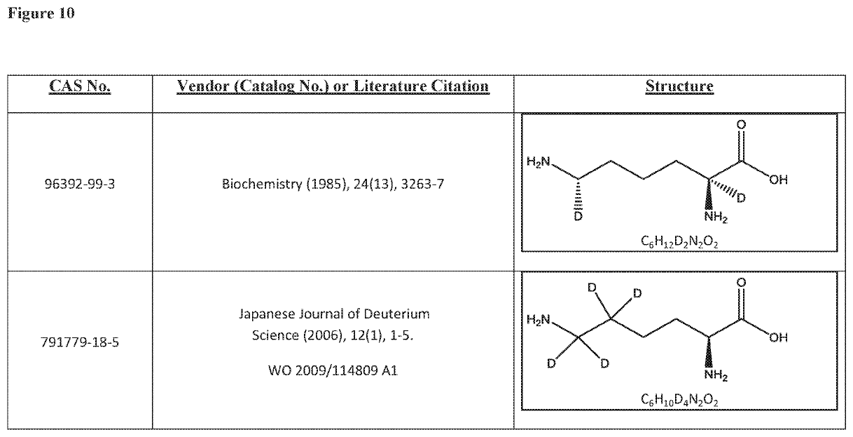

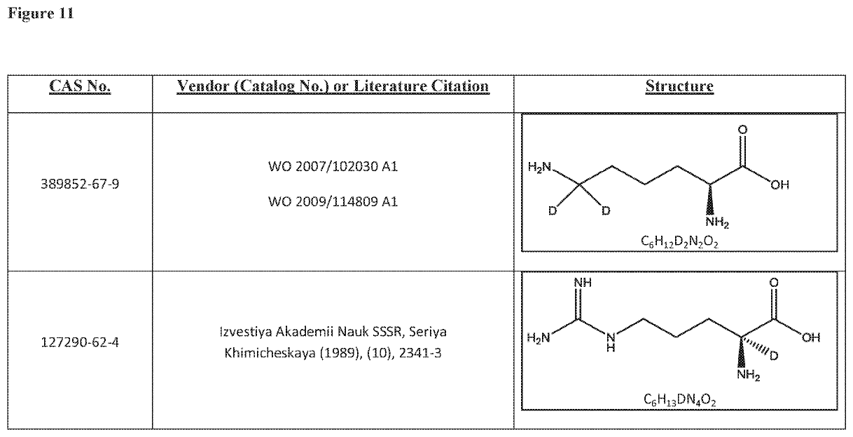

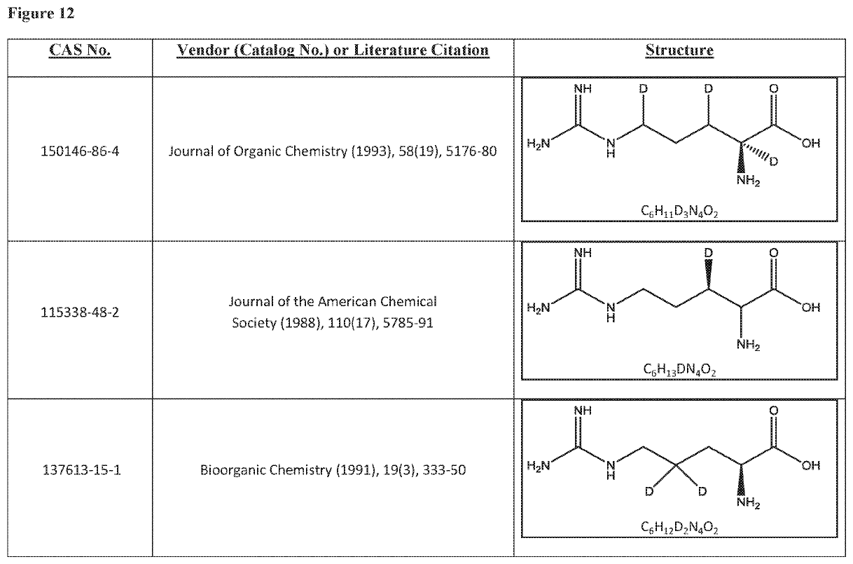

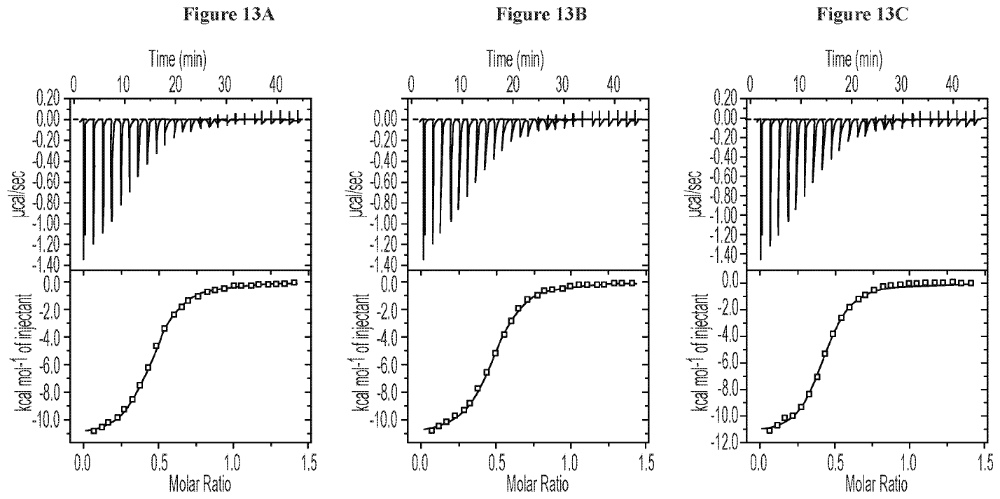

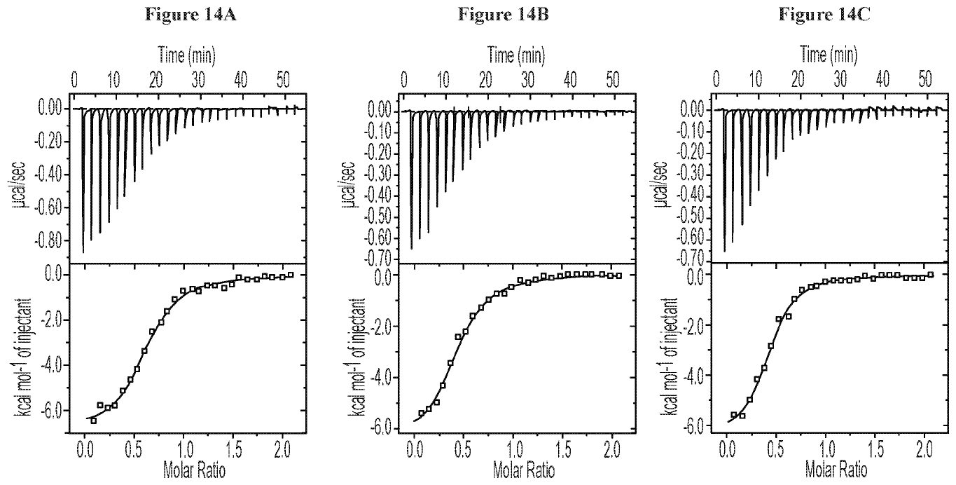

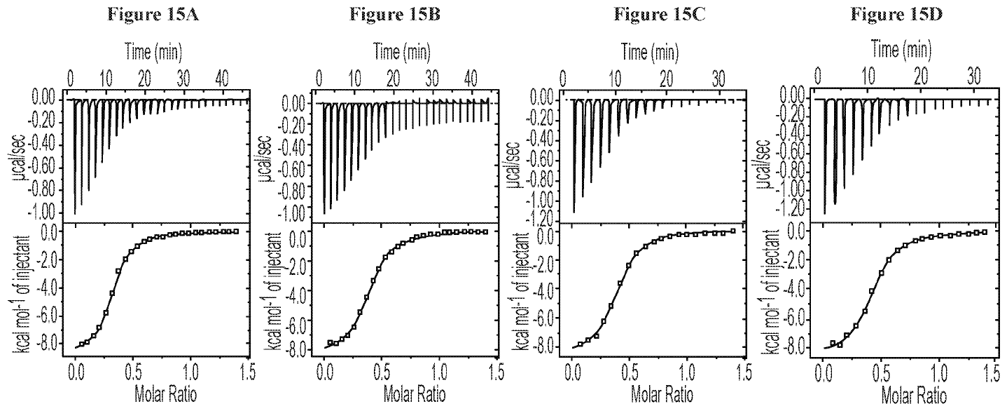

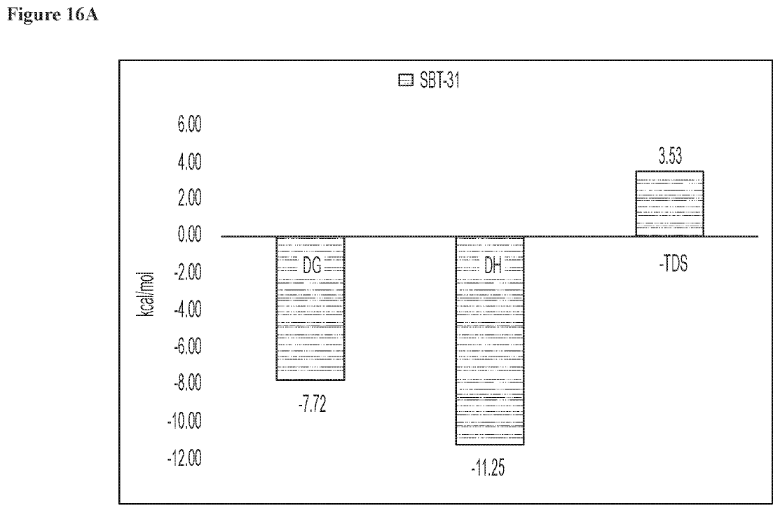

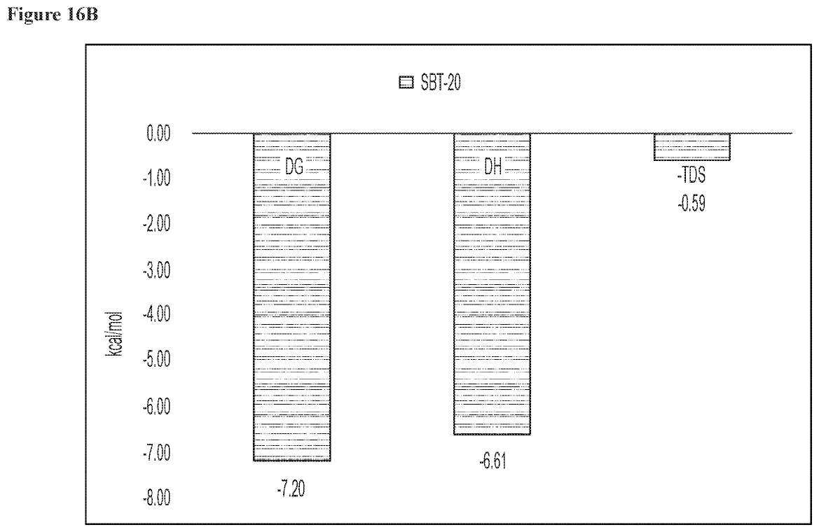

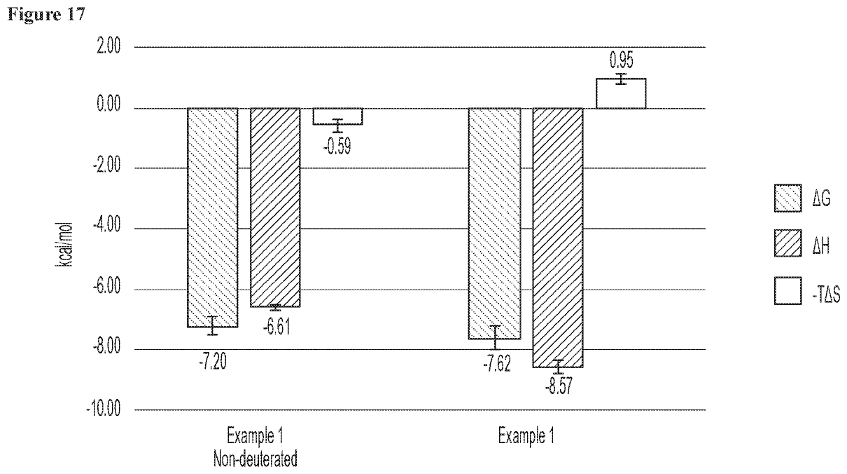

### DETAILED DESCRIPTION OF THE INVENTION

Elamipretide (MTP-131) and SBT-20 are mitochondria-targeting compounds with therapeutic potential for treating ischemia-reperfusion injury (e.g., cardiac ischemia-reperfusion injury), and myocardial infarction. Slowing the metabolism of these compounds may prove beneficial from a therapeutic standpoint.

Accordingly, in certain embodiments, the invention provides a compound of formula (I), or a pharmaceutically acceptable salt thereof:

Aaa¹-Aaa²-Aaa³-Aaa⁴-NH₂ (I).

wherein:
Aaa¹ is an L-phenylalanine residue;
Aaa² is a D-arginine residue;
Aaa³ is an L-phenylalanine residue;
Aaa⁴ is an L-lysine residue; and
at least one hydrogen atom is replaced by a deuterium atom.

In further embodiments, the invention provides a compound of formula (I) in which:

Aaa¹ is an L-phenylalanine residue or an amino acid residue selected from the group consisting of:

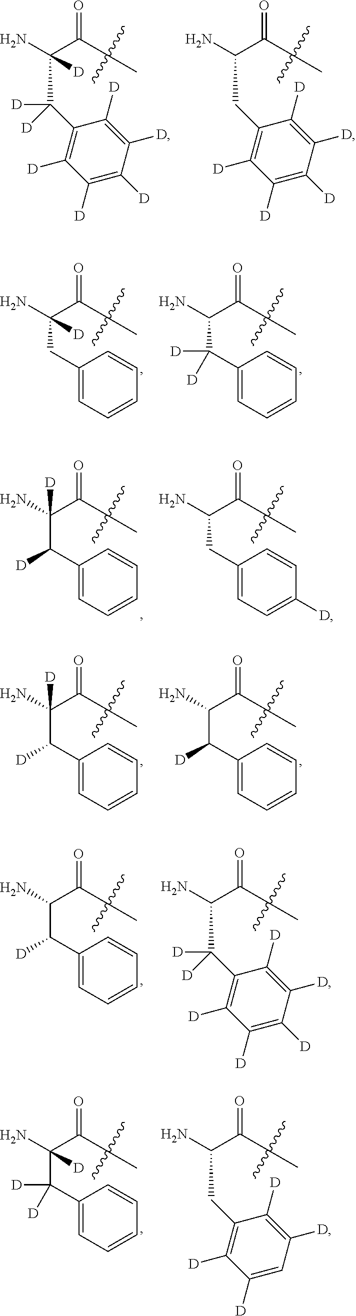

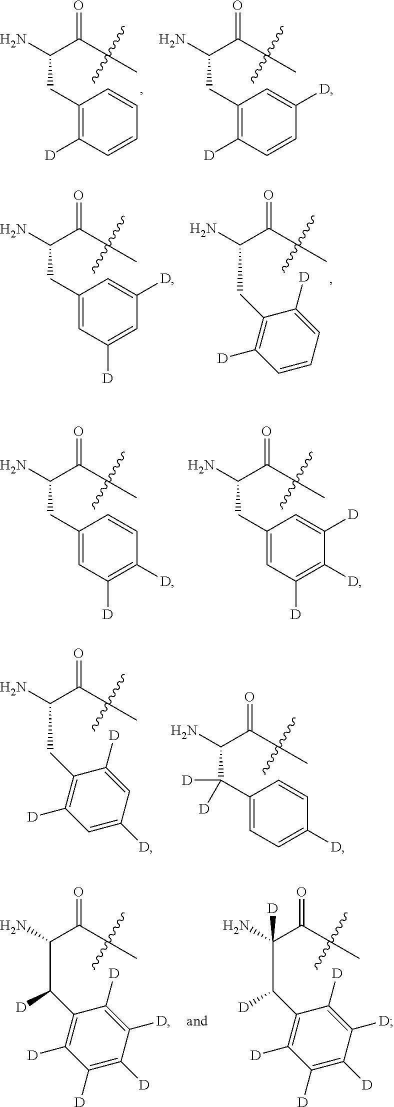

Aaa² is a D-arginine residue or an amino acid residue selected from the group consisting of:

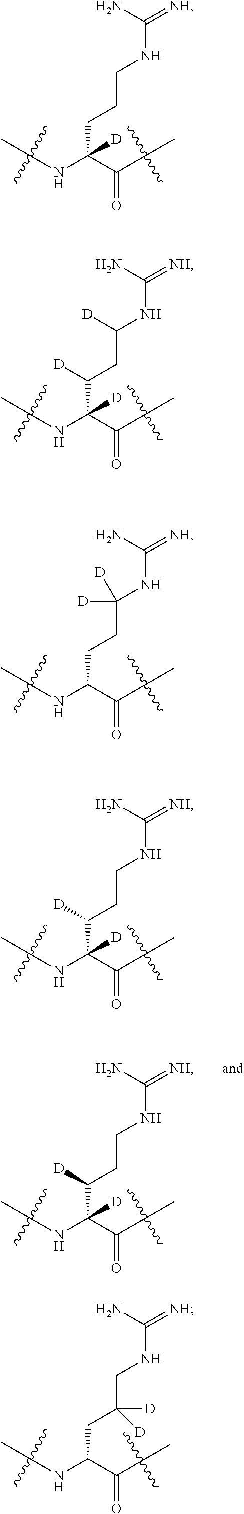

Aaa³ is an L-phenylalanine residue or an amino acid residue selected from the group consisting of:

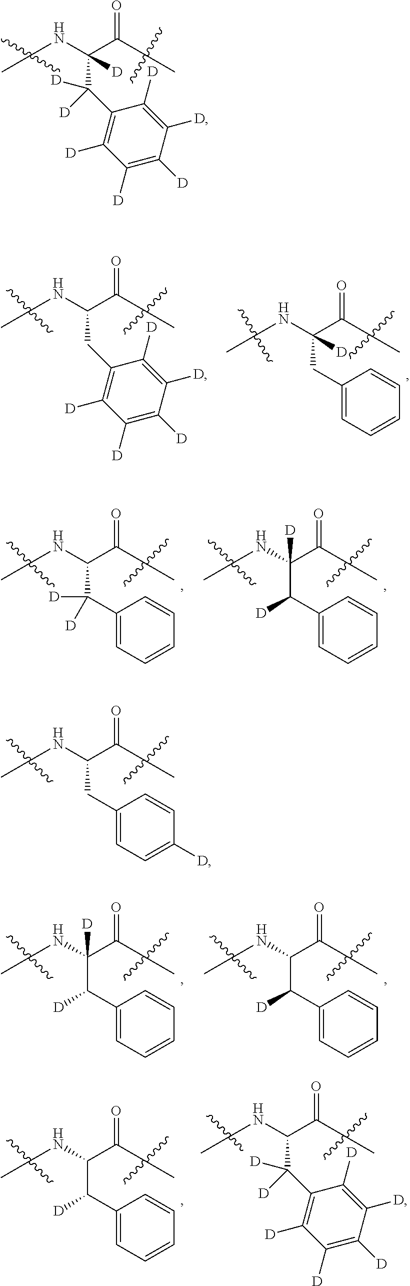

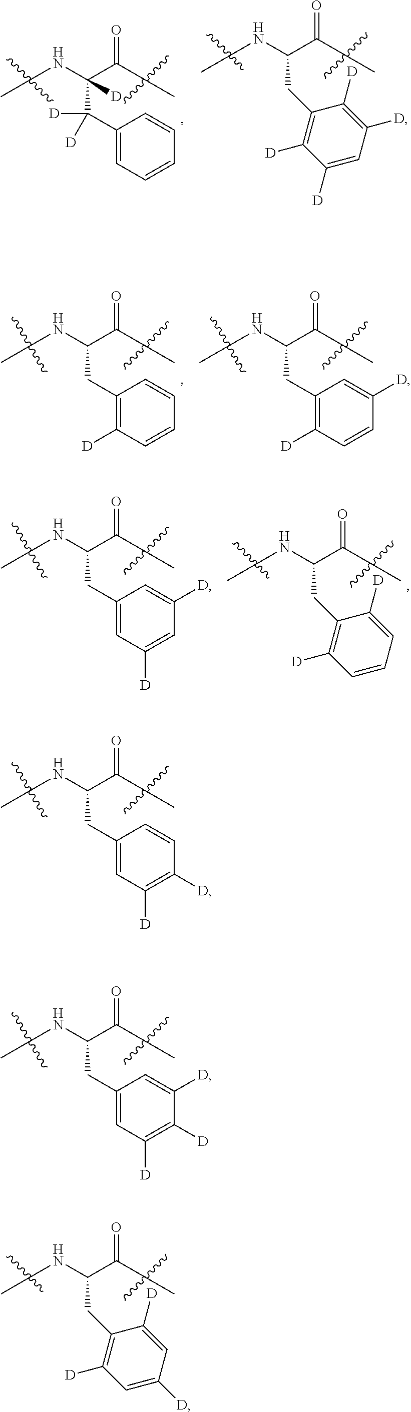

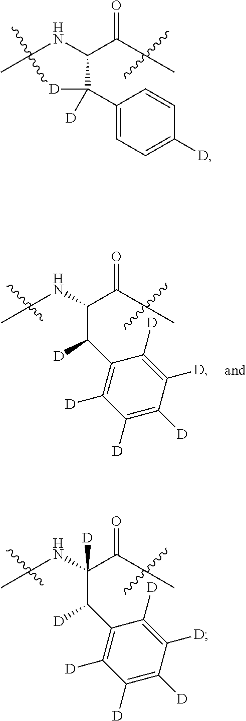

and

Aaa⁴ is an L-lysine residue or an amino acid residue selected from the group consisting of:

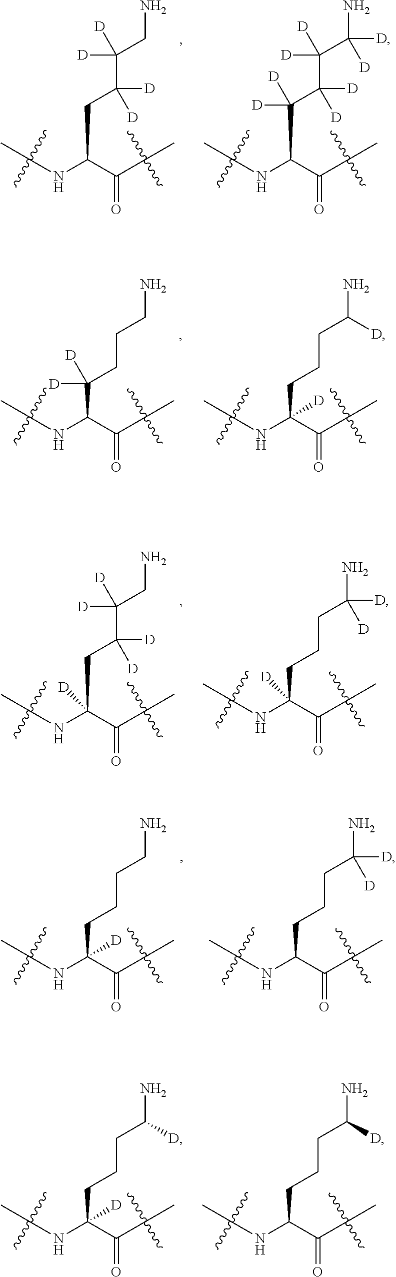

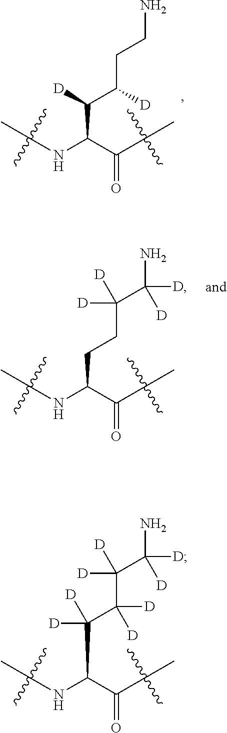

provided that the compound of formula (I) is not Phe-D-Arg-Phe-Lys-NH₂.

In certain embodiments, Aaa¹ is selected from the group consisting of:

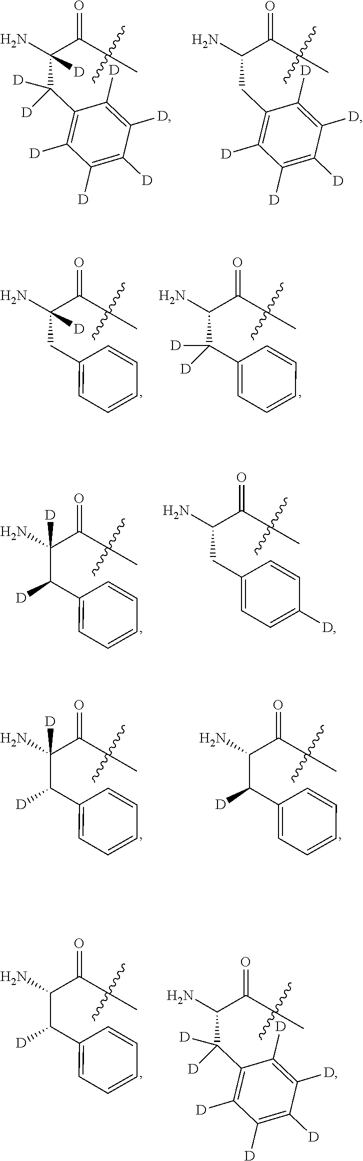

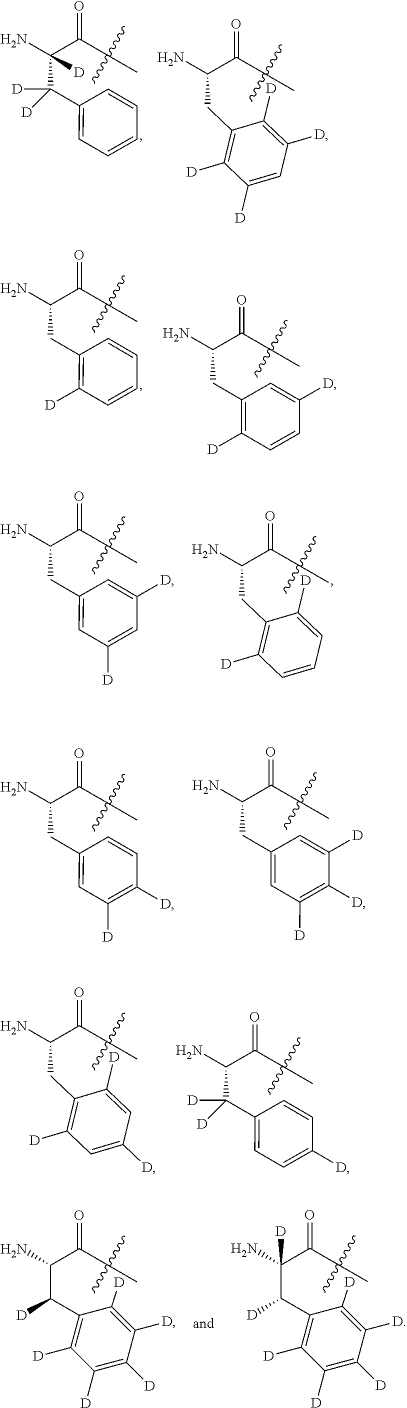

In certain embodiments, Aaa² is selected from the group consisting of:

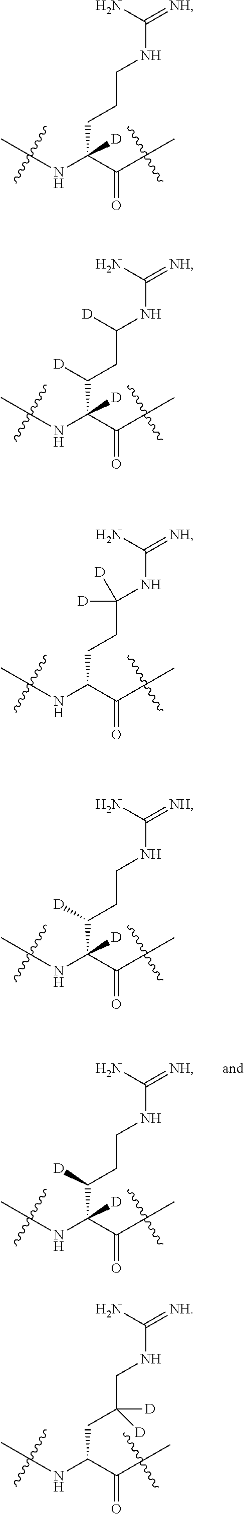

In certain embodiments, Aaa³ is selected from the group consisting of:

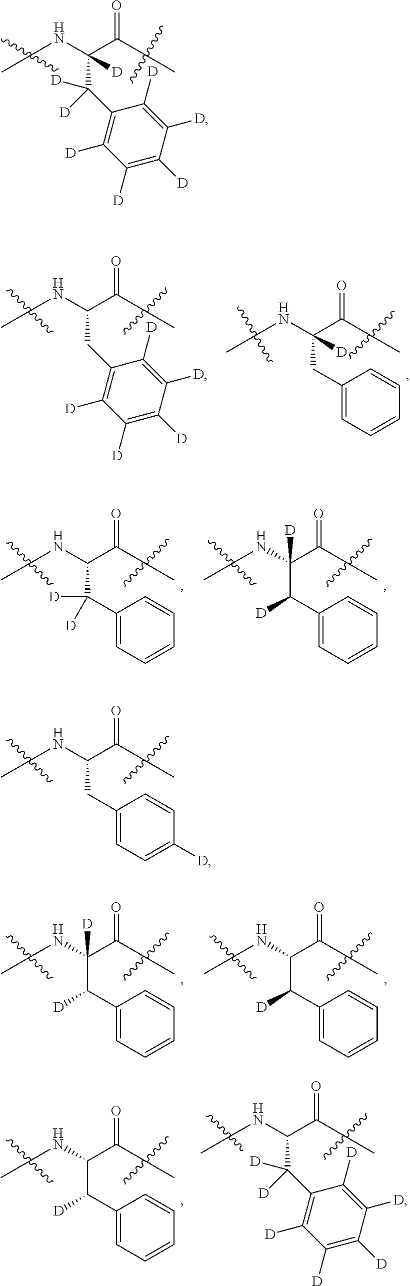

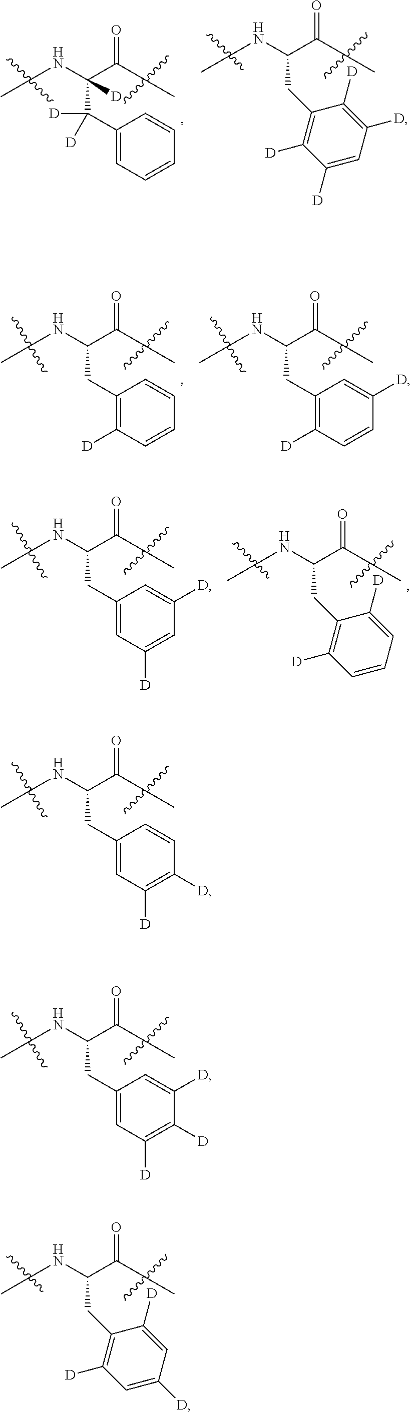

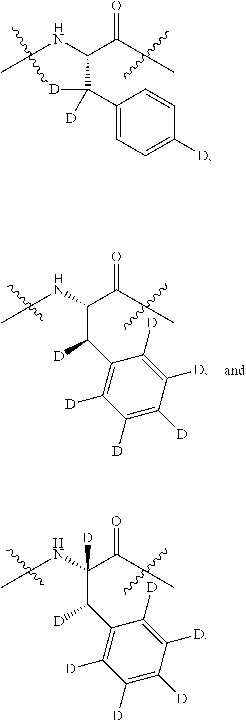

In certain embodiments, Aaa⁴ is selected from the group consisting of:

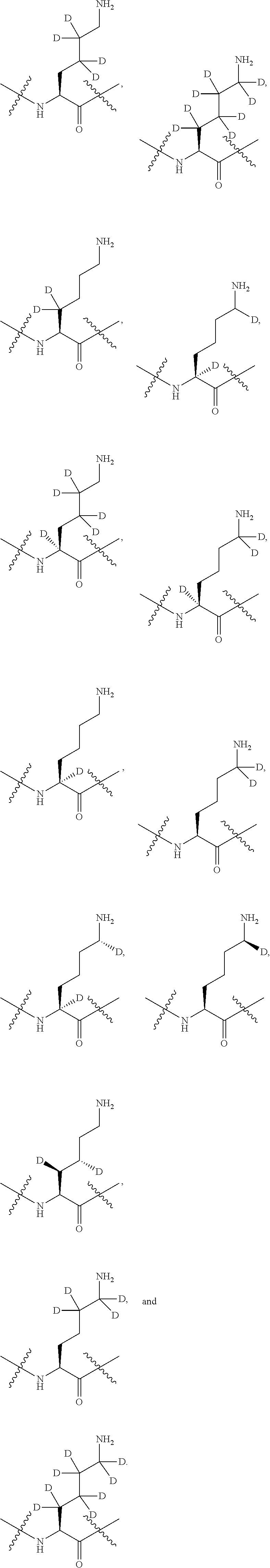

In exemplary embodiments, the compound is selected from the following table:

| | |
|---|---|
| 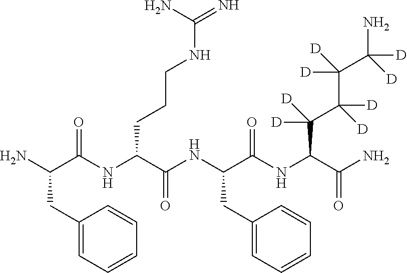 [SMILES: [2H]C([2H])(N)C([2H])([2H])C([2H])([2H])C([2H])([2H])[C@H](NC(=O)[C@H](Cc1ccccc1)NC(=O)[C@@H](CCCNC(=N)N)NC(=O)[C@@H](N)Cc1ccccc1)C(N)=O] | 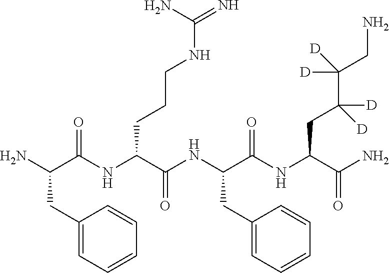 [SMILES: [2H]C([2H])(CN)C([2H])([2H])C[C@H](NC(=O)[C@H](Cc1ccccc1)NC(=O)[C@@H](CCCNC(=N)N)NC(=O)[C@@H](N)Cc1ccccc1)C(N)=O] |
| 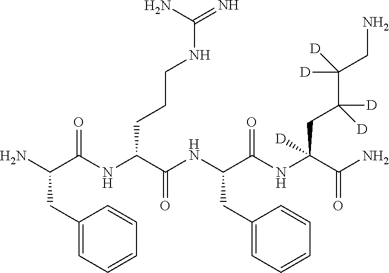 [SMILES: [2H]C([2H])(CN)C([2H])([2H])C[C@]([2H])(NC(=O)[C@H](Cc1ccccc1)NC(=O)[C@@H](CCCNC(=N)N)NC(=O)[C@@H](N)Cc1ccccc1)C(N)=O] | 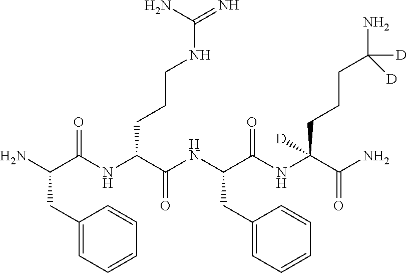 [SMILES: [2H]C([2H])(N)CCC[C@]([2H])(NC(=O)[C@H](Cc1ccccc1)NC(=O)[C@@H](CCCNC(=N)N)NC(=O)[C@@H](N)Cc1ccccc1)C(N)=O] |
| 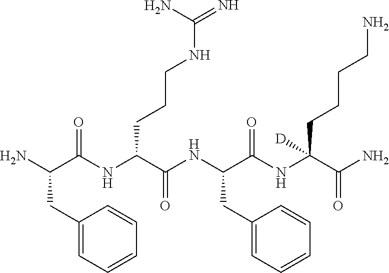 [SMILES: [2H][C@@](CCCCN)(NC(=O)[C@H](Cc1ccccc1)NC(=O)[C@@H](CCCNC(=N)N)NC(=O)[C@@H](N)Cc1ccccc1)C(N)=O] | 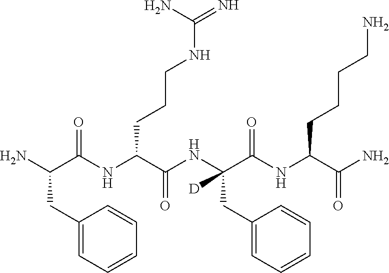 [SMILES: [2H][C@@](Cc1ccccc1)(NC(=O)[C@@H](CCCNC(=N)N)NC(=O)[C@@H](N)Cc1ccccc1)C(=O)N[C@@H](CCCCN)C(N)=O] |
| 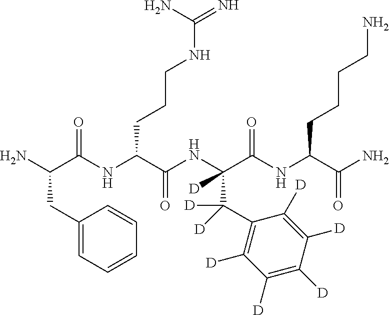 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@H](CCCCN)C(N)=O)c([2H])c1[2H]] | 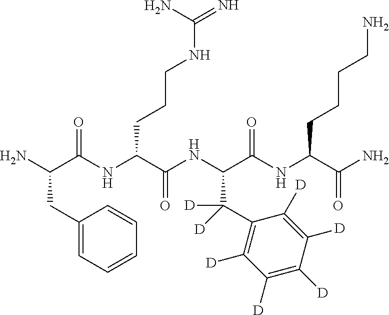 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@@H](CCCCN)C(N)=O)c([2H])c1[2H]] |
| 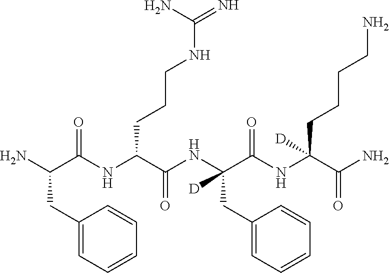 [SMILES: [2H][C@@](CCCCN)(NC(=O)[C@]([2H])(Cc1ccccc1)NC(=O)[C@@H](CCCNC(=N)N)NC(=O)[C@@H](N)Cc1ccccc1)C(N)=O] | 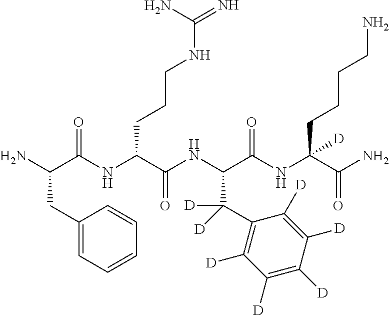 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@@]([2H])(CCCCN)C(N)=O)c([2H])c1[2H]] |
| 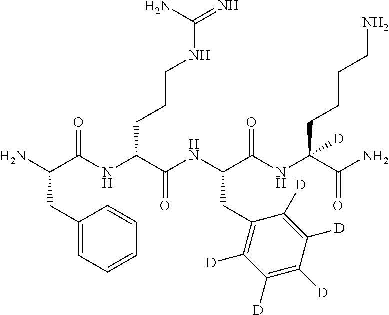 [SMILES: [2H]c1c([2H])c([2H])c(C[C@H](NC(=O)[C@@H](CCCNC(=N)N)NC(=O)[C@@H](N)Cc2ccccc2)C(=O)N[C@@]([2H])(CCCCN)C(N)=O)c([2H])c1[2H]] | 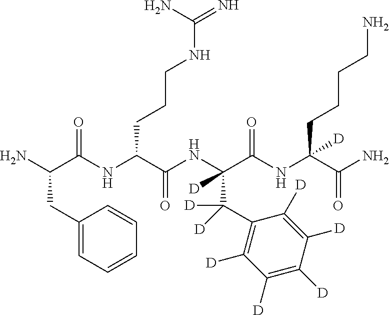 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@]([2H])(CCCCN)C(N)=O)c([2H])c1[2H]] |
| 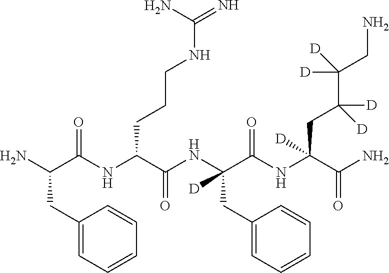 [SMILES: [2H]C([2H])(CN)C([2H])([2H])C[C@]([2H])(NC(=O)[C@]([2H])(Cc1ccccc1)NC(=O)[C@@H](CCCNC(=N)N)NC(=O)[C@@H](N)Cc1ccccc1)C(N)=O] | 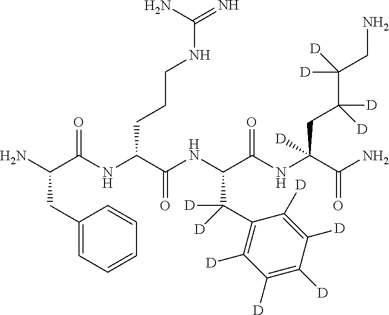 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@@]([2H])(CC([2H])([2H])C([2H])([2H])CN)C(N)=O)c([2H])c1[2H]] |
| 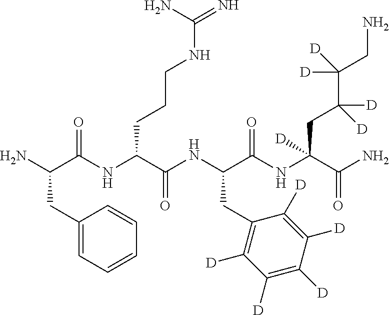 [SMILES: [2H]c1c([2H])c([2H])c(C[C@H](NC(=O)[C@@H](CCCNC(=N)N)NC(=O)[C@@H](N)Cc2ccccc2)C(=O)N[C@@]([2H])(CC([2H])([2H])C([2H])([2H])CN)C(N)=O)c([2H])c1[2H]] | 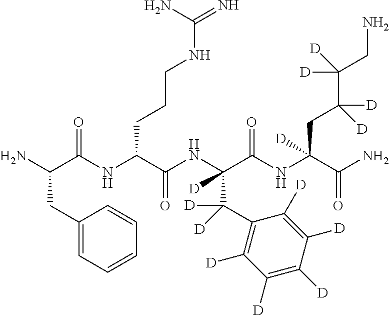 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@]([2H])(CC([2H])([2H])C([2H])([2H])CN)C(N)=O)c([2H])c1[2H]] |
| 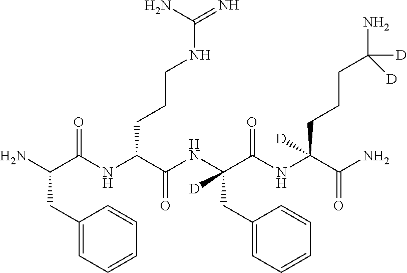 [SMILES: [2H]C([2H])(N)CCC[C@]([2H])(NC(=O)[C@]([2H])(Cc1ccccc1)NC(=O)[C@@H](CCCNC(=N)N)NC(=O)[C@@H](N)Cc1ccccc1)C(N)=O] | 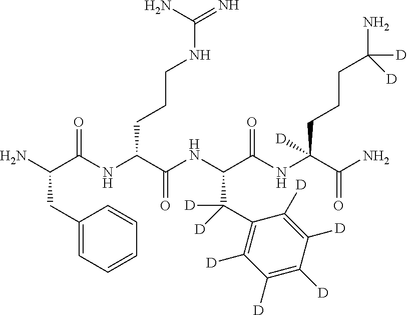 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@@]([2H])(CCCC([2H])([2H])N)C(N)=O)c([2H])c1[2H]] |
| 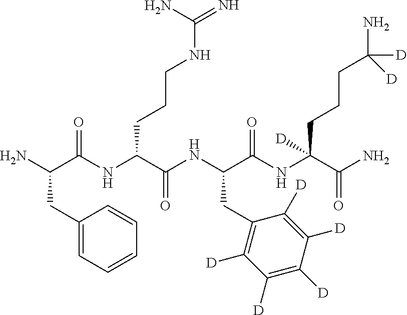 [SMILES: [2H]c1c([2H])c([2H])c(C[C@H](NC(=O)[C@@H](CCCNC(=N)N)NC(=O)[C@@H](N)Cc2ccccc2)C(=O)N[C@@]([2H])(CCCC([2H])([2H])N)C(N)=O)c([2H])c1[2H]] | 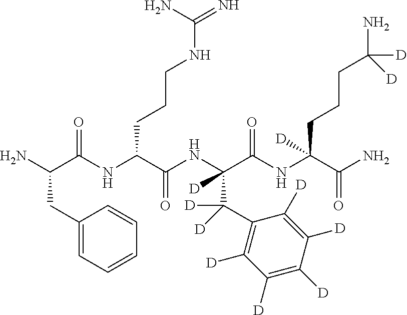 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@]([2H])(CCCC([2H])([2H])N)C(N)=O)c([2H])c1[2H]] |
| 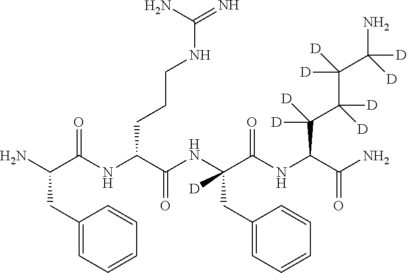 [SMILES: [2H]C([2H])(N)C([2H])([2H])C([2H])([2H])C([2H])([2H])[C@H](NC(=O)[C@]([2H])(Cc1ccccc1)NC(=O)[C@@H](CCCNC(=N)N)NC(=O)[C@@H](N)Cc1ccccc1)C(N)=O] | 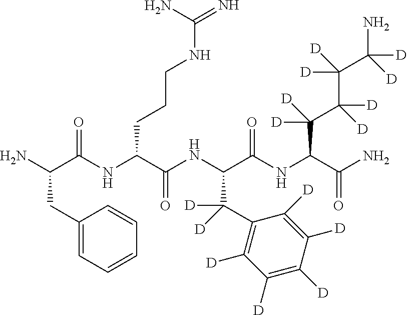 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@H](C(N)=O)C([2H])([2H])C([2H])([2H])C([2H])([2H])C([2H])([2H])N)c([2H])c1[2H]] |
| 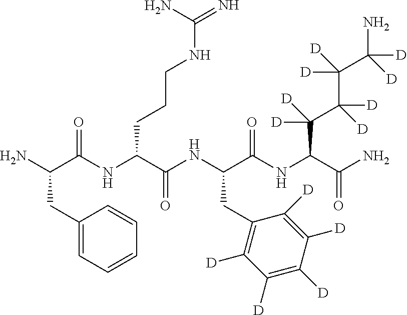 [SMILES: [2H]c1c([2H])c([2H])c(C[C@H](NC(=O)[C@@H](CCCNC(=N)N)NC(=O)[C@@H](N)Cc2ccccc2)C(=O)N[C@H](C(N)=O)C([2H])([2H])C([2H])([2H])C([2H])([2H])C([2H])([2H])N)c([2H])c1[2H]] | 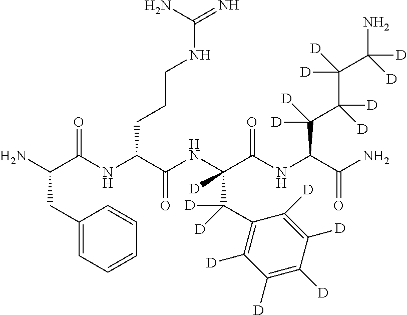 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@H](C(N)=O)C([2H])([2H])C([2H])([2H])C([2H])([2H])C([2H])([2H])N)c([2H])c1[2H]] |

In yet further exemplary embodiments, the compound is selected from the following table:

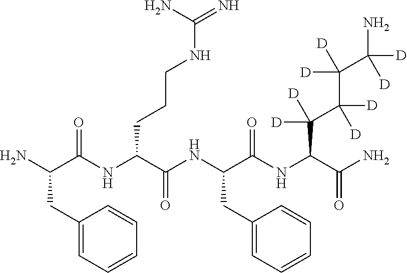 [SMILES: [2H]C([2H])(N)C([2H])([2H])C([2H])([2H])C([2H])([2H])[C@H](NC(=O)[C@H](Cc1ccccc1)NC(=O)[C@@H](CCCNC(=N)N)NC(=O)[C@@H](N)Cc1ccccc1)C(N)=O]

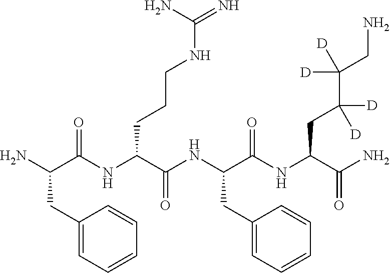 [SMILES: [2H]C([2H])(CN)C([2H])([2H])C[C@H](NC(=O)[C@H](Cc1ccccc1)NC(=O)[C@@H](CCCNC(=N)N)NC(=O)[C@@H](N)Cc1ccccc1)C(N)=O]

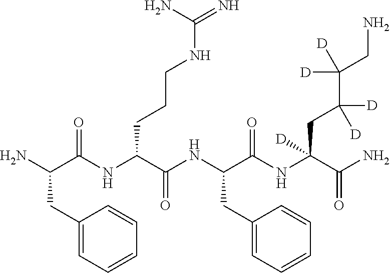 [SMILES: [2H]C([2H])(CN)C([2H])([2H])C[C@]([2H])(NC(=O)[C@H](Cc1ccccc1)NC(=O)[C@@H](CCCNC(=N)N)NC(=O)[C@@H](N)Cc1ccccc1)C(N)=O]

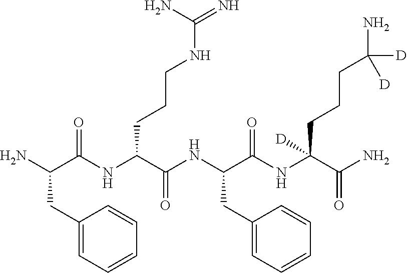 [SMILES: [2H]C([2H])(N)CCC[C@]([2H])(NC(=O)[C@H](Cc1ccccc1)NC(=O)[C@@H](CCCNC(=N)N)NC(=O)[C@@H](N)Cc1ccccc1)C(N)=O]

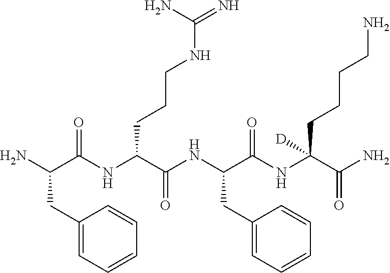 [SMILES: [2H][C@@](CCCCN)(NC(=O)[C@H](Cc1ccccc1)NC(=O)[C@@H](CCCNC(=N)N)NC(=O)[C@@H](N)Cc1ccccc1)C(N)=O]

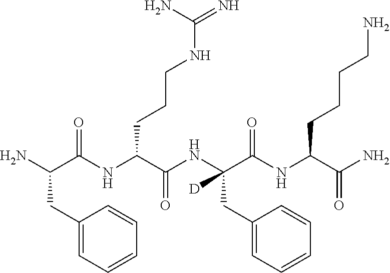 [SMILES: [2H][C@@](Cc1ccccc1)(NC(=O)[C@@H](CCCNC(=N)N)NC(=O)[C@@H](N)Cc1ccccc1)C(=O)N[C@@H](CCCCN)C(N)=O]

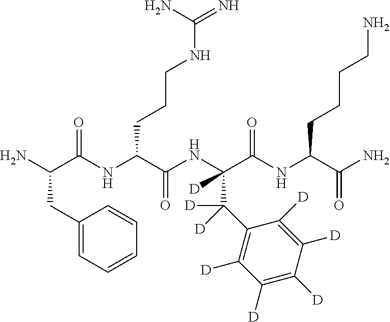 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@H](CCCCN)C(N)=O)c([2H])c1[2H]]

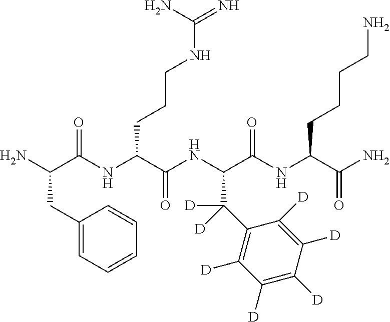 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@@H](CCCCN)C(N)=O)c([2H])c1[2H]]

In other aspects, the invention provides a compound of formula (II), or a pharmaceutically acceptable salt thereof:

Aaa⁵-Aaa⁶-Aaa⁷-Aaa⁸-NH₂ (II);

wherein:
Aaa⁵ is a D-arginine residue;

### Aaa⁶ is:

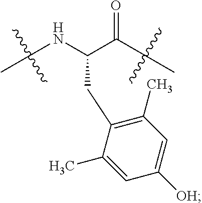 [SMILES: *N[C@@H](Cc1c(C)cc(O)cc1C)C(*)=O]

Aaa⁷ is an L-lysine residue;
Aaa⁸ is an L-phenylalanine residue; and
at least one hydrogen atom is replaced by a deuterium atom.

In further embodiments, the invention provides a compound of formula (II) in which:
Aaa⁵ is a D-arginine residue or an amino acid residue selected from the group consisting of:

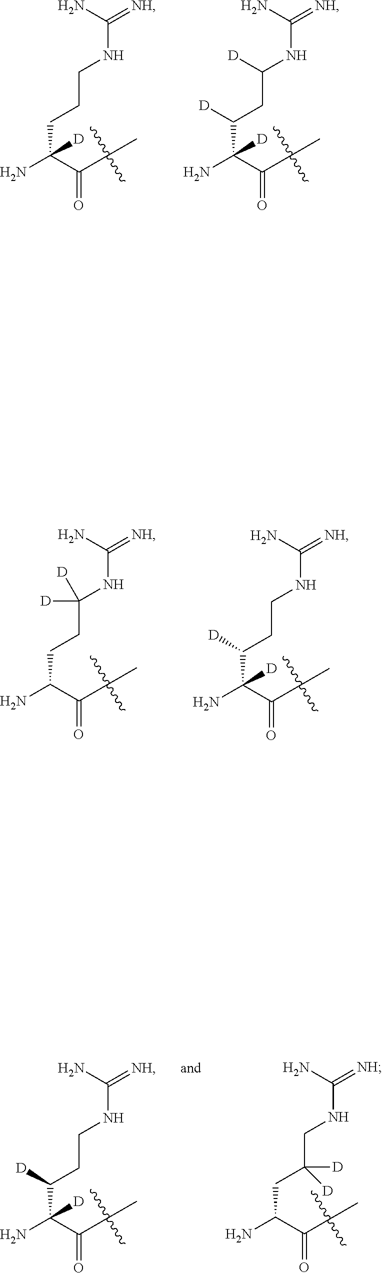

Aaa⁶ is:

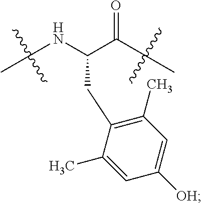 [SMILES: *N[C@@H](Cc1c(C)cc(O)cc1C)C(*)=O]

Aaa⁷ is an L-lysine residue or an amino acid residue selected from the group consisting of:

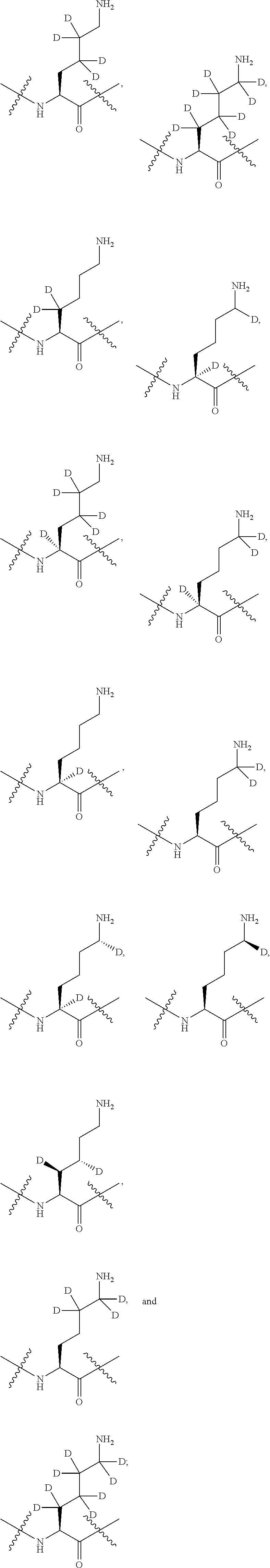

and
Aaa⁸ is an L-phenylalanine residue or an amino acid residue selected from the group consisting of:

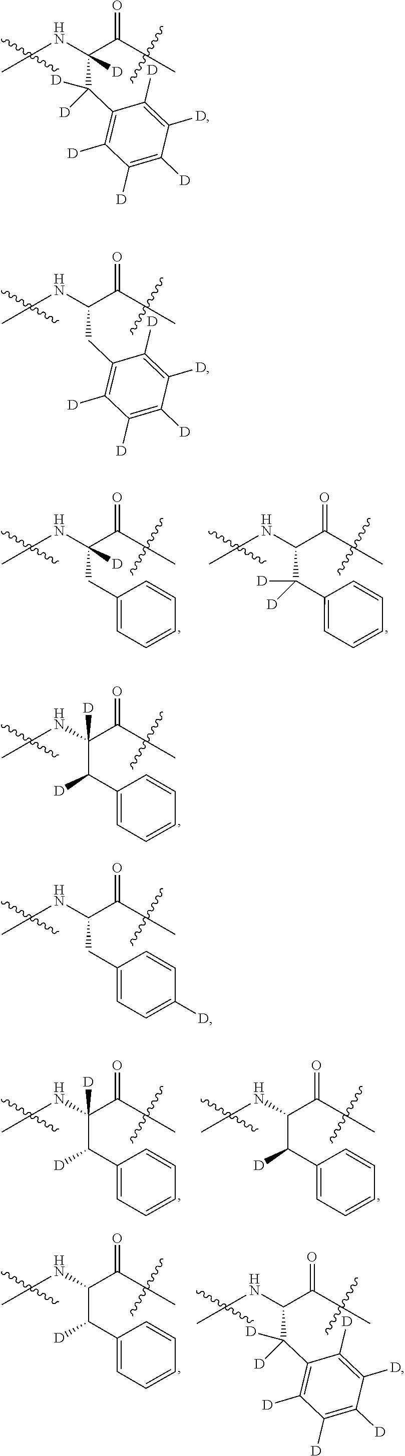

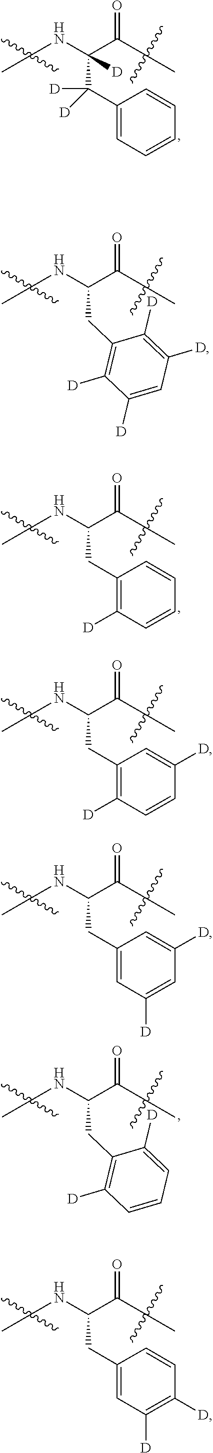

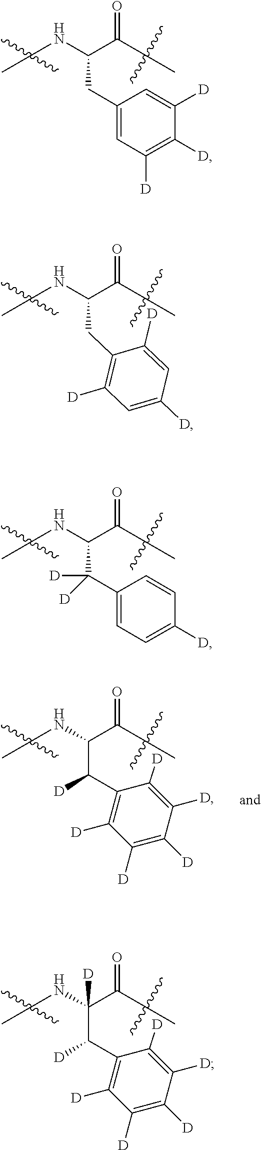

provided that the compound of formula (II) is not D-Arg-DMT-Lys-Phe-NH₂; wherein DMT represents

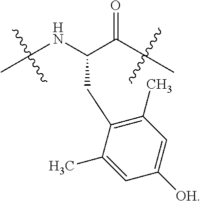 [SMILES: *N[C@@H](Cc1c(C)cc(O)cc1C)C(*)=O]

In certain embodiments, Aaa⁵ is an amino acid residue selected from the group consisting of:

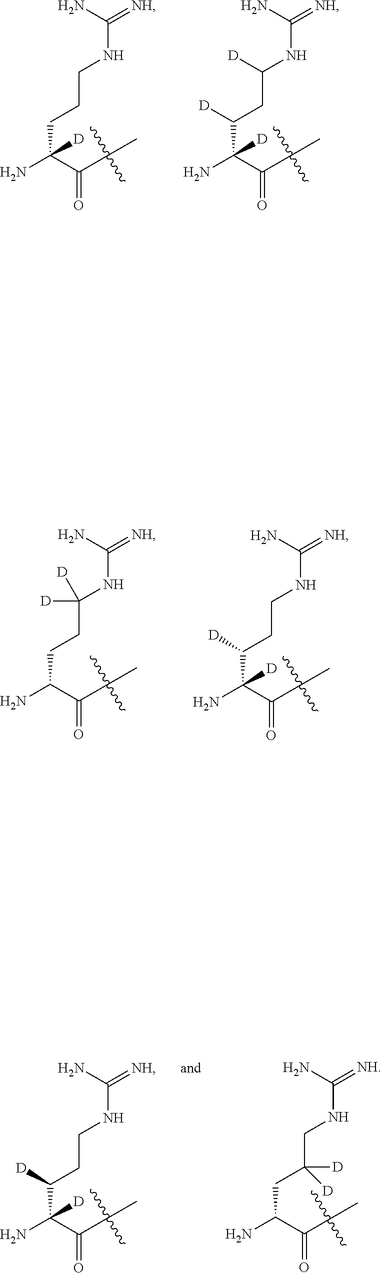

In certain embodiments, Aaa⁷ is an amino acid residue selected from the group consisting of:

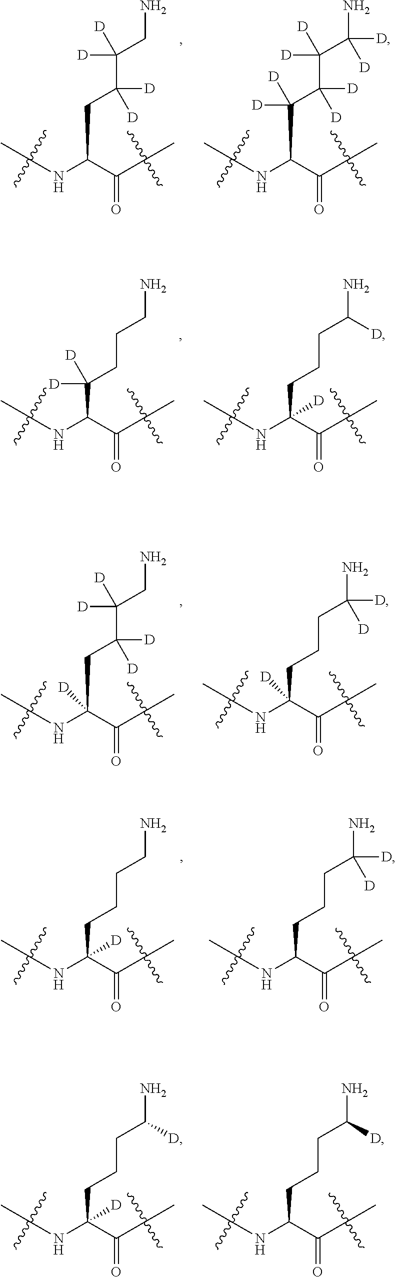

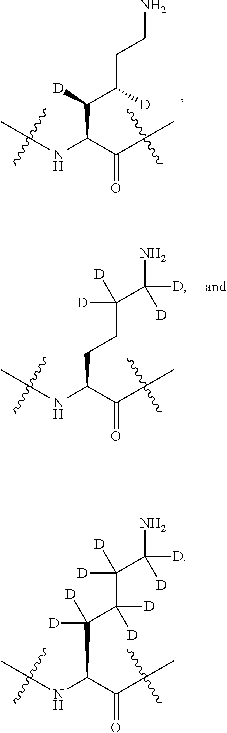

In certain embodiments, Aaa⁸ is an amino acid residue selected from the group consisting of:

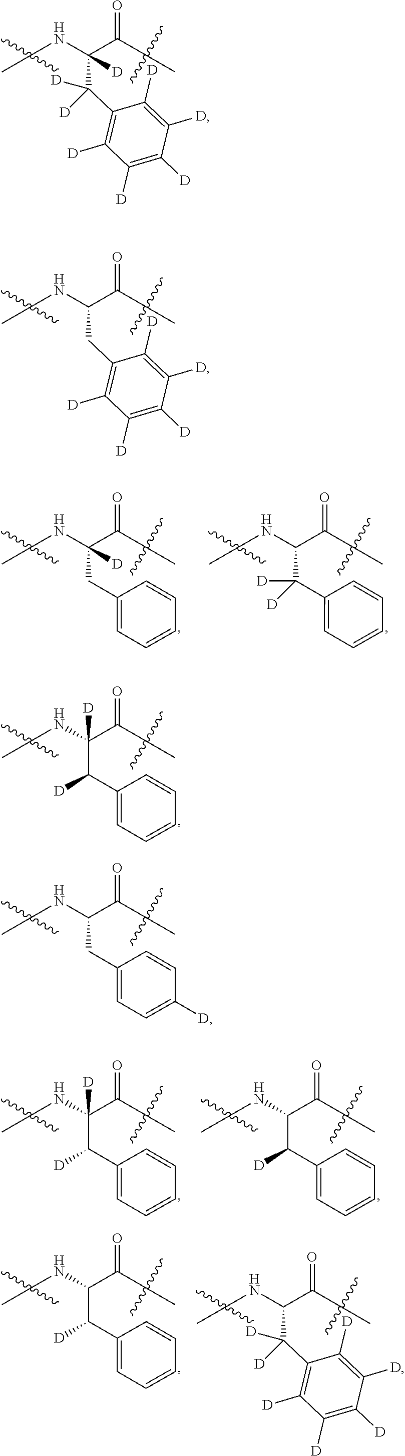

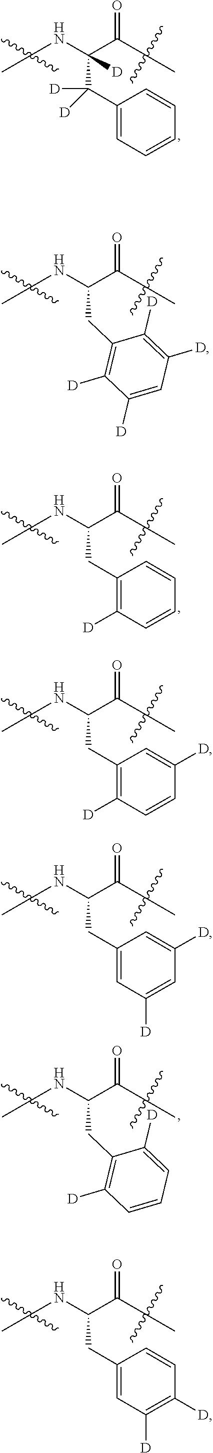

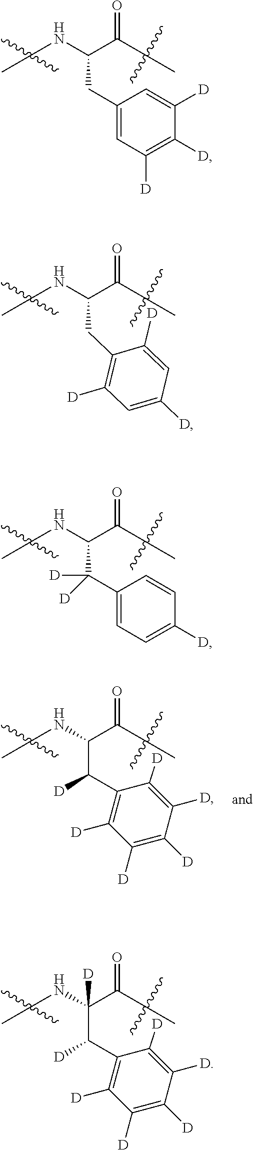

In certain exemplary embodiments, the compound is selected from the following table:

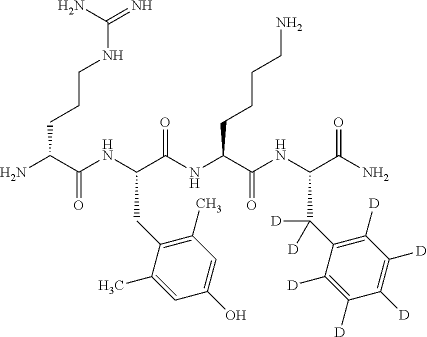 [SMILES: [2H]c1c([2H])c([2H])c(C([2H])([2H])[C@H](NC(=O)[C@H](CCCCN)NC(=O)[C@H](Cc2c(C)cc(O)cc2C)NC(=O)[C@H](N)CCCNC(=N)N)C(N)=O)c([2H])c1[2H]]

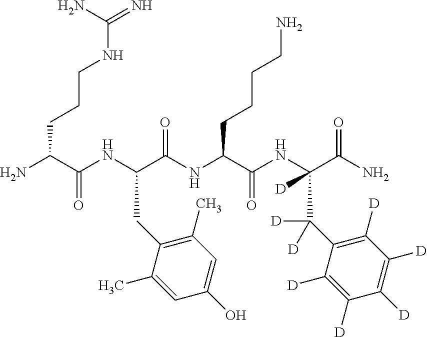 [SMILES: [2H]c1c([2H])c([2H])c(C([2H])([2H])[C@]([2H])(NC(=O)[C@H](CCCCN)NC(=O)[C@H](Cc2c(C)cc(O)cc2C)NC(=O)[C@H](N)CCCNC(=N)N)C(N)=O)c([2H])c1[2H]]

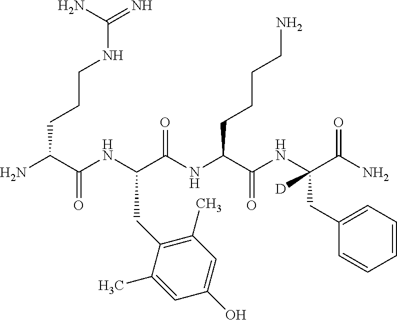 [SMILES: [2H][C@@](Cc1ccccc1)(NC(=O)[C@H](CCCCN)NC(=O)[C@H](Cc1c(C)cc(O)cc1C)NC(=O)[C@H](N)CCCNC(=N)N)C(N)=O]

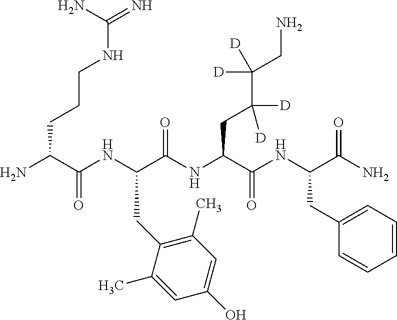 [SMILES: [2H]C([2H])(CN)C([2H])([2H])C[C@H](NC(=O)[C@H](Cc1c(C)cc(O)cc1C)NC(=O)[C@H](N)CCCNC(=N)N)C(=O)N[C@@H](Cc1ccccc1)C(N)=O]

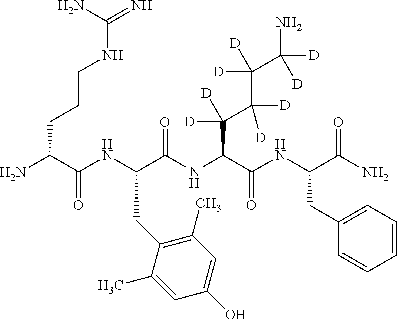 [SMILES: [2H]C([2H])(N)C([2H])([2H])C([2H])([2H])C([2H])([2H])[C@H](NC(=O)[C@H](Cc1c(C)cc(O)cc1C)NC(=O)[C@H](N)CCCNC(=N)N)C(=O)N[C@@H](Cc1ccccc1)C(N)=O]

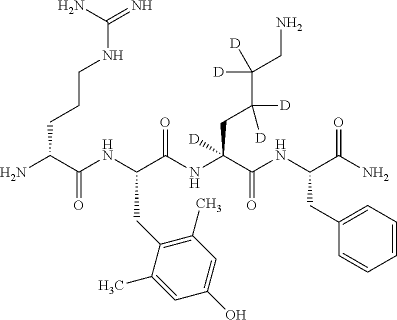 [SMILES: [2H]C([2H])(CN)C([2H])([2H])C[C@]([2H])(NC(=O)[C@H](Cc1c(C)cc(O)cc1C)NC(=O)[C@H](N)CCCNC(=N)N)C(=O)N[C@@H](Cc1ccccc1)C(N)=O]

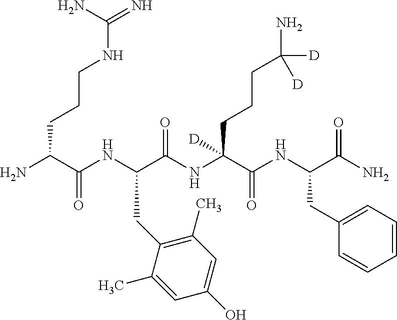 [SMILES: [2H]C([2H])(N)CCC[C@]([2H])(NC(=O)[C@H](Cc1c(C)cc(O)cc1C)NC(=O)[C@H](N)CCCNC(=N)N)C(=O)N[C@@H](Cc1ccccc1)C(N)=O]

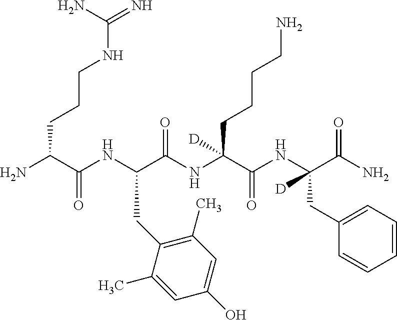 [SMILES: [2H][C@@](Cc1ccccc1)(NC(=O)[C@]([2H])(CCCCN)NC(=O)[C@H](Cc1c(C)cc(O)cc1C)NC(=O)[C@H](N)CCCNC(=N)N)C(N)=O]

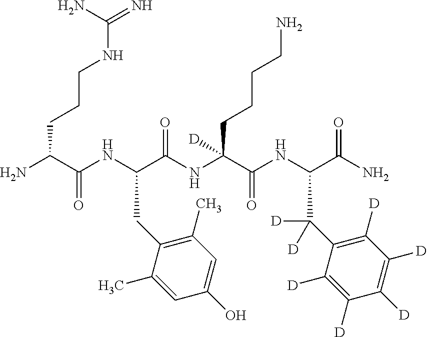 [SMILES: [2H]c1c([2H])c([2H])c(C([2H])([2H])[C@H](NC(=O)[C@]([2H])(CCCCN)NC(=O)[C@H](Cc2c(C)cc(O)cc2C)NC(=O)[C@H](N)CCCNC(=N)N)C(N)=O)c([2H])c1[2H]]

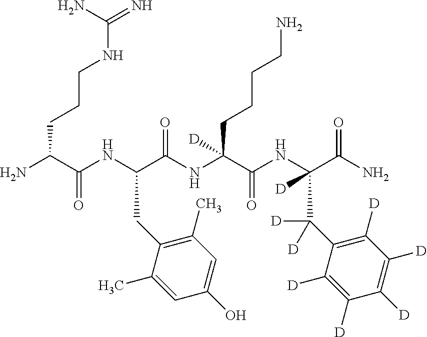 [SMILES: [2H]c1c([2H])c([2H])c(C([2H])([2H])[C@]([2H])(NC(=O)[C@]([2H])(CCCCN)NC(=O)[C@H](Cc2c(C)cc(O)cc2C)NC(=O)[C@H](N)CCCNC(=N)N)C(N)=O)c([2H])c1[2H]]

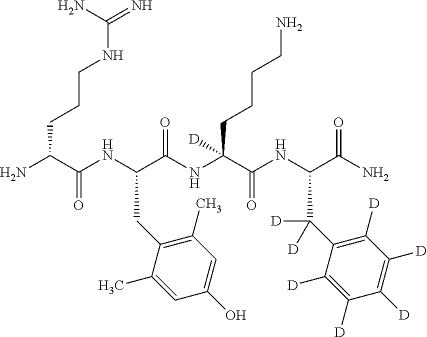 [SMILES: [2H]c1c([2H])c([2H])c(C([2H])([2H])[C@H](NC(=O)[C@]([2H])(CCCCN)NC(=O)[C@H](Cc2c(C)cc(O)cc2C)NC(=O)[C@H](N)CCCNC(=N)N)C(N)=O)c([2H])c1[2H]]

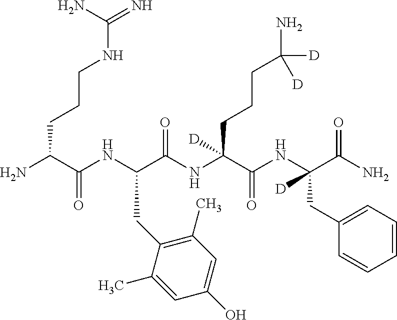 [SMILES: [2H]C([2H])(N)CCC[C@]([2H])(NC(=O)[C@H](Cc1c(C)cc(O)cc1C)NC(=O)[C@H](N)CCCNC(=N)N)C(=O)N[C@@]([2H])(Cc1ccccc1)C(N)=O]

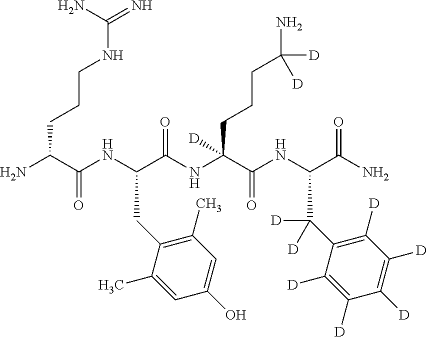 [SMILES: [2H]c1c([2H])c([2H])c(C([2H])([2H])[C@H](NC(=O)[C@]([2H])(CCCC([2H])([2H])N)NC(=O)[C@H](Cc2c(C)cc(O)cc2C)NC(=O)[C@H](N)CCCNC(=N)N)C(N)=O)c([2H])c1[2H]]

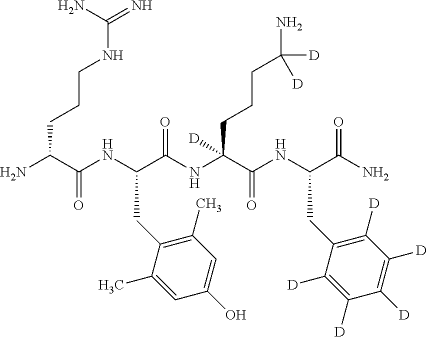 [SMILES: [2H]c1c([2H])c([2H])c(C[C@H](NC(=O)[C@]([2H])(CCCC([2H])([2H])N)NC(=O)[C@H](Cc2c(C)cc(O)cc2C)NC(=O)[C@H](N)CCCNC(=N)N)C(N)=O)c([2H])c1[2H]]

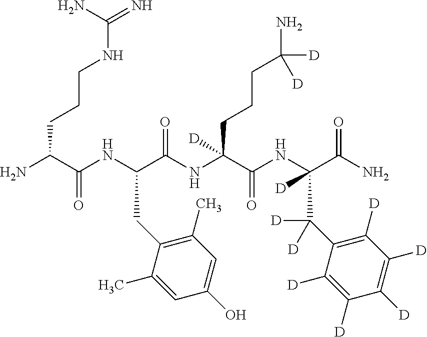 [SMILES: [2H]c1c([2H])c([2H])c(C([2H])([2H])[C@]([2H])(NC(=O)[C@]([2H])(CCCC([2H])([2H])N)NC(=O)[C@H](Cc2c(C)cc(O)cc2C)NC(=O)[C@H](N)CCCNC(=N)N)C(N)=O)c([2H])c1[2H]]

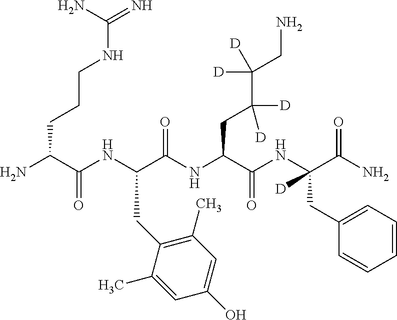 [SMILES: [2H]C([2H])(CN)C([2H])([2H])C[C@H](NC(=O)[C@H](Cc1c(C)cc(O)cc1C)NC(=O)[C@H](N)CCCNC(=N)N)C(=O)N[C@@]([2H])(Cc1ccccc1)C(N)=O]

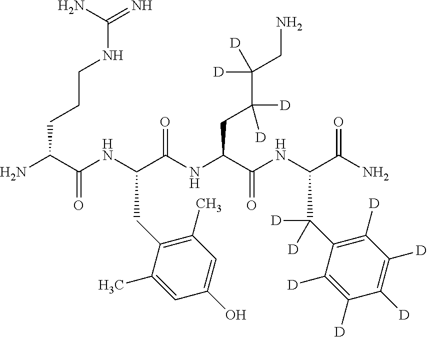 [SMILES: [2H]c1c([2H])c([2H])c(C([2H])([2H])[C@H](NC(=O)[C@H](CC([2H])([2H])C([2H])([2H])CN)NC(=O)[C@H](Cc2c(C)cc(O)cc2C)NC(=O)[C@H](N)CCCNC(=N)N)C(N)=O)c([2H])c1[2H]]

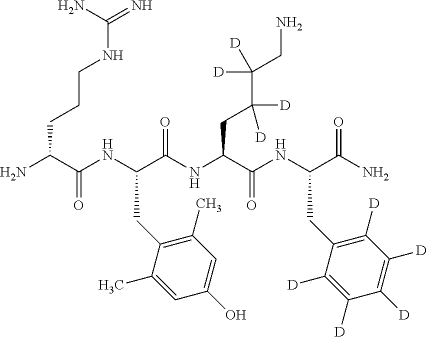 [SMILES: [2H]c1c([2H])c([2H])c(C[C@H](NC(=O)[C@H](CC([2H])([2H])C([2H])([2H])CN)NC(=O)[C@H](Cc2c(C)cc(O)cc2C)NC(=O)[C@H](N)CCCNC(=N)N)C(N)=O)c([2H])c1[2H]]

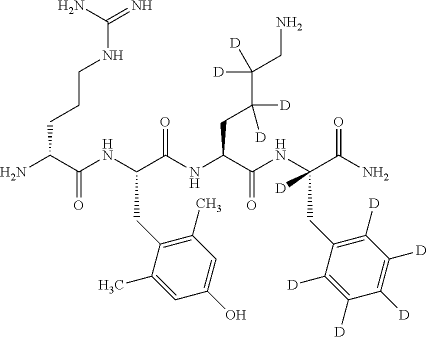 [SMILES: [2H]c1c([2H])c([2H])c(C[C@]([2H])(NC(=O)[C@H](CC([2H])([2H])C([2H])([2H])CN)NC(=O)[C@H](Cc2c(C)cc(O)cc2C)NC(=O)[C@H](N)CCCNC(=N)N)C(N)=O)c([2H])c1[2H]]

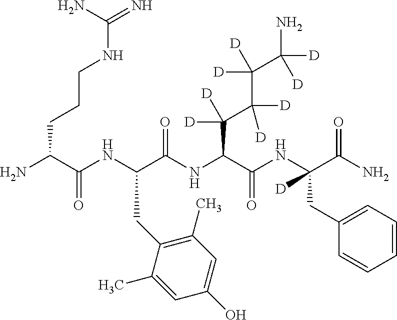 [SMILES: [2H]C([2H])(N)C([2H])([2H])C([2H])([2H])C([2H])([2H])[C@H](NC(=O)[C@H](Cc1c(C)cc(O)cc1C)NC(=O)[C@H](N)CCCNC(=N)N)C(=O)N[C@@]([2H])(Cc1ccccc1)C(N)=O]

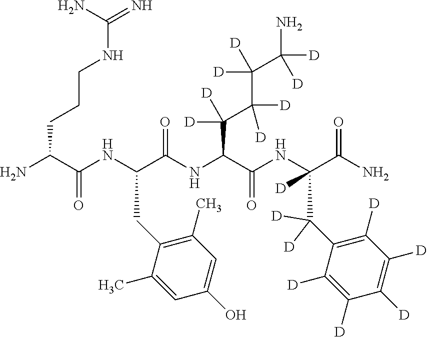 [SMILES: [2H]c1c([2H])c([2H])c(C([2H])([2H])[C@]([2H])(NC(=O)[C@@H](NC(=O)[C@H](Cc2c(C)cc(O)cc2C)NC(=O)[C@H](N)CCCNC(=N)N)C([2H])([2H])C([2H])([2H])C([2H])([2H])C([2H])([2H])N)C(N)=O)c([2H])c1[2H]]

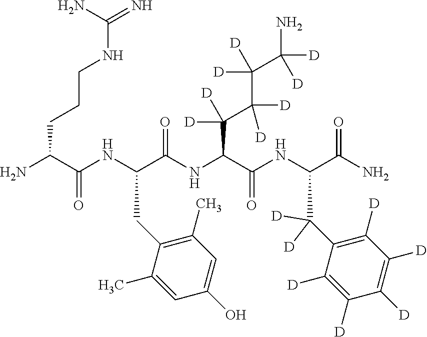 [SMILES: [2H]c1c([2H])c([2H])c(C([2H])([2H])[C@H](NC(=O)[C@@H](NC(=O)[C@H](Cc2c(C)cc(O)cc2C)NC(=O)[C@H](N)CCCNC(=N)N)C([2H])([2H])C([2H])([2H])C([2H])([2H])C([2H])([2H])N)C(N)=O)c([2H])c1[2H]]

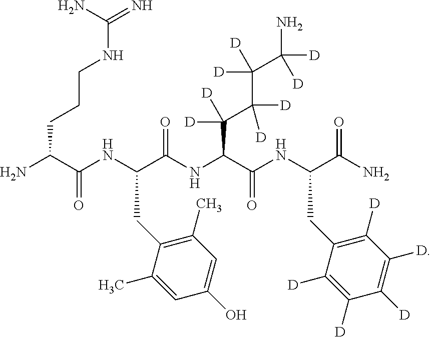

In exemplary embodiments, the compound is selected from the following table:

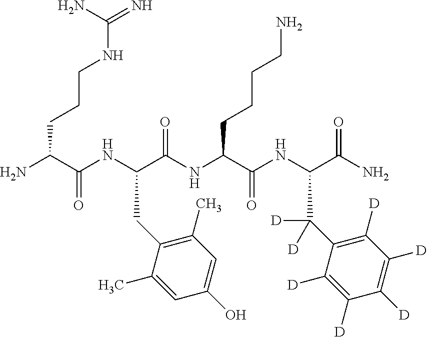 [SMILES: [2H]c1c([2H])c([2H])c(C([2H])([2H])[C@H](NC(=O)[C@H](CCCCN)NC(=O)[C@H](Cc2c(C)cc(O)cc2C)NC(=O)[C@H](N)CCCNC(=N)N)C(N)=O)c([2H])c1[2H]]

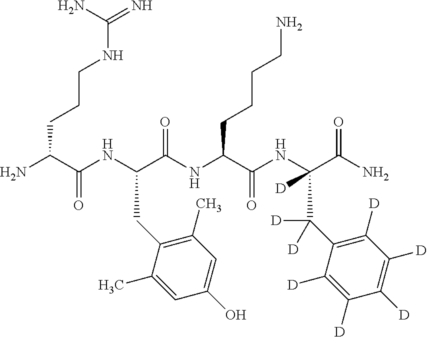 [SMILES: [2H]c1c([2H])c([2H])c(C([2H])([2H])[C@]([2H])(NC(=O)[C@H](CCCCN)NC(=O)[C@H](Cc2c(C)cc(O)cc2C)NC(=O)[C@H](N)CCCNC(=N)N)C(N)=O)c([2H])c1[2H]]

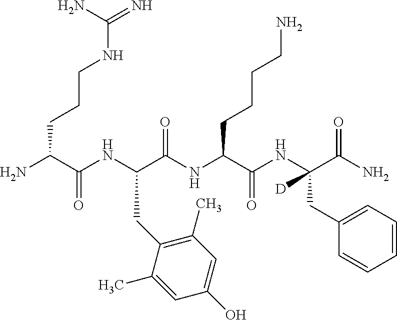 [SMILES: [2H][C@@](Cc1ccccc1)(NC(=O)[C@H](CCCCN)NC(=O)[C@H](Cc1c(C)cc(O)cc1C)NC(=O)[C@H](N)CCCNC(=N)N)C(N)=O]

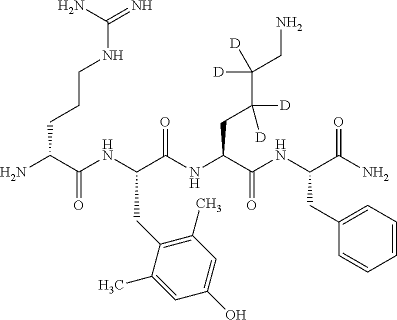 [SMILES: [2H]C([2H])(CN)C([2H])([2H])C[C@H](NC(=O)[C@H](Cc1c(C)cc(O)cc1C)NC(=O)[C@H](N)CCCNC(=N)N)C(=O)N[C@@H](Cc1ccccc1)C(N)=O]

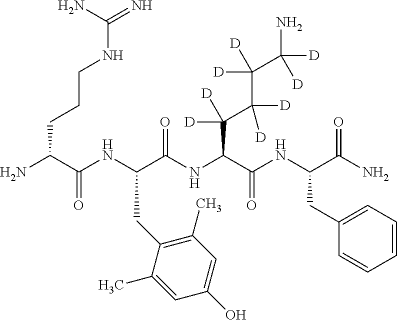 [SMILES: [2H]C([2H])(N)C([2H])([2H])C([2H])([2H])C([2H])([2H])[C@H](NC(=O)[C@H](Cc1c(C)cc(O)cc1C)NC(=O)[C@H](N)CCCNC(=N)N)C(=O)N[C@@H](Cc1ccccc1)C(N)=O]

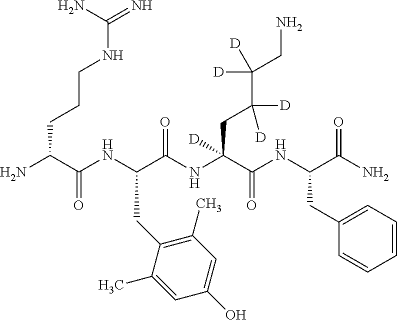 [SMILES: [2H]C([2H])(CN)C([2H])([2H])C[C@]([2H])(NC(=O)[C@H](Cc1c(C)cc(O)cc1C)NC(=O)[C@H](N)CCCNC(=N)N)C(=O)N[C@@H](Cc1ccccc1)C(N)=O]

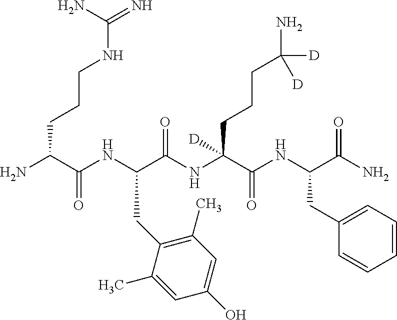 [SMILES: [2H]C([2H])(N)CCC[C@]([2H])(NC(=O)[C@H](Cc1c(C)cc(O)cc1C)NC(=O)[C@H](N)CCCNC(=N)N)C(=O)N[C@@H](Cc1ccccc1)C(N)=O]

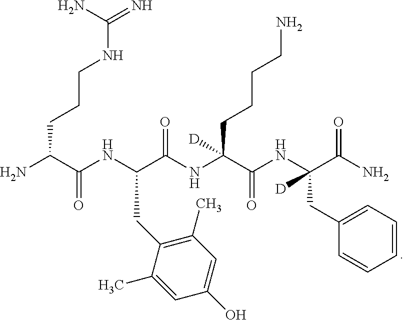 [SMILES: [2H][C@@](Cc1ccccc1)(NC(=O)[C@]([2H])(CCCCN)NC(=O)[C@H](Cc1c(C)cc(O)cc1C)NC(=O)[C@H](N)CCCNC(=N)N)C(N)=O]

Peptide Synthesis

The peptidic compounds of the invention may be prepared using a peptide synthesis method, such as conventional liquid-phase peptide synthesis or solid-phase peptide synthesis, or by peptide synthesis by means of an automated peptide synthesizer (Kelley et al., Genetics Engineering Principles and Methods, Setlow, J. K. eds., Plenum Press NY. (1990) Vol. 12, pp. 1 to 19; Stewart et al., Solid-Phase Peptide Synthesis (1989) W. H.; Houghten, Proc. Natl. Acad. Sci. USA (1985) 82: p. 5132). The peptide thus produced can be collected or purified by a routine method, for example, chromatography, such as gel filtration chromatography, ion exchange column chromatography, affinity chromatography, reverse phase column chromatography, and HPLC, ammonium sulfate fractionation, ultrafiltration, and immunoadsorption.

In a solid-phase peptide synthesis, peptides are typically synthesized from the carbonyl group side (C-terminus) to amino group side (N-terminus) of the amino acid chain. In certain embodiments, an amino-protected amino acid is covalently bound to a solid support material through the carboxyl group of the amino acid, typically via an ester or amido bond and optionally via a linking group. The amino group may be deprotected and reacted with (i.e., “coupled” with) the carbonyl group of a second amino-protected amino acid using a coupling reagent, yielding a dipeptide bound to a solid support. These steps (i.e., deprotection, coupling) may be repeated to form the desired peptide chain. Once the desired peptide chain is complete, the peptide may be cleaved from the solid support.

In certain embodiments, the protecting groups used on the amino groups of the amino acid residues include 9-fluorenylmethyloxycarbonyl group (Fmoc) and t-butyloxycarbonyl (Boc). The Fmoc group is removed from the amino terminus with base while the Boc group is removed with acid. In alternative embodiments, the amino protecting group may be formyl, acrylyl (Acr), benzoyl (Bz), acetyl (Ac), trifluoroacetyl, substituted or unsubstituted groups of aralkyloxycarbonyl type, such as the benzyloxycarbonyl (Z), p-chlorobenzyloxycarbonyl, p-bromobenzyloxycarbonyl, p-nitrobenzyloxycarbonyl, p-methoxybenzyloxycarbonyl, benzhydryloxycarbonyl, 2(p-biphenylyl)isopropyloxycarbonyl, 2-(3,5-dimethoxyphenyl)isopropyloxycarbonyl, p-phenylazobenzyloxycarbonyl, triphenylphosphonoethyloxycarbonyl or 9-fluorenylmethyloxycarbonyl group (Fmoc), substituted or unsubstituted groups of alkyloxycarbonyl type, such as the tert-butyloxycarbonyl (BOC), tert-amyloxycarbonyl, diisopropylmethyloxycarbonyl, isopropyloxycarbonyl, ethyloxycarbonyl, allyloxycarbonyl, 2 methylsulphonylethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl, mesitylenesulphonyl, methoxytrimethylphenylsulphonyl, 2-nitrobenzenesulfonyl, 2-nitrobenzenesulfenyl, 4-nitrobenzenesulfonyl or 4-nitrobenzenesulfenyl group.

Many amino acids bear reactive functional groups in the side chain. In certain embodiments, such functional groups are protected in order to prevent the functional groups from reacting with the incoming amino acid. The protecting groups used with these functional groups must be stable to the conditions of peptide synthesis, but may be removed before, after, or concomitantly with cleavage of the peptide from the solid support.

In certain embodiments, the solid support material used in the solid-phase peptide synthesis method is a gel-type support such as polystyrene, polyacrylamide, or polyethylene glycol. Alternatively, materials such as pore glass, cellulose fibers, or polystyrene may be functionalized at their surface to provide a solid support for peptide synthesis.

Coupling reagents that may be used in the solid-phase peptide synthesis described herein are typically carbodiimide reagents. Examples of carbodiimide reagents include, but are not limited to, N,N′-dicyclohexylcarbodiimide (DCC), 1-(3-dimethylaminopropyl)-3-ethylcarbodiimide (EDC), N-cyclohexyl-N′-isopropylcarbodiimide (CIC), N,N′-diisopropylcarbodiimide (DIC), N-tert-butyl-N′-methylcarbodiimide (BMC), N-tert-butyl-N′-ethylcarbodiimide (BEC), bis[[4-(2,2-dimethyl-1,3-dioxolyl)]-methyl]carbodiimide (BDDC), and N,N-dicyclopentylcarbodiimide. DCC is a preferred coupling reagent.

In certain exemplary embodiments, the compound 1 (pictured below) is synthesized in a linear sequential fashion, according to the solid phase synthesis depicted in Scheme 1.

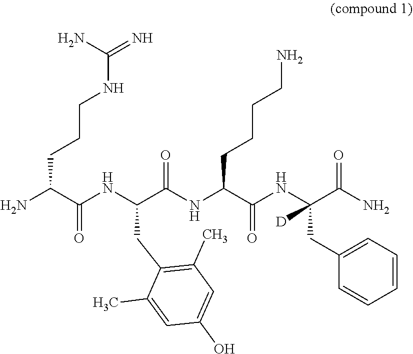 [SMILES: [2H][C@@](Cc1ccccc1)(NC(=O)[C@H](CCCCN)NC(=O)[C@H](Cc1c(C)cc(O)cc1C)NC(=O)[C@H](N)CCCNC(=N)N)C(N)=O]

For reference in the following schemes,

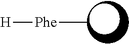

indicates

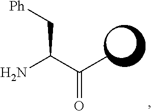

wherein [symbol] represents a solid support and optionally a linking group. Furthermore,

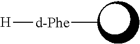

indicates a deuterated phenylalanine amino acid residue linked to a solid support, such as any of the deuterated phenylalanine residues described herein. More specifically, in Schemes 1 and 2 below,

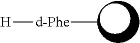

represents

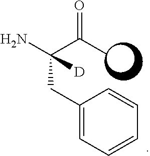

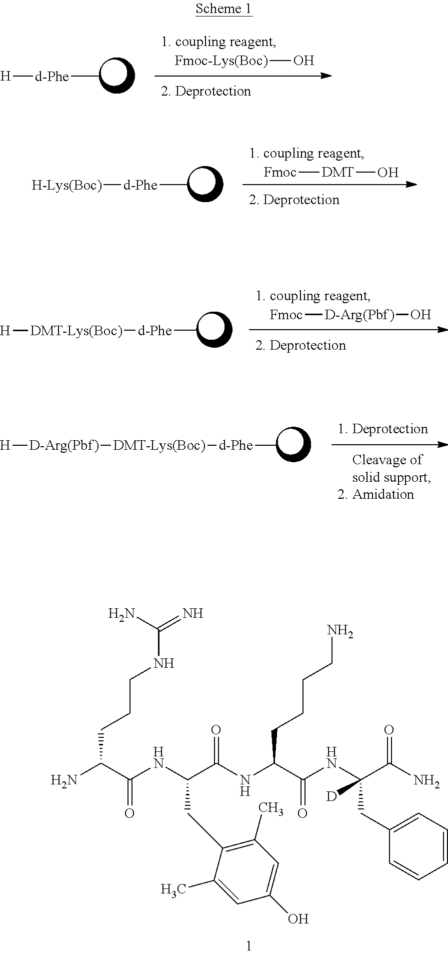

Alternatively, the compound 1 may be synthesized in a convergent fashion, according to Scheme 2:

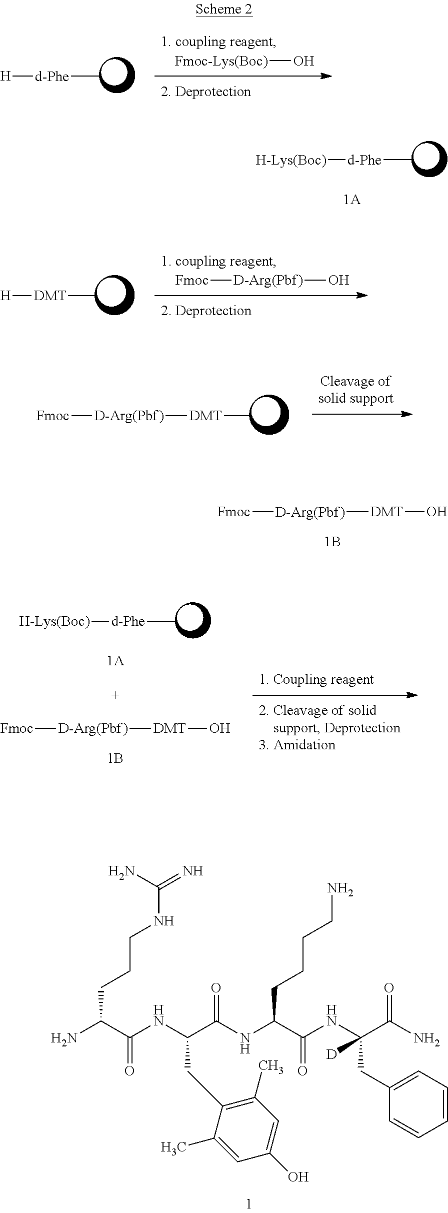

The compounds of the invention may also be synthesized according to conventional liquid-phase peptide synthetic routes. For example, compound 2 (pictured below) may be synthesized in a convergent liquid-phase synthesis, as depicted in Scheme 3.

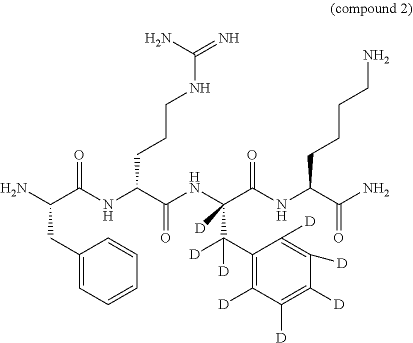 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@H](CCCCN)C(N)=O)c([2H])c1[2H]]

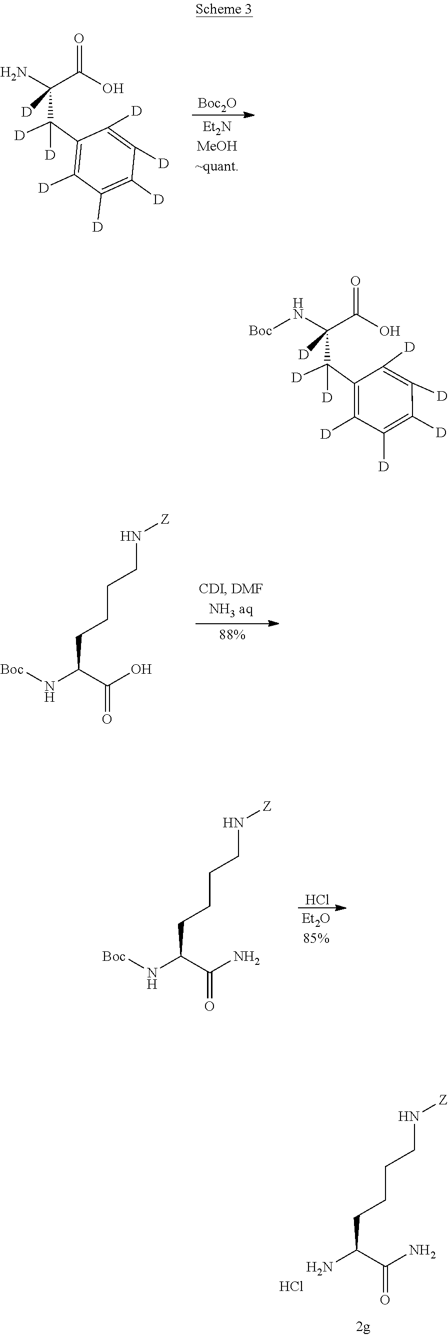

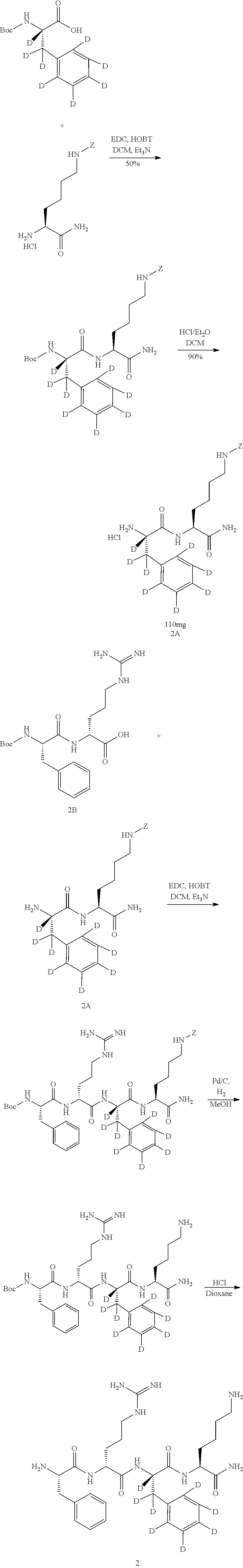

In another exemplary embodiment, compound 3 (pictured below) is made via the linear sequential liquid phase synthesis depicted in Scheme 4.

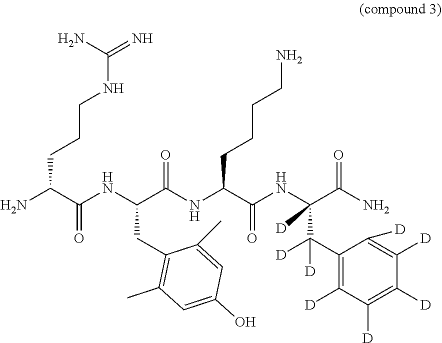 [SMILES: [2H]c1c([2H])c([2H])c(C([2H])([2H])[C@]([2H])(NC(=O)[C@H](CCCCN)NC(=O)[C@H](Cc2c(C)cc(O)cc2C)NC(=O)[C@H](N)CCCNC(=N)N)C(N)=O)c([2H])c1[2H]]

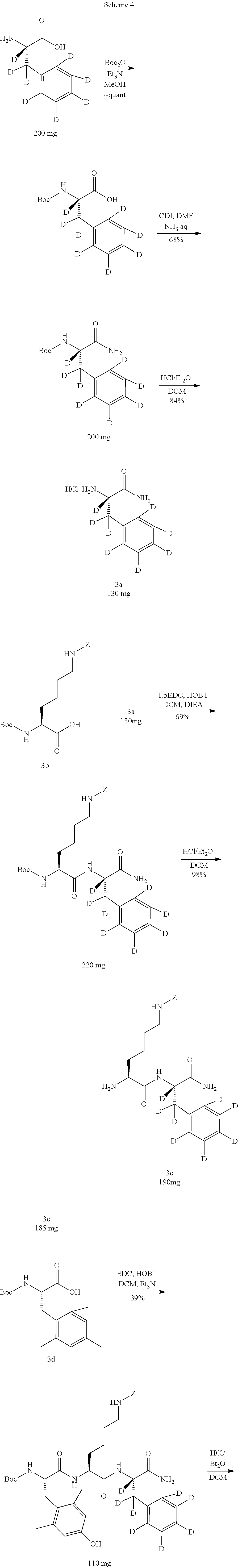

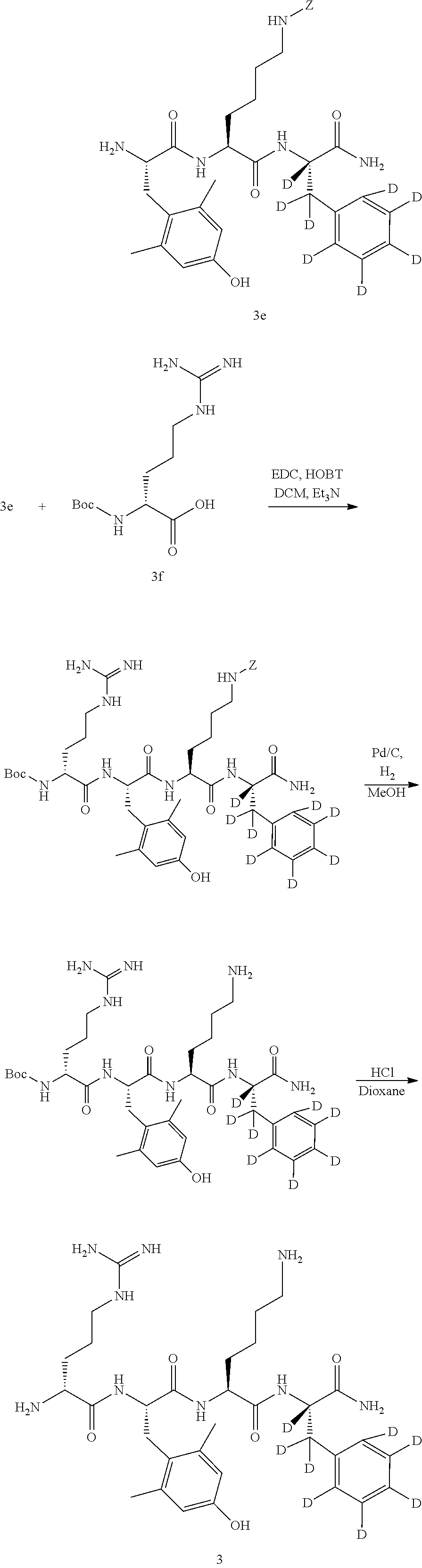

### Definitions

The nomenclature used to define the peptide compounds described herein is that typically used in the art wherein the amino group at the N-terminus appears to the left and the carboxyl group at the C-terminus appears to the right.

As used herein, the term “amino acid” includes both a naturally occurring amino acid and a non-natural amino acid. The term “amino acid,” unless otherwise indicated, includes both isolated amino acid molecules (i.e., molecules that include both, an amino-attached hydrogen and a carbonyl carbon-attached hydroxyl) and residues of amino acids (i.e., molecules in which either one or both an amino-attached hydrogen or a carbonyl carbon-attached hydroxyl are removed). The amino group can be alpha-amino group, beta-amino group, etc. For example, the term “amino acid alanine” can refer either to an isolated alanine H-Ala-OH or to any one of the alanine residues H-Ala-, -Ala-OH, or -Ala-. Unless otherwise indicated, all amino acids found in the compounds described herein can be either in D or L configuration. An amino acid that is in D configuration may be written such that “D” precedes the amino acid abbreviation. For example, “D-Arg” represents arginine in the D configuration. The term “amino acid” includes salts thereof, including pharmaceutically acceptable salts. Any amino acid can be protected or unprotected. Protecting groups can be attached to an amino group (for example alpha-amino group), the backbone carboxyl group, or any functionality of the side chain. As an example, phenylalanine protected by a benzyloxycarbonyl group (Z) on the alpha-amino group would be represented as Z-Phe-OH.

With the exception of the N-terminal amino acid, all abbreviations of amino acids (for example, Phe) in this disclosure stand for the structure of —NH—C(R)(R′)—CO—, wherein R and R′ each is, independently, hydrogen or the side chain of an amino acid (e.g., R=benzyl and R′=H for Phe). Accordingly, phenylalanine is H-Phe-OH. The designation “OH” for these amino acids, or for peptides (e.g., Lys-Val-Leu-OH) indicates that the C-terminus is the free acid. The designation “NH₂” in, for example, Phe-D-Arg-Phe-Lys-NH₂ indicates that the C-terminus of the protected peptide fragment is amidated. Further, certain R and R′, separately, or in combination as a ring structure, can include functional groups that require protection during the liquid phase synthesis.

Where the amino acid has isomeric forms, it is the L form of the amino acid that is represented unless otherwise explicitly indicated as D form, for example, D-Arg. Notably, many amino acid residues are commercially available in both D- and L-form. For example, D-Arg is a commercially available D-amino acid.

A capital letter “D” used in conjunction with an abbreviation for an amino acid residue refers to the D-form of the amino acid residue.

As used herein, the term “peptide” refers to two or more amino acids covalently linked by at least one amide bond (i.e., a bond between an amino group of one amino acid and a carboxyl group of another amino acid selected from the amino acids of the peptide fragment). The term “peptide” includes salts thereof, including pharmaceutically acceptable salts.

The articles “a” and “an” are used herein to refer to one or to more than one (i.e., to at least one) of the grammatical object of the article. By way of example, “an element” means one element or more than one element.

It will be recognized that some variation of natural isotopic abundance occurs in a synthesized compound depending upon the origin of chemical materials used in the synthesis. Thus, preparations of MTP-131 and SBT-20 will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Gannes L Z et al., Comp Biochem Physiol Mol Integr Physiol 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

A lowercase letter “d” used in conjunction with an abbreviation for an amino acid residue refers to a deuterated form of the amino acid residue, i.e., wherein at least one proton (H) is replaced by a deuterium (D).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in all will be less than 49.9% of the compound. In other embodiments, the relative amount of such isotopologues in all will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound. As another example, the salt may comprise less than one inorganic or organic acid molecule per molecule of base, such as two molecules of compound per molecule of tartaric acid.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, “inhibit” or “inhibiting” means reduce by an objectively measureable amount or degree compared to control. In one embodiment, inhibit or inhibiting means reduce by at least a statistically significant amount compared to control. In one embodiment, inhibit or inhibiting means reduce by at least 5 percent compared to control. In various individual embodiments, inhibit or inhibiting means reduce by at least 10, 15, 20, 25, 30, 33, 40, 50, 60, 67, 70, 75, 80, 90, 95, or 99 percent compared to control.

As used herein, the terms “treating” and “treat” refer to performing an intervention that results in (a) preventing a condition or disease from occurring in a subject that may be at risk of developing or predisposed to having the condition or disease but has not yet been diagnosed as having it; (b) inhibiting a condition or disease, e.g., slowing or arresting its development or progression; or (c) relieving or ameliorating a condition or disease, e.g., causing regression of the condition or disease. In one embodiment the terms “treating” and “treat” refer to performing an intervention that results in (a) inhibiting a condition or disease, e.g., slowing or arresting its development; or (b) relieving or ameliorating a condition or disease, e.g., causing regression of the condition or disease.

As used herein, a “subject” refers to a living animal. In various embodiments a subject is a mammal. In various embodiments a subject is a non-human mammal, including, without limitation, a mouse, rat, hamster, guinea pig, rabbit, sheep, goat, cat, dog, pig, horse, cow, or non-human primate. In certain embodiments, the subject is a human.

As used herein, “administering” has its usual meaning and encompasses administering by any suitable route of administration, including, without limitation, intravenous, intramuscular, intraperitoneal, subcutaneous, direct injection, mucosal, inhalation, oral, and topical.

As used herein, the phrase “effective amount” refers to any amount that is sufficient to achieve a desired biological effect. A “therapeutically effective amount” is an amount that is sufficient to achieve a desired therapeutic effect, e.g., to treat ischemia-reperfusion injury.

Compounds of the invention and the salts thereof can be combined with other therapeutic agents. The compounds of the invention and other therapeutic agent may be administered simultaneously or sequentially. When the other therapeutic agents are administered simultaneously, they can be administered in the same or separate formulations, but they are administered substantially at the same time. The other therapeutic agents are administered sequentially with one another and with compounds of the invention, when the administration of the other therapeutic agents and the compound of the invention is temporally separated. The separation in time between the administration of these compounds may be a matter of minutes or it may be longer.

Pharmaceutical Compositions, Routes of Administration, and Dosing

In certain embodiments, the invention is directed to a pharmaceutical composition, comprising a compound of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt. When used in medicine the salts should be pharmaceutically acceptable, but non-pharmaceutically acceptable salts may be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

Methods of Use

The present invention provides deuterated peptidic compounds that are useful for treating or preventing ischemia-reperfusion injury or myocardial infarction, or injury associated with myocardial infarction.

Accordingly, in certain embodiments, the invention is directed to a method of treating or preventing ischemia-reperfusion injury, comprising administering to a subject in need thereof a therapeutically effective amount of a compound of formula (I) or (II), described herein, or a pharmaceutically acceptable salt thereof. In certain such embodiments, the ischemia-reperfusion injury is cardiac ischemia-reperfusion injury. In some embodiments, the compound is administered orally, topically, systemically, intravenously, subcutaneously, intraperitoneally, or intramuscularly.

In other embodiments, the present invention provides a method for treating or preventing a myocardial infarction, comprising administering to a subject in need thereof a therapeutically effective amount of compound of formula (I) or (II), or a pharmaceutically acceptable salt thereof. Such methods may prevent injury to the heart upon reperfusion by preventing the initiation or progression of the infarction. In some embodiments, the compound is administered orally, topically, systemically, intravenously, subcutaneously, intraperitoneally, or intramuscularly.

Ischemia is reduction or decrease in blood supply to a tissue or an organ and has many different causes. Ischemia may be local, e.g., caused by thrombus or embolus, or more global, e.g., due to low perfusion pressure. An ischemic event can lead to hypoxia (reduced oxygen) and/or anoxia (absence of oxygen).

Ischemia in a tissue or organ of a mammal is a multifaceted pathological condition that is caused by oxygen deprivation (hypoxia) and/or glucose (e.g., substrate) deprivation. Oxygen and/or glucose deprivation in cells of a tissue or organ leads to a reduction or total loss of energy generating capacity and consequent loss of function of active ion transport across the cell membranes. Oxygen and/or glucose deprivation also leads to pathological changes in other cell membranes, including permeability transition in the mitochondrial membranes. In addition other molecules, such as apoptotic proteins normally compartmentalized within the mitochondria, may leak out into the cytoplasm and cause apoptotic cell death. Profound ischemia can lead to necrotic cell death.

Ischemia or hypoxia in a particular tissue or organ may be caused by a loss or severe reduction in blood supply to the tissue or organ. The loss or severe reduction in blood supply may, for example, be due to thromboembolic stroke, coronary atherosclerosis, or peripheral vascular disease. The tissue affected by ischemia or hypoxia is typically muscle, such as cardiac, skeletal, or smooth muscle.

The organ affected by ischemia or hypoxia may be any organ that is subject to ischemia or hypoxia. By way of example, but not by way of limitation, cardiac muscle ischemia or hypoxia is commonly caused by atherosclerotic or thrombotic blockages, which lead to the reduction or loss of oxygen delivery to the cardiac tissues by the cardiac arterial and capillary blood supply. Such cardiac ischemia or hypoxia may cause pain and necrosis of the affected cardiac muscle, and ultimately may lead to cardiac failure.

Reperfusion is the restoration of blood flow to any organ or tissue in which the flow of blood is decreased or blocked. For example, blood flow can be restored to any organ or tissue affected by ischemia. The restoration of blood flow (reperfusion) can occur by any method known to those in the art. For instance, reperfusion of ischemic cardiac tissues may arise from angioplasty, coronary artery bypass graft, or the use of thrombolytic drugs.

Ischemia-reperfusion injury is the cellular or tissue damage caused when blood supply returns to the affected area after a period of ischemia. The lack of oxygen and nutrients during ischemia creates a condition in which the restoration of circulation results damage to the tissues. By way of example, but not by way of limitation, forms of myocardial reperfusion injury including reperfusion-induced arrhythmias, myocardial stunning, microvascular obstruction manifesting in sluggish coronary blood flow, and lethal myocardial reperfusion injury (i.e., reperfusion-induced death of cardiomyocytes that were viable at the end of the index ischemic event). Studies have suggested that lethal myocardial reperfusion injury accounts for about 50% of the final myocardial infarct size.

In certain embodiments, the peptide is administered orally, intravenously, or parenterally.

In certain embodiments, the subject is a human.

A deuterated compound of the invention, or a pharmaceutically acceptable salt thereof, such as acetate, tartrate, or trifluoroacetate salt, may be administered to a subject suspected of, or already suffering from ischemic injury in an amount sufficient to cure, or at least partially arrest, the symptoms of the disease, including its complications and intermediate pathological phenotypes in development of the disease. Subjects suffering from ischemic injury can be identified by any or a combination of diagnostic or prognostic assays known in the art. By way of example, but by way of limitation, in some embodiments, the ischemic injury is related to cardiac ischemia, brain ischemia, renal ischemia, cerebral ischemia, intestinal ischemia, hepatic ischemia, or myocardial infarction.

By way of example, but not by way of limitation, typical symptoms of cardiac ischemia include, but are not limited to, angina (e.g., chest pain and pressure), shortness of breath, palpitations, weakness, dizziness, nausea, sweating, rapid heartbeat, and fatigue.

In some embodiments, treatment of subjects diagnosed with cardiac ischemia with at least one peptide disclosed herein ameliorates or eliminates of one or more of the following symptoms of cardiac ischemia: angina (e.g., chest pain and pressure), shortness of breath, palpitations, weakness, dizziness, nausea, sweating, rapid heartbeat, and fatigue.

By way of example, but not by way of limitation, typical symptoms of renal ischemia include, but are not limited to, uremia (i.e., high blood levels of protein by-products, such as, e.g., urea), acute episodes of dyspnea (labored or difficult breathing) caused by sudden accumulation of fluid in the lungs, hypertension, pain felt near the kidneys, weakness, hypertension, nausea, a history of leg pain, a stride that reflects compromised circulation to the legs, and bruits (sound or murmurs heard with a stethoscope) caused by turbulent blood flow within the arteries may be detected in the neck (e.g., carotid artery bruit), abdomen (which may reflect narrowing of the renal artery), and groin (femoral artery bruit).

In some embodiments, treatment of subjects diagnosed with renal ischemia with at least one peptide disclosed herein ameliorates or eliminates of one or more of the following symptoms of renal ischemia: uremia (i.e., high blood levels of protein by-products, such as, e.g., urea), acute episodes of dyspnea (labored or difficult breathing) caused by sudden accumulation of fluid in the lungs, hypertension, pain felt near the kidneys, weakness, hypertension, nausea, a history of leg pain, a stride that reflects compromised circulation to the legs, and bruits (sound or murmurs heard with a stethoscope) caused by turbulent blood flow within the arteries may be detected in the neck (e.g., carotid artery bruit), abdomen (which may reflect narrowing of the renal artery), and groin (femoral artery bruit).

By way of example, but not by way of limitation, typical symptoms of cerebral (or brain) ischemia include, but are not limited to, blindness in one eye, weakness in one arm or leg, weakness in one entire side of the body, dizziness, vertigo, double vision, weakness on both sides of the body, difficulty speaking, slurred speech, and the loss of coordination.

In some embodiments, treatment of subjects diagnosed with cerebral (or brain) ischemia with at least one peptide disclosed herein ameliorates or eliminates of one or more of the following symptoms of cerebral (or brain) ischemia: blindness in one eye, weakness in one arm or leg, weakness in one entire side of the body, dizziness, vertigo, double vision, weakness on both sides of the body, difficulty speaking, slurred speech, and the loss of coordination.

In another aspect, the present invention relates to methods of treating ischemia reperfusion injury and/or side effects associated with existing therapeutics against ischemia reperfusion injury. In therapeutic applications, a composition or medicament comprising at least one compound of the invention, or a pharmaceutically acceptable salt thereof, such as acetate, tartrate or trifluoroacetate, is administered to a subject suspected of, or already suffering from ischemic reperfusion injury in an amount sufficient to cure, or at least partially arrest, the symptoms of the disease, including its complications and intermediate pathological phenotypes in development of the disease. Subjects suffering from ischemic-reperfusion injury can be identified by any or a combination of diagnostic or prognostic assays known in the art. In some embodiments, the ischemia-reperfusion injury is related to cardiac ischemia, brain ischemia, renal ischemia, cerebral ischemia, intestinal ischemia, and hepatic ischemia. In some embodiments, the deuterated compounds disclosed herein are useful in the treatment of cardiac ischemia-reperfusion injury.

In some embodiments, the deuterated compounds disclosed herein are useful in treating myocardial infarction in a subject to prevent injury to the heart upon reperfusion. In some embodiments, the invention relates to methods of coronary revascularization, comprising administering to a mammalian subject a therapeutically effective amount of a deuterated compound of the invention, or a pharmaceutically acceptable salt thereof, and performing a coronary artery bypass graft (CABG) procedure on the subject.

In some embodiments, treatment of myocardial infarction with the deuterated compounds disclosed herein reduces infarct size, increases LVDP, and increases maximal rates of contraction and relaxation (±dP/dt).

### Prophylactic Methods

In some embodiments, the present invention provides methods for preventing or delaying the onset of ischemic injury or symptoms of ischemic injury in a subject at risk of having ischemia injury. In some embodiments, the present technology provides methods for preventing or reducing the symptoms of ischemic injury in a subject at risk of having ischemia injury.

In some embodiments, the present invention provides methods for preventing or delaying the onset of ischemia-reperfusion injury or symptoms of ischemia-reperfusion injury in a subject at risk of having ischemia-reperfusion injury. In some embodiments, the present invention provides methods for preventing or reducing the symptoms of ischemia reperfusion injury in a subject at risk of having ischemia-reperfusion injury.

In some embodiments, the ischemic injury, the ischemia-reperfusion injury, or symptoms of ischemic or ischemia-reperfusion injury is related to cardiac ischemia, brain ischemia, renal ischemia, cerebral ischemia, intestinal ischemia, and hepatic ischemia. In some embodiments, the ischemic injury is myocardial infarction.

In some embodiments, the deuterated compounds disclosed herein are useful in the treatment or prevention of cardiac ischemia-reperfusion injury. In some embodiments, the deuterated compounds disclosed herein are useful in the prevention of cardiac ischemia-reperfusion injury.

Subjects at risk for ischemic injury or ischemia-reperfusion injury can be identified by, e.g., any or a combination of diagnostic or prognostic assays known in the art. In prophylactic applications, a pharmaceutical composition or medicament of a compound of the invention, or a pharmaceutically acceptable salt thereof, such as acetate, tartrate, or trifluoroacetate salt, is administered to a subject susceptible to, or otherwise at risk of for ischemic injury or ischemia reperfusion injury in an amount sufficient to eliminate, reduce the risk, or delay the onset of the disease, including biochemical, histologic and/or behavioral symptoms of the disease, its complications and intermediate pathological phenotypes presenting during development of the disease or reduce the symptoms and/or complications and intermediate pathological phenotypes presenting during development of the disease. Administration of a prophylactic peptide can occur prior to the manifestation of symptoms characteristic of the disease or disorder, such that the disease or disorder is prevented, delayed in its progression, or the severity of the symptoms or side effects of the disease or disorder are reduced.

By way of example, in some embodiments, subjects may be at risk for cardiac ischemia if they have coronary artery disease (atherosclerosis), blood clots, or coronary artery spasm.

By way of example, but not by way of limitation, in some embodiments, subjects may be at risk for renal ischemia if they have kidney injury (e.g., acute kidney injury) and/or injuries or complications from surgeries in which the kidneys are deprived of normal blood flow for extended periods of time (e.g., heart-bypass surgery).

By way of example, but not by way of limitation, in some embodiments, subjects may be at risk for cerebral ischemia if they have sickle cell anemia, compressed blood vessels, ventricular tachycardia, plaque buildup in the arteries, blood clots, extremely low blood pressure as a result of heart attack, had a stroke, or congenital heart defects.

For therapeutic and/or prophylactic applications, a composition comprising at least one deuterated compound described herein, or a pharmaceutically acceptable salt thereof, such as acetate, tartrate, or trifluoroacetate salt, is administered to a subject in need thereof. In some embodiments, the peptide composition is administered one, two, three, four, or five times per day. In some embodiments, the peptide composition is administered more than five times per day. Additionally or alternatively, in some embodiments, the peptide composition is administered every day, every other day, every third day, every fourth day, every fifth day, or every sixth day. In some embodiments, the peptide composition is administered weekly, bi-weekly, tri-weekly, or monthly. In some embodiments, the peptide composition is administered for a period of one, two, three, four, or five weeks. In some embodiments, the peptide is administered for six weeks or more. In some embodiments, the peptide is administered for twelve weeks or more. In some embodiments, the peptide is administered for a period of less than one year. In some embodiments, the peptide is administered for a period of more than one year. In some embodiments, treatment with at least one peptide disclosed herein will prevent or delay the onset of one or more of the following symptoms of cardiac ischemia: angina (e.g., chest pain and pressure), shortness of breath, palpitations, weakness, dizziness, nausea, sweating, rapid heartbeat, and fatigue.

In some embodiments, treatment with at least one peptide disclosed herein will prevent or delay the onset of one or more of the following symptoms of renal ischemia: uremia (i.e., high blood levels of protein by-products, such as, e.g., urea), acute episodes of dyspnea (labored or difficult breathing) caused by sudden accumulation of fluid in the lungs, hypertension, pain felt near the kidneys, weakness, hypertension, nausea, a history of leg pain, a stride that reflects compromised circulation to the legs, and bruits (sound or murmurs heard with a stethoscope) caused by turbulent blood flow within the arteries may be detected in the neck (e.g., carotid artery bruit), abdomen (which may reflect narrowing of the renal artery), and groin (femoral artery bruit).

In some embodiments, treatment with at least one peptide disclosed herein will prevent or delay the onset of one or more of the following symptoms of cerebral (or brain) ischemia: blindness in one eye, weakness in one arm or leg, weakness in one entire side of the body, dizziness, vertigo, double vision, weakness on both sides of the body, difficulty speaking, slurred speech, and the loss of coordination.

Methods of Evaluating Metabolic Stability

In certain embodiments, the following methods can be used to evaluate the metabolic stability of the compounds of the invention.

Certain in vitro liver metabolism studies have been described previously in the following references: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay: Human liver microsomes (20 mg/mL) may be obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) may be purchased from Sigma-Aldrich.

Determination of Metabolic Stability: 7.5 mM stock solutions of test compounds are prepared in DMSO. The 7.5 mM stock solutions are diluted to 12.5-50 M in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the all-protonated (un-deuterated) compounds. Testing is done in triplicate.

Data analysis: The in vitro half-lives (t_{1/2S}) for test compounds are calculated from the slopes of the linear regression of % parent remaining (ln) vs incubation time relationship: in vitro t_{1/2}=0.693/k, where k=−[slope of linear regression of % parent remaining (ln) vs incubation time]

### EXAMPLES

### General Procedures for Key Intermediates

### A) Synthesis of (N2-Cbz-N6-Boc-3,3,4,4,5,5,6,6-d8)-Lys

### Step 1. Synthesis of (N6-Boc-3,3,4,4,5,5,6,6-d8)-lysine

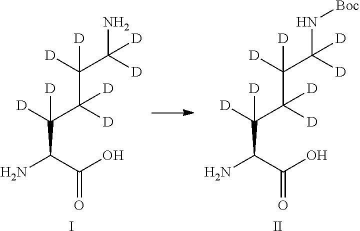

Lysine(D8)x2HCl (500 mg, 2.62 mmol) was dissolved in 2 M NaHCO₃ (2.60 mL, 5.24 mmol, 440 mg), to which a solution of CuSO₄x5H₂O (327 mg, 1.31 mmol) in H₂O (2.60 mL) was added. An additional NaHCO₃ (220 mg, 2.62 mmol) was added, followed by ditertbutyldicarbonate (915 mg, 4.19 mmol) dissolved in 3.2 mL acetone. The mixture stirred 24 h. Methanol (1 mL) was added to the solution, and stirring continued 14 h. To reaction mixture H₂O (2 mL) is added and the product was subsequently filtered, washed with H₂O (3×2 mL) and dried to give 712 mg (95%) of Cu[lys(D8)^{ε}(Boc)]₂ as pale blue solid.

To remove the copper, the pale blue Cu-chelate (712 mg, 1.25 mmol) was suspended in H₂O (27 mL). Thereafter, 8-quinolinol (465 mg, 3.20 mmol) was added, and the mixture stirred 18 hours (pale salad-green precipitate formation). The suspension was filtered, precipitates washed with H₂O (3×7 mL) and the filtrate washed with EtOAc (3×15 mL). The aqueous layer was evaporated to yield 573 mg (90%) of Lys(ε-Boc)(D8)-OH as a white solid. Overall yield—86%. ¹H NMR (300 MHz, D₂O) δ: 3.72 (s, 1H), 1.44 (s, 10H).

### Step 2. (N2-Cbz-N6-Boc-3,3,4,4,5,5,6,6-d8)-Lys

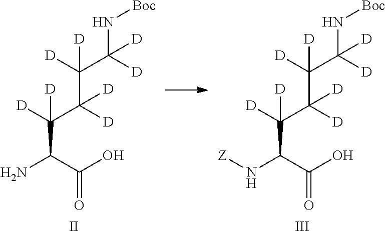

To a suspension of II (318 mg, 1.25 mmol) in mixture of dioxane (8.1 mL) and water (8.1 mL), NaHCO₃ (525 mg, 6.25 mmol) and then N-(Benzyloxycarbonyloxy)succinimide (374 mg, 1.50 mmol) was added. Biphasic mixture was allowed to stir at rt for 20 h. Then additional water (4.9 mL) was added and reaction mixture was extracted with Et₂O (3×13 mL), water layer then was added 5% solution of citric acid (17 mL) and product was extracted with DCM (3×17 mL). Organic layers was combined and additionally washed with brine (9 mL). Organic layer was dried on Na₂SO₄, filtered and evaporated to give product as colorless oil (444 mg, 91%). ¹H NMR (300 MHz, DMSO-d₆) δ: 7.51 (d, J=8.0 Hz, 1H), 7.41-7.27 (m, 5H), 6.73 (s, 1H), 5.02 (s, 2H), 3.88 (d, J=7.9 Hz, 1H), 1.36 (s, 9H).

### B) Synthesis of (N2-Cbz-N6-Boc-4,4,5,5-d4)-Lysine

### Step 1. Synthesis of (N6-Boc-4,4,5,5-d4)-lysine

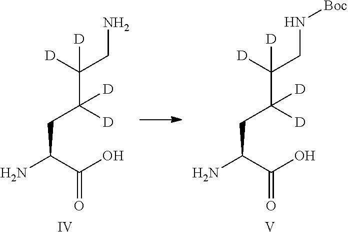

Lysine(D4)x2HCl (500 mg, 2.68 mmol) was dissolved in 2 M NaHCO₃ (2.60 mL, 5.36 mmol, 450 mg), to which a solution of CuSO₄x5H₂O (335 mg, 1.34 mmol) in H₂O (2.60 mL) was added. An additional NaHCO₃ (225 mg, 2.68 mmol) was added, followed by ditertbutyldicarbonate (936 mg, 4.29 mmol) dissolved in 3.2 mL acetone. The mixture stirred 24 hours. Methanol (1 mL) was added to the solution, and stirring continued 16 h. To reaction mixture H₂O (2 mL) is added and the product was subsequently filtered, washed with H₂O (3×2 mL) and dried to give 684 mg (93% yield) of Cu[lys(D4)^{ε}(Boc)]₂ as pale blue solid.

To remove the copper, the pale blue Cu-chelate (684 mg, 1.25 mmol) was suspended in H₂O (28 mL). Thereafter, 8-quinolinol (466 mg, 3.21 mmol) was added, and the mixture stirred 18 hours (pale salad-green precipitate formation). The suspension was filtered, precipitates washed with H₂O (3×7 mL) and the filtrate washed with EtOAc (3×14 mL). The aqueous layer was evaporated to yield 598 mg (96% yield) of Lys(ε-Boc)(D4)-OH as a white solid. ¹H NMR (400 MHz, D₂O) δ: 3.73 (dd, J=6.8, 5.5 Hz, 1H), 3.08 (s, 2H), 1.94-1.79 (m, 2H), 1.44 (s, 9H). Overall yield 89%.

### Step 2. Synthesis of (N2-Cbz-N6-Boc-4,4,5,5-d4)-lysine

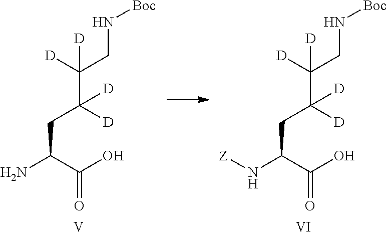

NaHCO₃ (2007 mg, 23.9 mmol) was suspended in water (19 mL), then V (598 mg, 2.39 mmol), THE (19 mL) and N-(Benzyloxycarbonyloxy)succinimide (833 mg, 3.34 mmol) was added. Biphasic mixture was allowed to vigorously stir at room temperature for 20 h. Then additional water (19 mL) was added and reaction mixture was washed with Et₂O (3×35 mL). To aqueous layer was added 5% solution of citric acid (50 mL) and product was extracted with DCM (4×50 mL). Organic layers were combined and dried on Na₂SO₄, filtered and evaporated to give product (918 mg, 99%) as colorless oil. ¹H NMR (300 MHz, DMSO-d₆) δ: 12.52 (s, 1H), 7.52 (d, J=7.9 Hz, 1H), 7.45-7.25 (m, 5H), 6.75 (t, J=5.6 Hz, 1H), 5.02 (s, 2H), 3.89 (td, J=9.2, 4.8 Hz, 1H), 2.86 (d, J=5.7 Hz, 2H), 1.69-1.46 (m, 2H), 1.36 (s, 9H).

### C) Solid Phase Synthesis of (Boc)-Phe-D-Arg-Phe-OH

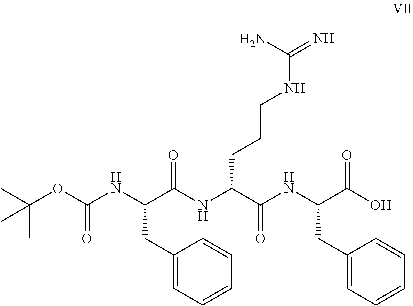 [SMILES: CC(C)(C)OC(=O)N[C@@H](Cc1ccccc1)C(=O)N[C@H](CCCNC(=N)N)C(=O)N[C@@H](Cc1ccccc1)C(=O)O]

1. Resin preparation

Weigh 200 g of RinkFmoc-Phe-OH Wang Resin (loading 0.5 mmol/g), dump the resins into a reaction column, and swell it for 30 min with DCM.

2. Deprotection

Deprotect Fmoc from the amino acid by 60% piperidine, mix them for 10 min, then wash it with DMF. Repeat this step.

3. Coupling

Add 100 mmol Boc-D-Arg-OH, and 100 mmol of HOBT into the resin for coupling for 20 min at RT.

4. Deprotection

Deprotect Fmoc from the amino acid by 60% piperidine, mix them for 10 min, then wash it with DMF. Repeat this step.

5. Coupling

Add 100 mmol BOC-Phe-OH, and 100 mmol of HOBT into the resin for coupling for 20 min at room temperature (RT).

6. Washing

Wash the resins 1-2 times by DMF after the coupling is completed.

7. Deprotect the peptide after the last amino acid has been coupled to the chain, then wash the resin with MeOH for 3 times. Dry the resin.

8. Cleavage

Weight out the dried resin, put them into a tube, add appropriate amount of cleavage solution, and incubate it at 40 degree for 3.5 hours. Filter the reaction solution and then precipitate the solution by adding it into Ether.

Centrifuge the solution twice for 2 min (4000/s).

9. Drying

Air-dry the peptide sample for a few minutes, then lyophilize the peptide sample.

### Instrument: 030049

10. Purification

Wavelength: 220 nm

Flow rate: 180 ml/min

Column: 10 cm (C18)

Mobile Phase A: ACN+TFA, B: water+TFA

Gradient:

| time (min) | A | B |
|---|---|---|
| 0 | 29% | 71% |
| 50 | 44% | 56% |
| 100 | 59% | 61% |

11. Pre-analysis

Take appropriate amount of sample into a 0.5 mL tube, dissolve it using acetonitrile and ultrapure water. Filter the sample using a 0.45 um membrane, then analyze the sample using a fast gradient HPLC (10-100%)

12. Sample preparation

Add 8000 mg sample into a 500 mL beaker, then add 100 mL acetonitrile and 200 mL H₂O. Sonicate the sample until the sample is completely dissolved, then filter the solution using a 0.45 um membrane.

13. HPLC purification

Purify the sample using HPLC with the above gradient, collect the fractions at 28-35 min. Analyze the collected fraction using analytical HPLC to check purity

14. Drying and Lyophilization

Dry the collected fraction using Rotary evaporator then lyophilized it for two days.

15. Storage

Weight and inspect the dried sample, then store it in a tube. Below 10° C., avoid light.

### D) Solid Phase Synthesis of (Boc)-Phe-D-Arg-OH

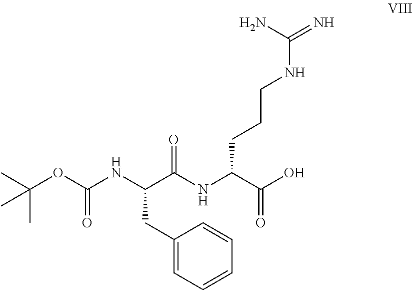 [SMILES: CC(C)(C)OC(=O)N[C@@H](Cc1ccccc1)C(=O)N[C@H](CCCNC(=N)N)C(=O)O]

1. Resin preparation

Weigh 200 g of RinkFmoc-Phe-OH Wang Resin (loading 0.5 mmol/g), dump the resins into a reaction column, and swell it for 30 min with DCM.

2. Deprotection

Deprotect Fmoc from the amino acid by 60% piperidine, mix them for 10 min, then wash it with DMF. Repeat this step.

3. Coupling

Add 100 mmol Boc-D-Arg-OH, and 100 mmol of HOBT into the resin for coupling for 20 min at RT.

4. Deprotection

Deprotect Fmoc from the amino acid by 60% piperidine, mix them for 10 min, then wash it with DMF. Repeat this step.

5. Purification and sample preparation

The same procedures as described in schedule C), steps 4-15.

### E) Solid Phase Synthesis of (Boc)-D-Arg-DMT-OH

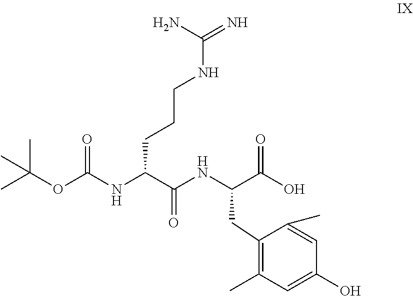 [SMILES: Cc1cc(O)cc(C)c1C[C@H](NC(=O)[C@@H](CCCNC(=N)N)NC(=O)OC(C)(C)C)C(=O)O]

1. Resin preparation

Weigh 200 g of RinkFmoc-DMT-OH Wang Resin (loading 0.5 mmol/g), dump the resins into a reaction column, and swell it for 30 min with DCM.

2. Deprotection

Deprotect Fmoc from the amino acid by 60% piperidine, mix them for 10 min, then wash it with DMF. Repeat this step.

3. Coupling

Add 100 mmol Boc-D-Arg-OH, and 100 mmol of HOBT into the resin for coupling for 20 min at RT.

4. Deprotection

Deprotect Fmoc from the amino acid by 60% piperidine, mix them for 10 min, then wash it with DMF. Repeat this step.

5. Purification and sample preparation

The same procedures as described in schedule C), steps 4-15.

### Example 1: Synthesis of Phe-D-Arg-Phe-(3,3,4,4,5,5,6,6-octadeuterium)-Lys-NH2

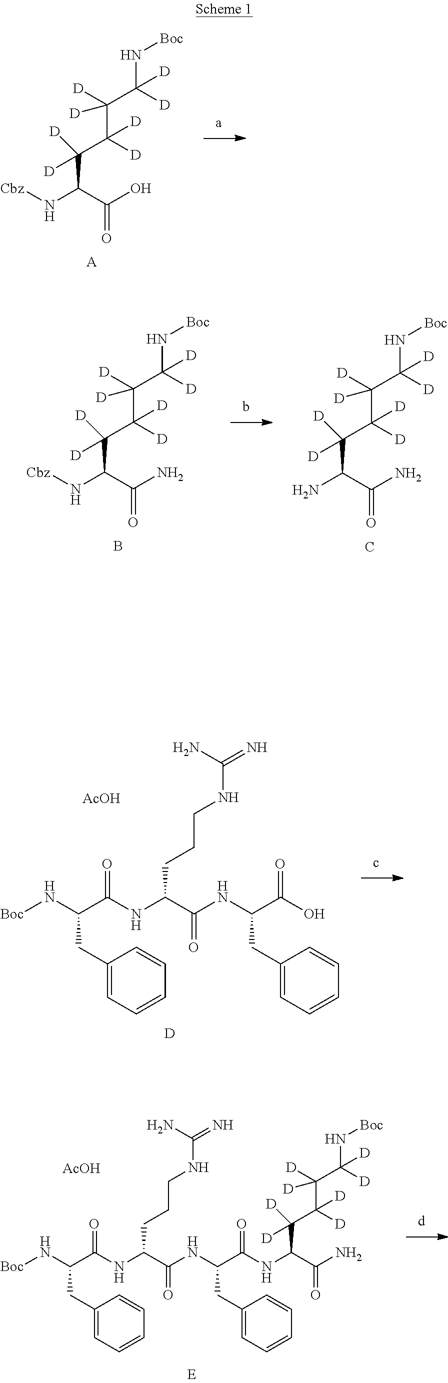

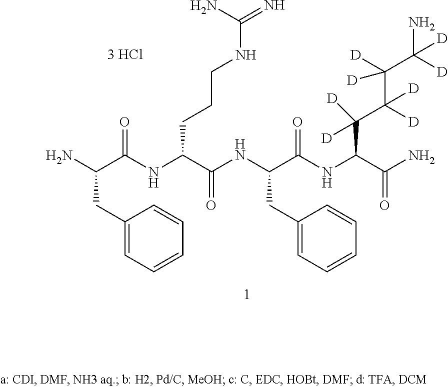 [SMILES: [2H]C([2H])(N)C([2H])([2H])C([2H])([2H])C([2H])([2H])[C@H](NC(=O)[C@H](Cc1ccccc1)NC(=O)[C@@H](CCCNC(=N)N)NC(=O)[C@@H](N)Cc1ccccc1)C(N)=O]

### Step 1. Synthesis of (N2-Cbz-N6-Boc-3,3,4,4,5,5,6,6-d8)-Lys-NH2

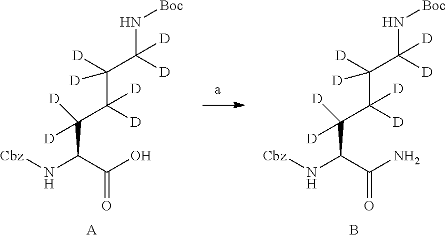

To a stirring solution of (N2-Cbz-N6-Boc-3,3,4,4,5,5,6,6-d8)-L-Lys-OH (A, 1717 mg, 4.42 mmol) in dry DMF (22 mL) was added carbonyldiimidazole (932 mg, 5.75 mmol) in one portion. The mixture stirred for 1.5 h at rt then aqueous ammonia (0.45 mL, 6.63 mmol, 14.8 M) was added dropwise. After stirring an additional 3 h, no starting material remained (LC/MS). The mixture was concentrated. The resulting residue was partitioned between DCM (130 mL) and sat. NaHCO₃ (50 mL). The organic layer was washed with sat. NaHCO₃ (2×50 mL), water (50 mL), 5% citric acid (2×50 mL), then brine (50 mL). The organic layer was then dried over anh. Na₂SO₄, and concentrated to yield product (1599 mg, 93%) as white solid. Used further without additional purification. ¹H NMR (300 MHz, CD₃OD-d₄) δ: 7.43-7.24 (m, 5H), 5.16-5.01 (m, 2H), 4.06 (s, 1H), 1.42 (s, 9H).

### Step 2. Synthesis of (N6-Boc-3,3,4,4,5,5,6,6-d8)-Lys-NH2

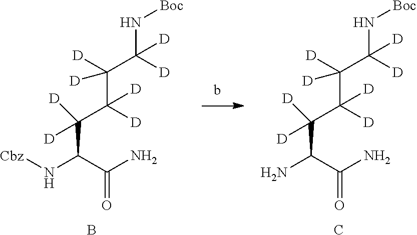

(N2-Cbz-N6-Boc-3,3,4,4,5,5,6,6-d8)-L-lysine-amide (B, 1641 mg, 4.23 mmol) was dissolved in MeOH (148 mL), and 10% Pd/C (113 mg, 0.025 equiv.) was added to this mixture. Hydrogen gas was bubbled for 3 h (TLC control: DCM-MeOH, 10:1, R_{f}(SM) 0.6, R_{f}(PR) 0.1-0.2). Then reaction mixture was filtered through Celite® and evaporated to give crude product as a white solid (1072 mg, 99%). Used further without additional purification. ¹H NMR (300 MHz, CD3OD-d₄) δ: 3.35 (s, 1H), 3.28 (s, 1H), 1.43 (s, 9H).

### Step 3. Synthesis of (Boc)-Phe-D-Arg-Phe-(N6-Boc-3,3,4,4,5,5,6,6-d8)-Lys-NH2

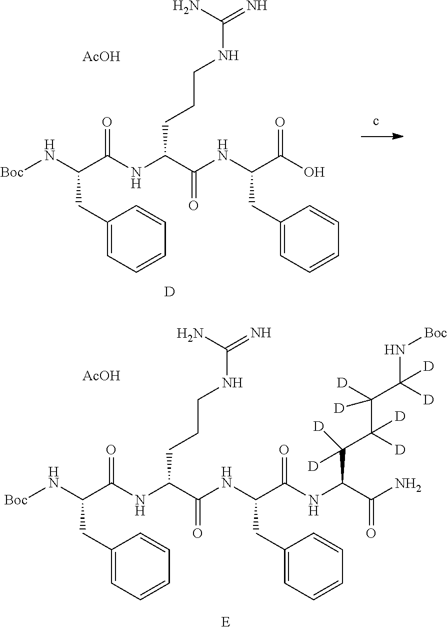

To a (Boc)-Phe-D-Arg-Phe (D, 2.66 g, 4.23 mmol), (N6-Boc-3,3,4,4,5,5,6,6-d8)-Lys-NH₂ (C, 1.07 g, 4.23 mmol) and HOBt (686 mg, 5.08 mmol) in dry DMF (34 mL) was added EDC.HCl (1.46 g, 7.61 mmol) at 0° C. and allowed to stir at r.t. for 20 hours. Then DMF was evaporated, and crude product was washed with Et₂O (3×3 mL), dissolved in MeOH, added Celite® and evaporated. Product purified by reverse phase flash chromatography (eluent: H₂O (0.2% AcOH)/MeOH from 5% to 85% of methanol) to yield product as a white foam (2.54 g, 69%). ¹H NMR (300 MHz, CD3OD-d₄) δ: 7.34-7.15 (m, 10H), 4.57 (dd, J=9.3, 5.6 Hz, 1H), 4.33-4.22 (m, 2H), 4.14 (t, J=6.8 Hz, 1H), 3.27-3.18 (m, 1H), 3.08-2.80 (m, 5H), 1.70-1.52 (m, 1H), 1.50-1.26 (m, 1H), 1.40 (s, 18H), 1.18-0.91 (m, 2H).

### Step 4. Synthesis of Phe-D-Arg-Phe-(3,3,4,4,5,5,6,6-d8)-Lys-NH2 (Example 1)

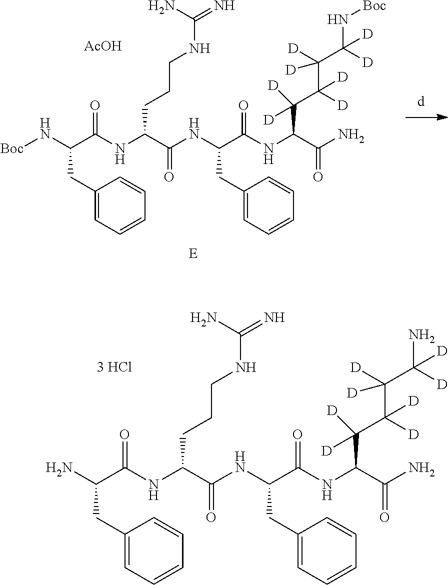 [SMILES: "AcOH" ; [2H]C([2H])(NC(=O)OC(C)(C)C)C([2H])([2H])C([2H])([2H])C([2H])([2H])[C@H](NC(=O)[C@H](Cc1ccccc1)NC(=O)[C@@H](CCCNC(=N)N)NC(=O)[C@H](Cc1ccccc1)NC(=O)OC(C)(C)C)C(N)=O ; [2H]C([2H])(N)C([2H])([2H])C([2H])([2H])C([2H])([2H])[C@H](NC(=O)[C@H](Cc1ccccc1)NC(=O)[C@@H](CCCNC(=N)N)NC(=O)[C@@H](N)Cc1ccccc1)C(N)=O]

(Boc)-Phe-D-Arg-Phe-(N6-Boc-3,3,4,4,5,5,6,6-d8)-Lys-NH2 (E, 2.54 g, 2.94 mmol) was dissolved in DCM (45 mL) and cooled to 0° C., TFA (4.5 mL, 2.66 mmol) was added dropwise and the solution was allowed to stir at 0° C. for 3 h (LC/MS shows no starting material). Then reaction mixture was evaporated (at 0-5° C.) and additionally re-evaporated from DCM (20 mL, at 0-5° C.). Purification by reverse phase flash chromatography (eluent: H₂O (HCl, pH 4)/MeOH from 2% to 85% of methanol) gave 1.98 g (94%) of product Phe-D-Arg-Phe-(3,3,4,4,5,5,6,6-d8)-Lys-NH2 hydrochloride salt. ¹H NMR (300 MHz, CD₃OD-d₄) δ: 7.39-7.17 (m, 10H), 4.62 (dd, J=11.0, 4.4 Hz, 1H), 4.40 (t, J=4.1 Hz, 1H), 4.13 (q, J=7.3 Hz, 2H), 3.40-3.33 (m, 1H), 3.22-3.05 (m, 2H), 3.02-2.84 (m, 3H), 1.53-1.23 (m, 2H), 1.17-0.84 (m, 2H). Molecular formula: C₃₀H₃₇D₈N₉O₄ 3HCl; Molecular weight: 713.17; Free base molecular weight: 603.80. EI-MS: m/z 604.5 [M+1].

### Example 2: Synthesis of Phe-D-Arg-Phe-(4,4,5,5-d4)-Lys-NH2 Trifluoroacetate

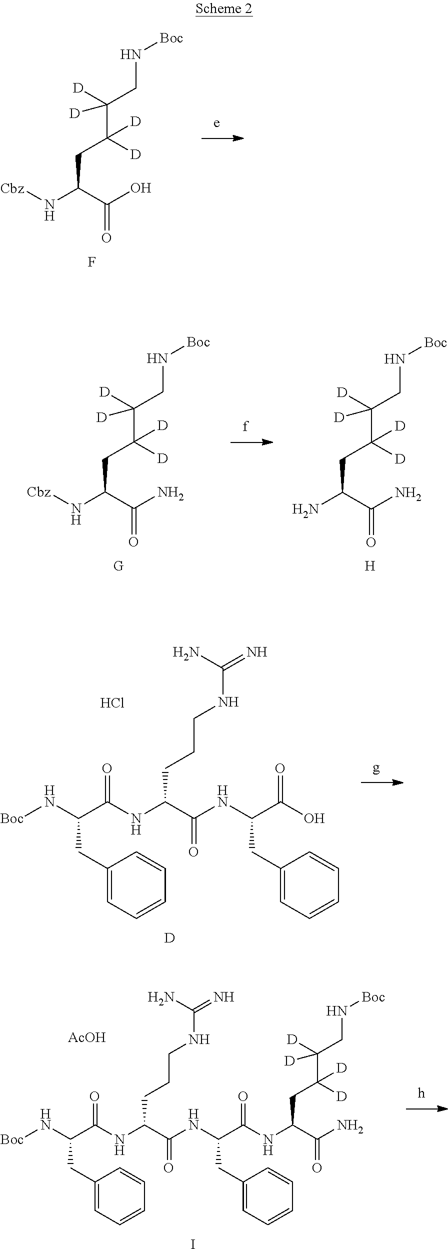

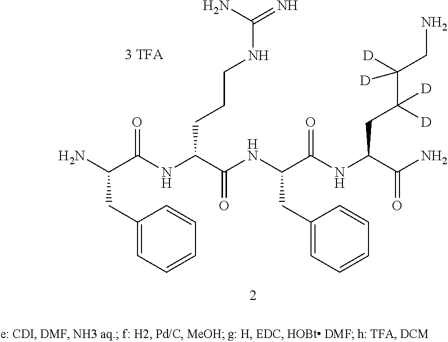 [SMILES: [2H]C([2H])(CN)C([2H])([2H])C[C@H](NC(=O)[C@H](Cc1ccccc1)NC(=O)[C@@H](CCCNC(=N)N)NC(=O)[C@@H](N)Cc1ccccc1)C(N)=O]

### Step 1. Synthesis of (N2-benzyloxycarboxyl-N6-tert-butoxycarboxyl-4,4,5,5-d4)-Lys-NH₂

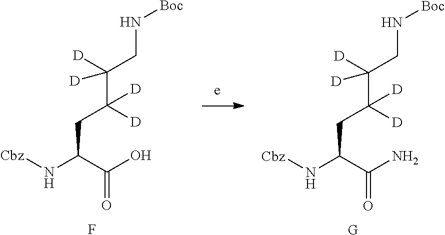

To a stirring solution of (N2-Cbz-N6-Boc-4,4,5,5-d4)-Lys-OH (F, 384 mg, 1.0 mmol) in dry DMF (5 mL) was added carbonyldiimidazole (162 mg, 1.3 mmol) in one portion. The mixture stirred for 1.5 h at rt then aqueous ammonia (0.10 mL, 1.5 mmol, 14.8 M) was added drop-wise. After stirring an additional 3 h, no starting material remained (LC/MS). The mixture was concentrated. The resulting residue was partitioned between DCM (30 mL) and sat. NaHCO₃ (12 mL). The organic layer was washed with sat. NaHCO₃ (2×12 mL), water (12 mL), 5% citric acid (2×12 mL), then brine (12 mL). The organic layer was then dried over anh. Na₂SO₄, and concentrated (TLC, DCM-MeOH (10:1) R_{f}(PR) 0.5). (N2-Cbz-N6-Boc-4,4,5,5-d4)-Lys-OH (376 mg, 98%), which was used without further purification. ¹H NMR (300 MHz, CD₃OD-d₄) δ: 7.43-7.20 (m, 5H), 5.17-5.00 (m, 2H), 4.07 (dd, J=9.1, 5.0 Hz, 1H), 3.01 (s, 2H), 1.69 (ddd, J=22.8, 13.6, 7.0 Hz, 2H), 1.42 (s, 9H).

### Step 2. Synthesis of (N6-tert-butoxycarboxyl-4,4,5,5,6,6-d8)-Lys-NH2

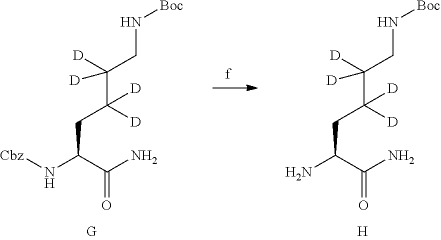

(N2-benzyloxycarboxyl-N6-tert-butoxycarboxyl-4,4,5,5-d4)-Lys-NH2 (G, 376 mg, 0.98 mmol) was dissolved in MeOH (36 mL) and 10% Pd/C (26 mg, 0.025 equiv.) was added. Hydrogen gas was bubbled for 3 h (TLC control: DCM-MeOH, 10:1, R_{f}(SM) 0.5, R_{f}(PR) 0.1-0.2). Then reaction mixture was filtered through Celite® and evaporated to give crude product as a white amorphous solid (237 mg, 97%). Used further without additional purification. ¹H NMR (300 MHz, CD3OD-d₄) δ: 3.35 (s, 1H), 3.02 (s, 2H), 1.60 (ddd, J=20.6, 13.5, 6.6 Hz, 2H), 1.43 (s, 9H).

### Step 3. Synthesis of (Boc)-Phe-D-Arg-Phe-(N6-Boc-3,3,4,4,5,5,6,6-d8)-Lys-NH2

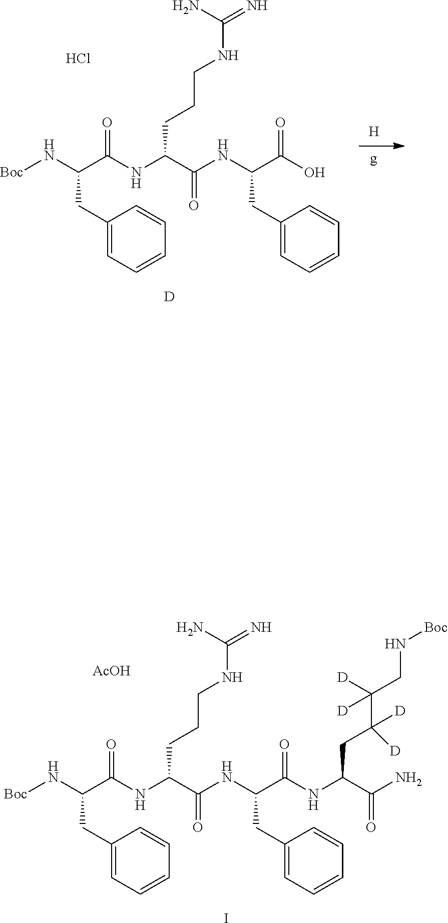

Tripeptide (D, 300 mg, 0.477 mmol), (N6-tert-butoxycarboxyl-4,4,5,5,6,6-d8)-Lys-NH₂ (H, 121 mg, 0.477 mmol), HOBt (90 mg, 0.668 mmol) and EDC.HCl (165 mg, 0.859 mmol) were mixed in mixture of dry DMF (3.8 mL) at 10° C. and allowed to warm to r.t. Reaction mixture continued stirring at r.t. for 20 hours. Then DMF was evaporated. Crude product was washed with Et₂O (3×3 mL), dissolved in MeOH, added Celite and evaporated. Product purified by reverse phase flash chromatography (eluent: H₂O (0.2% AcOH)/MeOH from 5% to 85% of methanol), to give product (243 mg, 59%) as a white foam.

### Step 4. Synthesis of Phe-D-Arg-Phe-(4,4,5,5-d4)-Lys-NH2 Trifluoroacetate (Example 2)

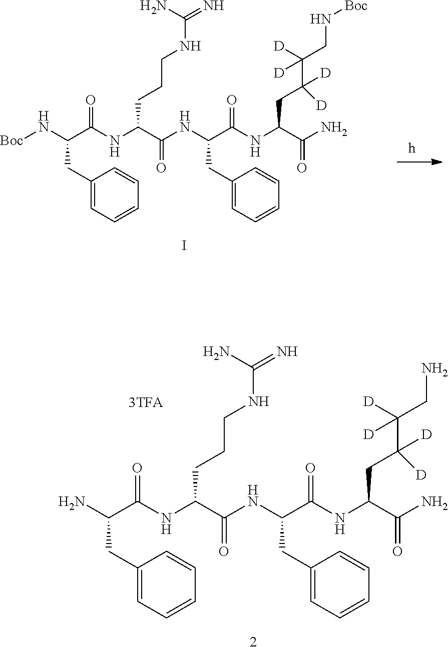

(Boc)-Phe-D-Arg-Phe-(N6-Boc-3,3,4,4,5,5,6,6-d8)-Lys-NH2 (I, 243 mg, 0.283 mmol) was dissolved in DCM (2.8 mL) and cooled to 0° C. TFA (0.87 mL, 11.32 mmol) was added drop-wise and the solution was allowed to stir at 0° C. for 3 h. Then reaction mixture was evaporated (at 0-5° C.) and additionally re-evaporated from DCM (4 mL, at 0-5° C.). LC/MS shows no starting material. Purification on PHPLC gave product (100 mg, HPLC purity: 99.7%) as colorless foam. ¹H NMR (400 MHz, CD3OD-d₄) δ: 7.36-7.17 (m, 10H), 4.62 (dd, J=11.0, 4.5 Hz, 1H), 4.41 (dd, J=9.0, 5.4 Hz, 1H), 4.16-4.03 (m, 2H), 3.37-3.32 (m, 1H), 3.18-3.06 (m, 2H), 3.00-2.81 (m, 5H), 1.83 (ddd, J=22.7, 13.7, 7.2 Hz, 2H), 1.47-1.24 (m, 2H), 1.10-0.83 (m, 2H). Molecular formula: C₃₀H₄₁D₄N₉O₄.3TFA; Molecular weight: 941.84; Free base molecular weight: 599.77. EI-MS: m/z 600.6 [M+1].

### Example 3: Synthesis of Phe-D-Arg-Phe-(2,5,5-d3)-Lys-NH2 Trifluoroacetate

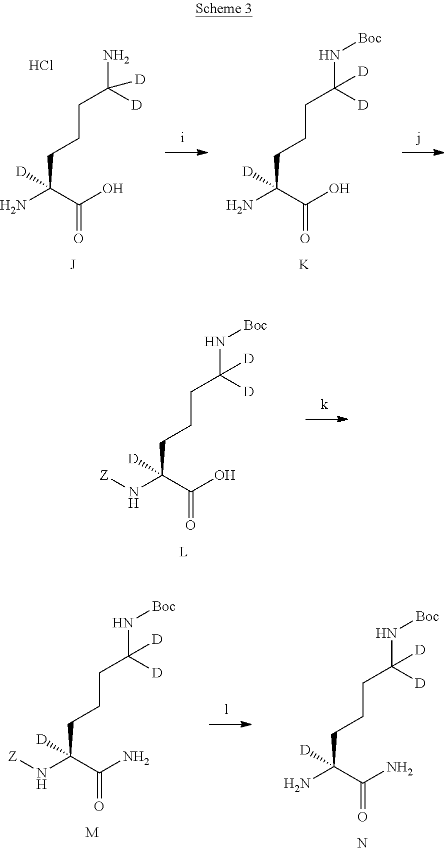

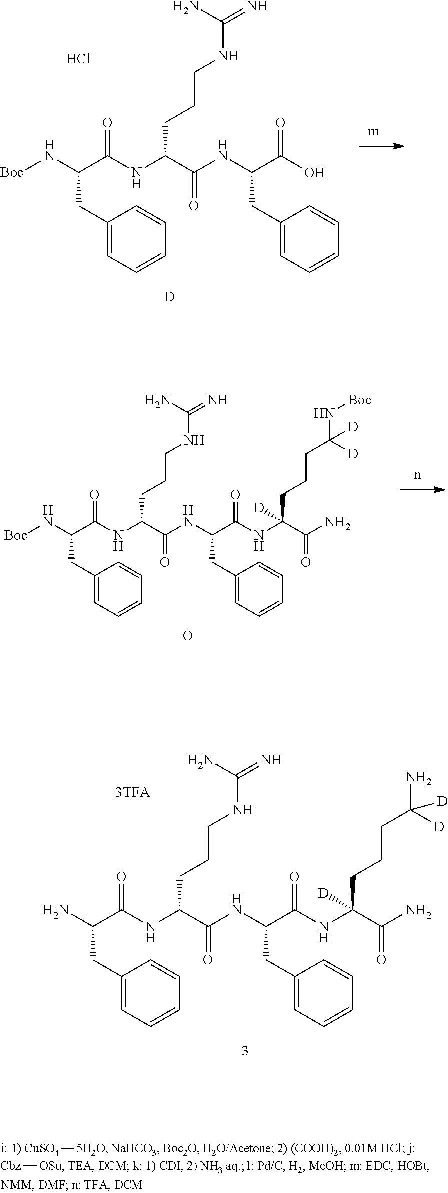

### Step 1: Synthesis of (N6-Boc-2,5,5-d3)-Lys

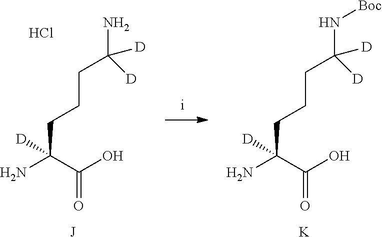

L-(2,5,5-d3)-Lysine hydrochloride (J, 1.30 mmol, 241 mg) was dissolved in 2 M NaHCO₃ (1.30 mL, 2.6 mmol, 218 mg), to which a solution of CuSO₄x5H₂O (0.65 mmol, 162 mg) in H₂O (1.30 mL) was added. An additional NaHCO₃ (109 mg, 1.30 mmol) was added, followed by ditertbutyldicarbonate (1.43 mmol, 312 mg) dissolved in 1.6 mL acetone. The mixture stirred 24 h, then methanol (0.50 mL) was added to the solution, and stirring continued 14 h. To the reaction mixture H₂O (1 mL) is added and the product was subsequently filtered, washed with H₂O (3×1 mL) and dried to give 286 mg (79% yield) of [(N6-Boc-2,5,5-d3)-lysine]2Cu as pale blue solid. To remove the copper, the pale blue Cu-chelate (286 mg, 0.511 mmol) was suspended in H₂O (11 mL). Thereafter, 8-quinolinol (200 mg, 1.38 mmol) was added, and the mixture stirred 20 h (pale salad-green precipitate formation). The suspension was filtered, precipitates washed with H₂O (3×4 mL) and the filtrate washed with EtOAc (3×8 mL). The aqueous layer was evaporated to yield 141 mg (55% yield) of L-(N6-Boc-2,5,5-d3)-lysine as a white solid. Overall yield—43%. ¹H NMR (400 MHz, Methanol-d₄) δ 1.93-1.71 (m, 2H), 1.43-1.35 (m, 4H), 1.43 (s, 9H).

### Step 2: Synthesis of (N2-Cbz-N6-Boc-2,5,5-d3)-Lys

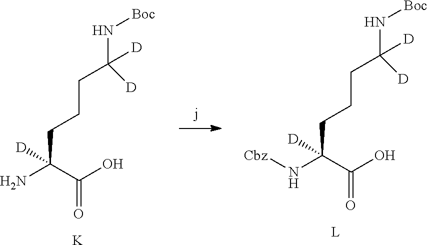

To a suspension of (N6-Boc-2,5,5-d3)-lysine (K, 141 mg, 0.566 mmol) in mixture of dioxane (3.7 mL) and water (3.7 mL), NaHCO₃ (238 mg, 2.83 mmol) and then N-(Benzyloxycarbonyloxy)succinimide (169 mg, 0.678 mmol) was added. Biphasic mixture stirred at room temperature for 20 h. Then additional water (2.2 mL) was added and reaction mixture was extracted with Et₂O (3×6 mL). Then to the aqueous layer was added 5% solution of citric acid (7.5 mL) and product was extracted with DCM (3×8 mL). Organic layers were combined, dried on Na₂SO₄, filtered and evaporated to give product as colorless oil (168 mg, 770%).

### Step 3: Synthesis of (N2-Cbz-N6-Boc-2,5,5-d3)-Lys-NH2

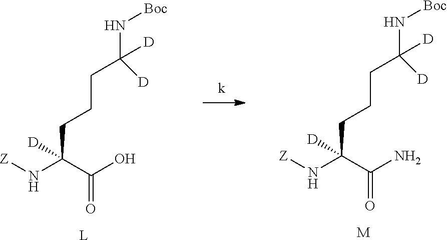

To the mixture of (N2-Cbz-N6-Boc-2,5,5-d3)-lysine (L, 293 mg, 0.765 mmol) in DMF (5 mL) carbonyl diimidazole (CDI) (186 mg, 1.47 mmol) was added. Reaction mixture was stirred at rt for 0.5 h. Then 25% NH₄OH (1.5 mL) was added and stirred at rt overnight. To the reaction mixture 5% NaHCO₃ aq. solution (3 mL) and DCM (5 mL) were added. The organic layer was separated and washed with 5% KHSO₄ aq. solution (7 mL), brine (7 mL). DCM was removed to afford (N2-Cbz-N6-Boc-2,5,5-d3)-lysine amide as white solid (262 mg, 89%). Product was used without further purification.

### Step 4: Synthesis of (N6-Boc-2,5,5-d3)-Lys-NH2

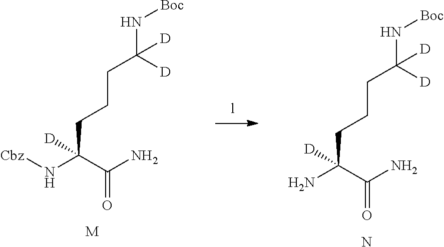

To a solution of (N2-Cbz-N6-Boc-2,5,5-d3)-lysine amide (M, 234 mg, 0.611 mmol) in MeOH (25 mL) Pd/C (50% w/w, 120 mg) was added Pd/C (50% w/w, 120 mg). The hydrogen was purged in reaction mixture at rt for 3 h. Then reaction mixture was filtered through a Celite pad and washed with MeOH (10 mL). The solvent was removed by evaporation. It was obtained white powder of (N6-Boc-2,5,5-d3)-lysine amide (146 mg, 96%). Product was used without further purification.

### Step 5: Synthesis of (Boc)-Phe-D-Arg-Phe-(N6-Boc-2,5,5-d3)-Lys-NH2

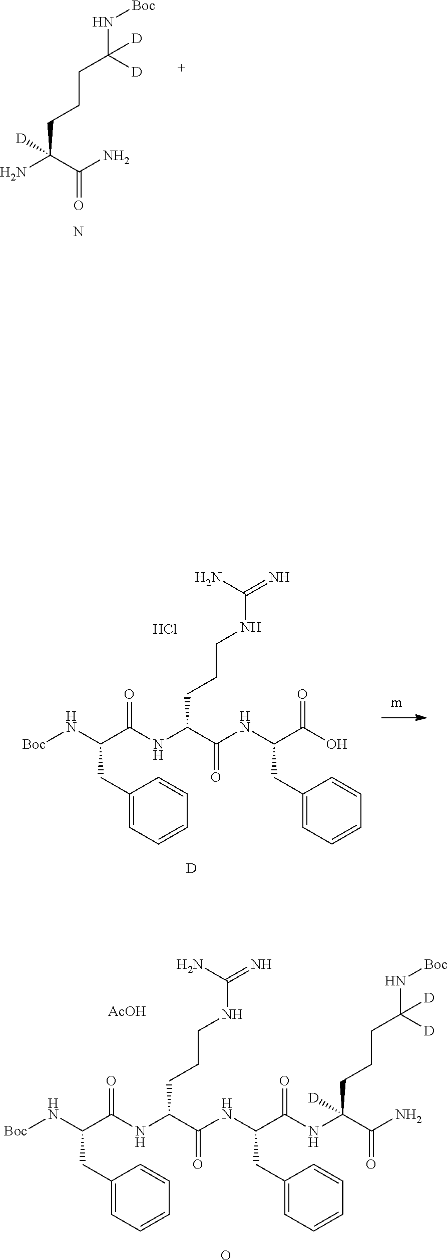

To mixture of (N6-Boc-2,5,5-d3)-lysine amide (N, 100 mg, 0.403 mmol) and Boc-Phe-D-Arg-Phe-OH (D, 268 mg, 0.443 mmol) in DMF (10 mL) were added HOBT*H₂O (74 mg, 0.483 mmol), EDC*HCl (109 mg, 0.604 mmol) and NMM (66 μl, 0.604 mmol). Reaction mixture was stirred at rt. After 48 h DMF was removed. Crude product was purified by reverse phase flash chromatography (15-65% MeOH in H₂O, 0.1% AcOH) to afford (Boc)-Phe-D-Arg-Phe-(N6-Boc-2,5,5-d3)-Lys-NH2 white foam (160 mg, purity—70%). Additional purification was performed by HPLC (Waters SFO, column: Xterra Prep RP18 OBD 10 um, 19×150 mm, 20 mL/min, water (0.05% TFA)/MeOH, solvent gradient from 2% to 10% of MeOH]. (Boc)-Phe-D-Arg-Phe-(N6-Boc-2,5,5-d3)-Lys-NH2 was isolated as a white solid (63 mg, impurities<8%). Additional purification was performed by HPLC. The desired product was isolated as a white solid (44 mg, impurities<5%). ¹H NMR (400 MHz, Methanol-d₄) δ 7.35-7.13 (m, 10H), 4.56 (dd, J=9.6, 5.4 Hz, 1H), 4.27 (m, 1H), 4.14 (m, 1H), 3.23 (d, J=5.4 Hz, 1H), 3.10-2.85 (m, 5H), 1.91-1.67 (m, 2H), 1.42 (s, 9H), 1.39 (s, 9H), 1.66-1.27 (m, 7H), 1.07 (s, 1H).

### Step 6: Synthesis of Phe-D-Arg-Phe-(2,5,5-d3)-Lys-NH2 Trifluoroacetate (Example 3)

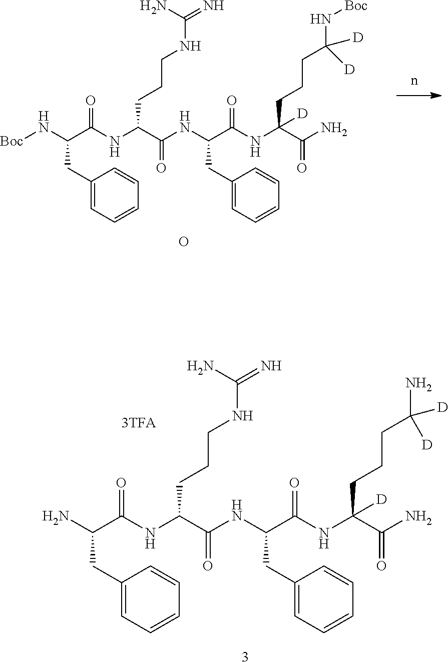

To a cooled (0° C.) solution of (Boc)-Phe-D-Arg-Phe-(N6-Boc-2,5,5-d3)-Lys-NH2 (0, 44 mg) in DCM (5 mL) TFA (1.0 mL) was added. After 5 min, the ice bath was removed and the mixture stirred at ambient temperature for 2 h. Volatiles was removed under reduced pressure. It was obtained 36 mg of Phe-D-Arg-Phe-(2,5,5-d3)-Lys-NH2 trifluoroacetate. ¹H NMR (400 MHz, Methanol-d₄) δ 7.41-7.14 (m, 10H), 4.63 (dd, J=11.0, 4.4 Hz, 1H), 4.15-4.06 (m, 2H), 3.39-3.32 (m, 1H), 3.12 (qd, J=13.7, 7.6 Hz, 2H), 2.92 (td, J=7.0, 3.4 Hz, 2H), 2.85 (dd, J=14.1, 11.0 Hz, 1H), 1.93-1.63 (m, 4H), 1.57-1.37 (m, 3H), 1.36-1.23 (m, 1H), 1.04 (dt, J=11.9, 6.2 Hz, 1H), 0.89 (tt, J=13.1, 6.7 Hz, 1H). Molecular formula: C₃₀H₄₂D₃N₉O₄.3TFA; Molecular weight: 940.84; Free base molecular weight: 598.77. EI-MS: m/z 599.7 [M+1].

### Example 4: Synthesis of Synthesis of Phe-D-Arg-Phe-(2-d)-Lys-NH2

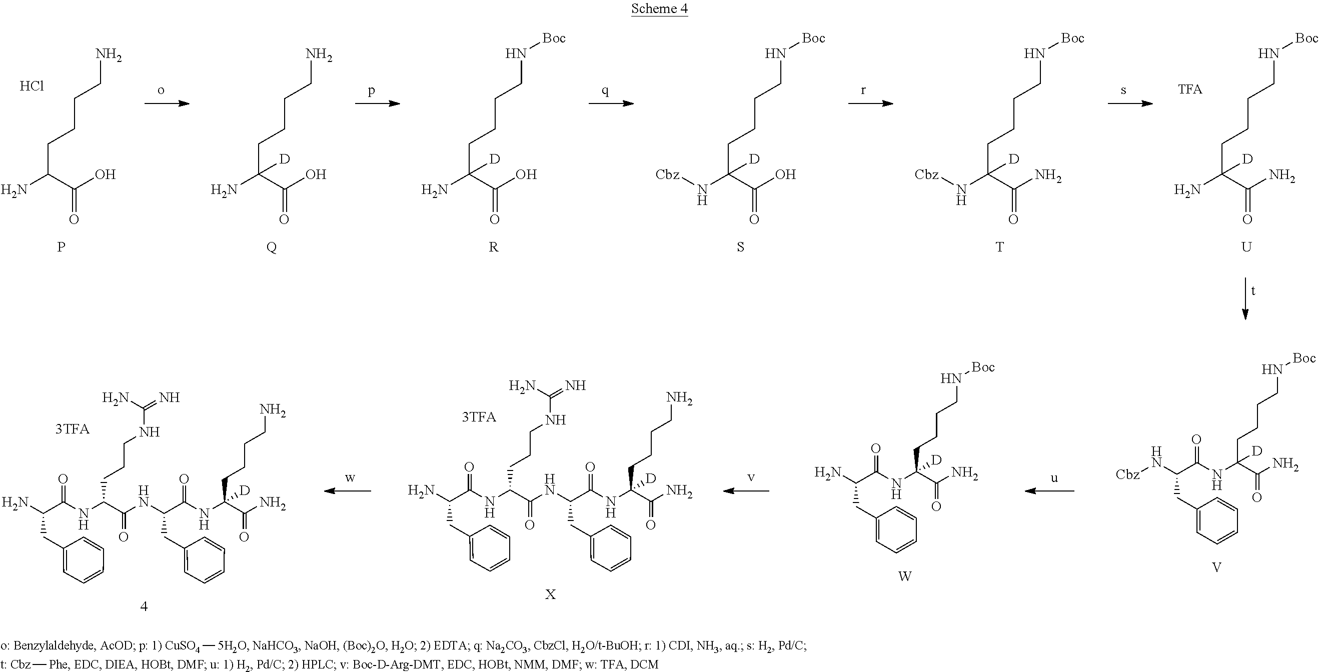

### Step 1: Synthesis of DL-(2-d)-Lysine

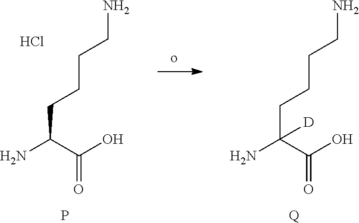

L-lysine (1.8 g), was dissolved in D₂O (4 mL) and evaporated to dryness, then acetic acid-dl (CH₃COOD, 22.5 mL) and benzaldehyde (0.08 mL) were added and the flask was fitted with a reflux condenser. The resulting mixture was thoroughly purged with nitrogen and then refluxed for 1 h. The resulting solution was evaporated under reduced pressure to give compound dl-(2-d)-lysine (1.8 g, 99%). ¹H NMR (300 MHz, D₂O) δ: 3.43-3.38 (m, 2H), 2.40-2.21 (m, 2H), 2.12-2.06 (m, 2H), 1.96-1.83 (m, 2H).

### Step 2: Synthesis of dl-(N6-Boc-2-d)-lysine

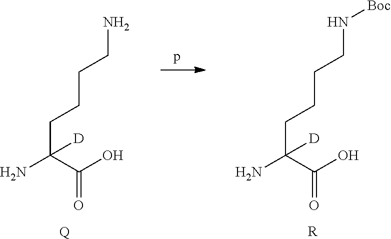

DL-(2-d)-lysine (Q, 0.96 g 5.2 mmol) was dissolved in 2 M NaHCO₃ (5.2 mL, 10.4 mmol, 872 mg), to which a solution of CuSO₄x5H₂O (2.6 mmol, 648 mg) in H₂O (5.2 mL) was added. An additional NaHCO₃ (438 mg, 5.2 mmol) was added, followed by Boc₂O (5.72 mmol, 1.25 g) dissolved in 6.5 mL acetone. The mixture stirred 24 h, then methanol (3 mL) was added to the solution, and stirring continued 14 h. To reaction mixture H₂O (5 mL) was added and the product was subsequently filtered, washed with H₂O (3×1 mL) and dried to give Cu[lys(D3)^{ε}(Boc)]₂ as pale blue solid. To remove the copper, the pale blue Cu-chelate was suspended in H₂O (25 mL). Thereafter, EDTA (800 mg, 5.52 mmol) was added, and the mixture stirred 20 h. The suspension was filtered, precipitates washed with H₂O (3×4 mL) and the filtrate washed with EtOAc (3×8 mL). The aqueous layer was evaporated to yield 440 mg of dl-(N6-Boc-2-d)-lysine as a white solid. Overall yield—34%. ¹H NMR (300 MHz, D₂O) δ: 3.09-2.96 (m, 2H), 1.88-1.78 (m, 2H), 1.56-1.32 (m, 4H), 1.40 (s, 9H).

### Step 3: Synthesis of dl-(N2-Cbz-N6-Boc-2-d)-lysine

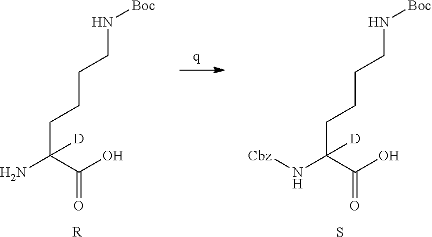

To a suspension of dl-(N6-Boc-2-d)-lysine (R, 440 mg, 1.76 mmol) in mixture of dioxane (15 mL) and water (12 mL), NaHCO₃ (742 mg, 8.82 mmol) and then N-(Benzyloxy-carbonyloxy)succinimide (527 mg, 2.11 mmol) was added. Biphasic mixture stirred at rt for 20 h. Then additional water (7 mL) was added and reaction mixture was extracted with Et₂O (3×6 mL). Then to the aqueous layer was added 5% solution of citric acid (20 mL) and product was extracted with DCM (3×15 mL). Organic layers were combined, dried on Na₂SO₄, filtered and evaporated to give product as colorless oil (468 mg, 68%). ¹H NMR (300 MHz, CDCl₃) δ: 7.48-7.32 (m, 5H), 5.1 (s, 2H), 3.28-3.17 (m, 2H), 1.87-1.68 (m, 2H), 1.56-1.32 (m, 4H), 1.51 (s, 9H).

### Step 4: Synthesis of dl-(N2-Cbz-N6-Boc-2-d)-Lys-NH2

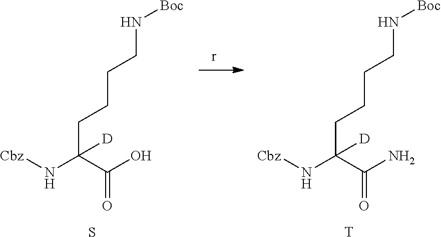

Compound dl-(N2-Cbz-N6-Boc-2-d)-lysine (S, 400 mg, 1 mmol) and CDI (210 mg, 1.3 mmol) were added to a round-bottom flask and the flask was filled with argon. 10 mL of dry DMF were added and the mixture was stirred for 0.5 h at ambient temperature. 25% Aqueous ammonia (2 mL) was added and the mixture was stirred overnight at ambient temperature. The mixture was diluted with 50 mL DCM, the solution was washed with 5% aq. NaHCO₃ (20 mL), water (20 mL), 5% aq. KHSO₄ (20 mL) and brine (20 mL). The organic layer was dried over anh. Na₂SO₄ and dried via oil pump to remove DMF and yield compound dl-(N2-Cbz-N6-Boc-2-d)-Lys-NH2. It was used in next step without purification. ¹H NMR (300 MHz, CDCl₃) δ: 7.46-7.38 (m, 5H), 6.1 (br s, 1H), 5.56 (br d, 2H), 5.1 (s, 2H), 4.65-4.59 (m, 1H); 3.18-3.09 (m, 2H), 1.81-1.38 (m, 6H), 1.40 (s, 9H).

### Step 5: Synthesis of dl-(N6-Boc-2-d)-Lys-NH2

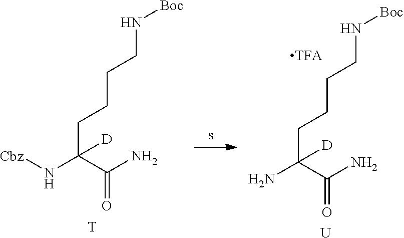

Compound dl-(N2-Cbz-N6-Boc-2-d)-lysine amide (T, 400 mg, 1.0 mmol) was added to a round-bottom flask and dissolved in 50 mL MeOH. 20 mg of 10% Pd(C) were added and hydrogen was bubbled through a stirred solution for 30 min. The mixture was left to stir under hydrogen atmosphere for 2 hours. Reaction control was maintained with LC-MS, end point was determined by disappearance of signal of compound dl-(N6-Boc-2-d)-lysine amide. Mixture was then filtrated through Celite and evaporated to dryness.

### Step 6: Synthesis of (Cbz)-Phe-DL-(2-d)-Lys-NH2

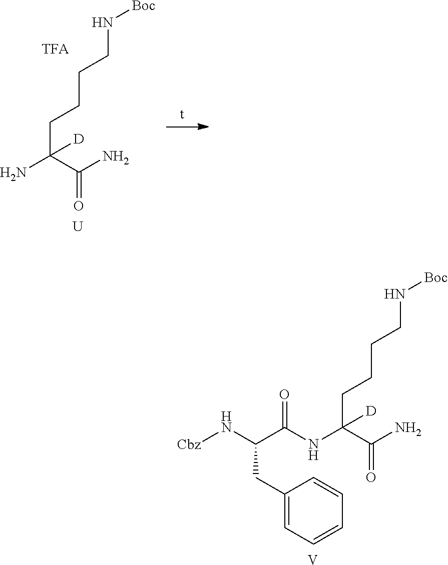

HOBt (340 mg, 2.2 mmol), EDC.HCl (0.42 g, 2.2 mmol) and L-Cbz-Phe-OH (660 mg, 2.2 mmol) were added to the compound dl-(N6-Boc-2-d)-lysine amide. The mixture was put under an argon atmosphere and dissolved in 8 mL dry DMF and stirred overnight. After that reaction mixture was diluted with 100 mL EtOAc, washed with 5% citric acid (3×20 mL), sat. NaHCO₃ (3×20 mL) and sat. NaCl (20 mL). The organic phase was dried on Na₂SO₄ evaporated and dried via an oil pump to remove traces of DMF. Purification was done via flash chromotography (DCM/MeOH), to yield white solid (600 mg, 57%).

### Step 7: Synthesis of L-Phe-L-(N6-Boc-2-d)-Lys-NH2

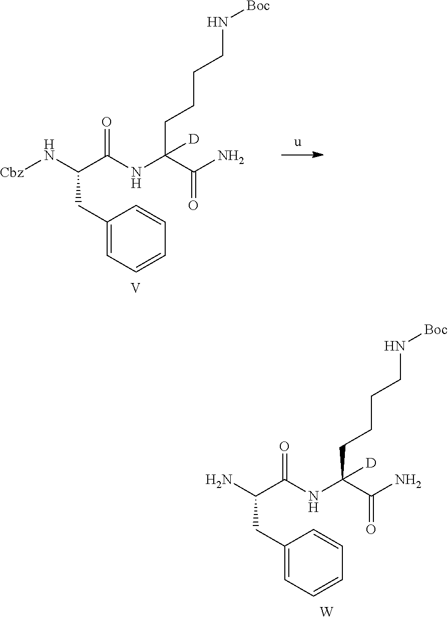

Compound L-(Cbz)-Phe-DL-(N6-Boc-2-d)-lysine amide (V, 600 mg) was added to a round-bottom flask and dissolved in 50 mL MeOH. 40 mg of 10% Pd(C) were added and hydrogen was bubbled through a stirred solution for 30 min. The mixture was left to stir under hydrogen atmosphere for 2 hours. Reaction control was maintained with LC-MS, end point was determined by disappearance of signal of compound L-(Cbz)-Phe-DL-(N6-Boc-2-d)-lysine amide. Mixture was then filtrated through Celite and evaporated to dryness. The diastereomers were separated using preparative HPLC (column: XTerra Prep RP18 OBD, 10 μm, 19×150 mm; mobile phase: water (+0.1% AcOH)/MeOH; flowrate: 20 mL/min). To detect correct isomer the analogue of LIOS-010-8 (hydrogen instead of deuterium) was synthesized and analysed using HPLC (column: XTerra RP18 5 μm, 4.6×150 mm; mobile phase: water (+0.1% TFA)/MeOH; flowrate: 1 mL/min; retention time: 11.71 min; method: 10%-95% MeOH in 18 min). ¹H NMR (400 MHz, CD3OD-d₄) δ: 7.38-7.29 (m, 5H), 4.15 (dd, J=5.6 Hz, J=14.0 Hz, 1H), 3.34-3.025 (m, 1H), 3.06-3.00 (m, 3H), 1.84-1.77 (m, 1H), 1.71-1.64 (m, 1H), 1.52-1.36 (m, 4H), 1.42 (s, 9H).

### Steps 8: Synthesis of Phe-D-Arg-Phe-(2-d)-Lys-NH2 Trifluoroacetate (Example 4)

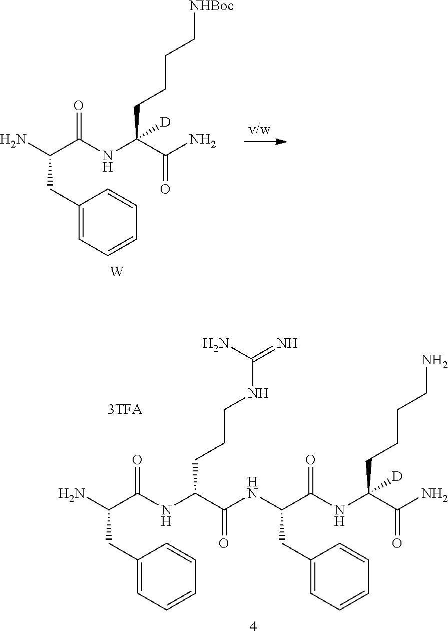

To a mixture of Phe-L-(N6-Boc-2-d)-lysine amide (W, 250 mg, 0.635 mmol), dipeptide (Boc)-Phe-D-Arg-OH (268 mg, 0.635 mmol), HOBt monohydrate (195 mg, 1.27 mmol), EDC hydrochloride (244 mg, 1.27 mmol) in DMF (5 mL) was added NMM (0.28 mL, 2.54 mmol) at 0° C. After 5 min ice bath was removed and the reaction mixture was stirred overnight at RT. Additional 1 equiv. of NMM (0.07 mL, 0.635 mmol) was added and the reaction mixture was stirred for 2 days, then evaporated and purified by flash column chromatography (eluent H₂O (0.1% AcOH)/MeOH). Additional purification was performed by preparative HPLC (column: XTerra Prep RP18 OBD, 10 μm, 19×150 mm; mobile phase: water (+0.1% AcOH)/MeOH; flowrate: 20 mL/min) and 40 mg of Boc protected tetrapeptide was isolated. To a solution of isolated tetrapeptide (40 mg, 0.050 mmol) in DCM (1 mL) was added TFA (0.5 mL) at 0° C. After 5 min ice bath was removed and the reaction mixture was stirred 60 min at ambient temperature. The volatiles were evaporated and the crude product was purified by preparative HPLC (mobile phase: water (0.1% TFA)/MeOH (0.1% TFA)). 28 mg of Phe-D-Arg-Phe-(2-d)-Lys-NH2 trifluoroacetate was isolated as a white solid (HPLC purity>96%). ¹H NMR (400 MHz, CD₃OD) δ 7.41-7.16 (m, 10H), 4.63 (dd, J=11.1, 4.4 Hz, 1H), 4.17-4.03 (m, 2H), 3.40-3.32 (m, 1H), 3.12 (dd, J=12.4, 7.6 Hz, 2H), 3.01-2.79 (m, 6H), 1.98-1.64 (m, 4H), 1.59-1.24 (m, 5H), 1.14-0.80 (m, 2H). Molecular formula: C₃₀H₄₄DN₉O₄x3TFA; Molecular weight: 938.82; Free base molecular weight: 596.76. EI-MS: m/z 597.7 [M+1].

### Example 5: Synthesis of Phe-D-Arg-(α-d)-Phe-Lys-NH2 Hydrochloride

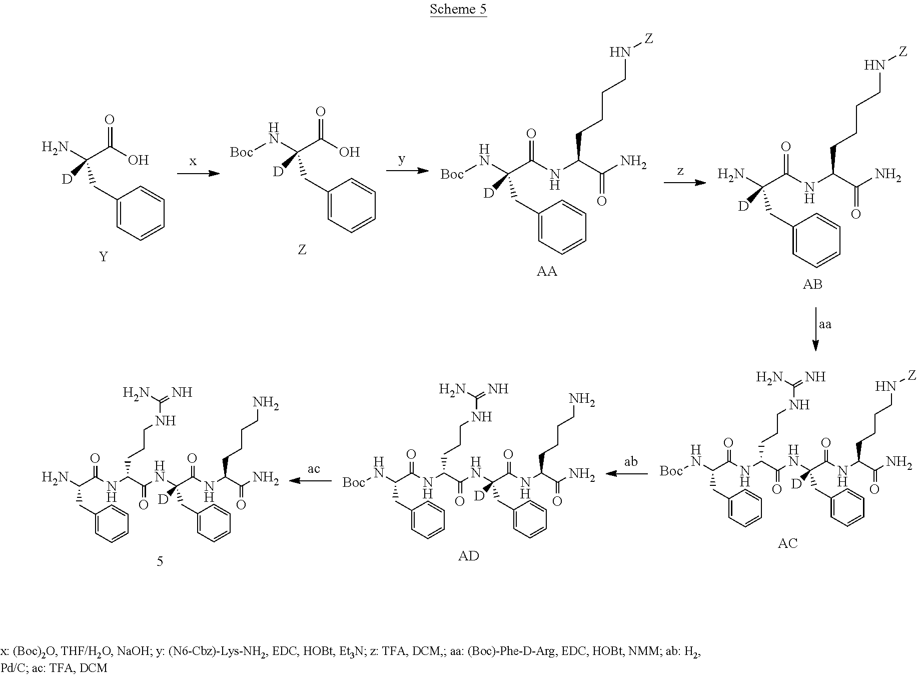

### Step 1: Synthesis of (N-Boc-α-d)-Phe-OH

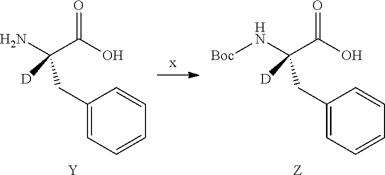

To a solution of (α-d)-Phe-OH (Y, 250 mg, 1.51 mmol) in THF/H₂O (5 mL/2.5 mL) 20% NaOH (250 μl) was added followed by Boc₂O (360 mg, 1.66 mmol). The reaction mixture was stirred at rt for 16 h (checked by LC-MS). Then THE was removed, DCM (30 mL) was added and organic layer was washed with satd. NaHCO₃, brine, dried over Na₂SO₄ and concentrated to afford (N-Boc-2-d)-Phe-OH (270 mg, 67%). Product was used without further purification. ¹H NMR (400 MHz, Chloroform-d) δ 7.34-7.23 (m, 3H), 7.23-7.15 (m, 2H), 4.91 (s, 1H), 3.19 (d, J=14.0 Hz, 1H), 3.08 (d, J=14.0 Hz, 1H), 1.42 (s, 9H).

### Step 2: Synthesis of (N-Boc-α-d)-Phe-(N6-Cbz)-Lys-NH2

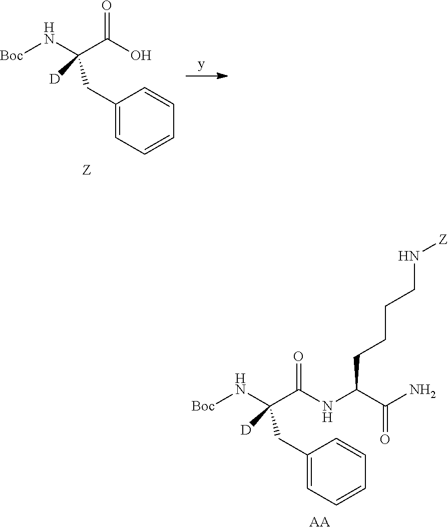

To the mixture of (Boc-α-d)-Phe (Z, 270 mg, 1.13 mmol) and (N6-Cbz)-Lys-NH2 (393 mg, 1.24 mmol) in DCM (5 mL) HOBt*H₂O (208 mg, 1.36 mmol), EDC*HCl (260 mg, 1.36 mmol) and Et₃N (472 μl, 3.39 mmol) were added. Reaction mixture was stirred at rt overnight. Then DCM was removed and crude product was purified by flash chromatography (0-5% MeOH in DCM) to afford AA as white solid (416 mg, 69%). ¹H NMR (400 MHz, Chloroform-d) δ 7.43-7.10 (m, 10H), 6.47 (s, 1H), 6.10 (s, 1H), 5.26-4.85 (m, 5H), 4.38 (td, J=8.3, 4.7 Hz, 1H), 3.31-2.95 (m, 4H), 1.89 (s, 1H), 1.69-1.43 (m, 4H), 1.42-1.38 (s, 9H), 1.30 (m, 3H).

### Step 3: Synthesis of (α-d)-Phe-(N6-Cbz)-Lys-NH2

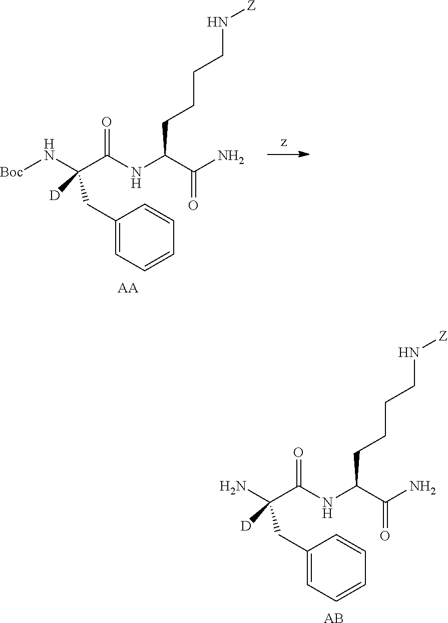

To a cooled (0° C.) solution of AA (410 mg, 0.777 mmol) in DCM (12 mL) TFA (4.0 mL) was added. After 5 min, the ice bath was removed and the mixture stirred at ambient temperature for 2 h. Volatiles were removed under reduced pressure and the residue concentrated twice with toluene. (α-d)-Phe-(N6-Cbz)-Lys-NH2 (411 mg) was obtained as white solid that was used without further purification.

### Step 4: Synthesis of (Boc)-Phe-D-Arg-(α-d)-Phe-(N6-Cbz)-Lys-NH2

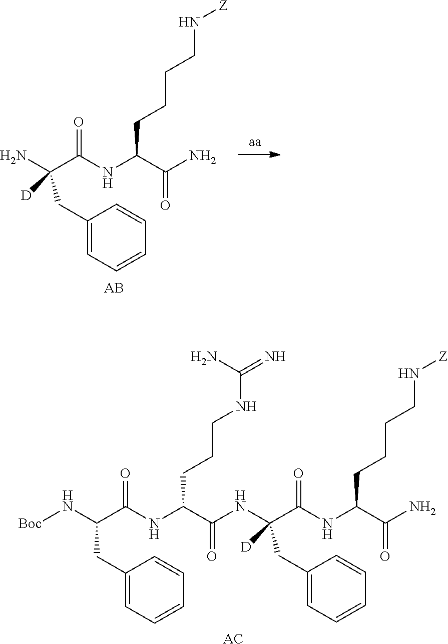

To mixture of (α-d)-Phe-(N6-Cbz)-Lys-NH2 (AB, 411 mg, 0.759 mmol) and (Boc)-Phe-D-Arg (365 mg, 0.797 mmol) in DCM (10 mL) were added HOBt*H₂O (128 mg, 0.835 mmol), EDC*HCl (160 mg, 0.835 mmol) and NMM (92 μL, 1.14 mmol). Reaction mixture was stirred at rt. After 16 h DCM was removed. Crude product was purified by reverse phase flash chromatography (20-65% MeOH in H₂O) to afford (Boc)-Phe-D-Arg-(α-d)-Phe-(N6-Cbz)-Lys-NH2 white solid (400 mg, purity—90%). Additional purification was performed by HPLC (Waters SFO, column: Xterra Prep RP18 OBD 10 um, 19×150 mm, 20 mL/min, water (0.05% TFA)/MeOH, solvent gradient from 2% to 10% of MeOH]. The desired product was isolated as a white solid (130 mg, impurities<2%). ¹H NMR (400 MHz, DMSO-d₆) δ 7.54-6.68 (m, 15H), 4.99 (s, 2H), 4.36-3.99 (m, 3H), 3.09 (d, J=13.8 Hz, 1H), 3.02-2.80 (m, 7H), 2.72 (d, J=13.3 Hz, 2H), 1.72-1.61 (m, 1H), 1.57-1.51 (m, 1H), 1.45-1.38 (m, 4H), 1.28 (s, 9H), 1.08-0.86 (m, 2H).

### Step 5: Synthesis of Phe-D-Arg-(α-d)-Phe-Lys-NH2 Acetate

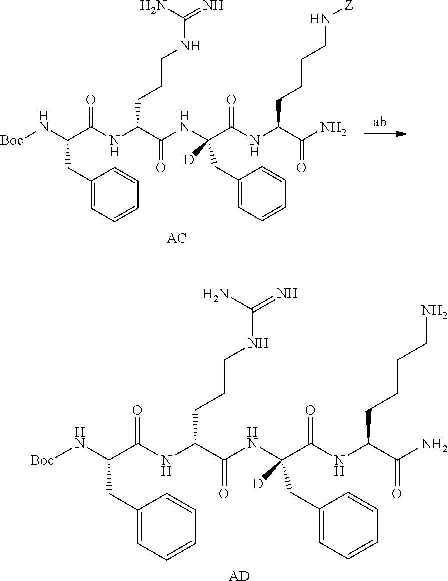

To a solution of (Boc)-Phe-D-Arg-(α-d)-Phe-(N6-Cbz)-Lys-NH2 (AC, 130 mg, 0156 mmol) in MeOH (10 mL) Pd/C (10% w/w, 15 mg) was added followed by acetic acid (36 μL, 0.625 mmol). The hydrogen was purged in reaction mixture at rt for 3 h. Then reaction mixture was filtrated through Celite pad and washed with MeOH (10 mL). The solvent was removed by evaporation followed by two co-evaporations with MeCN. It was obtained white solid (100 mg) that was used without further purification. ¹H NMR (400 MHz, Methanol-d₄) δ 7.36-7.14 (m, 10H), 4.40-4.20 (m, 2H), 4.13 (t, J=7.0 Hz, 1H), 3.30-3.24 (m, 1H), 3.08-2.74 (m, 7H), 1.89 (t, J=5.8 Hz, 1H), 1.81-1.62 (m, 3H), 1.38 (s, 9H), 1.18-0.94 (m, 2H).

### Step 6: Phe-D-Arg-(α-d)-Phe-Lys-NH2 Hydrochloride (Example 5)

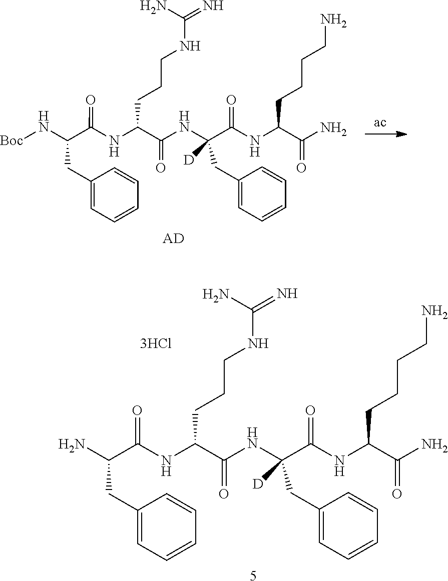

To a cooled (0° C.) solution of Phe-D-Arg-(α-d)-Phe-Lys-NH₂ acetate (AD, 100 mg, 0.189 mmol) in DCM (9 mL) TFA (3.0 mL) was added. After 5 min, the ice bath was removed and the mixture stirred at ambient temperature for 2 h. Volatiles were removed under reduced pressure and the residue concentrated from 2 N HCl/Et₂O (3×2.0 mL). It was obtained 81 mg of Phe-D-Arg-(α-d)-Phe-Lys-NH2 hydrochloride. ¹H NMR (400 MHz, Methanol-d₄) δ 7.41-7.10 (m, 10H), 4.48-4.33 (m, 1H), 4.14 (td, J=7.0, 3.2 Hz, 2H), 3.13 (qd, J=13.7, 7.5 Hz, 2H), 3.03-2.86 (m, 5H), 1.97-1.64 (m, 4H), 1.62-1.26 (m, 5H), 1.16-1.02 (m, 1H), 1.01-0.84 (m, 1H). Molecular formula: C₃₀H₄₄DN₉O₄.3HCl; Molecular weight: 706.14; Free base molecular weight: 596.76. EI-MS: m/z 597.7 [M+1].

### Example 6: Synthesis of Phe-D-Arg-(α,β,β,2,3,4,5,6-d8)-Phe-Lys-NH2 Hydrochloride

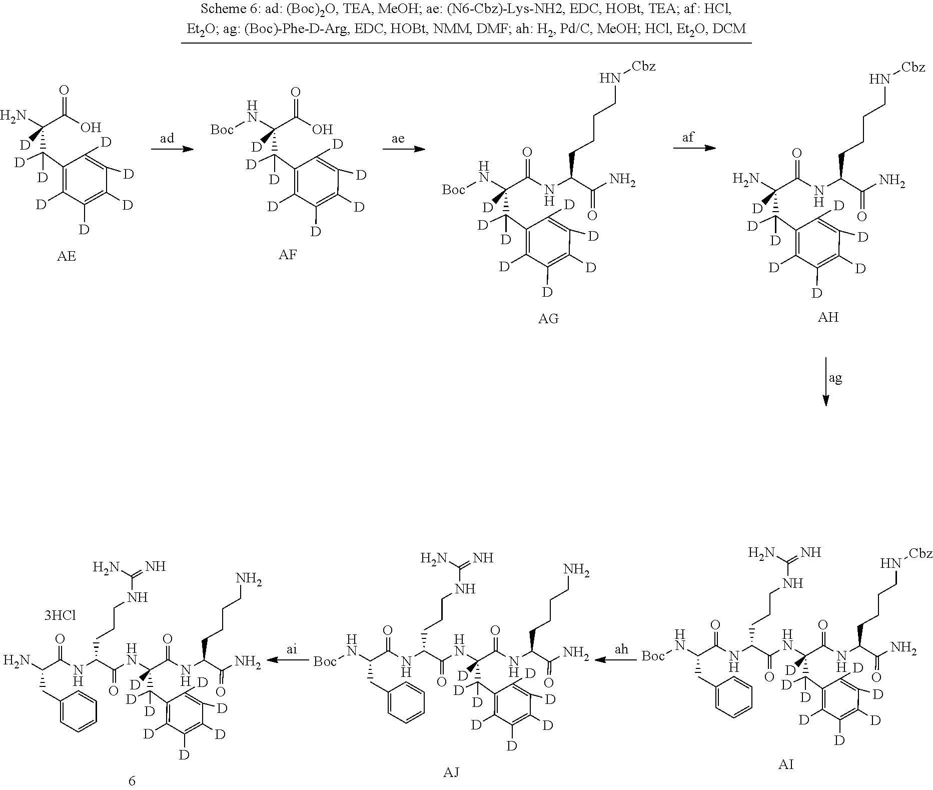

### Step 1: Synthesis of (N-Boc-α,β,β,2,3,4,5,6-d8)-Phe

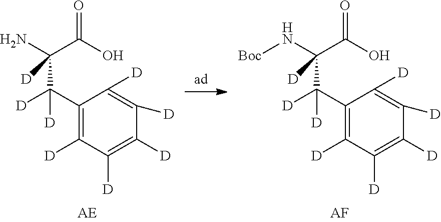 [SMILES: [2H]c1c([2H])c([2H])c(C([2H])([2H])[C@]([2H])(N)C(=O)O)c([2H])c1[2H] ; [2H]c1c([2H])c([2H])c(C([2H])([2H])[C@]([2H])(NC(=O)OC(C)(C)C)C(=O)O)c([2H])c1[2H] ; "AF"]

To a solution of L-Phenyl-d8-alanine (AE, 0.25 g, 1.44 mmol) and Boc₂O (0.34 g, 1.58 mmol) in methanol (2.5 mL) TEA (0.22 mL, 1.58 mmol) was added at 0° C. and the reaction mixture was stirred at this temperature for 5 min. Then the reaction mixture was stirred 1.5 h at room temperature. LCMS data showed full conversion. The reaction mixture was concentrated, dried in vacuo, to give 0.54 g of (N-Boc-α,β,β,2,3,4,5,6-d8)-Phe and used in the next step without further purification. ¹H NMR (400 MHz, Chloroform-d) δ 5.32 (s, 1H), 1.41 (s, 9H).

### Step 2: Synthesis of (N-Boc-α,β,β,2,3,4,5,6-d8)-Phe-(N6-Cbz)-Lys-NH2

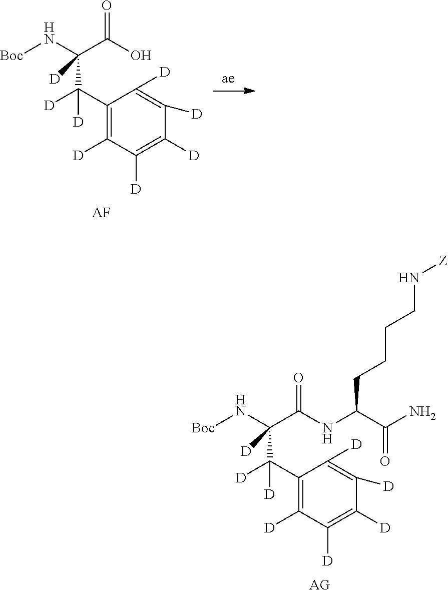

To a mixture of (N-Boc-α,β,β,2,3,4,5,6-d8)-Phe (AF, 0.54 g, 1.44 mmol), (N6-Cbz)-Lys-NH2 (0.45 g, 1.44 mmol), HOBt monohydrate (0.32 g, 1.87 mmol), EDC hydrochloride (0.35 g, 1.87 mmol) in DCM (6.0 mL) TEA (0.6 mL, 4.32 mmol) was added at 0° C. After 5 min ice bath was removed and the reaction mixture was stirred at RT over night. Next day the mixture was diluted with DCM, washed with 5% NaHCO₃, water, 5% KHSO₄, brine. The organic layer was dried over Na₂SO₄, filtered, concentrated, dried in vacuo. Crude product was purified by column chromatography on silica gel (DCM-MeOH 10:1 was used as eluent), to give 0.55 g (52%) of (N-Boc-α,β,β,2,3,4,5,6-d8)-Phe-(N6-Cbz)-Lys-NH2. ¹H NMR (400 MHz, DMSO-d₆) δ 7.78 (d, J=8.1 Hz, 1H), 7.43-7.14 (m, 6H), 7.07-6.92 (m, 2H), 4.99 (s, 2H), 4.19 (td, J=8.2, 4.9 Hz, 1H), 2.97 (q, J=6.7 Hz, 2H), 1.65 (q, J=10.4, 9.2 Hz, 1H), 1.58-1.34 (m, 4H), 1.32-1.18 (m, 1H), 1.29 (s, 9H).

### Step 3: Synthesis of (α,β,β,2,3,4,5,6-d8)-Phe-(N6-Cbz)-Lys-NH2

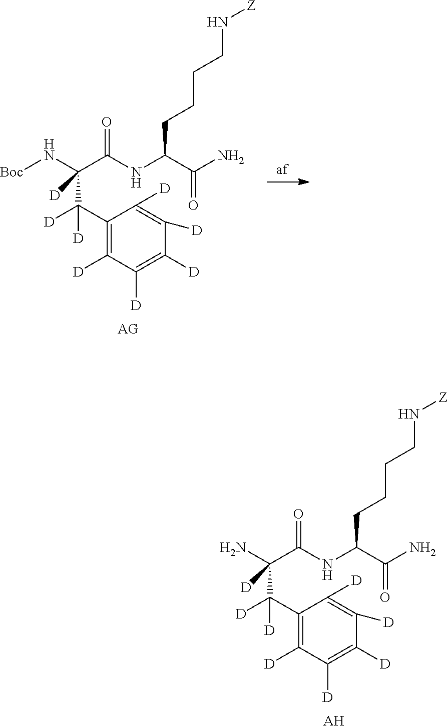

To a solution of (N-Boc-α,β,β,2,3,4,5,6-d8)-Phe-(N6-Cbz)-Lys-NH2 (AG, 0.55 g, 1.02 mmol) in DCM (10.0 mL) 2 M HCl/Et₂O (4.0 mL) was added at 0° C. After 5 min ice bath was removed and the reaction mixture was stirred at ambient temperature. After 2 h TLC showed full consumption of starting material. The reaction mixture was concentrated, concentrated with Et₂O (3 times), filtered off, washed with Et₂O, dried in vacuo, to give 0.43 g (97%) of (α,β,β,2,3,4,5,6-d8)-Phe-(N6-Cbz)-Lys-NH2. ¹H NMR (400 MHz, DMSO-d₆) δ 8.71 (d, J=8.1 Hz, 1H), 8.28 (t, J=18.4 Hz, 2H), 7.43-7.18 (m, 6H), 7.06 (d, J=2.3 Hz, 1H), 5.00 (s, 2H), 4.20 (ddd, J=8.1, 6.7, 3.9 Hz, 1H), 3.08-2.89 (m, 2H), 1.76-1.48 (m, 2H), 1.47-1.15 (m, 4H).

### Step 4: Synthesis of (Boc)-Phe-D-Arg-(α,β,β,2,3,4,5,6-d8)-Phe-(N6-Cbz)-Lys-NH2

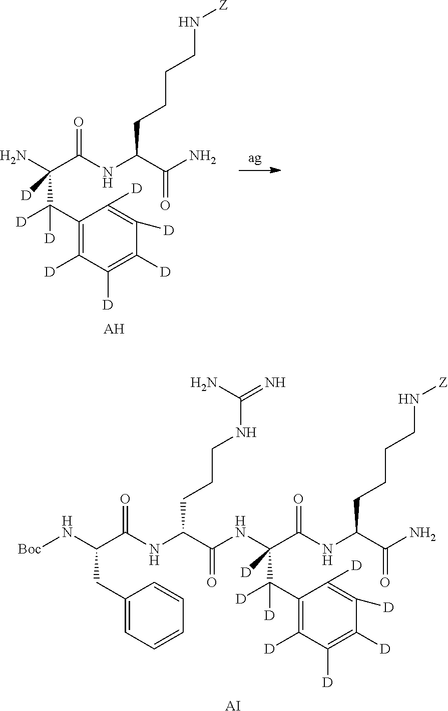

To a mixture of (α,β,β,2,3,4,5,6-d8)-Phe-(N6-Cbz)-Lys-NH2 (AH, 0.43 g, 0.92 mmol), (Boc)-Phe-D-Arg (0.465 g, 1.02 mmol), HOBt monohydrate (0.18 g, 1.1 mmol), EDC hydrochloride (0.21 g, 1.1 mmol) in DMF (5 mL) NMM (0.12 mL, 1.1 mmol) was added at 0° C. After 5 min ice bath was removed and the reaction mixture was stirred overnight at RT. After purification by column chromatography (DCM-MeOH 10/1) and additional purification by preparative HPLC, 0.18 g (23%) of (Boc)-Phe-D-Arg-(α,β,β,2,3,4,5,6-d8)-Phe-(N6-Cbz)-Lys-NH2 was isolated as a white solid. ¹H NMR (400 MHz, DMSO-d₆) δ 8.27 (s, 1H), 7.99 (dd, J=32.8, 8.0 Hz, 2H), 7.41-7.11 (m, 10H), 6.97 (d, J=2.1 Hz, 2H), 6.89 (d, J=8.3 Hz, 2H), 5.00 (s, 2H), 4.34-4.06 (m, 3H), 3.04-2.82 (m, 5H), 2.77-2.61 (m, 1H), 1.75-1.48 (m, 2H), 1.47-1.13 (m, 4H), 1.32 (s, 9H), 1.11-0.90 (m, 2H).

### Step 5: Synthesis of (Boc)-Phe-D-Arg-(α,β,β,2,3,4,5,6-d8)-Phe-Lys-NH2

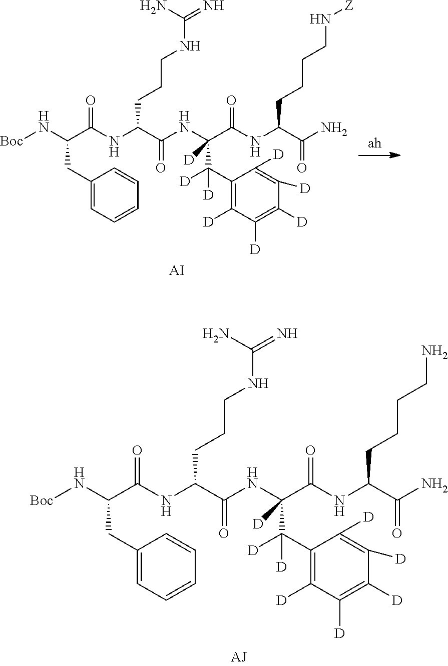

(Boc)-Phe-D-Arg-(α,β,β,2,3,4,5,6-d8)-Phe-(N6-Cbz)-Lys-NH2 (AI, 0.18 g, 0.21 mmol) was dissolved in MeOH (3 mL) and acetic acid (0.04 mL) and 10% Pd/C was added. The flask was subjected to 3 cycles of evacuation-hydrogen gas backfill, and the mixture stirred for 3 h at RT. Then the reaction mixture was filtered off, combined washes were concentrated, dried, to give 0.16 g (94%) of (Boc)-Phe-D-Arg-(α,β,β,2,3,4,5,6-d8)-Phe-Lys-NH2. ¹H NMR (400 MHz, Methanol-d₄) δ 7.37-7.13 (m, 5H), 4.38-4.19 (m, 2H), 4.13 (t, J=7.1 Hz, 1H), 3.08-2.77 (m, 6H), 1.96-1.84 (m, 3H), 1.82-1.24 (m, 5H), 1.38 (s, 9H), 1.21-0.87 (m, 2H).

### Step 6: Synthesis of Phe-D-Arg-(α,β,β,2,3,4,5,6-d8)-Phe-Lys-NH2 (Example 6)

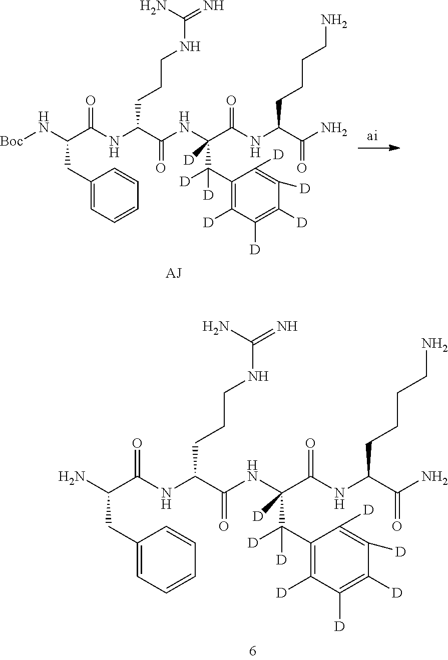 [SMILES: [2H]c1c([2H])c([2H])c(C([2H])([2H])[C@]([2H])(NC(=O)[C@@H](CCCNC(=N)N)NC(=O)[C@H](Cc2ccccc2)NC(=O)OC(C)(C)C)C(=O)N[C@@H](CCCCN)C(N)=O)c([2H])c1[2H] ; [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@H](CCCCN)C(N)=O)c([2H])c1[2H]]

To a suspension of (Boc)-Phe-D-Arg-(α,β,β,2,3,4,5,6-d8)-Phe-Lys-NH2 (AJ, 0.15 g, 0.18 mmol) in DCM (3.0 mL) 2 M HCl/Et₂O (1.0 mL) was added at 0° C. After 5 min ice bath was removed and the reaction mixture was stirred 4 h at ambient temperature. The reaction mixture was concentrated; the residue was purified by preparative HPLC to give 0.08 g (61%) of Phe-D-Arg-(α,β,β,2,3,4,5,6-d8)-Phe-Lys-NH2. ¹H NMR (400 MHz, Methanol-d₄) δ 7.42-7.21 (m, 5H), 4.40 (dd, J=9.0, 5.3 Hz, 1H), 4.13 (td, J=7.4, 3.1 Hz, 2H), 3.24-3.03 (m, 2H), 2.94 (q, J=9.2, 8.3 Hz, 4H), 1.94-1.63 (m, 4H), 1.59-1.24 (m, 3H), 1.06 (dq, J=11.7, 6.2 Hz, 1H), 0.93 (ddt, J=16.3, 9.6, 5.0 Hz, 1H). Molecular formula: C₃₀H₃₇D₈N₉O₄.3 HCl; Molecular weight: 713.17; Free base molecular weight: 603.80; EI-MS: m/z 604.7 [M+1].

### Example 7: Synthesis of Phe-D-Arg-(2,3,4,5,6-d5)-Phe-Lys-NH2

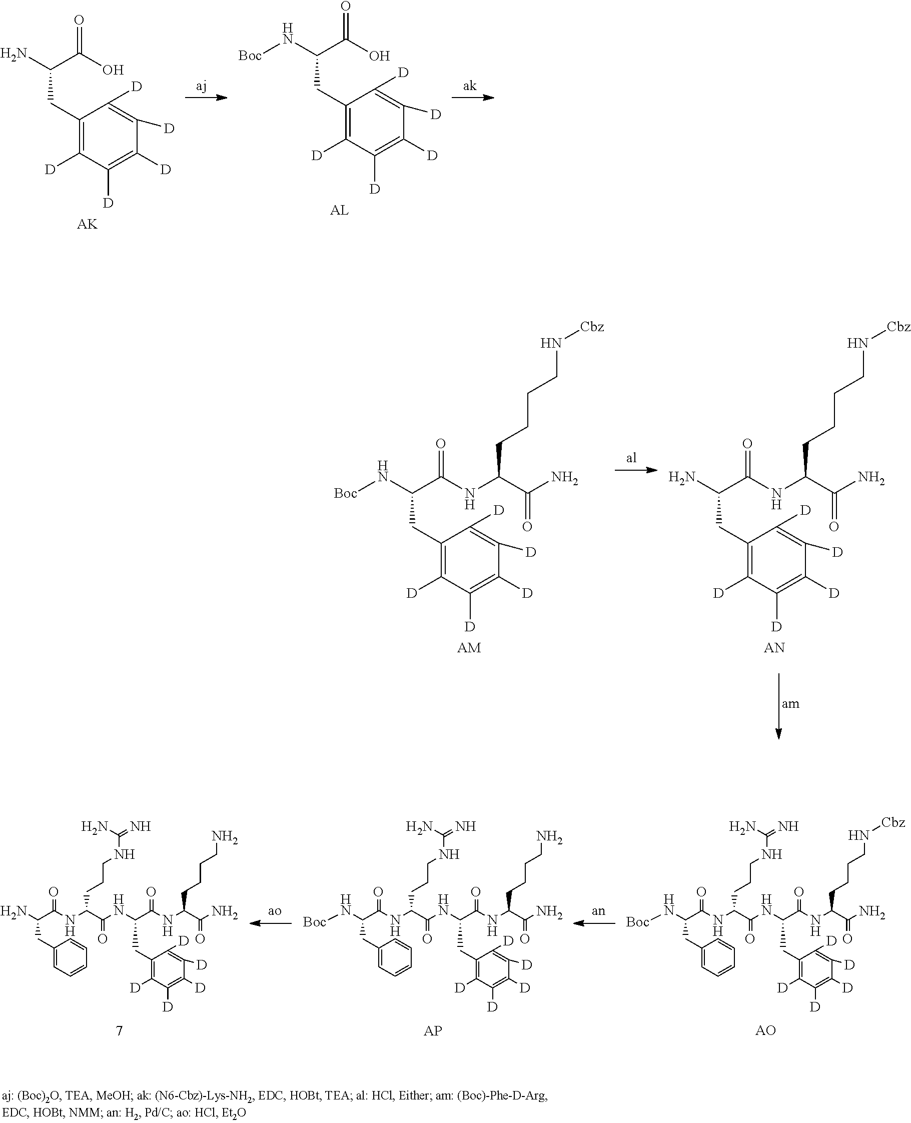

### Step 1: Synthesis of (N-Boc-2,3,4,5,6-d5)-Phe

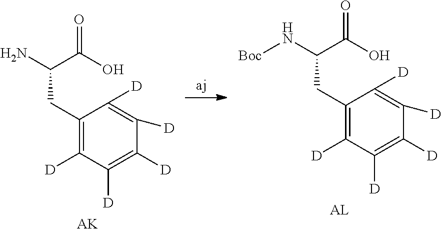

To a solution of L-Phenyl-d5-alanine (AK, 0.1 g, 0.58 mmol) and Boc₂O (0.15 g, 0.7 mmol) in methanol (1.0 mL) TEA (0.1 mL, 0.7 mmol) was added at 0° C. and the reaction mixture was stirred at this temperature for 5 min. Then the reaction mixture was stirred 1.5 h at room temperature. LCMS showed full conversion of product. The reaction mixture was concentrated, dried in vacuo, to give 0.16 g of (N-Boc-2,3,4,5,6-d5)-Phe and used in the next step. ¹H NMR (300 MHz, CDCl₃) δ: 5.36 (br d, J=6.4 Hz), 4.38-4.35 (m, 1H), 3.27-3.22 (m, 1H), 3.12-3.07 (m, 1H), 1.40 (s, 9H).

### Step 2: Synthesis of (N-Boc-2,3,4,5,6-d5)-Phe-(N6-Cbz)-Lys-NH2

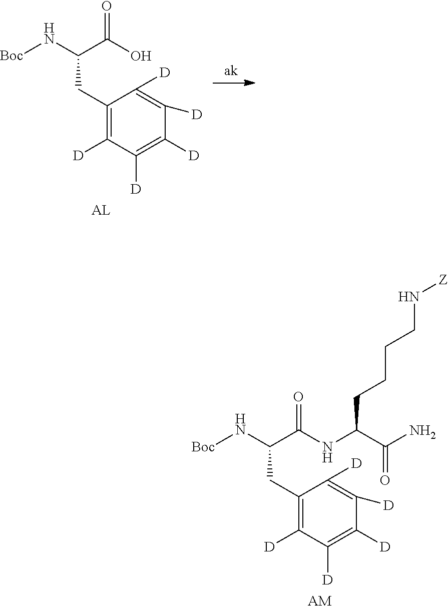

To a mixture of (N-Boc-2,3,4,5,6-d5)-Phe (AL, 0.16 g, 0.58 mmol), (N6-Cbz)-Lys-NH2 (0.18 g, 0.58 mmol), HOBt monohydrate (0.15 g, 0.87 mmol), EDC hydrochloride (0.16 g, 0.87 mmol) in DCM (2.5 mL) TEA (0.2 mL, 2.32 mmol) was added at 0° C. After 5 min ice bath was removed and the reaction mixture was stirred at RT over night. Next day the mixture was diluted with DCM, washed with 5% NaHCO₃, water, 5% KHSO₄, brine. The organic layer was dried over Na₂SO₄, filtered off, concentrated, dried in vacuo. Crude product was purified by column chromatography on silica gel (DCM-MeOH 10:1 was used as eluent), to give 0.21 g (67%) of (N-Boc-2,3,4,5,6-d5)-Phe-(N6-Cbz)-Lys-NH2. ¹H NMR (400 MHz, Chloroform-d) δ 7.52-7.20 (m, 5H), 6.61 (d, J=7.9 Hz, 1H), 6.15 (s, 1H), 5.12 (s, 2H), 4.38 (ddd, J=14.8, 9.9, 5.8 Hz, 2H), 3.10 (dqd, J=34.4, 14.2, 7.3 Hz, 5H), 1.98-1.04 (m, 5H), 1.39 (s, 9H).

### Step 3: Synthesis of (2,3,4,5,6-d5)-Phe-(N6-Cbz)-Lys-NH2

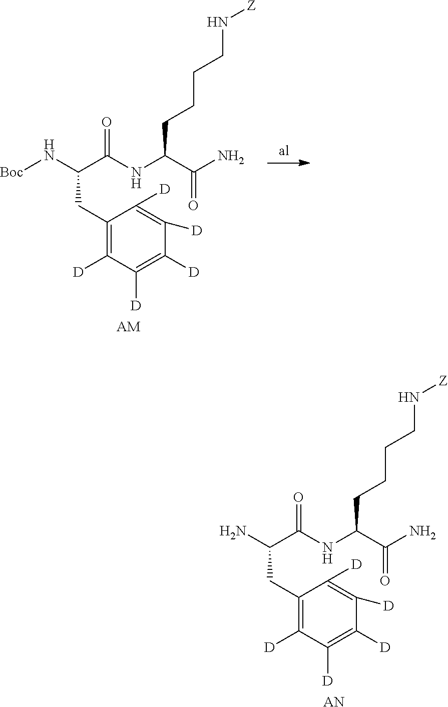

To a solution of (N-Boc-2,3,4,5,6-d5)-Phe-(N6-Cbz)-Lys-NH2 (AM, 0.21 g, 0.39 mmol) in DCM (4.0 mL) 2 M HCl/Et₂O (1.5 mL) was added at 0° C. After 5 min ice bath was removed and the reaction mixture was stirred at ambient temperature. After 2 h TLC showed full consumption of starting material. The reaction mixture was concentrated, re-evaporated with Et₂O (3 times), filtered off, washed with Et₂O, dried, to give 0.16 g (86%) of (2,3,4,5,6-d5)-Phe-(N6-Cbz)-Lys-NH2. ¹H NMR (400 MHz, DMSO-d₆) δ 8.66 (d, J=8.0 Hz, 1H), 7.43-7.18 (m, 5H), 7.13-7.01 (m, 1H), 4.99 (s, 2H), 4.20 (td, J=8.1, 5.2 Hz, 1H), 4.09 (d, J=7.0 Hz, 1H), 3.13 (dd, J=14.1, 5.5 Hz, 1H), 3.03-2.91 (m, 2H), 1.77-1.48 (m, 2H), 1.46-1.16 (m, 4H).

### Step 4: Synthesis of (Boc)-Phe-D-Arg-(2,3,4,5,6-d5)-Phe-(N6-Cbz)-Lys-NH2

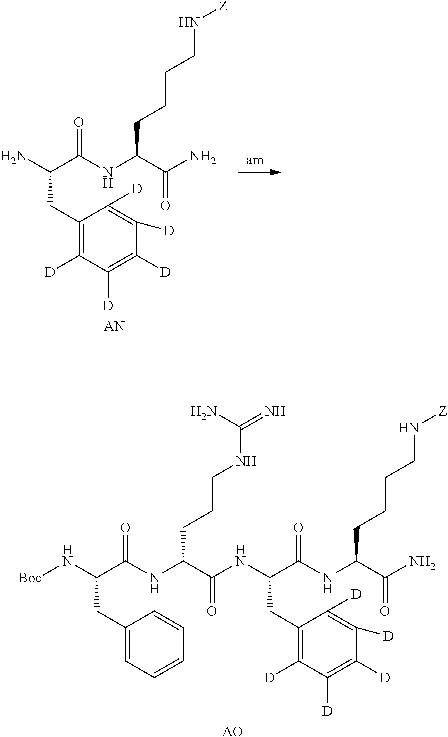

To a mixture of (Boc)-Phe-D-Arg (AN, 0.18 g, 0.38 mmol), (2,3,4,5,6-d5)-Phe-(N6-Cbz)-Lys-NH2 (0.16 g, 0.32 mmol), HOBt monohydrate (0.065 g, 0.38 mmol), EDC hydrochloride (0.072 g, 0.38 mmol) in DMF (1.5 mL) NMM (0.038 mL, 0.35 mmol) was added at 0° C. After 5 min ice bath was removed and the reaction mixture was stirred overnight at RT. After purification by column chromatography (DCM-MeOH 10/1) and additional purification by preparative HPLC, 0.09 g (30%) of (Boc)-Phe-D-Arg-(2,3,4,5,6-d5)-Phe-(N6-Cbz)-Lys-NH2 was isolated. ¹H NMR (300 MHz, Methanol-d₄) δ 7.31-7.17 (m, 10H), 5.03 (s, 2H), 4.59-4.54 (m, 1H), 4.29-4.24 (m, 2H), 4.13-4.09 (m, 1H), 3.70-3.62 (m, 1H), 3.30-2.89 (m, 6H), 1.84-1.33 (m, 7H), 1.35 (s, 9H).

### Step 5: Synthesis of (Boc)-Phe-D-Arg-(2,3,4,5,6-d5)-Phe-Lys-NH2

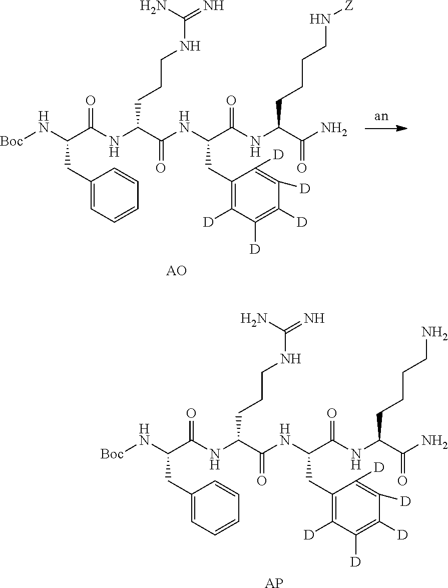

(Boc)-Phe-D-Arg-(2,3,4,5,6-d5)-Phe-(N6-Cbz)-Lys-NH₂ (AO, 0.09 g, 0.1 mmol) was dissolved in MeOH (3 mL) and acetic acid (0.04 mL) and 10% Pd/C was added. The flask was subjected to 3 cycle of evacuation-hydrogen gas backfill and the mixture stirred at 1 atm for 3 h at RT. Then the reaction mixture was filtered off, combined washes were concentrated, dried, to give 0.07 g (85%) of (Boc)-Phe-D-Arg-(2,3,4,5,6-d5)-Phe-Lys-NH2.

### Step 6: Synthesis of Phe-D-Arg-(2,3,4,5,6-d5)-Phe-Lys-NH2 (Example 7)

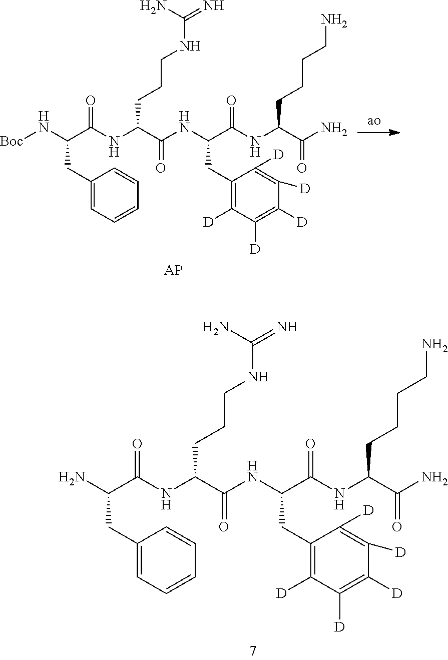 [SMILES: [2H]c1c([2H])c([2H])c(C[C@H](NC(=O)[C@@H](CCCNC(=N)N)NC(=O)[C@H](Cc2ccccc2)NC(=O)OC(C)(C)C)C(=O)N[C@@H](CCCCN)C(N)=O)c([2H])c1[2H] ; "AP" ; [2H]c1c([2H])c([2H])c(C[C@H](NC(=O)[C@@H](CCCNC(=N)N)NC(=O)[C@@H](N)Cc2ccccc2)C(=O)N[C@@H](CCCCN)C(N)=O)c([2H])c1[2H]]

To a suspension of (Boc)-Phe-D-Arg-(2,3,4,5,6-d5)-Phe-Lys-NH2 (AP, 0.07 g, 0.09 mmol) in DCM (2.0 mL) 2 M HCl/Et₂O (0.5 mL) was added at 0° C. After 5 min ice bath was removed and the reaction mixture was stirred 4 h at ambient temperature. The reaction mixture was concentrated, re-evaporated with Et₂O, washed with Et₂O. The residue was purified by preparative HPLC to give 0.018 g (61%) of Phe-D-Arg-(2,3,4,5,6-d5)-Phe-Lys-NH2. ¹H NMR (400 MHz, Methanol-d₄) δ 7.39-7.17 (m, 5H), 4.61 (dt, J=11.1, 3.7 Hz, 1H), 4.47-4.36 (m, 1H), 4.09 (q, J=7.2 Hz, 2H), 3.36-3.30 (m, 1H), 3.20-3.02 (m, 2H), 3.00-2.77 (m, 4H), 1.96-1.60 (m, 4H), 1.60-1.19 (m, 5H), 1.14-0.77 (m, 2H). Molecular formula: C₃₀H₄₀D₅N₉O₄.3HCl; Molecular weight: 710.16; Free base molecular weight: 600.78; EI-MS: m/z 601.9 [M+1].

### Example 8: Synthesis of D-Arg-DMT-Lys-(2,3,4,5,6-d5)-Phe-NH2

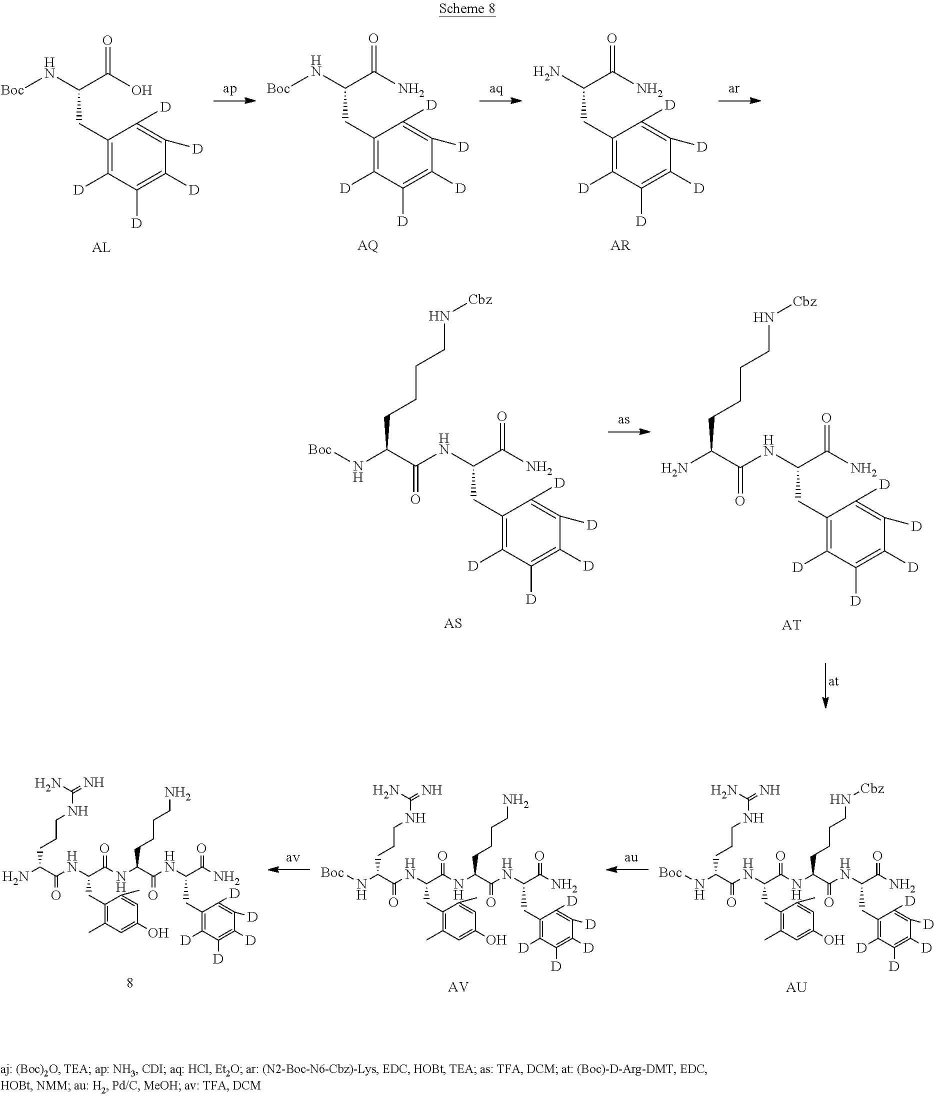

### Step 1: Synthesis of (N-Boc-2,3,4,5,6-d5)-Ph-NH2

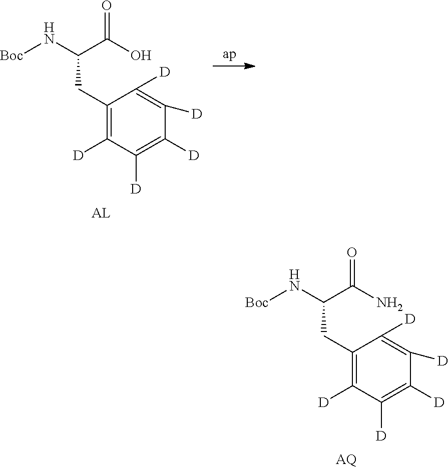

To a solution of (N-Boc-2,3,4,5,6-d5)-Phe (AL, 0.54 g, 1.45 mmol) in DMF (6 mL), CDI (0.29 g, 1.82 mmol) was added at 0° C. After stirring for 2 h at RT, the reaction mixture was cooled again and 25% aq ammonia (0.5 mL) was added dropwise. An ice-water bath was removed, after 2 h of stirring at RT LC-MS showed full conversion of product. The mixture was diluted with DCM, washed with 5% NaHCO₃, water, 5% KHSO₄, brine. The organic layer was dried over Na₂SO₄, concentrated, dried in vacuo. After crystallization (MeOH-Et₂O), 0.31 g (79%) of (N-Boc-2,3,4,5,6-d5)-Phe-NH2 was isolated. ¹H NMR (400 MHz, Chloroform-d) δ 5.36 (d, J=7.0 Hz, 1H), 4.37 (q, J=5.8 Hz, 1H), 3.24 (dd, J=13.5, 5.3 Hz, 1H), 3.09 (dd, J=13.6, 5.3 Hz, 1H), 1.40 (s, 9H).

### Step 2: Synthesis of (2,3,4,5,6-d5)-Phe-NH2

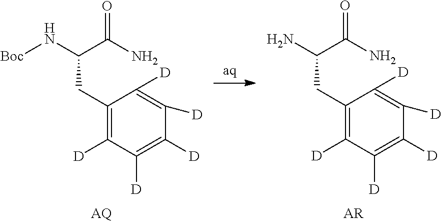

To a solution of (N-Boc-2,3,4,5,6-d5)-Phe-NH2 (AQ, 0.3 g, 1.1 mmol) in DCM (5 mL) 2 M HCl/Et₂O (2.0 mL) was added at 0° C. After stirring for 3 h, the reaction mixture was concentrated, and then concentrated with Et₂O (3 times). Et₂O was added, the formed precipitate was filtered off. After drying in vacuo, 0.21 g (91%) of (2,3,4,5,6-d5)-Phe-NH2 was obtained. ¹H NMR (400 MHz, DMSO-d₆) δ 8.28 (s, 2H), 7.98 (s, 1H), 7.51 (s, 1H), 3.97 (t, J=6.7 Hz, 1H), 3.07 (qd, J=13.9, 6.8 Hz, 2H).

### Step 3: Synthesis of (N2-Boc-N6-Cbz)-Lys-(2,3,4,5,6-d5)-Phe-NH2

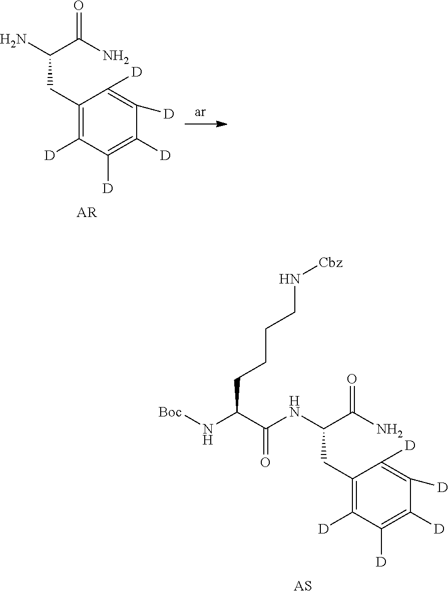 [SMILES: [2H]c1c([2H])c([2H])c(C[C@H](N)C(N)=O)c([2H])c1[2H] ; "AR" ; [2H]c1c([2H])c([2H])c(C[C@H](NC(=O)[C@H](CCCCNC(=O)OCc2ccccc2)NC(=O)OC(C)(C)C)C(N)=O)c([2H])c1[2H] ; "AS"]

To a mixture of (2,3,4,5,6-d5)-Phe-NH2 (AR, 0.21 g, 1.02 mmol), Boc-Lys(Cbz) (0.46 g, 1.2 mmol), HOBt monohydrate (0.26 g, 1.53 mmol), EDC hydrochloride (0.29 g, 1.53 mmol) in DCM (5 mL) TEA (0.42 mL, 3.06 mmol) was added at 0° C. After 5 min ice bath was removed and the reaction mixture was stirred at RT. After 3 h the mixture was diluted with DCM, washed with 5% NaHCO₃, water, 5% KHSO₄, brine. The organic layer was dried over Na₂SO₄, concentrated, dried in vacuo. Crude product was purified by column chromatography on silica gel (DCM-MeOH 10:1 was used as eluent), to give 0.31 g (60%) of (N2-Boc-N6-Cbz)-Lys-(2,3,4,5,6-d5)-Phe-NH2. ¹H NMR (400 MHz, Chloroform-d) δ 7.41-7.28 (m, 5H), 6.58-6.31 (m, 2H), 5.35 (d, J=14.4 Hz, 2H), 5.18-5.03 (m, 2H), 4.92 (s, 1H), 4.72 (q, J=7.2 Hz, 1H), 3.93 (dt, J=9.1, 5.0 Hz, 1H), 3.14 (dp, J=20.2, 6.5 Hz, 4H), 1.70 (d, J=10.3 Hz, 1H), 1.53-1.30 (m, 3H), 1.36 (s, 9H), 1.30-1.14 (m, 3H).

### Step 4: Synthesis of (N6-Cbz)-Lys-(2,3,4,5,6-d5)-Phe-NH2

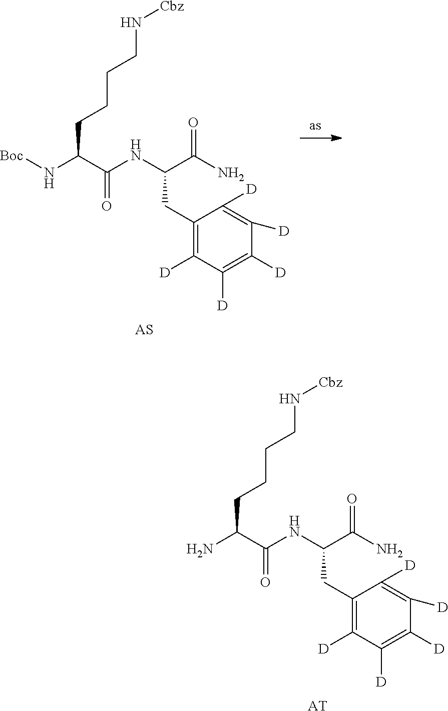 [SMILES: [2H]c1c([2H])c([2H])c(C[C@H](NC(=O)[C@H](CCCCNC(=O)OCc2ccccc2)NC(=O)OC(C)(C)C)C(N)=O)c([2H])c1[2H] ; "AS" ; [2H]c1c([2H])c([2H])c(C[C@H](NC(=O)[C@@H](N)CCCCNC(=O)OCc2ccccc2)C(N)=O)c([2H])c1[2H] ; "AT"]

To a solution of (N2-Boc-N6-Cbz)-Lys-(2,3,4,5,6-d5)-Phe-NH2 (AS, 0.31 g, 0.58 mmol) in DCM (6 mL) TFA (3 mL) was added at 0° C. After 5 min ice bath was removed and the reaction mixture was stirred 1 h at ambient temperature. The volatiles were evaporated, and then crude product was evaporated with toluene, dried, to give 0.28 g (88%) of (N6-Cbz)-Lys-(2,3,4,5,6-d5)-Phe-NH2.

### Step 5: Synthesis of (Boc)-D-Arg-DMT-(N6-Cbz)-Lys-(2,3,4,5,6-d5)-Phe-NH2

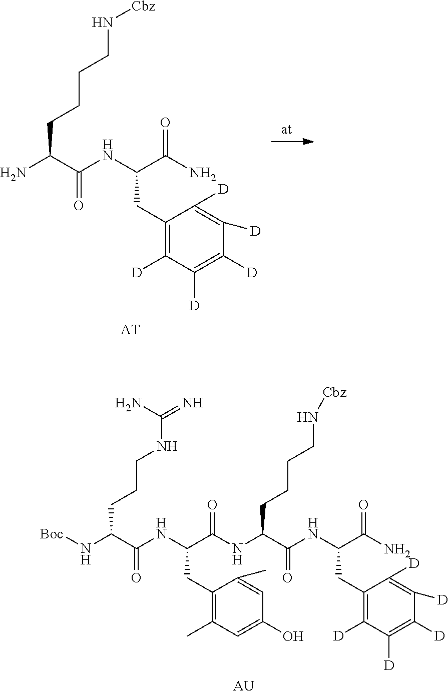 [SMILES: [2H]c1c([2H])c([2H])c(C[C@H](NC(=O)[C@@H](N)CCCCNC(=O)OCc2ccccc2)C(N)=O)c([2H])c1[2H] ; "AT" ; [2H]c1c([2H])c([2H])c(C[C@H](NC(=O)[C@H](CCCCNC(=O)OCc2ccccc2)NC(=O)[C@H](Cc2c(C)cc(O)cc2C)NC(=O)[C@@H](CCCNC(=N)N)NC(=O)OC(C)(C)C)C(N)=O)c([2H])c1[2H] ; "AU"]

To a mixture of (N6-Cbz)-Lys-(2,3,4,5,6-d5)-Phe-NH2 (AT, 0.28 g, 0.51 mmol), Boc-D-Arg-DMT (0.3 g, 0.61 mmol), HOBt monohydrate (0.1 g, 0.61 mmol), EDC hydrochloride (0.11 g, 0.61 mmol) in DMF (4 mL) NMM (67 μL, 0.61 mmol) was added at 0° C. After 5 min ice bath was removed and the reaction mixture was stirred overnight at RT. After purification by column chromatography (DCM-MeOH 10/1) and additional purification by preparative HPLC, 0.17 g of (Boc)-D-Arg-DMT-(N6-Cbz)-Lys-(2,3,4,5,6-d5)-Phe-NH2 was isolated as a white solid.

### Step 6: Synthesis of (Boc)-D-Arg-DMT-Lys-(2,3,4,5,6-d5)-Phe-NH2

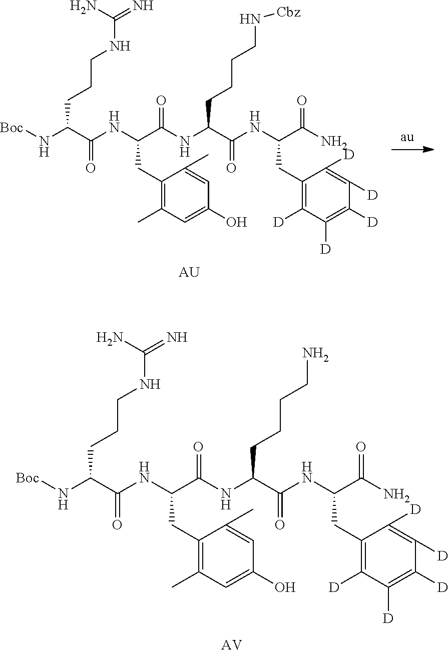 [SMILES: [2H]c1c([2H])c([2H])c(C[C@H](NC(=O)[C@H](CCCCNC(=O)OCc2ccccc2)NC(=O)[C@H](Cc2c(C)cc(O)cc2C)NC(=O)[C@@H](CCCNC(=N)N)NC(=O)OC(C)(C)C)C(N)=O)c([2H])c1[2H] ; "AU" ; [2H]c1c([2H])c([2H])c(C[C@H](NC(=O)[C@H](CCCCN)NC(=O)[C@H](Cc2c(C)cc(O)cc2C)NC(=O)[C@@H](CCCNC(=N)N)NC(=O)OC(C)(C)C)C(N)=O)c([2H])c1[2H] ; "AV"]

(Boc)-D-Arg-DMT-(N6-Cbz)-Lys-(2,3,4,5,6-d5)-Phe-NH2 (AU, 0.17 g, 0.18 mmol) was dissolved in MeOH (3 mL) and acetic acid (0.04 mL) and 10% Pd/C were added. The flask was subjected to 3 cycle of evacuation—hydrogen gas backfill and the mixture stirred at 1 atm for 3 h at RT. Then the reaction mixture was filtered off, combined washes were concentrated and purified by preparative HPLC, to give 0.095 g of (Boc)-D-Arg-DMT-Lys-(2,3,4,5,6-d5)-Phe-NH2. ¹H NMR (400 MHz, Methanol-d₄) δ 7.95 (d, J=7.8 Hz, 1H), 6.43 (s, 2H), 4.64-4.50 (m, 2H), 4.20 (t, J=7.1 Hz, 1H), 3.95 (dd, J=7.8, 5.5 Hz, 1H), 3.23-3.02 (m, 4H), 3.01-2.81 (m, 4H), 2.24 (s, 6H), 1.81-1.55 (m, 4H), 1.43 (s, 9H), 1.55-1.38 (s, 6H).

### Step 7: Synthesis of D-Arg-DMT-Lys-(2,3,4,5,6-d5)-Phe-NH2 Trifluoroacetate (Example 8)

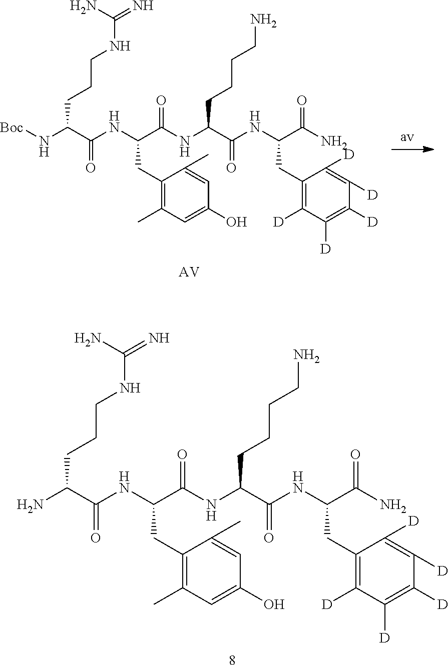 [SMILES: [2H]c1c([2H])c([2H])c(C[C@H](NC(=O)[C@H](CCCCN)NC(=O)[C@H](Cc2c(C)cc(O)cc2C)NC(=O)[C@@H](CCCNC(=N)N)NC(=O)OC(C)(C)C)C(N)=O)c([2H])c1[2H] ; "AV" ; [2H]c1c([2H])c([2H])c(C[C@H](NC(=O)[C@H](CCCCN)NC(=O)[C@H](Cc2c(C)cc(O)cc2C)NC(=O)[C@H](N)CCCNC(=N)N)C(N)=O)c([2H])c1[2H] ; "8"]

To a suspension of (Boc)-D-Arg-DMT-Lys-(2,3,4,5,6-d5)-Phe-NH₂ (AV, 0.09 g, 0.1 mmol) in DCM (3 mL) TFA (1.5 mL) was added at 0° C. After 5 min ice bath was removed and the reaction mixture was stirred 4 h at ambient temperature. The reaction mixture was concentrated; the volatiles were evaporated, then re-evaporated with toluene and purified by preparative HPLC to give 0.028 g (%) of D-Arg-DMT-Lys-(2,3,4,5,6-d5)-Phe-NH2. ¹H NMR (400 MHz, Methanol-d₄) δ 6.51-6.34 (s, 2H), 4.78 (dd, J=9.3, 7.0 Hz, 1H), 4.59 (dd, J=8.6, 6.0 Hz, 1H), 4.25 (dd, J=7.7, 6.5 Hz, 1H), 3.96 (t, J=6.2 Hz, 1H), 3.18-3.03 (m, 4H), 3.01-2.85 (m, 4H), 2.27 (s, 6H), 1.82-1.54 (m, 5H), 1.44-1.19 (m, 5H). Molecular formula: C₃₂H₄₄D₅N₉O₅x3TFA; Molecular weight: 986.90; Free base molecular weight: 644.83. EI-MS: m/z 645.8 [M+1].

### Example 9: Synthesis of D-Arg-DMT-Lys-(α,β,β,2,3,4,5,6-d8)-Phe-NH2

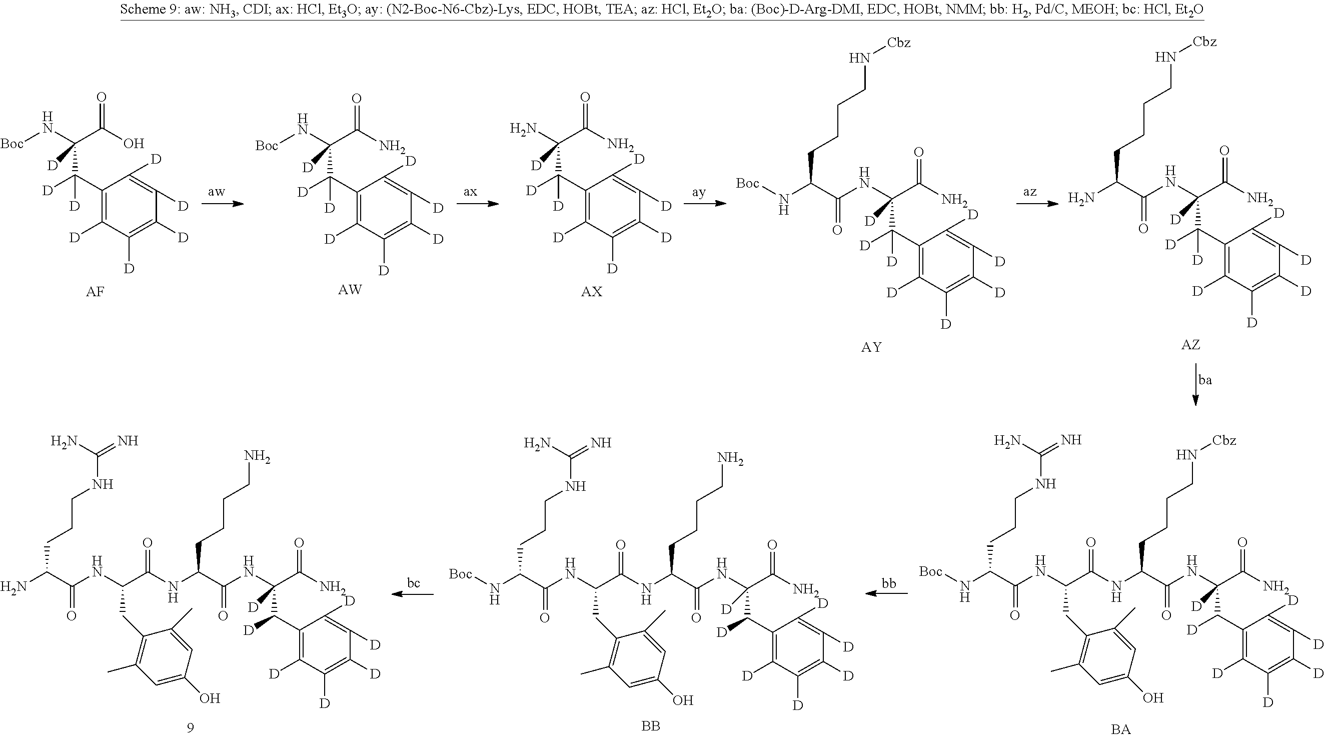

### Step 1: Synthesis of Synthesis of (N-Boc-α,β,β,2,3,4,5,6-d8)-Phe-NH2

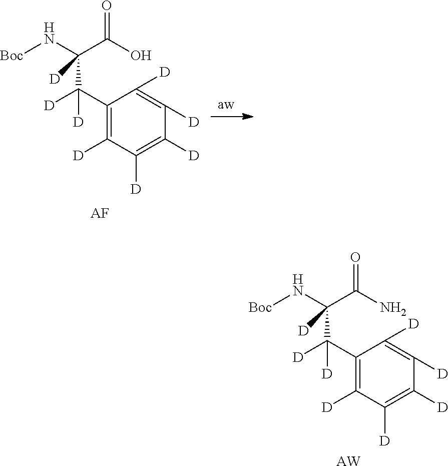 [SMILES: [2H]c1c([2H])c([2H])c(C([2H])([2H])[C@]([2H])(NC(=O)OC(C)(C)C)C(=O)O)c([2H])c1[2H] ; "AF" ; [2H]c1c([2H])c([2H])c(C([2H])([2H])[C@]([2H])(NC(=O)OC(C)(C)C)C(N)=O)c([2H])c1[2H] ; "AW"]

To a solution of (N-Boc-α,β,β,2,3,4,5,6-d8)-Phe (AF, 0.3 g, 1.13 mmol) in DMF (5.0 mL), CDI (0.23 g, 1.42 mmol) was added at 0° C. After stirring for 2 h at RT, the reaction mixture was cooled again and 25% aq ammonia (0.5 mL) was added dropwise. An ice-water bath was removed and after stirring at RT for 2 h, LCMS showed full conversion of product. The mixture was diluted with DCM, washed with 5% NaHCO₃, water, 5% KHSO₄, brine. The organic layer was dried over Na₂SO₄, concentrated, dried in vacuo, to give 0.27 g (93%) of (N-Boc-α,β,β,2,3,4,5,6-d8)-Phe-NH2.

### Step 2: Synthesis of (α,β,β,2,3,4,5,6-d8)-Phe-NH2

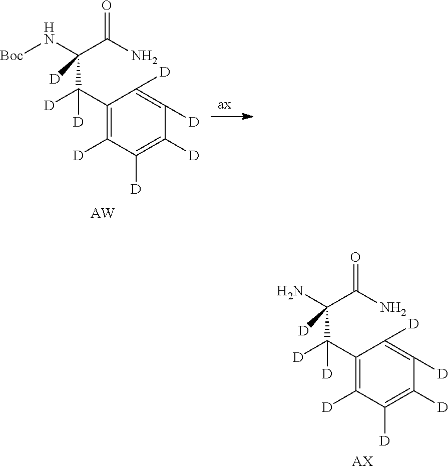

To a solution of (N-Boc-α,β,β,2,3,4,5,6-d8)-Phe-NH2 (AW, 0.27 g, 0.99 mmol) in DCM (5.0 mL) 2 M HCl/Et₂O (2.5 mL) was added at 0° C. The reaction mixture was stirred 3 h at RT, and then was concentrated, re-evaporated with ether (3 times). The formed solid was filtered off, washed with Et₂O, dried in vacuo, to give 0.15 g (72%) of (α,β,β,2,3,4,5,6-d8)-Phe-NH₂.

¹H NMR (400 MHz, Chloroform-d) δ 5.86 (s, 1H), 5.53 (s, 1H), 5.07 (s, 1H), 1.40 (s, 9H).

### Step 3: Synthesis of (N2-Boc-N6-Cbz)-Lys-(α,β,β,2,3,4,5,6-d8)-Phe-NH2

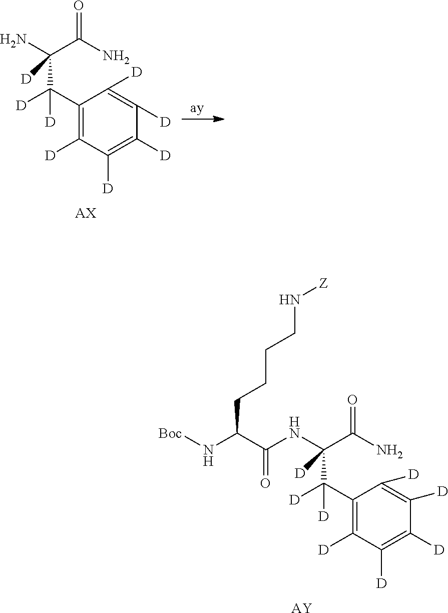

To a mixture of (α,β,β,2,3,4,5,6-d8)-Phe-NH2 (AX, 0.15 g, 0.69 mmol), (N2-Boc-N6-Cbz)-Lys (0.32 g, 0.83 mmol), HOBt monohydrate (0.17 g, 1.03 mmol), EDC hydrochloride (0.19 g, 1.03 mmol) in DCM (5 mL) TEA (0.38 mL, 2.75 mmol) was added at 0° C. After 5 min ice bath was removed and the reaction mixture was stirred at RT over night. Next day the mixture was diluted with DCM, washed with 5% NaHCO₃, water, 5% KHSO₄, brine. The organic layer was dried over Na₂SO₄, filtered, concentrated, dried in vacuo. Crude product was purified by column chromatography on silica gel (DCM-MeOH 10:1 was used as eluent), to give 0.26 g (72%) of (N2-Boc-N6-Cbz)-Lys-(α,β,β,2,3,4,5,6-d8)-Phe-NH2: ¹H NMR (400 MHz, Methanol-d₄) δ 7.54-7.12 (m, 5H), 5.07 (s, 2H), 3.84 (dd, J=8.2, 5.8 Hz, 1H), 3.06 (t, J=6.9 Hz, 2H), 1.67-1.03 (m, 6H), 1.41 (s, 9H).

### Step 4: Synthesis of (N6-Cbz)-Lys-(α,β,β,2,3,4,5,6-d8)-Phe-NH2

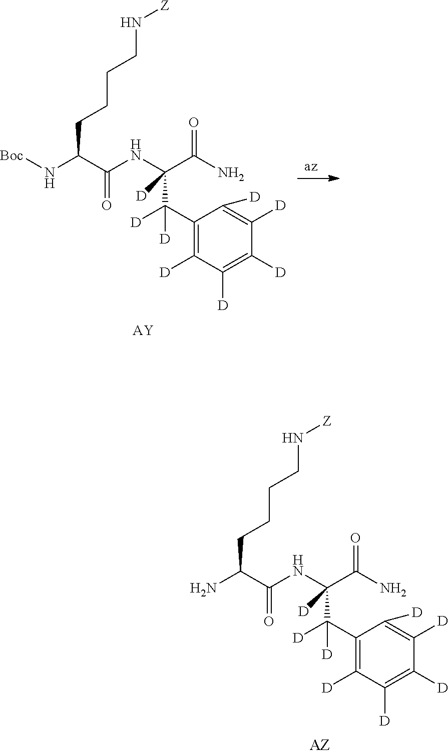

To a solution of (N2-Boc-N6-Cbz)-Lys-(α,β,β,2,3,4,5,6-d8)-Phe-NH2 (AY, 0.26 g, 0.48 mmol) in DCM (3 mL) 2 M HCl/Et₂O (1.5 mL) was added at 0° C. After 5 min ice bath was removed and the reaction mixture was stirred at ambient temperature. After 2 h TLC showed full consumption of starting material. The reaction mixture was concentrated, re-evaporated with ether (3 times), washed with Et₂O, filtered off, dried, to give 0.22 g (97%) of (N6-Cbz)-Lys-(α,β,β,2,3,4,5,6-d8)-Phe-NH2. ¹H NMR (400 MHz, Methanol-d₄) δ 7.48-7.09 (m, 5H), 5.06 (s, 2H), 3.79 (td, J=6.3, 5.8, 1.1 Hz, 1H), 3.12 (t, J=6.9 Hz, 2H), 1.84 (ddt, J=21.6, 14.3, 6.6 Hz, 2H), 1.59-1.47 (m, 2H), 1.47-1.34 (m, 2H).

### Step 5: Synthesis of D-(Boc)-Arg-DMT-(N6-Cbz)-Lys-(α,β,β,2,3,4,5,6-d8)-Phe-NH2

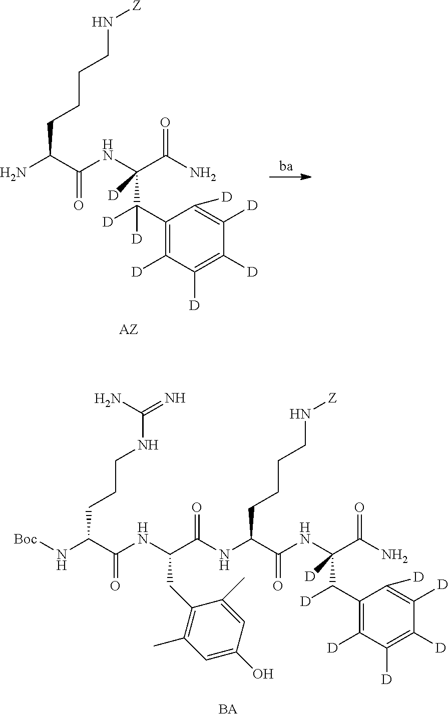

To a mixture of (N6-Cbz)-Lys-(α,β,β,2,3,4,5,6-d8)-Phe-NH2 (AZ, 0.17 g, 0.37 mmol), Boc-D-Arg-DMT (0.22 g, 0.41 mmol), HOBt monohydrate (0.076 g, 0.44 mmol), EDC hydrochloride (0.084 g, 0.44 mmol) in DMF (3 mL) NMM (60 μL, 0.44 mmol) was added at 0° C. After 5 min ice bath was removed and the reaction mixture was stirred overnight at RT. After purification by column chromatography (DCM-MeOH 10/1) and additional purification by preparative HPLC, 0.11 (30%) g of D-(Boc)-Arg-DMT-(N6-Cbz)-Lys-(α,β,β, 2,3,4,5,6-d8)-Phe-NH2 were isolated as a white solid. ¹H NMR (400 MHz, Methanol-d4) δ 7.42-7.20 (m, 5H), 6.43 (s, 2H), 5.06 (s, 2H), 4.69-4.53 (m, 1H), 4.18 (t, J=7.2 Hz, 1H), 3.92 (t, J=6.7 Hz, 1H), 3.17-2.99 (m, 5H), 2.97-2.82 (m, 1H), 1.78-1.54 (m, 3H), 1.54-1.16 (m, 7H), 1.14 (s, 9H).

### Step 6: Synthesis of D-(Boc)-Arg-DMT-(N6-Lys-(α,β,β,2,3,4,5,6-d8)-Phe-NH2

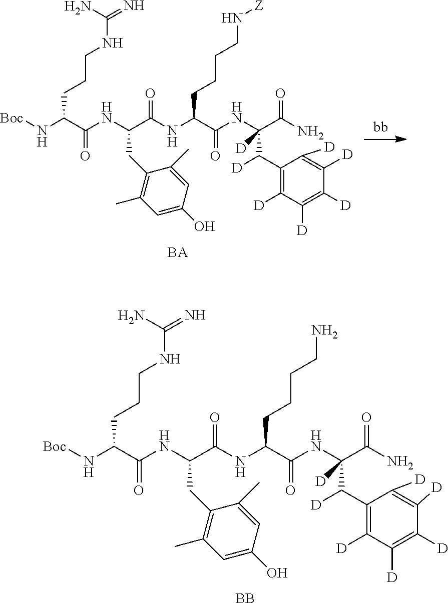

D-(Boc)-Arg-DMT-(N6-Cbz)-Lys-(α,β,β,2,3,4,5,6-d8)-Phe-NH2 (BA, 0.11 g, 0.12 mmol) was dissolved in MeOH (3 mL) and acetic acid (0.04 mL) and 10% Pd/C was added. The flask was subjected to 3 cycle of evacuation-hydrogen gas backfill and the mixture stirred at 1 atm for 3 h at RT. Then the reaction mixture was filtered off, combined washes were concentrated and purified by preparative HPLC, to give 0.09 g (89%) of D-(Boc)-Arg-DMT-Lys-(α,β,β, 2,3,4,5,6-d8)-Phe-NH2. ¹H NMR (400 MHz, Methanol-d₄) δ 6.43 (s, 2H), 4.60 (dd, J=8.9, 6.9 Hz, 1H), 4.21 (t, J=7.1 Hz, 1H), 3.95 (dd, J=7.8, 5.4 Hz, 1H), 3.18-3.08 (m, 3H), 2.96-2.82 (m, 3H), 2.25 (s, 6H), 1.83-1.54 (m, 4H), 1.53-1.22 (m, 6H), 1.42 (s, 9H).

### Step 7: Synthesis of D-Arg-DMT-Lys-(α,β,β,2,3,4,5,6-d8)-Phe-NH2 (Example 9)

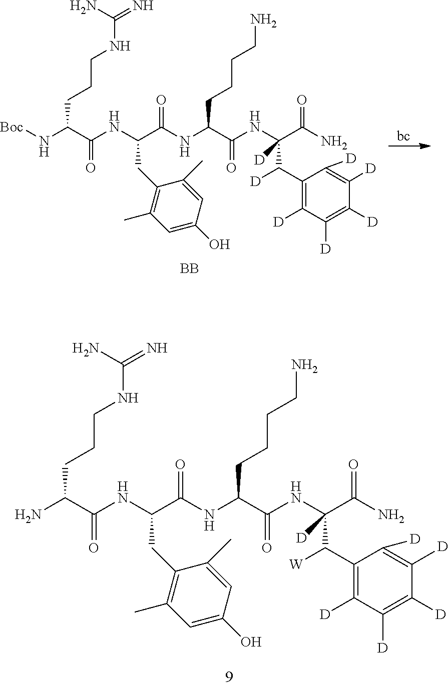

To a suspension of D-(Boc)-Arg-DMT-Lys-(α,β,β,2,3,4,5,6-d8)-Phe-NH2 (BB, 0.09 g, 0.1 mmol) in DCM (2 mL) 2 M HCl/Et₂O (1 mL) was added at 0° C. After 5 min ice bath was removed and the reaction mixture was stirred 4 h at ambient temperature. The reaction mixture was concentrated; the residue was purified by preparative HPLC to give 0.036 g (34%) of D-Arg-DMT-Lys-(α,β,β,2,3,4,5,6-d8)-Phe-NH2. ¹H NMR (400 MHz, Methanol-d₄) δ ¹H NMR (400 MHz, Methanol-d₄) δ 6.51-6.28 (m, 2H), 4.78 (dd, J=9.2, 7.2 Hz, 1H), 4.24 (dd, J=7.7, 6.6 Hz, 1H), 3.93 (t, J=6.2 Hz, 1H), 3.15-3.01 (m, 3H), 2.99-2.83 (m, 3H), 2.26 (s, 6H), 1.82-1.52 (m, 5H), 1.48-1.15 (m, 5H). Molecular formula: C₃₂H₄₁D₈N₉O₅x3TFA; Molecular weight: 989.92; Free base molecular weight: 647.85. EI-MS: m/z 648.8 [M+1].

### Example 10: Synthesis of D-Arg-DMT-Lys-(α-d)-Phe-NH2

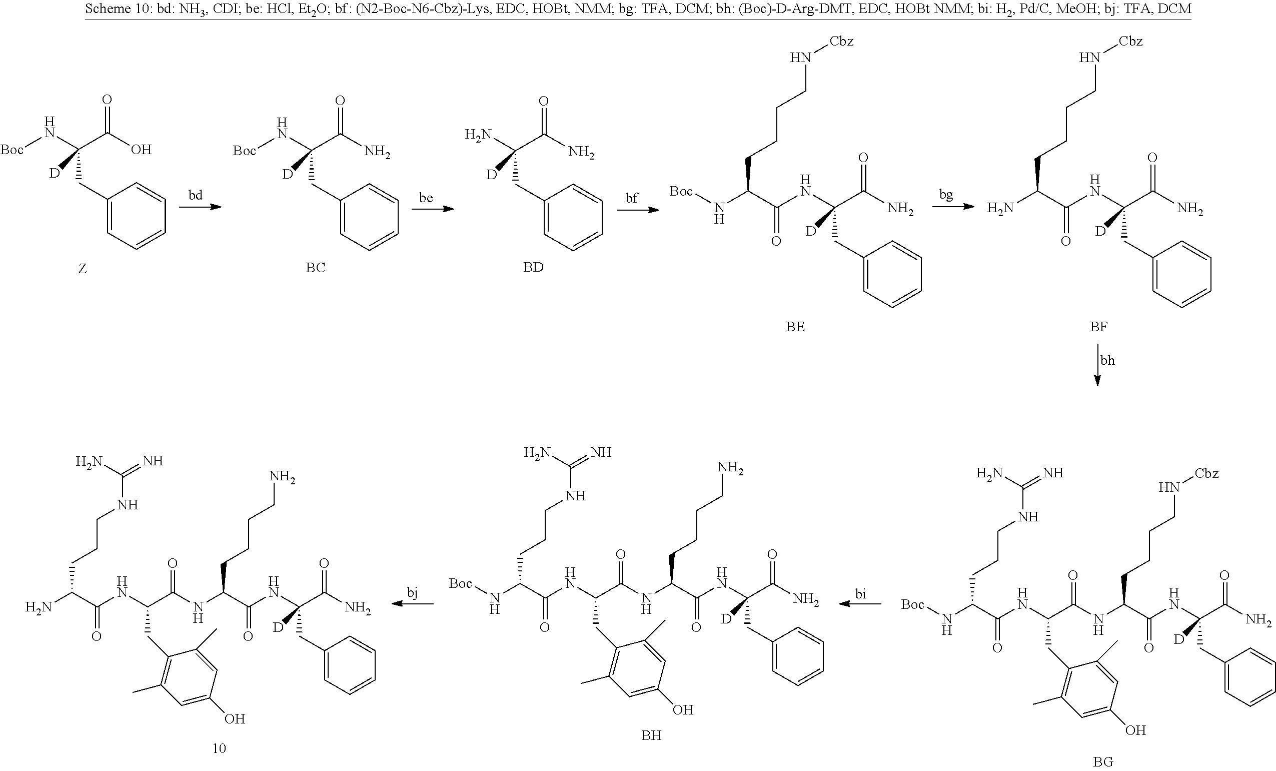

### Step 1: Synthesis of (N-Boc-α-d)-Phe-NH2

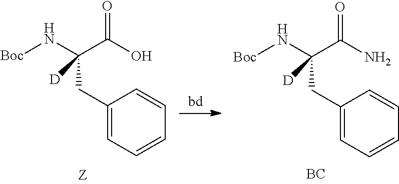 [SMILES: [2H][C@@](Cc1ccccc1)(NC(=O)OC(C)(C)C)C(=O)O ; [2H][C@@](Cc1ccccc1)(NC(=O)OC(C)(C)C)C(N)=O ; "BC"]

To the mixture of (N-Boc-α-d)-Phe (Z, 275 mg, 1.36 mmol) in DMF (5 mL) carbonyl diimidazole (CDI) (276 mg, 1.70 mmol) was added. Reaction mixture was stirred at rt for 0.5 h. Then 25% NH₄OH (550 μL) was added and stirred at rt over night. To the reaction mixture 5% NaHCO₃ water solution (3 mL) and DCM (5 mL) were added. The organic layer was separated and washed with 5% KHSO₄ waters solution (7 mL), brine (7 mL). DCM was removed to afford (N-Boc-α-d)-Phe-NH2 as white solid (245 mg, 87%). Product was used without further purification.

### Step 2: Synthesis of (α-d)-Phe-NH2

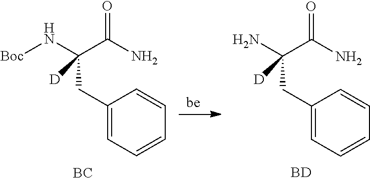

To a cooled (0° C.) solution of (N-Boc-α-d)-Phe-NH2 (BC, 243 mg, 0.886 mmol) in DCM (12 mL) 2 N HCl in Et₂O (3.5 mL) was added. After 5 min, the ice bath was removed and the mixture stirred at ambient temperature for 2 h. Volatiles were removed under reduced pressure and the residue concentrated twice with toluene. (α-d)-Phe-NH2 (235 mg, quant.) was obtained as white solid that was used without further purification.

### Step 3: Synthesis of (N2-Boc-N6-Cbz)-Lys-(α-d)-Phe-NH2

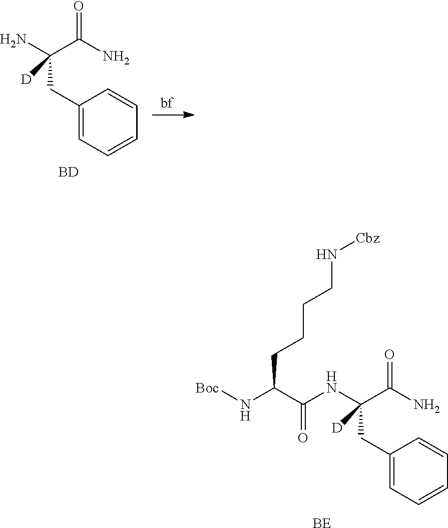

To mixture of (α-d)-Phe-NH₂ (BD, 230 mg, 0.885 mmol) and (N2-Boc-N6-Cbz)-Lys (477 mg, 1.25 mmol) in DCM (12 mL) were added HOBT*H₂O (210 mg, 1.37 mmol), EDC*HCl (265 mg, 1.37 mmol) and NMM (475 μl, 3.42 mmol). Reaction mixture was stirred at room temperature. After 72 h DCM was removed. Crude product was purified by reverse phase flash chromatography (15-65% MeOH in H₂O, 0.1% AcOH) to afford (N2-Boc-N6-Cbz)-Lys-(α-d)-Phe-NH2 white solid (360 mg, 60%).

### Step 4: Synthesis of (N6-Cbz)-Lys-(α-d)-Phe-NH2

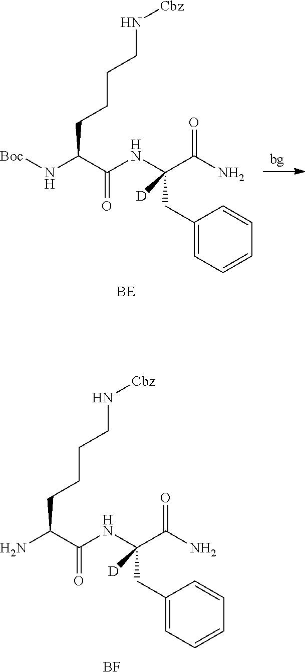 [SMILES: [2H][C@@](Cc1ccccc1)(NC(=O)[C@H](CCCCNC(=O)OCc1ccccc1)NC(=O)OC(C)(C)C)C(N)=O ; "BE" ; [2H][C@@](Cc1ccccc1)(NC(=O)[C@@H](N)CCCCNC(=O)OCc1ccccc1)C(N)=O ; "BF"]

To a cooled (0° C.) solution of (N2-Boc-N6-Cbz)-Lys-(α-d)-Phe-NH2 (BE, 360 mg, 0.682 mmol) in DCM (9 mL) TFA (2.0 mL) was added. After 5 min, the ice bath was removed and the mixture stirred at ambient temperature for 2 h. Volatiles was removed under reduced pressure. (N6-Cbz)-Lys-(α-d)-Phe-NH2 (370 mg, quant.) was obtained as white solid that was used without further purification. ¹H NMR (400 MHz, Methanol-d₄) δ 7.40-7.13 (m, 10H), 5.07 (s, 2H), 3.77 (t, J=6.3 Hz, 1H), 3.20-3.06 (m, 2H), 2.95 (d, J=14.0 Hz, 2H), 1.83 (tt, J=14.4, 7.5 Hz, 2H), 1.52 (q, J=7.1 Hz, 1H), 1.40 (q, J=7.9 Hz, 1H).

### Step 5: Synthesis of (Boc)-D-Arg-DMT-(N6-Cbz)-Lys-(α-d)-Phe-NH2

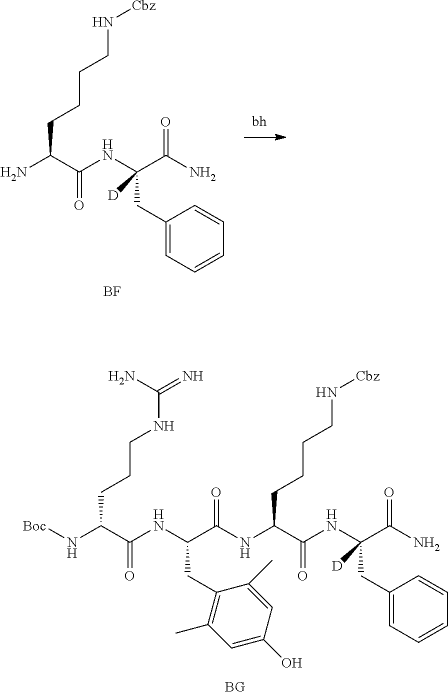

To mixture of (N6-Cbz)-Lys-(α-d)-Phe-NH2 (BF, 180 mg, 0.332 mmol) and (Boc)-D-Arg-DMT (192 mg, 0.366 mmol) in DMF (12 mL), HOBT*H₂O (61 mg, 0.400 mmol), EDC*HCl (96 mg, 0.500 mmol) and NMM (120 μL, 1.20 mmol) was added. Reaction mixture was stirred at room temperature. After 48 h DMF was removed. Crude product was purified by reverse phase flash chromatography (15-65% MeOH in H₂O, 0.1% AcOH) to afford (Boc)-D-Arg-DMT-(N6-Cbz)-Lys-(α-d)-Phe-NH2 as white foam (180 mg, purity—70%). Additional purification was performed by HPLC (Waters SFO, column: Xterra Prep RP18 OBD 10 um, 19×150 mm, 20 mL/min, water (0.05% TFA)/MeOH, solvent gradient from 2% to 10% of MeOH]. SBT-LIOS-018-9 was isolated as a white solid (64 mg, impurities<5%). ¹H NMR (400 MHz, Methanol-d₄) δ 7.42-7.12 (m, 10H), 6.43 (s, 2H), 5.06 (s, 2H), 4.59 (dd, J=9.1, 6.6 Hz, 1H), 4.16 (d, J=7.6 Hz, 1H), 3.94 (m, 1H), 3.24-2.89 (m, 10H), 2.25 (s, 6H), 1.71-1.58 (m, 4H), 1.42 (s, 9H), 1.48-1.32 (m, 4H).

### Step 6: Synthesis of (Boc)-D-Arg-DMT-Lys-((α-d)-Phe-NH2

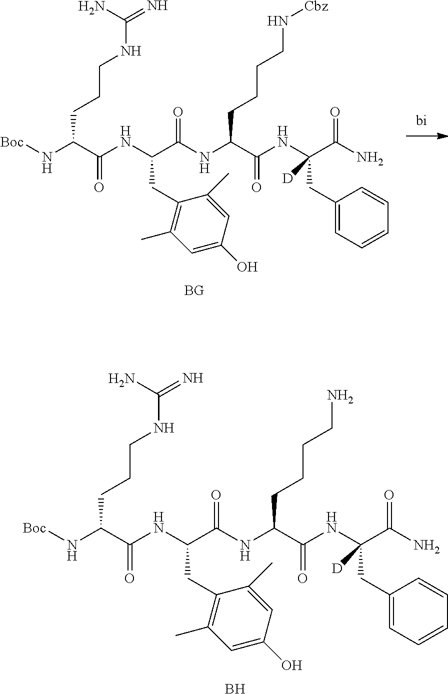

To a solution of (Boc)-D-Arg-DMT-(N6-Cbz)-Lys-(α-d)-Phe-NH₂ (BG, 64 mg, 0.156 mmol) in MeOH (10 mL) Pd/C (10% w/w, 15 mg) was added followed by acetic acid (36 μL, 0.625 mmol). The hydrogen was purged in reaction mixture at room temperature for 3 h. Then reaction mixture was filtrated through Celite pad and washed with MeOH (10 mL). The solvent was removed by evaporation. It was obtained white foam (53 mg, purity—70%). Additional purification was performed by HPLC (Waters SFO, column: Xterra Prep RP18 OBD 10 um, 19×150 mm, 20 mL/min, water (0.05% TFA)/MeOH, solvent gradient from 2% to 10% of MeOH]. (Boc)-D-Arg-DMT-Lys-(α-d)-Phe-NH2 was isolated as a white solid (45 mg, impurities<5%). ¹H NMR (400 MHz, Methanol-d₄) δ 7.41-7.07 (m, 5H), 6.41 (s, 2H), 4.66-4.48 (m, 1H), 4.30-4.09 (m, 1H), 4.03-3.85 (m, 1H), 3.19-3.00 (m, 4H), 2.98-2.81 (m, 4H), 2.22 (s, 6H), 1.58 (d, J=16.9 Hz, 6H), 1.41 (s, 9H), 1.37-1.23 (m, 4H).

### Step 7: Synthesis of D-Arg-DMT-Lys-(α-d)-Phe-NH2 (Example 10)

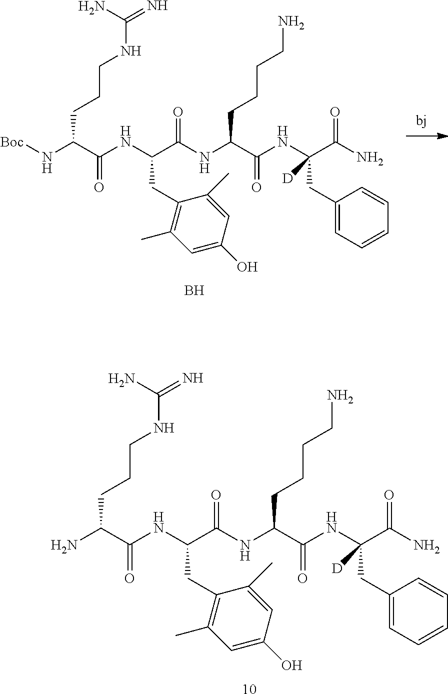

To a cooled (0° C.) solution of (Boc)-D-Arg-DMT-Lys-(α-d)-Phe-NH2 (BH, 34 mg, 0.083 mmol) in DCM (5 mL) TFA (1.0 mL) was added. After 5 min, the ice bath was removed and the mixture stirred at ambient temperature for 2 h. Volatiles was removed under reduced pressure. It was obtained 36 mg of D-Arg-DMT-Lys-(α-d)-Phe-NH2. ¹H NMR (400 MHz, Methanol-d₄) δ 7.35-7.14 (m, 5H), 6.44 (s, 2H), 4.83-4.73 (m, 1H), 4.24 (t, J=7.1 Hz, 1H), 3.48 (p, J=1.7 Hz, 1H), 3.20-2.83 (m, 6H), 2.26 (s, 6H), 1.87-1.54 (m, 5H), 1.45-1.21 (m, 5H). Molecular formula: C₃₂H₄₈DN₉O₅x3TFA; Molecular weight: 982.88; Free base molecular weight: 640.81. EI-MS: m/z 641.7 [M+1].

### Example 11: Synthesis of D-Arg-DMT-(4,4,5,5-d4)-Lys-Phe-NH2

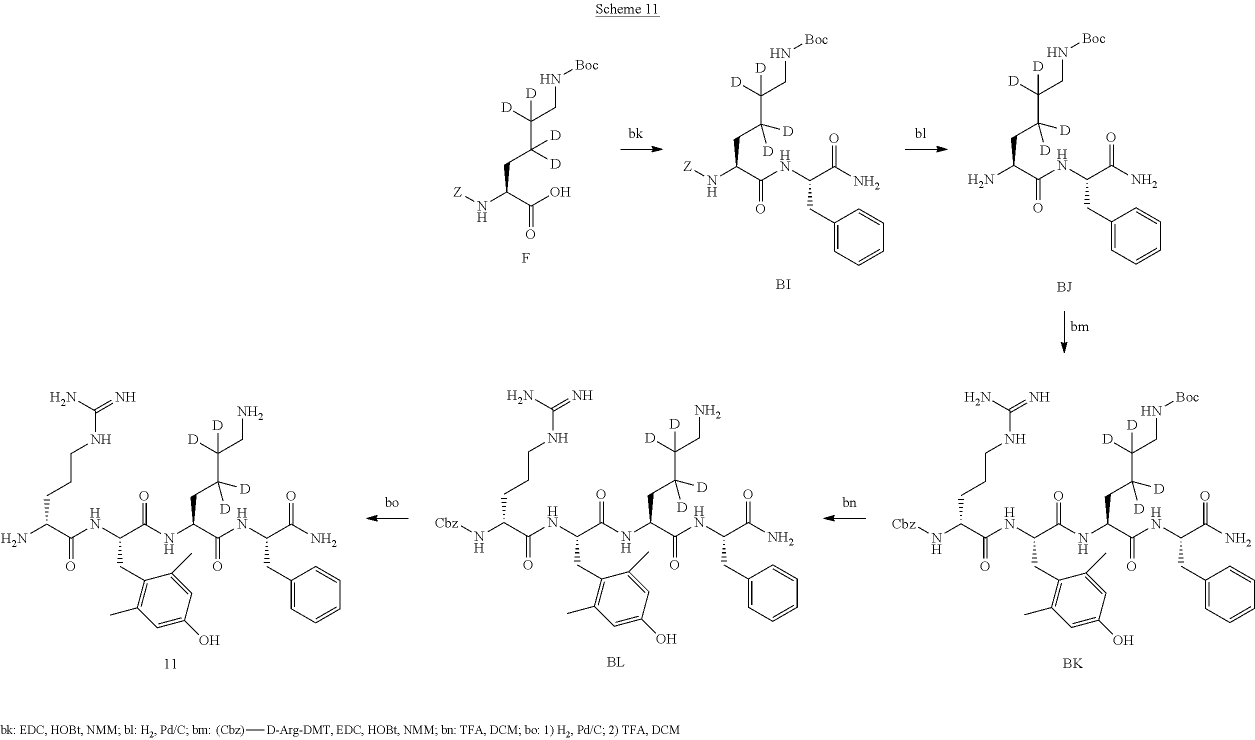

### Step 1: Synthesis of (N2-Cbz-N6-Boc-4,4,5,5-d4)-Lys-Phe-NH2

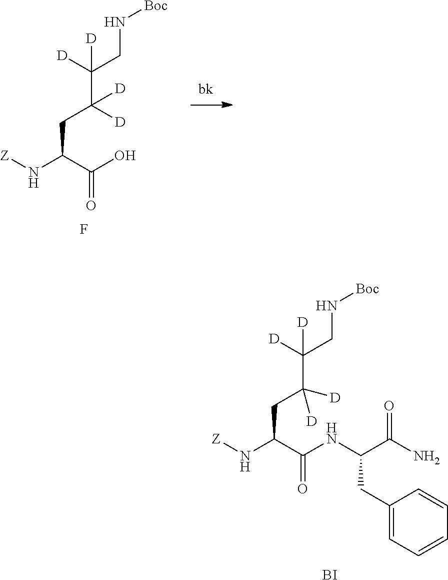

(N2-Cbz-N6-Boc-4,4,5,5-d4)-Lys (F, 165 mg, 0.429 mmol), HOBt (81 mg, 0.601 mmol), NMM (0.045 mL, 0.429 mmol) and EDC.HCl (148 mg, 0.773 mmol) were mixed in mixture of dry DMF (1.5 mL) at 10° C. and allowed to stir for 30 minutes. To this, a mixture of L-Phe-NH₂.HCl (95 mg, 0.472 mmol) and NMM (0.045 mL, 0.429 mmol) in DMF (1.3 mL) was added and the stirring continued at r.t. for 20 hours. Then DMF was evaporated and crude product was dissolved in DCM (20 mL). The reaction mixture was then treated with aqueous solutions of 5% citric acid solution in water (2×10 mL), aqueous layer was repeatedly extracted with DCM (8 mL). Organic layers was combined and washed with brine (10 mL), dried over Na₂SO₄, filtered and evaporated to dryness under reduced pressure. Product was purified by flash column chromatography (SiO₂, DCM-MeOH (10:1) R_{f}(PR) 0.3 to give product (174 mg, 76%) as a white amorphous solid. ¹H NMR (300 MHz, CD3OD-d₄) δ: 7.43-7.11 (m, 10H), 5.16-5.00 (m, 2H), 4.62 (dd, J=8.9, 5.6 Hz, 1H), 3.96 (t, J=6.9 Hz, 1H), 3.19 (dd, J=14.0, 5.1 Hz, 1H), 3.01-2.79 (m, 6H), 1.65-1.45 (m, 2H), 1.42 (s, 9H).

### Step 2: Synthesis of (N6-Boc-4,4,5,5-d4)-Lys-Phe-NH2

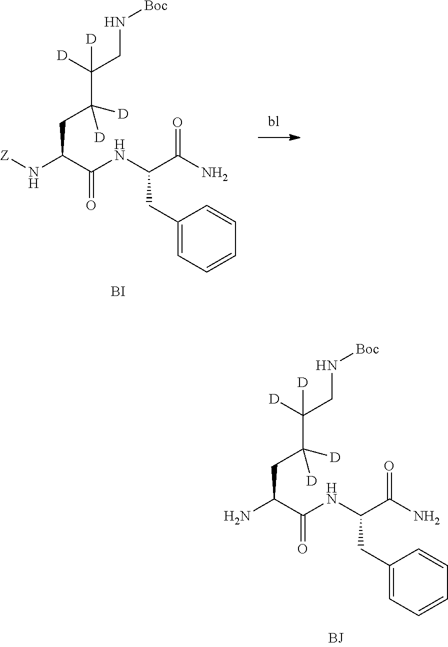

(N2-Cbz-N6-Boc-4,4,5,5-d4)-Lys-Phe-NH2 (BI, 174 mg, 0.329 mmol) was dissolved in MeOH (12 mL) and to this mixture 10% Pd/C (8.7 mg, 0.025 equiv.) was added. Hydrogen gas was bubbled for 4 h (TLC control: DCM-MeOH, 5:1, R_{f}(SM) 0.5, R_{f}(PR) 0.2-0.3). Then reaction mixture was filtered through Celite pad and evaporated to give crude product (128 mg, 98%) as transparent colorless oil. Used further without additional purification.

### Step 3: Synthesis of (Cbz)-D-Arg-DMT-(N6-Boc-4,4,5,5-d4)-Lys-Phe-NH2

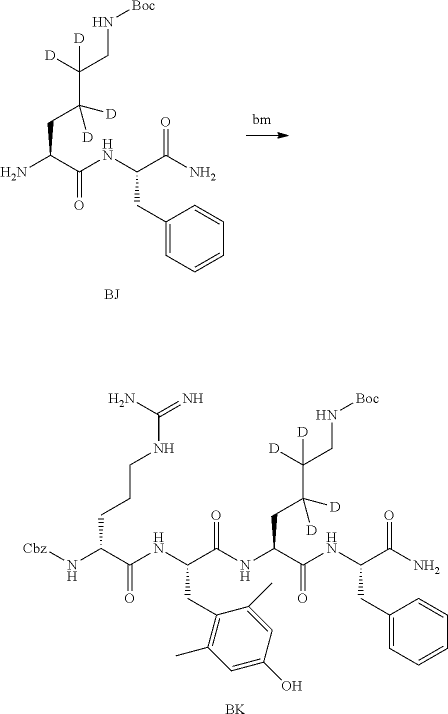

(Cbz)-D-Arg-DMT (191 mg, 0.356 mmol), (N6-Boc-4,4,5,5-d4)-Lys-Phe-NH2 (BJ, 128 mg, 0.324 mmol), HOBt (67 mg, 0.498 mmol) and EDC HC (123 mg, 0.641 mmol) were mixed in mixture of dry DMF (2.8 mL) at 10° C. and allowed to warm to r.t. and the stirring continued at r.t. for 20 h. Then DMF was evaporated from the reaction mixture. Crude product was washed with Et₂O (3×3 mL), dissolved in MeOH, added Celite and evaporated. Product purified by reverse phase flash chromatography (eluent: H₂O (0.2% AcOH)/MeOH from 5% to 85% of methanol) to give product (248 mg, 76%) as a white amorphous solid.

### Step 4: Synthesis of (Cbz)-D-Arg-DMT-(4,4,5,5-d4)-Lys-Phe-NH2

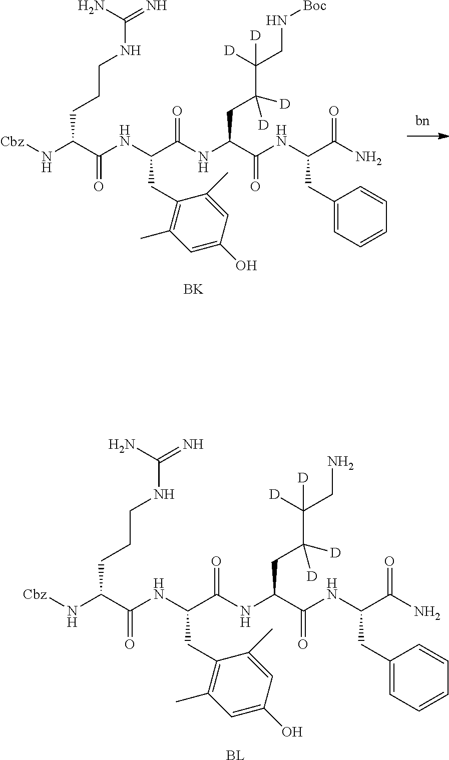

(Cbz)-D-Arg-DMT-(N6-Boc-4,4,5,5-d4)-Lys-Phe-NH2 (BK, 248 mg, 0.271 mmol) was dissolved in DCM (2.7 mL) and cooled to 0° C. TFA (0.42 mL, 5.424 mmol) was added dropwise and the solution was allowed to stir at 0° C. for 3 h. Then reaction mixture was evaporated (at 0-5° C.) and additionally re-evaporated from DCM (4 mL, at 0-5° C.). LC/MS shows no starting material, only product and HOBt impurity. Purification on PHPLC gave 180 mg (66%, HPLC purity—97.3% (210 nm)). ¹H NMR (400 MHz, CD3OD-d₄) δ: 7.40-7.13 (m, 10H), 6.44 (s, 2H), 5.05 (dd, J=36.5, 12.4 Hz, 2H), 4.63-4.53 (m, 2H), 4.20 (t, J=7.1 Hz, 1H), 4.01 (t, J=7.0 Hz, 1H), 3.24-3.03 (m, 4H), 2.99-2.89 (m, 2H), 2.84 (s, 2H), 2.24 (s, 6H), 1.77-1.59 (m, 3H), 1.58-1.28 (m, 3H).

### Step 5: Synthesis of D-Arg-DMT-(4,4,5,5-d4)-Lys-Phe-NH2 (Example 11)

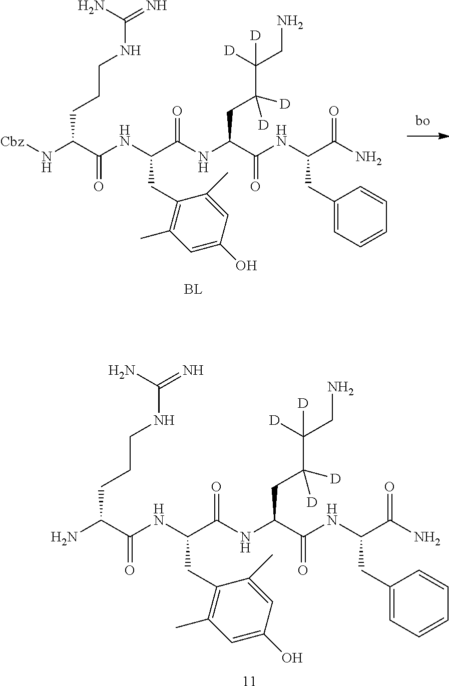

(Cbz)-D-Arg-DMT-(4,4,5,5-d4)-Lys-Phe-NH2 (BL, 180 mg, 0.179 mmol) was dissolved in MeOH (6.6 mL) and to this mixture 10% Pd/C (4.8 mg, 0.025 equiv.) was added. Hydrogen gas was bubbled for 3 h. Then reaction mixture was filtered and evaporated. Crude product was dissolved in DCM (4 mL) and cooled to 0-5° C. To mixture was added TFA (0.019 mL, 0.246 mmol) and after 10 min evaporated (ice bath) resulting (120 mg, 99%, HPLC shows purity—96.1 (210 nm)). ¹H NMR (400 MHz, CD3OD-d₄) δ: 7.32-7.14 (m, 5H), 6.44 (s, 2H), 4.78 (dd, J=9.2, 7.2 Hz, 1H), 4.59 (dd, J=8.6, 6.0 Hz, 1H), 4.24 (dd, J=7.8, 6.6 Hz, 1H), 3.94 (t, J=6.2 Hz, 1H), 3.17-3.02 (m, 4H), 3.00-2.89 (m, 2H), 2.86 (s, 2H), 2.26 (s, 6H), 1.78-1.54 (m, 4H), 1.43-1.16 (m, 2H). Molecular formula: C₃₂H₄₅D₄N₉O₅.3TFA; Molecular weight: 985.89; Free base molecular weight: 643.82. EI-MS: m/z 644.5 [M+1].

### Example 12: Synthesis of D-Arg-DMT-(3,3,4,4,5,5,6,6-d8)-Lys-Phe-NH2

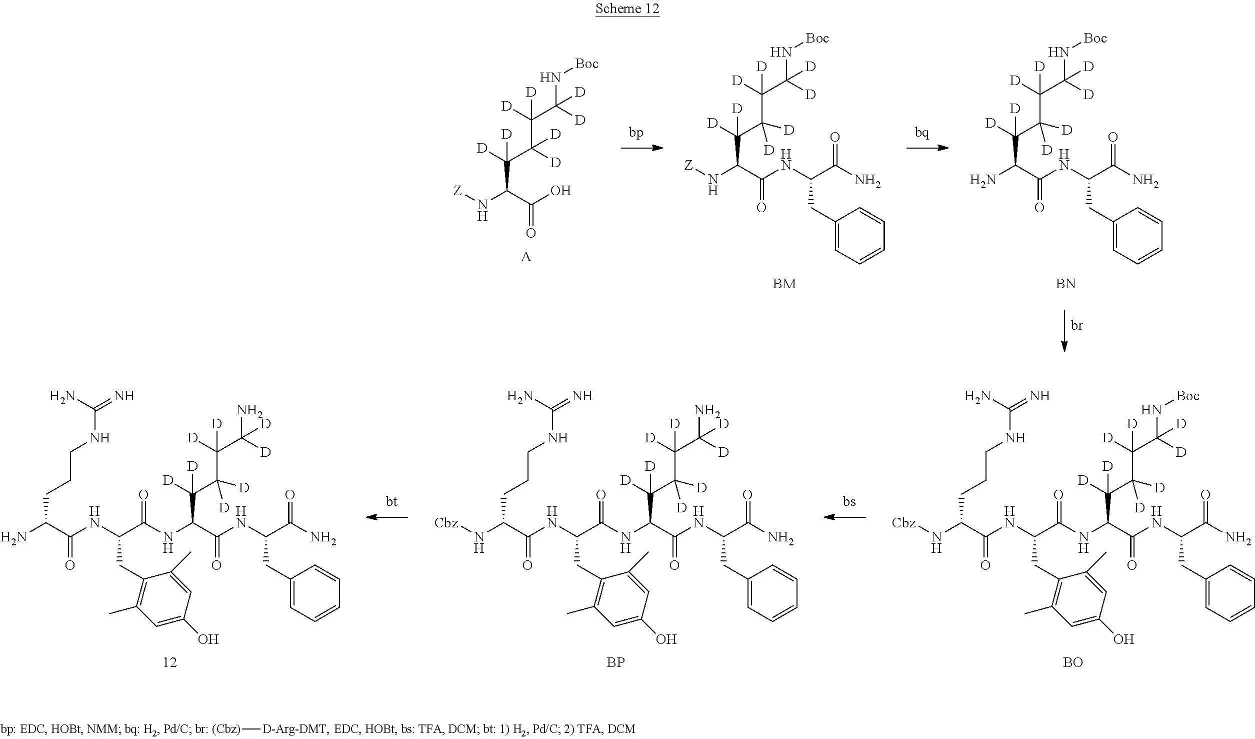

### Step 1: Synthesis of (N2-Cbz-N6-Boc-3,3,4,4,5,5,6,6-d8)-Lys-Phe-NH2

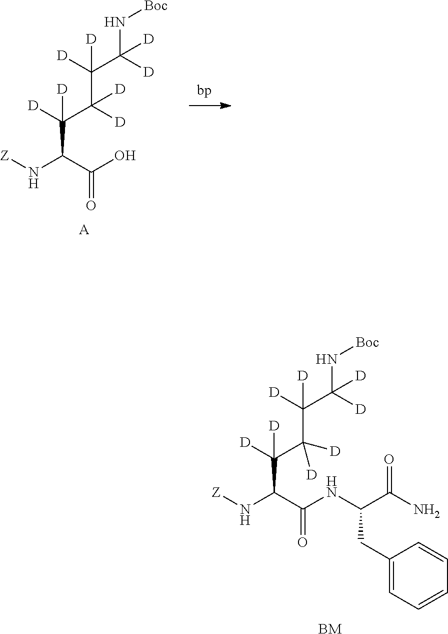

(N2-Cbz-N6-Boc-3,3,4,4,5,5,6,6-d8)-Lys (A, 440 mg, 1.13 mmol), HOBt (214 mg, 1.58 mmol), NMM (0.125 mL, 1.13 mmol) and EDC.HCl (390 mg, 2.03 mmol) were mixed in mixture of dry DMF (5 mL) at 10° C. and allowed to stir for 30 minutes. To this, a mixture of L-Phe-NH₂.HCl (251 mg, 1.25 mmol) and NMM (0.125 mL, 1.13 mmol) in DMF (2.3 mL) was added and the stirring continued at r.t. for 20 hours. Then DMF was evaporated and crude product was dissolved in DCM (50 mL). The reaction mixture was then treated with aqueous solutions of 5% citric acid solution in water (2×25 mL), aqueous layer was repeatedly extracted with DCM (20 mL). Organic layers was combined and washed with brine (25 mL), dried over Na₂SO₄, filtered and evaporated to dryness under reduced pressure to yield 646 mg crude product. Product was purified by flash column chromatography (SiO₂, DCM-MeOH (10:1) R_{f}(PR) 0.3) to give product as an amorphous solid (472 mg, 78%). ¹H NMR (300 MHz, CD3OD-d₄) δ: 7.40-7.14 (m, 10H), 5.12-5.01 (m, 2H), 4.62 (dd, J=9.0, 5.6 Hz, 1H), 3.95 (s, 1H), 3.19 (dd, J=13.8, 5.3 Hz, 1H), 3.01-2.83 (m, 2H), 1.42 (s, 9H).

### Step 2: Synthesis of (N6-Boc-3,3,4,4,5,5,6,6-d8)-Lys-Phe-NH2

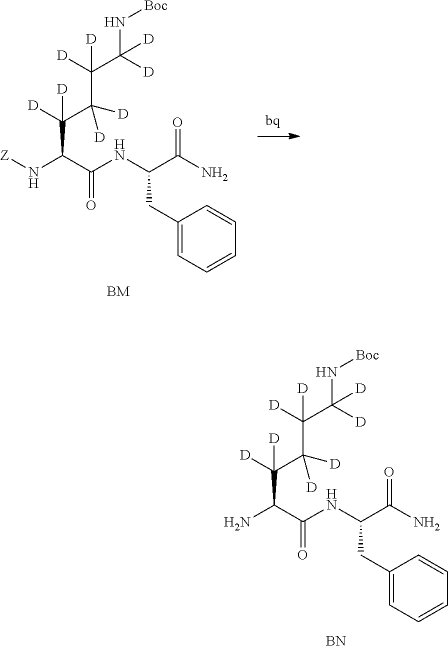

(N2-Cbz-N6-Boc-3,3,4,4,5,5,6,6-d8)-Lys-Phe-NH2 (BM, 200 mg, 0.374 mmol) was dissolved in MeOH (4 mL) and to this mixture 10% Pd/C (4 mg, 0.01 equiv.) was added. Hydrogen gas was bubbled for 4 h (TLC control: DCM-MeOH, 5:1, R_{f}(SM) 0.5, R_{f}(PR) 0.2-0.3). Then reaction mixture was filtered through Celite and evaporated to give crude product as transparent colorless oil (139 mg, 93%). Used further without additional purification. ¹H NMR (300 MHz, CD3OD-d₄) δ: 7.35-7.13 (m, 5H), 4.65 (dd, J=9.1, 5.6 Hz, 1H), 3.35 (s, 1H), 3.26-3.12 (m, 2H), 3.01-2.82 (m, 2H), 1.43 (s, 9H).

### Step 3: Synthesis of (Cbz)-D-Arg-DMT-(N6-Boc-3,3,4,4,5,5,6,6-d8)-Lys-Phe-NH2

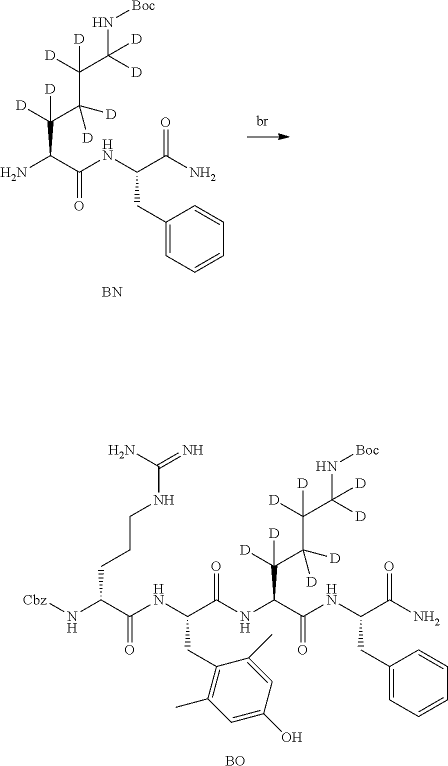

(Cbz)-D-Arg-DMT (205 mg, 0.382 mmol), (N6-Boc-3,3,4,4,5,5,6,6-d8)-Lys-Phe-NH2 (BN, 139 mg, 0.347 mmol), HOBt (66 mg, 0.486 mmol) and EDC.HCl (120 mg, 0.625 mmol) were mixed in dry DMF (3 mL) at 10° C. Reaction mixture allowed warming to r.t and the stirring continued at r.t. for 20 h. Then DMF was evaporated from the reaction mixture. Crude product washed with Et₂O (3×3 mL), dissolved in MeOH, added Celite and evaporated. Product purified by reverse phase flash chromatography (eluent: H₂O (0.2% AcOH)/MeOH from 5% to 85% of methanol) to give product as a white amorphous solid (255 mg, 80%). ¹H NMR (300 MHz, CD3OD-d₄) δ: 7.40-7.11 (m, 10H), 6.44 (s, 2H), 5.05 (dd, J=30.2, 12.4 Hz, 2H), 4.69-4.50 (m, 2H), 4.13 (s, 1H), 3.99 (t, J=6.5 Hz, 1H), 3.35 (s, 2H), 3.27-2.82 (m, 6H), 2.25 (s, 6H), 1.74-1.24 (m, 13H).

### Step 4: Synthesis of (Cbz)-D-Arg-DMT-(3,3,4,4,5,5,6,6-d8)-Lys-Phe-NH2

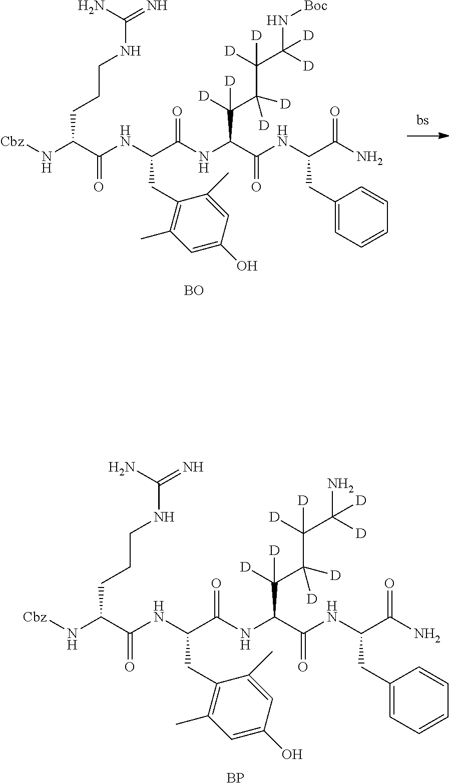

(Cbz)-D-Arg-DMT-(N6-Boc-3,3,4,4,5,5,6,6-d8)-Lys-Phe-NH2 (BO, 255 mg, 0.278 mmol) was dissolved in DCM (4.2 mL) and cooled to 0° C. TFA (0.42 mL, 5.55 mmol) was added drop-wise and the solution was allowed to stir at 0° C. for 3 h. Then reaction mixture was evaporated (at 0-5° C.) and additionally re-evaporated from DCM (4 mL, at 0-5° C.). LC/MS shows no starting material, only product and HOBt impurity. Purification on PHPLC gave product (206 mg, 79%, HPLC purity—97.34% (210 nm)) as a colorless amorphous solid. ¹H NMR (400 MHz, CD3OD-d₄) δ: 7.41-7.13 (m, 10H), 6.44 (s, 2H), 5.05 (dd, J=36.8, 12.5 Hz, 2H), 4.65-4.51 (m, 2H), 4.01 (t, J=7.0 Hz, 1H), 3.24-3.04 (m, 4H), 3.00-2.84 (m, 2H), 2.24 (s, 6H), 1.72-1.26 (m, 4H).

### Step 5: Synthesis of D-Arg-DMT-(3,3,4,4,5,5,6,6-d8)-Lys-Phe-NH2 (Example 12)

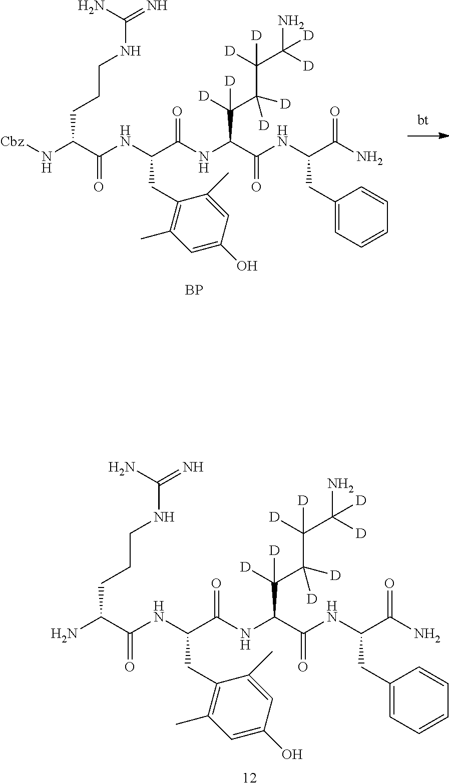

(Cbz)-D-Arg-DMT-(3,3,4,4,5,5,6,6-d8)-Lys-Phe-NH2 (BP, 206 mg, 0.204 mmol) was dissolved in MeOH (7.5 mL) and to this mixture 10% Pd/C (5.4 mg, 0.025 equiv.) was added. Hydrogen gas was bubbled for 3 h. Then reaction mixture was filtered and evaporated to give crude product as colorless transparent solid (151 mg). Crude product was dissolved in DCM (5 mL) and cooled to 0-5° C. To mixture was added TFA and after 10 min evaporated (ice bath) resulting (198 mg, 99%, HPLC shows purity: 92.01 (256 nm), 81.59 (210 nm)). Product was additionally purified by prep. HPLC, yielding 51 mg (25%, HPLC shows purity: 99.70% (256 nm), 98.95% (210 nm)). ¹H NMR (300 MHz, CD3OD-d₄) δ: 7.30-7.15 (m, 5H), 6.44 (s, 2H), 4.78 (dd, J=9.1, 7.2 Hz, 1H), 4.59 (dd, J=8.6, 5.9 Hz, 1H), 4.23 (s, 1H), 3.99-3.91 (m, 1H), 3.19-2.87 (m, 6H), 2.26 (s, 6H), 1.81-1.52 (m, 2H), 1.45-1.15 (m, 2H). Molecular formula: C₃₂H₄₁D₈N₉O₅.3TFA; Molecular weight: 989.92; Free base molecular weight: 647.85. EI-MS: m/z 648.6 [M+1].

### Example 13: Synthesis of D-Arg-DMT-(2,6,6-d3)-Lys-Phe-NH2

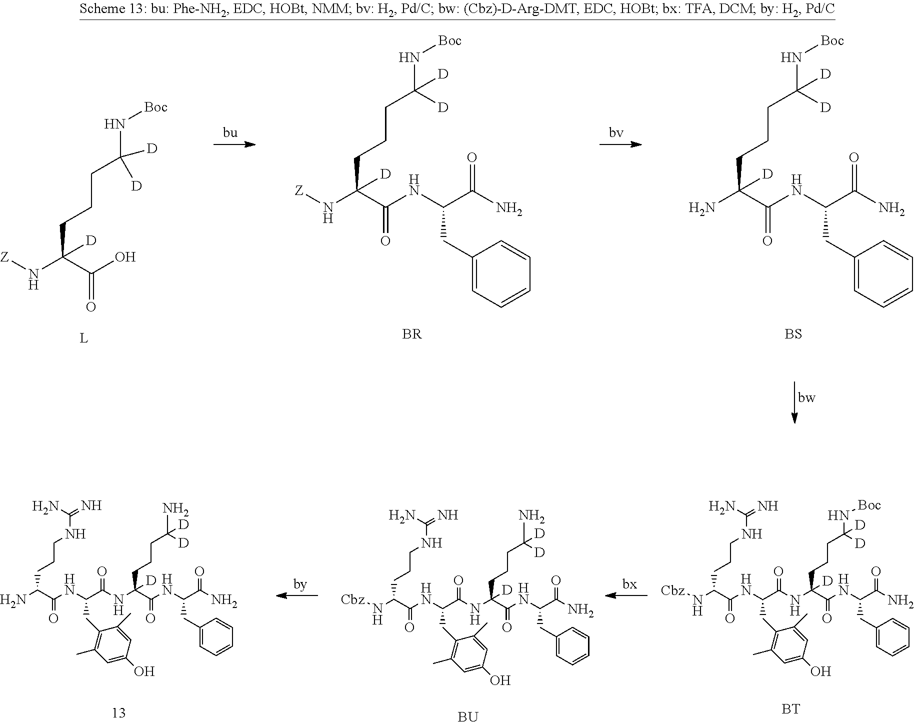

### Step 1. Synthesis of (N2-Cbz-N6-Boc-2,6,6-d3)-Lys-Phe-NH2

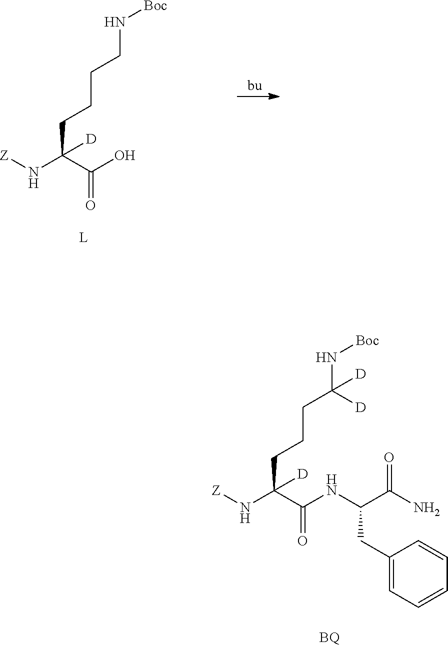

(N6-Boc-2,6,6-d3)-Lys (L, 168 mg, 0.438 mmol), HOBt (83 mg, 0.613 mmol), NMM (0.05 mL, 0.438 mmol) and EDC.HCl (151 mg, 0.788 mmol) were mixed in dry DMF (1.5 mL) at 10° C. and allowed to stir for 30 minutes. To this, a mixture of L-Phe-NH₂xHCl (97 mg, 0.482 mmol) and NMM (0.05 mL, 0.438 mmol) in DMF (1.3 mL) was added and the stirring continued at r.t. for 20 hours. Then DMF was evaporated and crude product was dissolved in DCM (20 mL). The reaction mixture was then treated with aqueous solutions of 5% citric acid solution in water (2×10 mL), aqueous layer was repeatedly extracted with DCM (8 mL). Organic layers was combined and washed with brine (10 mL), dried over Na₂SO₄, filtered and evaporated to dryness under reduced pressure to yield 241 mg technical grade product. Product was purified by flash column chromatography (SiO₂, DCM-MeOH (10:1) R_{f}(PR) 0.3 to give product as a white amorphous solid (143 mg, 62%). ¹H NMR (300 MHz, CD3OD-d₄) δ: 7.44-7.11 (m, 10H), 5.14-5.01 (m, 2H), 4.66-4.56 (m, 1H), 3.19 (dd, J=13.9, 5.3 Hz, 1H), 3.01-2.82 (m, 2H), 1.68-1.08 (m, 15H).

### Step 2. Synthesis of (N6-Boc-2,6,6-d3)-Lys-Phe-NH2

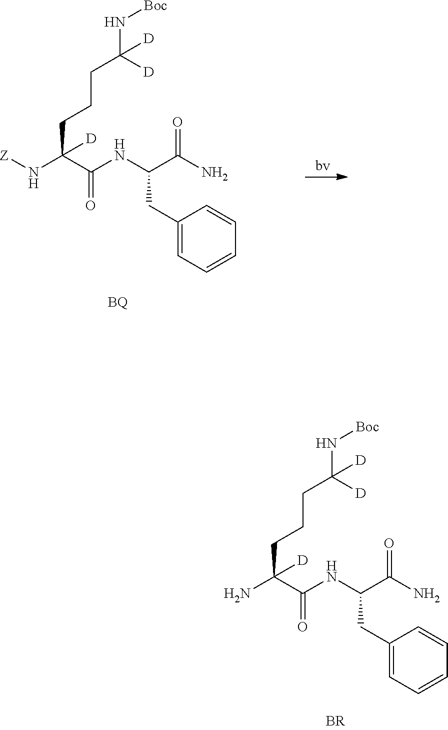

(N2-Cbz-N6-Boc-2,6,6-d3)-Lys-Phe-NH2 (BQ, 142 mg, 0.268 mmol) was dissolved in MeOH (10 mL) and to this mixture 10% Pd/C (7.1 mg, 0.025 equiv.) was added. Hydrogen gas was bubbled for 4 h (TLC control: DCM-MeOH, 5:1, R_{f}(SM) 0.5, R_{f}(PR) 0.2-0.3). Then reaction mixture was filtered through Celite and evaporated to give crude product as transparent colorless oil (105 mg, 99%). Used further without additional purification. ¹H NMR (300 MHz, CD3OD-d₄) δ: 7.34-7.14 (m, 5H), 4.64 (dd, J=9.1, 5.7 Hz, 1H), 3.35 (s, 2H), 3.17 (dd, J=13.9, 5.7 Hz, 1H), 3.02-2.83 (m, 1H), 1.65-1.07 (m, 15H).

### Step 3. Synthesis of (Cbz)-D-Arg-DMT-(N6-Boc-2,6,6-d3)-Lys-Phe-NH2

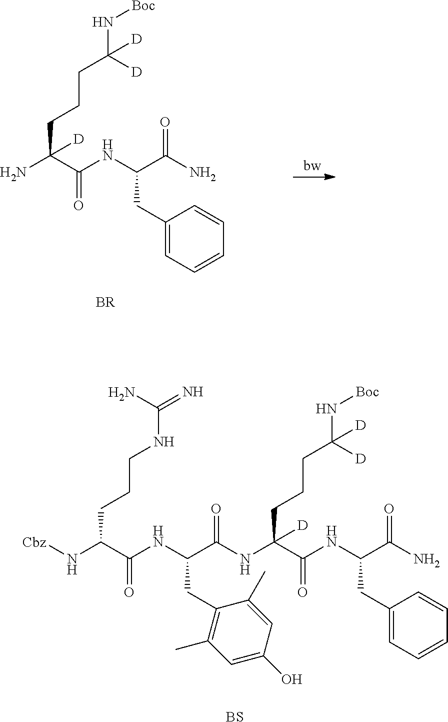 [SMILES: [2H]C([2H])(CCC[C@]([2H])(N)C(=O)N[C@@H](Cc1ccccc1)C(N)=O)NC(=O)OC(C)(C)C ; [2H]C([2H])(CCC[C@]([2H])(NC(=O)[C@H](Cc1c(C)cc(O)cc1C)NC(=O)[C@@H](CCCNC(=N)N)NC(=O)OCc1ccccc1)C(=O)N[C@@H](Cc1ccccc1)C(N)=O)NC(=O)OC(C)(C)C ; "BS"]

(Cbz)-D-Arg-DMT (157 mg, 0.292 mmol), (N6-Boc-2,6,6-d3)-Lys-Phe-NH2 (BR, 139 mg, 0.347 mmol), HOBt (50 mg, 0.371 mmol) and EDC.HCl (91 mg, 0.477 mmol) were mixed in dry DMF (2.1 mL) at 10° C., then allowed to warm to r.t. and the stirring continued at r.t. for 20 hours. Then DMF was evaporated from the reaction mixture. The crude product was washed with Et₂O (3×3 mL), dissolved in MeOH, added Celite and evaporated. Product purified by reverse phase flash chromatography (eluent: H₂O (0.2% AcOH)/MeOH from 5% to 85% of methanol) to give product (168 mg, 69%) as a white amorphous solid.

### Step 4. Synthesis of (Cbz)-D-Arg-DMT-(2,6,6-d3)-Lys-Phe-NH2

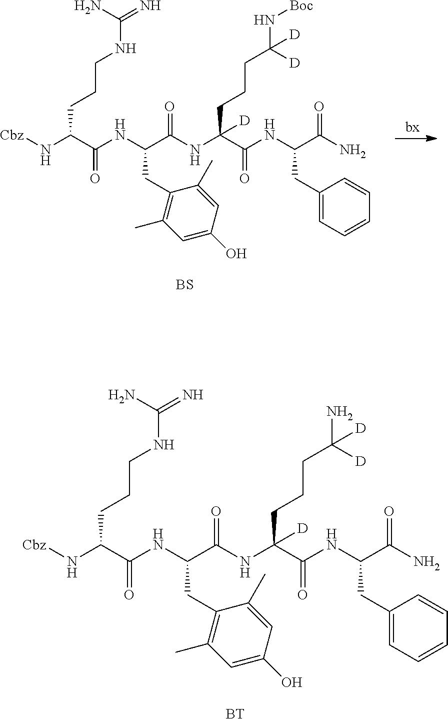 [SMILES: [2H]C([2H])(CCC[C@]([2H])(NC(=O)[C@H](Cc1c(C)cc(O)cc1C)NC(=O)[C@@H](CCCNC(=N)N)NC(=O)OCc1ccccc1)C(=O)N[C@@H](Cc1ccccc1)C(N)=O)NC(=O)OC(C)(C)C ; "BS" ; [2H]C([2H])(N)CCC[C@]([2H])(NC(=O)[C@H](Cc1c(C)cc(O)cc1C)NC(=O)[C@@H](CCCNC(=N)N)NC(=O)OCc1ccccc1)C(=O)N[C@@H](Cc1ccccc1)C(N)=O ; "BT"]

(Cbz)-D-Arg-DMT-(N6-Boc-2,6,6-d3)-Lys-Phe-NH2 (BS, 168 mg, 0.184 mmol) was dissolved in DCM (2.8 mL) and cooled to 0° C. TFA (0.28 mL, 3.68 mmol) was added dropwise and the solution was allowed to stir at 0° C. for 3 h. Then reaction mixture was evaporated (at 0-5° C.) and additionally re-evaporated from DCM (4 mL, at 0-5° C.). LC/MS shows no starting material, only product and HOBt impurity. Purification on PHPLC gave product (124 mg, 73%, HPLC purity—97.1% (210 nm)) as a white amorphous solid. ¹H NMR (300 MHz, CD3OD-d₄) δ: 7.39-7.07 (m, 10H), 6.44 (s, 2H), 5.05 (dd, J=28.3, 12.4 Hz, 21H), 4.67-4.50 (m, 2H), 4.08-3.95 (m, 1H), 3.27-3.02 (m, 4H), 3.02-2.81 (m, 21H), 2.24 (s, 6H), 1.80-1.21 (m, 10H).

### Step 5. Synthesis of D-Arg-DMT-(2,6,6-d3)-Lys-Phe-NH2 (Example 13)

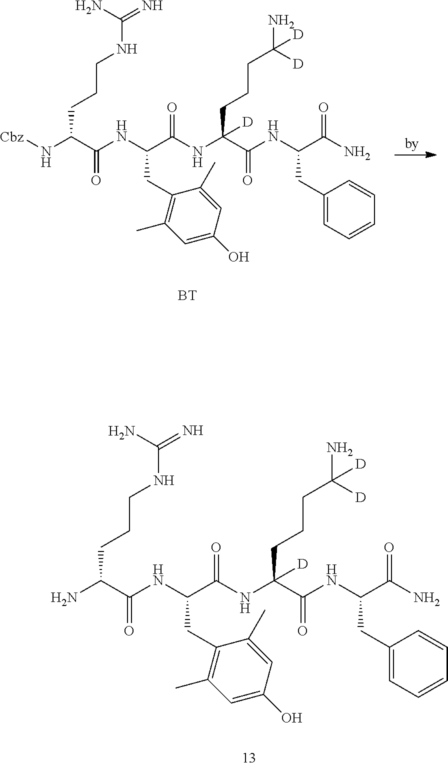 [SMILES: [2H]C([2H])(N)CCC[C@]([2H])(NC(=O)[C@H](Cc1c(C)cc(O)cc1C)NC(=O)[C@@H](CCCNC(=N)N)NC(=O)OCc1ccccc1)C(=O)N[C@@H](Cc1ccccc1)C(N)=O ; "BT" ; [2H]C([2H])(N)CCC[C@]([2H])(NC(=O)[C@H](Cc1c(C)cc(O)cc1C)NC(=O)[C@H](N)CCCNC(=N)N)C(=O)N[C@@H](Cc1ccccc1)C(N)=O]

(Cbz)-D-Arg-DMT-(2,6,6-d3)-Lys-Phe-NH2 (BT, 124 mg, 0.123 mmol) was dissolved in MeOH (4.6 mL) and to this mixture 10% Pd/C (3.3 mg, 0.025 equiv.) was added. Hydrogen gas was bubbled for 3 h. Then reaction mixture was filtered and evaporated to give crude product as colorless transparent solid (115 mg). Crude product was dissolved in DCM (3 mL) and cooled to 0-5° C. To mixture was added TFA (0.019 mL, 0.246 mmol) and after 10 min evaporated (ice bath) resulting a foam (120 mg, 99%, HPLC shows purity: 94.19 (256 nm), 96.07 (210 nm)). ¹H NMR (300 MHz, CD3OD-d₄) δ: 7.32-7.14 (m, 5H), 6.44 (s, 2H), 4.78 (dd, J=9.2, 7.2 Hz, 1H), 4.59 (dd, J=8.6, 6.0 Hz, 1H), 3.94 (t, J=6.1 Hz, 1H), 3.19-2.87 (m, 6H), 2.26 (s, 6H), 1.79-1.53 (m, 6H), 1.44-1.13 (m, 4H). Molecular formula: C₃₂H₄₆D₃N₉O₆.3TFA; Molecular weight: 984.89; Free base molecular weight: 642.82. EI-MS: m/z 643.5 [M+1].

### Example 14: Synthesis of (α,β,β,2,3,4,5,6-d8)-Phe-D-Arg-Phe-Lys-NH2

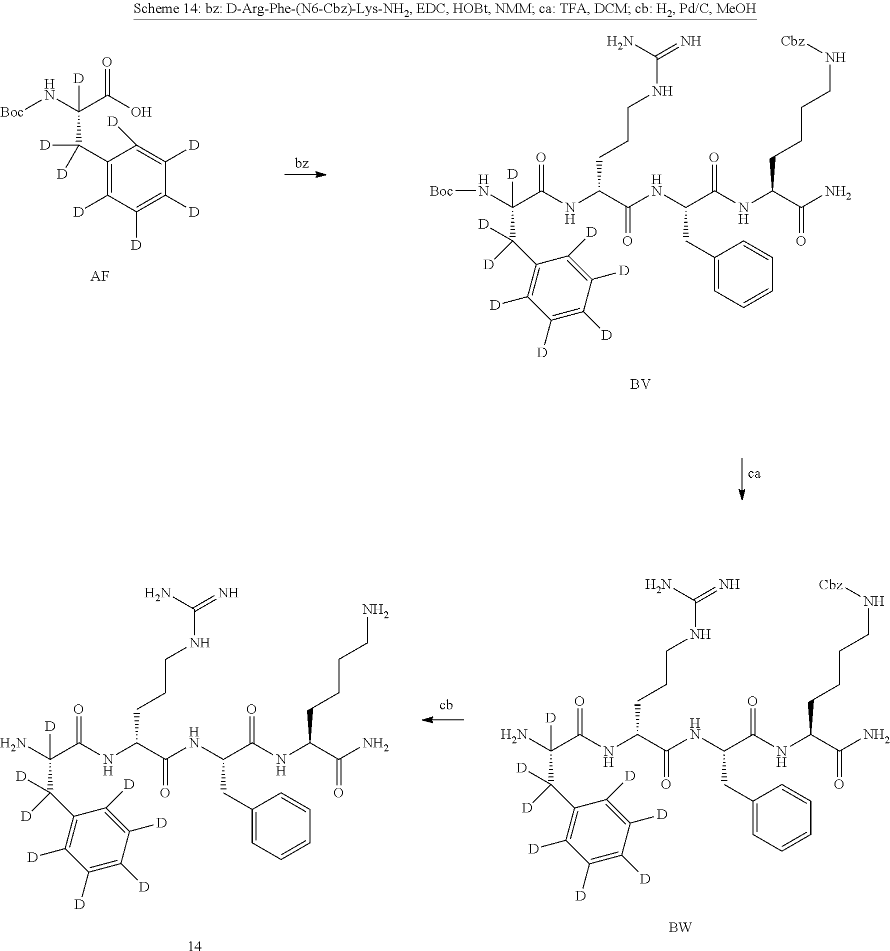

### Step 1. Synthesis of (N-Boc-α,β,β,2,3,4,5,6-d8)-Phe-D-Arg-Phe-(N6-Cbz)-Lys-NH2

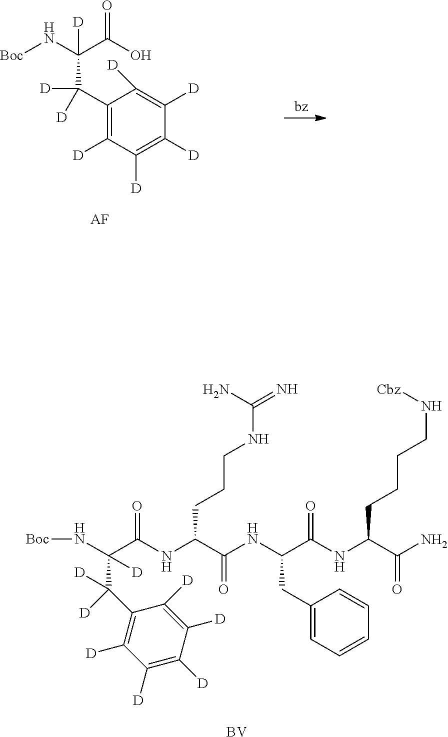 [SMILES: [2H]c1c([2H])c([2H])c(C([2H])([2H])[C@@]([2H])(NC(=O)OC(C)(C)C)C(=O)O)c([2H])c1[2H] ; "AF" ; [2H]c1c([2H])c([2H])c(C([2H])([2H])[C@]([2H])(NC(=O)OC(C)(C)C)C(=O)N[C@H](CCCNC(=N)N)C(=O)N[C@@H](Cc2ccccc2)C(=O)N[C@@H](CCCCNC(=O)OCc2ccccc2)C(N)=O)c([2H])c1[2H] ; "BV"]

To the mixture of (N-Boc-α,β,β,2,3,4,5,6-d8)-Phe (AF, 125 mg, 0.458 mmol) and D-Arg-Phe-(N6-Cbz)-Lys-NH₂ (310 mg, 0.445 mmol) in DMF (7 mL) HOBT*H₂O (82 mg, 0.534 mmol), EDC*HCl (145 mg, 0.801 mmol) and NMM (245 μL, 2.23 mmol) were added. Reaction mixture was stirred at rt overnight. Then DMF was removed and crude product was purified by reverse phase flash chromatography (20-65% MeOH in H₂O) to afford (N-Boc-α,β, β,2,3,4,5,6-d8)-Phe-D-Arg-Phe-(N6-Cbz)-Lys-NH2 white solid (165 mg, purity—80%). Additional purification was performed by HPLC (Waters SFO, column: Xterra Prep RP18 OBD 10 um, 19×150 mm, 20 mL/min, water (0.05% TFA)/MeOH, solvent gradient from 2% to 10% of MeOH]. The desired product was isolated as a white solid (135 mg, impurities<5%). ¹H NMR (300 MHz, CD3OD-d₄) δ: 7.33-7.19 (m, 10H), 5.05 (s, 2H), 4.57-4.53 (m, 1H), 4.29-4.26 (m, 1H), 4.15-4.11 (m, 1H), 3.26-3.21 (m, 1H), 3.13-3.11 (m, 2H), 3.10-2.96 (m, 3H), 1.92-1.82 (m, 1H), 1.78-1.72 (m, 1H), 1.60-1.30 (m, 4H), 1.37 (s, 9H), 1.13-1.02 (m, 2H).

### Step 2. Synthesis of (α,β,β,2,3,4,5,6-d8)-Phe-D-Arg-Phe-(N6-Cbz)-Lys-NH2

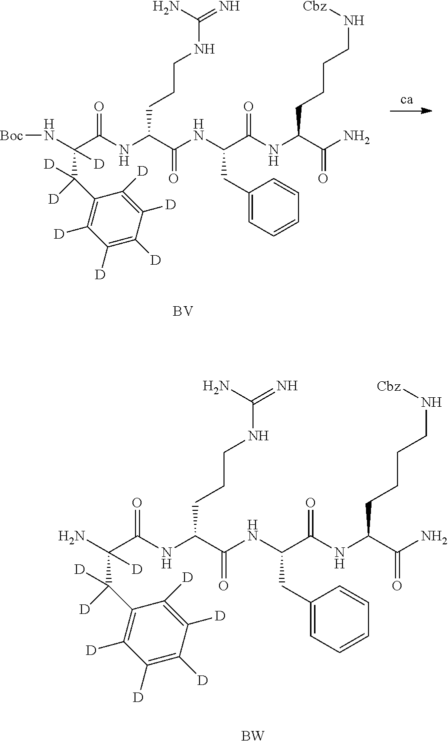 [SMILES: [2H]c1c([2H])c([2H])c(C([2H])([2H])[C@]([2H])(NC(=O)OC(C)(C)C)C(=O)N[C@H](CCCNC(=N)N)C(=O)N[C@@H](Cc2ccccc2)C(=O)N[C@@H](CCCCNC(=O)OCc2ccccc2)C(N)=O)c([2H])c1[2H] ; "BV" ; [2H]c1c([2H])c([2H])c(C([2H])([2H])[C@]([2H])(N)C(=O)N[C@H](CCCNC(=N)N)C(=O)N[C@@H](Cc2ccccc2)C(=O)N[C@@H](CCCCNC(=O)OCc2ccccc2)C(N)=O)c([2H])c1[2H] ; "BW"]

To a cooled (0° C.) solution of (N-Boc-α,β,β,2,3,4,5,6-d8)-Phe-D-Arg-Phe-(N6-Cbz)-Lys-NH2 (BV, 134 mg, 0.160 mmol) in DCM (9 mL) TFA (1.0 mL) was added. After 5 min, the ice bath was removed and the mixtures stirred at ambient temperature for 2 h. Volatiles were removed under reduced pressure. (α,β,β,2,3,4,5,6-d8)-Phe-D-Arg-Phe-(N6-Cbz)-Lys-NH2 (156 mg, purity—80%) was obtained. Purification was performed by HPLC (Waters SFO, column: Xterra Prep RP18 OBD 10 um, 19×150 mm, 20 mL/min, water (0.05% TFA)/MeOH, solvent gradient from 2% to 10% of MeOH]. The desired product was isolated as a white solid (112 mg, impurities<5%). ¹H NMR (400 MHz, Methanol-d₄) δ 7.41-7.12 (m, 10H), 5.06 (s, 2H), 4.64 (d, J=11.2 Hz, 2H), 4.39 (s, 1H), 4.06 (d, J=7.8 Hz, 1H), 3.21-3.03 (m, 2H), 2.99-2.73 (m, 3H), 1.94-1.67 (m, 2H), 1.64-1.23 (m, 6H), 1.04 (d, J=18.3 Hz, 1H), 0.92-0.74 (m, 1H).

### Step 3. Synthesis of (N-Boc-(α,β,β,2,3,4,5,6-d8)-Phe-D-Arg-Phe-(N6-Cbz)-Lys-NH2 (Example 14)

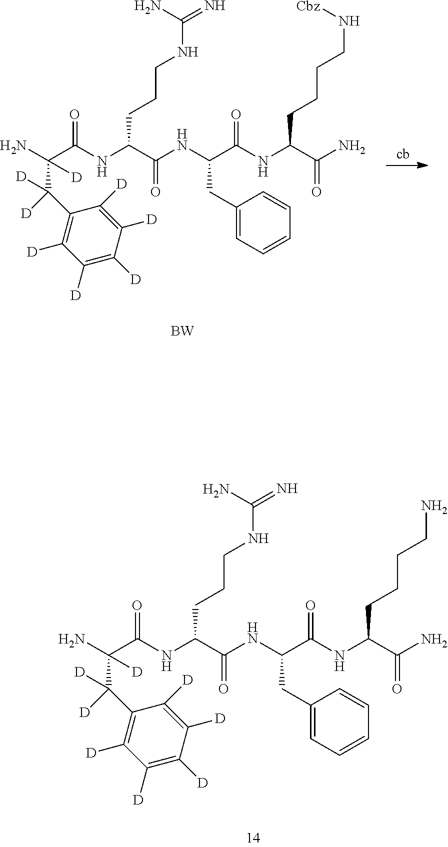 [SMILES: [2H]c1c([2H])c([2H])c(C([2H])([2H])[C@]([2H])(N)C(=O)N[C@H](CCCNC(=N)N)C(=O)N[C@@H](Cc2ccccc2)C(=O)N[C@@H](CCCCNC(=O)OCc2ccccc2)C(N)=O)c([2H])c1[2H] ; "BW" ; [2H]c1c([2H])c([2H])c(C([2H])([2H])[C@]([2H])(N)C(=O)N[C@H](CCCNC(=N)N)C(=O)N[C@@H](Cc2ccccc2)C(=O)N[C@@H](CCCCN)C(N)=O)c([2H])c1[2H]]

To a solution of (α,β,β,2,3,4,5,6-d8)-Phe-D-Arg-Phe-(N6-Cbz)-Lys-NH2 (BW, 110 mg, 0129 mmol) in MeOH (10 mL) Pd/C (10% w/w, 15 mg) was added followed by acetic acid (36 μl, 0.625 mmol). The hydrogen was purged in reaction mixture at room temperature for 3 h. Then reaction mixture was filtrated through Celite pad and washed with MeOH (10 mL). The solvent was removed by evaporation. It was obtained 49 mg white solid of (α,β,β,2,3,4,5,6-d8)-Phe-D-Arg-Phe-Lys-NH2. Additional purification was performed by HPLC (Waters SFO, column: Xterra Prep RP18 OBD 10 um, 19×150 mm, 20 mL/min, water (0.05% TFA)/MeOH, solvent gradient from 2% to 10% of MeOH]. The desired product was isolated as a white solid (38 mg, impurities<5%). ¹H NMR (400 MHz, Methanol-d₄) δ 7.35-7.14 (m, 5H), 4.62 (dd, J=11.1, 4.4 Hz, 1H), 4.42 (dd, J=8.8, 5.5 Hz, 1H), 4.15-4.04 (m, 1H), 3.05-2.79 (m, 6H), 1.95-1.62 (m, 4H), 1.59-1.22 (m, 4H), 1.13-0.98 (m, 1H), 0.97-0.78 (m, 1H). Molecular formula: C₃₀H₃₇D₈N₉O₄.3TFA; Molecular weight: 945.87; Free base molecular weight: 603.80. EI-MS: m/z 604.5 [M+1].

### Example 15: Pharmacokinetic Studies

### General Methods and Procedures of Rat PK Studies (Aptuit)

1. Formulation of test substances

All test formulations will be prepared on the day of administration prior to dosing and used as soon as possible. All test formulations will be stored at room temperature, prior to administration.

The dose formulation(s) will be prepared and administered by volume.
The target dose levels are detailed below:

| | Dose level | Dose volume | Dose concentration |
|---|---|---|---|
| Dose route | (mg/kg) | (ml/kg) | (mg/mL) |
| Intravenous | 1 | 2 | 0.5 |

2. Preparation of IV dose formulations

Test substances discrete doses will be formulated in saline water at the final concentration of 0.5 mg/mL and intravenously administered at the volume of 2 mL/kg to rats.

3. Dose administration

Actual body weights of animals will be recorded on the day of dose administration. Doses volumes will be adjusted taking into account the weight of the animal at the time of dose administration. Doses formulations will be administered intravenously via tail vein (2 mL/kg); an aliquot of each dose formulation will be taken before administration. The actual dose received by each animal will be registered and residual volumes discarded.

4. Sample collection and handling

After administration, serial blood samples will be collected from the lateral tail vein of each rat, then animals will be sacrificed humanely. Approximately 300 μL of Blood will be collected into EDTA tubes. Then exactly 240 μL of blood will be transferred into a micronic tube containing 10 μL of a solution (30 mg/mL NaF and 239 mg/mL Pefabloc) at each of the following time points post-dose (actual times will be recorded): 0.083, 0.25, 0.5, 1, 2, 4 and 8 hours after dosing. All blood samples will be thoroughly but gently mixed following collection and placed on wet ice. Blood will be centrifuged (3000 g for 10 min at approximately 4° C.) as soon as possible and two 50 μL aliquots of plasma will be transferred into micronic tubes. Residual blood will be discarded.

5. Storage of samples

Plasma samples will be frozen at approximately −80° C. as soon as possible after preparation and stored until analysis.

6. Plasma sample preparation and analysis

Plasma samples will be assayed using a method based on protein precipitation with methanol followed by HPLC/MS-MS analysis with an optimized analytical method. Calibration standards (CS) and Quality control samples (QC) will be prepared on the day of analysis. Study samples, CS, QC and blanks will be spiked with an internal standard (IS) to improve the precision of the assay. Study samples will be analyzed together with CS, QC and blank samples (including also double blanks). From the calibration curve, the linear range of the analytical method will be determined and lower and upper limits of quantitation specified. Details of the conditions and methods used will be included in the report. All dosing solutions will be checked for accuracy and actual concentration will be reported.

7. Data handling and analysis

The results for test substances in plasma will be subject to non-compartmental pharmacokinetic analysis using Phoenix WinNonlin 6.3 (see Section 8). The following parameters will be reported:

Calculated concentration of test item in the corresponding formulation (expressed as mg/mL).
Dose levels (expressed as mg/kg).
Actual collection times (expressed as hh:mm).
Plasma concentrations (expressed as ng/mL).
Quantitative information about 2 metabolites in Plasma.
PK parameters (iv: Cmax, AUClast, AUCinf, CL, Vss, t1/2, if applicable;
Abnormal clinical signs and relevant findings, if any.

TABLE 1
PK of Example 1

| | Cmax | Tmax | AUClast | AUCINF_obs | Cl_obs | t1/2 | Vss_obs |
|---|---|---|---|---|---|---|---|
| Example | (ng/mL) | (h) | (h * ng/mL) | (h * ng/mL) | (mL/min/kg) | (h) | (L/kg) |
| Example 1 | 3200 | 0.083 | 1950 | 1960 | 8.55 | 0.50 | 0.27 |
| non-deuterated | | | | | | | |
| Example 1 | 4300 | 0.083 | 2300 | 2310 | 7.24 | 0.45 | 0.21 |

TABLE 2
PK of Example 11

| | Cmax | Tmax | AUClast | AUCINF_obs | Cl_obs | t1/2 | Vss_obs |
|---|---|---|---|---|---|---|---|
| Example | (ng/mL) | (h) | (h * ng/mL) | (h * ng/mL) | (mL/min/kg) | (h) | (L/kg) |
| Example 11 | 2932 | 0.083 | 2123 | 2125 | 7.97 | 0.8 | 0.29 |
| non-deuterated | | | | | | | |
| Example 11 | 4683 | 0.083 | 2921 | 2942 | 5.7 | 0.5884 | 0.23 |

### Example 16: Langendorff Study

Ischemia-Reperfusion Injury-Langendorff Heart Preparation Protocol (Latvian Institute of Organic Synthesis)

The infarction study is performed according to the Langendorff technique as described previously (Kuka J, Vilskersts R, Cirule H, Makrecka M, Pugovics O, Kalvinsh I, et al. The cardioprotective effect of mildronate is diminished after co-treatment with L-carnitine. J Cardiovasc Pharmacol Ther. 2012; 17:215-222), with some modifications. Rats are anaesthetized with sodium pentobarbital (60 mg/kg) and heparin is administered intraperitoneally. For the infarction studies, the hearts are perfused with oxygenated (95% O2-5% CO2) Krebs-Henseleit (KH) buffer solution (118 mmol/L NaCl, 4.7 mmol/L KCl, 1.24 mmol/L CaCl2, 1.64 mmol/L MgCl2, 24.88 mmol/L NaHCO3, 1.18 mmol/L KH2PO4, and 0.05 mmol/L EDTA; pH 7.3-7.5; 36.8-37.0° C.) supplemented with 10 mM glucose at a constant perfusion pressure of 60 mmHg. A water-ethanol mixture (1:1)-filled balloon connected to a physiological pressure transducer (ADInstruments) is inserted into the left ventricle, and the baseline end-diastolic pressure set at 5-10 mmHg. The heart rate (HR), flow, left-ventricle developed pressure (LVDP), contractility (+dp/dt) are continuously recorded using a PowerLab 8/35 system from ADInstruments. The isolated rat hearts are adapted for 20 min and the left anterior descending coronary artery (LAD) is subsequently occluded for 30 min followed by 120 min of reperfusion. KH perfusion solution with or without added compound of interest (vehicle or 1 μM concentration) will be used for the whole time of isolated heart perfusion. Occlusion is confirmed by ˜40% drop in coronary flow. The infarct size is determined as described previously (Kuka, 2012; Liepinsh, 2013). Briefly, at the end of the reperfusion, the LAD is re-occluded, and the heart is perfused with 0.1% methylene blue dissolved in KH buffer solution. Afterwards, hearts are sectioned transversely from the apex to the base in 6 slices (5 if smaller heart) of 2 mm thickness and incubated in 1% triphenyl-tetrazolium chloride in phosphate buffer (pH 7.4, 37° C.) for 10 min to stain viable tissue red and necrotic tissue white. The planemetric analysis of cross-sectional images is performed using Image-Pro Plus v6.3 software to determine the area at risk (AR) and area of necrosis (AN), each expressed as a percentage of cross-sectional slice area. The obtained values are then used to calculate the infarct size (IS) as a percentage of the risk area according to the formula: IS=AN/AR×100%.

### Study Outline

20 min. adaptation+30 min. ischemia (LAD ligation)+120 min. reperfusion. Vehicle or compound 1 μM
The test article concentration(s) may be adjusted. Any changes will be recorded in the study file and the final report.
Endpoints: HR, flow, LVDP, dP/dt, infarct size-area of necrosis
CTRL (vehicle)+4 SBT compounds (n=8 per treatment) tested per set (up to 11 sets (up to 44 compounds))

The protocol and the number of compounds to be tested may be modified based on the experimental results and discussions with the Sponsor. Any changes to the protocol will be documented in the study file and in the protocol amendment.

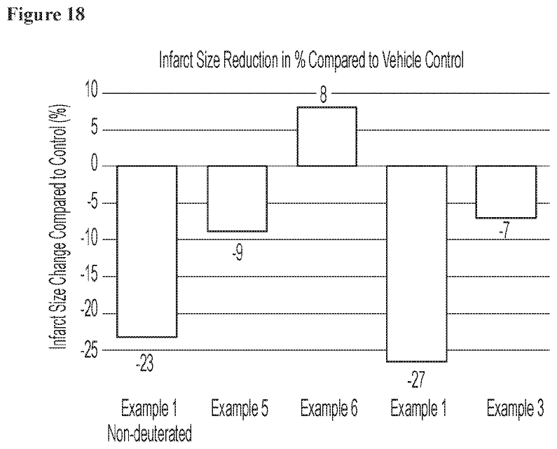

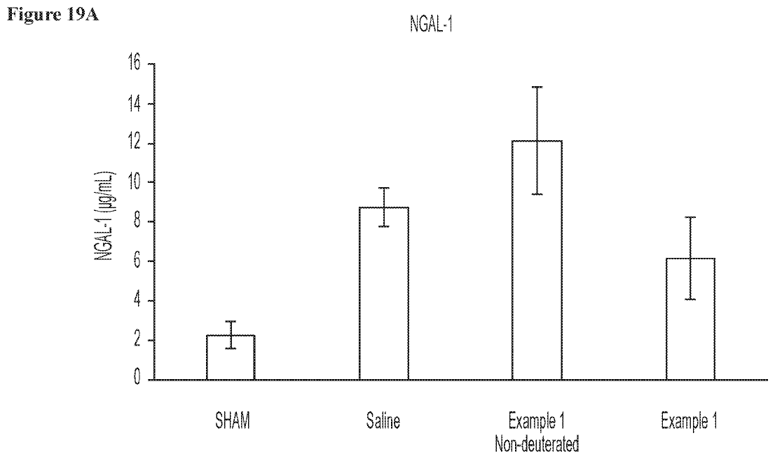

Results are shown in FIG. 18.

### Example 17: Renal Ischemic Study

Mouse Renal I/R Protocol (CL Lab)

C57BL6 mice will be marked with ear tags and divided into four groups, right nephrectomy plus left renal ischemia-reperfusion injury (IR), IR plus treatment with TA-001, or TA-002, or losartan (positive control). The treatments will be applied as indicated in the following table. Mice will be anesthetized with ketamine (100 mg/kg) and xylazine (10 mg/kg). Body temperature will be maintained at 37° C. by a temperature control system throughout the procedure. An incision will be made at the abdominal midline. Ischemia will be induced by clamping the left renal pedicle for 15 minutes using a non-traumatic vascular clip. Reperfusion will be followed by removing the clip. Sham animals will be subjected to no ischemia. Next, the contralateral kidney will be removed in all mice and snap-frozen (later it will be sent to Stealth Biotherapeutic Inc). The abdomen will be closed in two layers. After awake, mice will return to their cages and receive the pain reliever buprenorphine 0.1 mg/kg s.c. At the end of 24-hr reperfusion, the mice will be administered with ketamine (100 mg/kg) and xylazine (10 mg/kg). The blood will be collected from the right carotid artery. After blood collection, the mice will be euthanized by heart removal. Serum will be separated from the blood cells by centrifuging at 2,000×g for 10 min. Serum will be assayed for creatinine and BUN, and KIM-1 and Ngal.

C57BL6 mice at age of 10 weeks old will be imported from the Jackson Laboratory. They will be housed in groups of four to five per cage in a room maintained at 23±1° C. and 55±5% humidity with a 12-h light/dark cycle and will be given ad libitum access to food and water. The animal protocol is approved by Institutional Animal Care and Use.

1. Test Article(s) Dosing

| Dosing method | SQ (s.c.) |
|---|---|
| Dose 2 mg/kg | |
| Formulation | 1 mg/mL in saline |
| Vehicle | saline |
| Dosing times | 1 hr before clamping & |
| | at a time of reperfusion |
| Test article(s) storage (solid) | −20° C. |
| Formulation(s) storage | 0-5° C., up to 7 days |

2. Clinical Chemistry and Biomarkers

Approximately 0.7 mL of blood samples will be collected from all animals and put into tubes without anticoagulant for serum separation. The tubes will be kept at room temperature for a minimum of 90 min and then centrifuged (approximately 3000 rpm, 10 min, at room temperature) to obtain serum. Serum creatinine, BUN, KIM-1, and Ngal-1 will be measured as follow:

2.1 Serum Creatinine Assay

Serum creatinine will be measured by using QuantiChrom™ Creatinine Assay Kit (DICT-500, BioAssay Systems). Briefly, creatinine standard solutions from 4 to 0.125 mg/dl will be made by 1:2 dilution with distilled water. 30 μL of diluted standard and serum in duplicate into wells of a clear bottom 96-well plate. Then prepare enough Working Reagent by mixing per well reaction at least 100 μL Reagent A and 100 μL Reagent B. Next, add 200 μL Working Reagent quickly to all wells. Tap plate briefly to mix. Lastly, read optical density immediately (OD0) and then at 5 min (OD5) at 510 nm.

2.2 Serum BUN Assay

Serum BUN will be measured by using QuantiChrom™ Urea Assay Kit (DIUR-100, BioAssay Systems) Briefly, urea standard solutions will be made by dilution with distilled water. Then 5 μL of water (blank), 5 μL standard and 5 μL samples in duplicate will be transferred into wells of a clear bottom 96-well plate. Next, 200 μL working reagent will be added. Tap plate lightly to mix and incubate 20 min at room temperature. The plate will be read optical density immediately (OD0) and then at 5 min (OD5) at 520 nm.

2.3 Mouse KIM-1 and Ngal-1 ELISA

Serum KIM-1 and Ngal-1 will be measured by their respective mouse ELISA kits. Briefly, 0.1 mL of mouse KIM1 standard solutions and serum and sample buffer will be transferred into the pre-coated 96-well plate. The plate will be sealed with the cover and incubated at 37° C. for 90 min, then the plate content will be discarded by blotting it onto paper towels. 0.1 ml of biotinylated anti-mouse KIM1 antibody or Ngal-1 antibody working solution will be added into each well and incubate the plate at 37° C. for 60 min, followed by three washes with 0.01 M TBS. Add 0.1 mL of prepared ABC working solution into each well and incubate the plate at 37° C. for 30 min, followed by five washes. Add 90 of prepared TMB color developing agent into each well and incubate plate at 37° C. in dark for 20 min. Add 0.1 ml of prepared TMB stop solution into each well. The color changes into yellow immediately. Read the O.D. absorbance at 450 nm in a microplate reader within 30 min after adding the stop solution.

3. Calculations

Creatinine concentration of the sample is calculated as

=(OD_{SAMPLE 5}−OD_{SAMPLE 0})/(OD_{STD 5}−OD_{STD 0})×[STD] (mg/dL)

OD_{SAMPLE5}, OD_{SAMPLE0}, OD_{STD5} and OD_{STD0} are OD₅₁₀ₙₘ values of sample and standard at 5 and 0 min, respectively. [STD] is 2 mg/dL for blood assay.
Urea concentration (mg/dL) of the sample is calculated as

[Urea]=(OD_{SAMPLE}−OD_{BLANK})/(OD_{STANDARD}−OD_{BLANK})×n×[STD](mg/dL)

OD_{SAMPLE}, OD_{BLANK} and OD_{STANDARD} are OD values of sample, standard and water, respectively. n is the dilution factor. [STD]=50 (or 5 for low urea samples), urea standard concentration (mg/dL).
KIM-1 and Ngal-1 concentration will be calculated as:
(the relative O.D.450)=(the O.D.450 of each well)−(the O.D.450 of Zero well). The standard curve can be plotted as the relative O.D.450 of each standard solution (Y) vs. the respective concentration of the standard solution (X). The Mouse KIM1 concentration of the samples can be interpolated from the standard curve.

4. Replicates

All samples will be run in N=2-5 replicates for test articles TA-001, TA-002, and vehicle control, N=3 for losartan and N=2 for sham.

5. Acceptance Criteria

After renal ischemia-reperfusion, creatinine and BUN increase by >100% for vehicle control. KIM-1 and Ngal-1 raise by >50% for vehicle control.

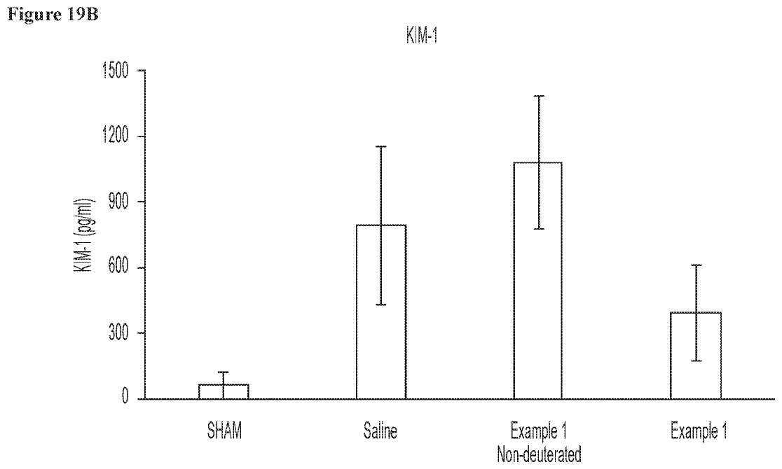

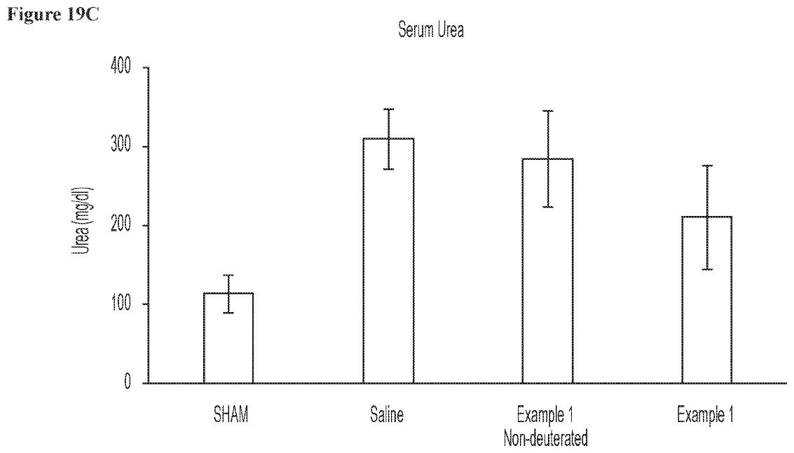

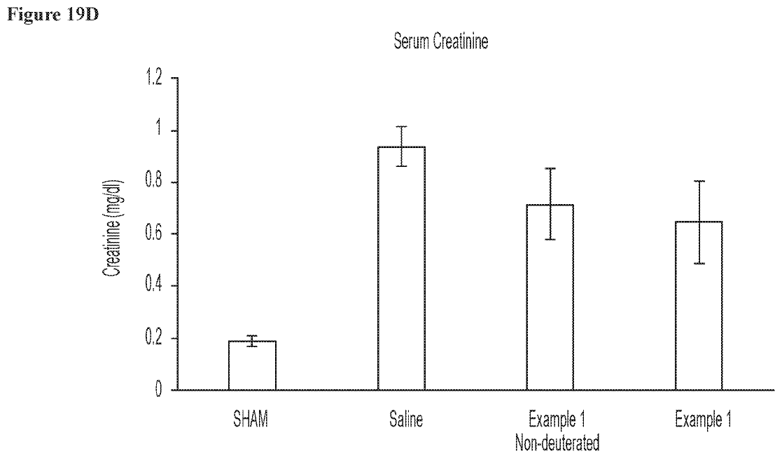

Results are shown in FIGS. 19A-19D.

### Example 18: Isothermal Titration Calorimetry (ITC) Study

1. Preparation of CL:DOPC 1:3 Liposomes (CL Concentration 0.1 mM)

146 μl of cardiolipin solution (5.1 mg/mL in EtOH, Sigma-Aldrich, C1649-10MG) were mixed with 1.2 mg 0.05 mg (exact weight, see Table 1) of DOPC (Avanti Polar Lipids, 850375P) and 20 mM HEPES buffer pH=7.4. Resulting solution was diluted up to 5 mL with the same buffer, vortexed for 2 min and sonicated in bath sonicator for 30 min at room temperature. Liposomes were produced by manual extrusion (Avestin, The LiposoFast-Stabilizer with a LiposoFast-Basic) through a 100 nm filter (Avestin, PC membranes 0.1 m or Avanti Polar Lipids, PC membranes 0.1 μm, 610005). Extrusion was performed for 21 times. EtOH concentration in sample is 2.9%. Obtained CL:DOPC solutions were tested for homogeneity by DLS (Malvern Instruments Ltd, Zetasizer Nano ZSP, instrument with Malvern Instruments Ltd software 7.11) using the following parameters—medium: water; refractive index: 1.33; viscosity: 0.8872 cP; temperature: 25° C.; nanoparticles: liposomes; refractive index of materials: 1.60; detection angle of 173°, a wavelength of 633 nm. All measurements were performed in triplicate.

2. Preparation of Test Compound Solutions

2 mg or 14 mg (exact weight, see Table 1) of test compound were dissolved in 20 mM HEPES buffer (pH=7.4, contains 2,9% (V/V) of EtOH) to obtain 0.7 mM concentration (for exact volumes see Table 3). Obtained solution was vortexed for 2 min.

TABLE 3
Sample preparation of test compounds Example 1 non-deuterated,
Example 1 and Example 8 non-deuterated

| | | | | | | Volume |
|---|---|---|---|---|---|---|
| | | | DOPC | | Test | of |
| | | | sample | Z | compound | HEPES |
| | Test | Replicate | weight, | average, | sample weight | buffer, |
| # | compound | sample | mg | nm | for ITC, mg | mL |
| 1 | Example 8, | 1 | 1.17 | 135.9 | 14.02 | 25.00 |
| 2 | non- | 2 | | 138.9 | | |
| 3 | deuterated | 3 | 1.21 | 139.2 | 14.00 | 25.00 |
| 4 | Example 1, | 1 | 1.22 | 127.6 | 2.013 | 4.83 |
| 5 | non- | 2 | | 126.6 | | |
| 6 | deuterated | 3 | | 127.5 | | |
| 7 | Example 1 | 1 | 1.22 | 113.8 | 2.003 | 4.01 |
| 8 | | 2 | | 115.5 | | |
| 9 | | 3 | | 116.9 | | |

3. ITC Titration Experiments

Instrument cell was washed and purged with ≈100 μL of CL:DOPC liposomes solution. After washing cell was filled with CL:DOPC liposomes solution. Injection syringe was loaded with test compound solution. Injection volumes were set to values shown in Table 2 and DP values were set to 2.5 μcal/sec in all experiments. Time between injections—105 seconds. Titration of each compound is performed in triplicate, results are reported as mean value±standard deviation (see Table 4).

TABLE 4
ITC titration parameters.

| | | | Injection | |
|---|---|---|---|---|
| | Test | Replicate | volume, | Approximate first peak |
| # | compound | sample | μL | energy response, μcal/sec |
| 1 | Example 8 | 1 | 1.5 | −1.4 |
| 2 | non- | 2 | 1.5 | −1.3 |
| 3 | deuterated | 3 | 1.5 | −1.4 |
| 4 | Example 1 | 1 | 1.5 | −0.9 |
| 5 | non- | 2 | 1.5 | −0.8 |
| 6 | deuterated | 3 | 1.5 | −0.8 |
| 7 | Example 1 | 1 | 1.5 | −1.1 |
| 8 | | 2 | 2 | −1.1 |
| 9 | | 3 | 2 | −1.2 |

TABLE 5
ITC titration results

| | Comp. | | | | ΔH, | ΔS, | ΔG | -TΔS |
|---|---|---|---|---|---|---|---|---|
| | (SBT) | N | Ka, M⁻¹ | Kd, M | cal/mol | cal/mol | kcal/mol | kcal/mol |
| 1 | Example 8 | 0.45 | 4.44E+05 | 2.25E-06 | -11380 | -12.3 | -7.71 | 3.67 |
| 2 | non- | 0.47 | 4.27E+05 | 2.34E-06 | -11240 | -11.9 | -7.69 | 3.55 |
| 3 | deuterated | 0.42 | 5.59E+05 | 1.79E-06 | -11550 | -12.4 | -7.85 | 3.70 |
| 4 | Average | 0.45 | 4.77E+05 | 2.13E-06 | -11390.0 | -12.20 | -7.75 | 3.64 |
| 5 | SD | 0.03 | 7.18E+04 | 2.97E-07 | 155.24 | 0.26 | 0.09 | 0.08 |
| 6 | Example 1 | 0.62 | 2.30E+05 | 4.35E-06 | -6660 | 2.19 | -7.31 | -0.65 |
| 7 | non- | 0.44 | 1.41E+05 | 7.09E-06 | -6666 | 1.20 | -7.02 | -0.36 |
| 8 | deuterated | 0.43 | 2.11E+05 | 4.74E-06 | -6508 | 2.54 | -7.27 | -0.76 |
| 9 | Average | 0.50 | 1.94E+05 | 5.39E-06 | -6611 | 1.98 | -7.20 | -0.59 |
| 10 | SD | 0.10 | 4.69E+04 | 1.48E-06 | 90 | 0.70 | 0.299 | 0.21 |
| 11 | Example 1 | 0.32 | 4.73E+05 | 2.11E-06 | -8821 | -3.63 | -7.74 | 1.08 |
| 12 | | 0.39 | 3.84E+05 | 2.60E-06 | -8 317 | -2.32 | -7.63 | 0.69 |
| 13 | | 0.39 | 3.66E+05 | 2.73E-06 | -8 575 | -3.31 | -7.59 | 0.99 |
| 14 | | 0.42 | 3.31E+05 | 3.02E-06 | -8 577 | -3.49 | -7.54 | 1.04 |
| 15 | Average | 0.38 | 3.89E+05 | 2.62E-06 | -8573 | -3.2 | -7.62 | 0.95 |
| 16 | SD | 0.05 | 6.05E+04 | 3.78E-07 | 206 | 0.59 | 0.38 | 0.18 |

### EQUIVALENTS

Having described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

### INCORPORATION BY REFERENCE

All U.S. patents, and U.S. and PCT published patent applications mentioned in the description above are incorporated by reference herein in their entirety.

We claim:

1. A compound of formula (I), or a pharmaceutically acceptable salt thereof:
Aaa¹-Aaa²-Aaa³-Aaa⁴-NH₂ (I);
wherein:
Aaa¹ is an L-phenylalanine residue;
Aaa² is a D-arginine residue;
Aaa³ is an L-phenylalanine residue;
Aaa⁴ is an L-lysine residue; and
at least one hydrogen atom is replaced by a deuterium atom.

2. The compound of claim 1, wherein:
Aaa¹ is an L-phenylalanine residue or an amino acid residue selected from the group consisting of: 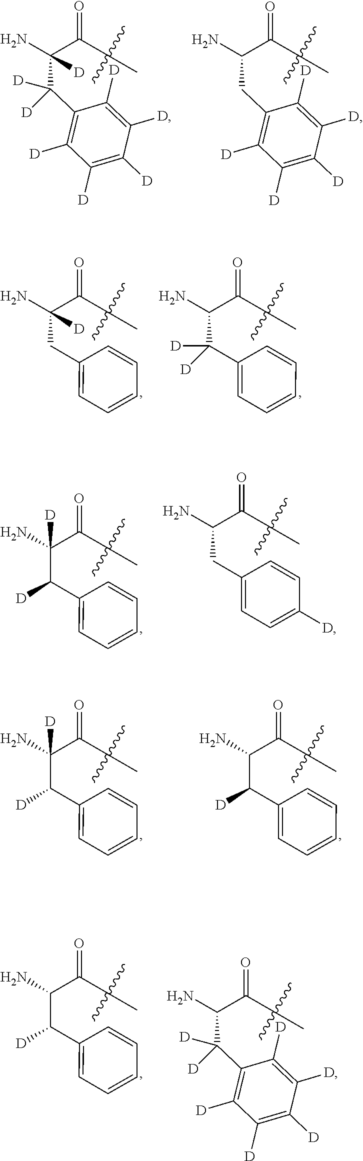 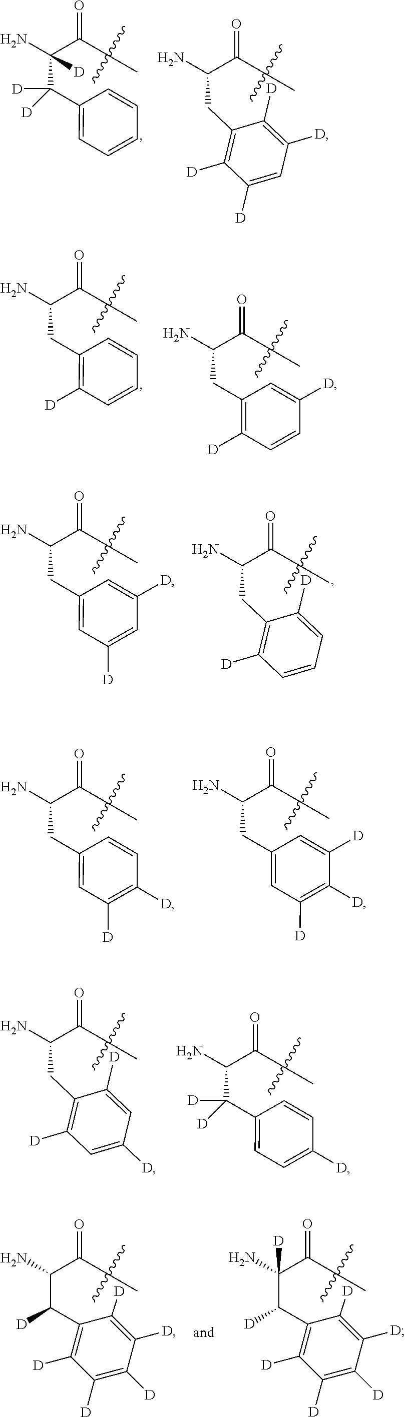
Aaa² is a D-arginine residue or an amino acid residue selected from the group consisting of: 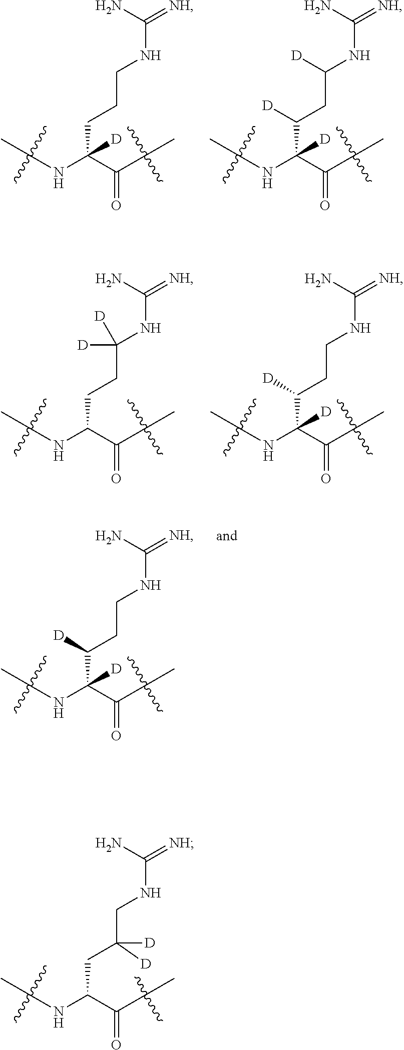
Aaa³ is an L-phenylalanine residue or an amino acid residue selected from the group consisting of: 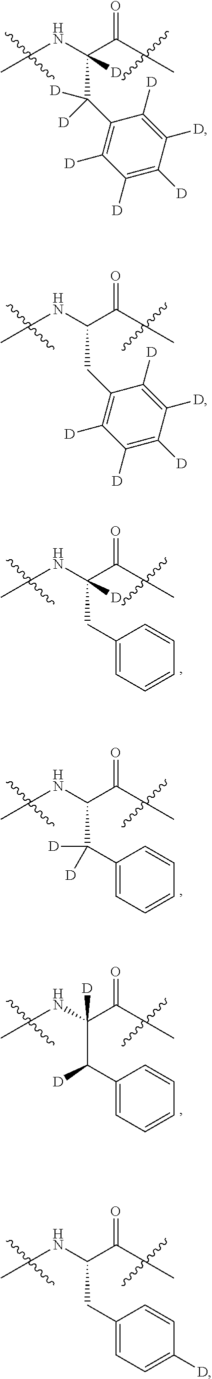 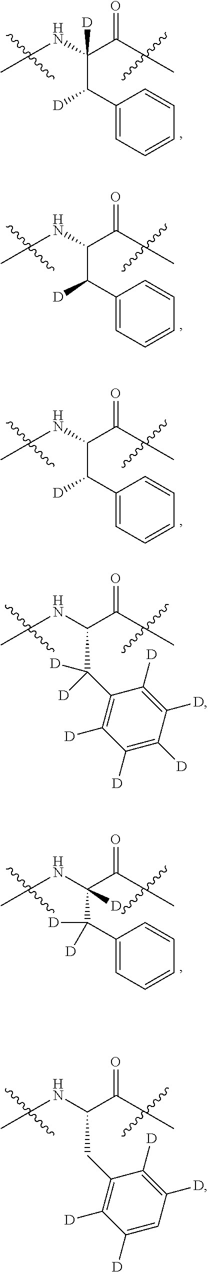 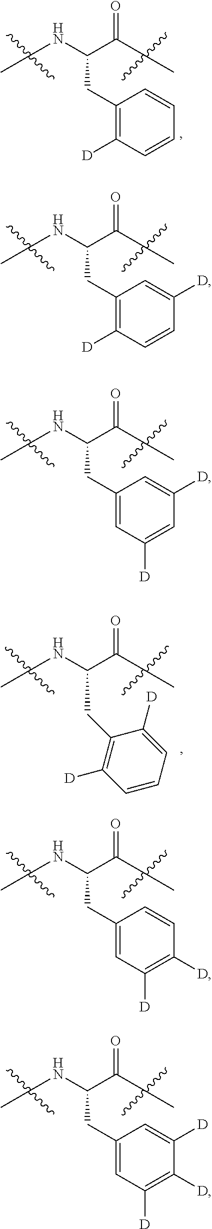 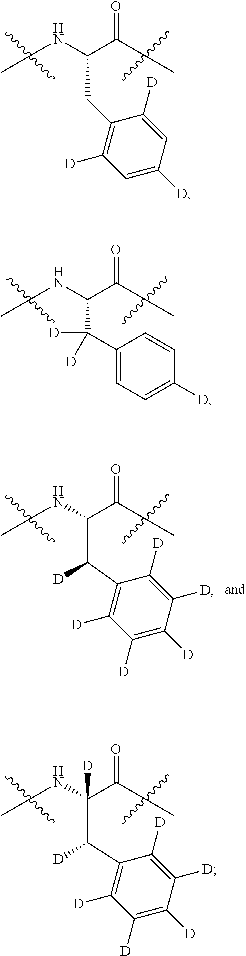
and
Aaa⁴ is an L-lysine residue or an amino acid residue selected from the group consisting of: 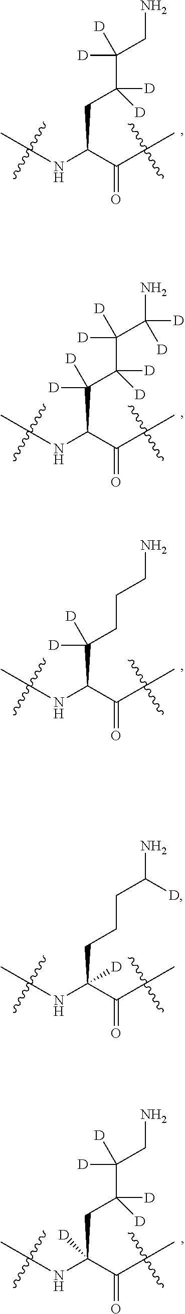 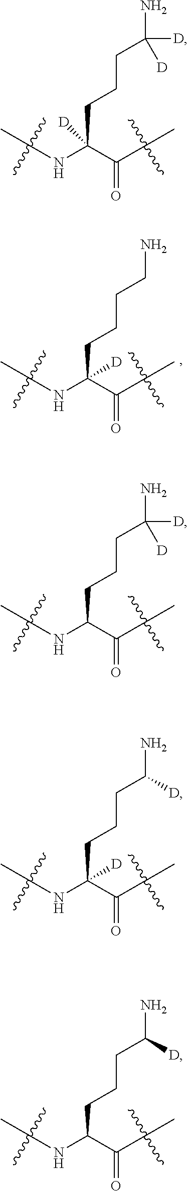 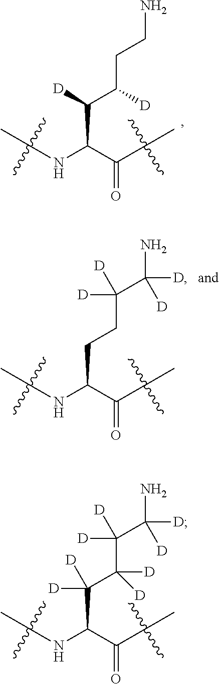
provided that the compound of formula (I) is not Phe-D-Arg-Phe-Lys-NH₂.

3. The compound of claim 1 or 2, wherein Aaa¹ is selected from the group consisting of:

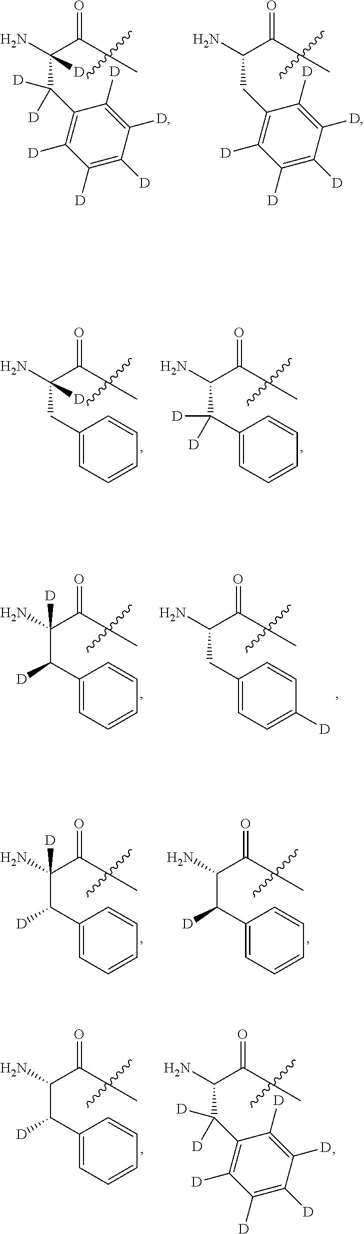

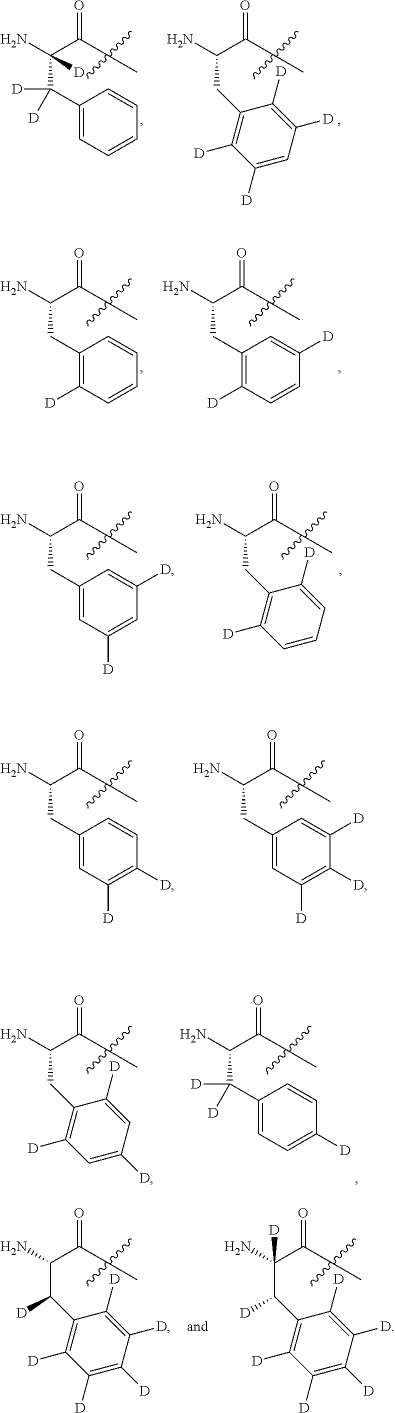

4. The compound of any one of claims 1-3, wherein Aaa² is selected from the group consisting of:

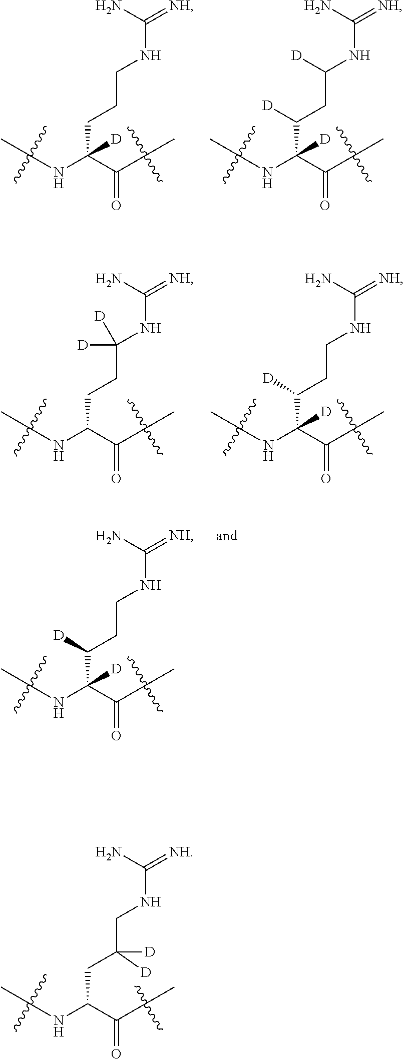

5. The compound of any one of claims 1-4, wherein Aaa³ is selected from the group consisting of:

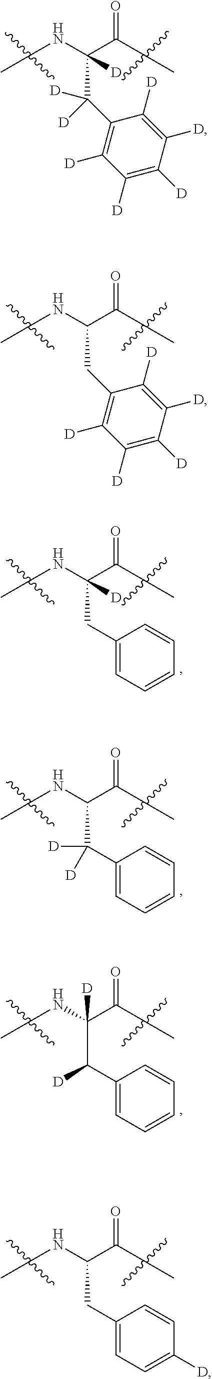

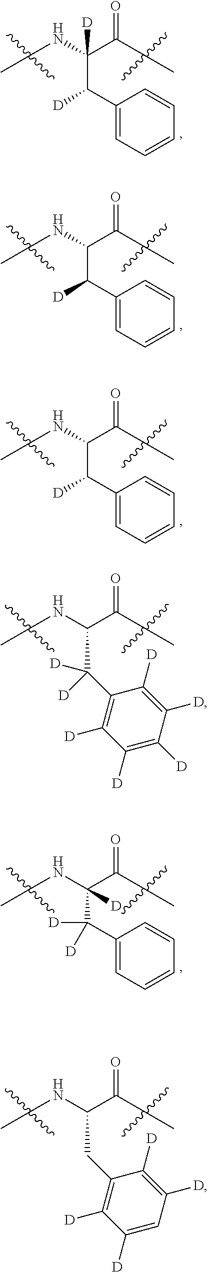

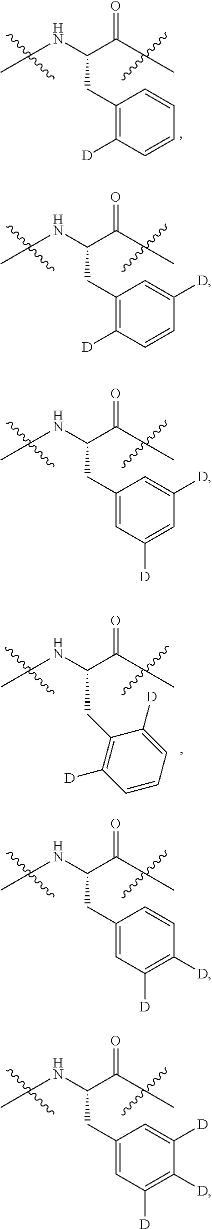

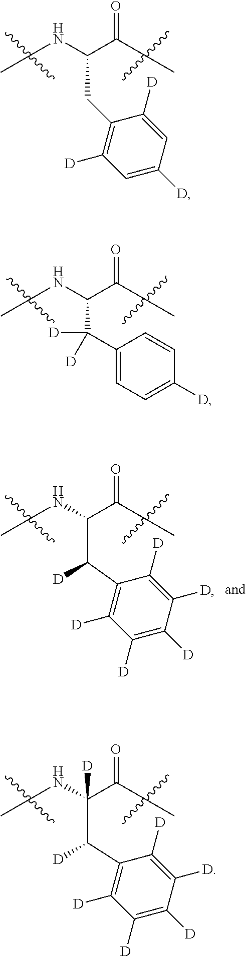

6. The compound of any one of claims 1-5, wherein Aaa⁴ is selected from the group consisting of:

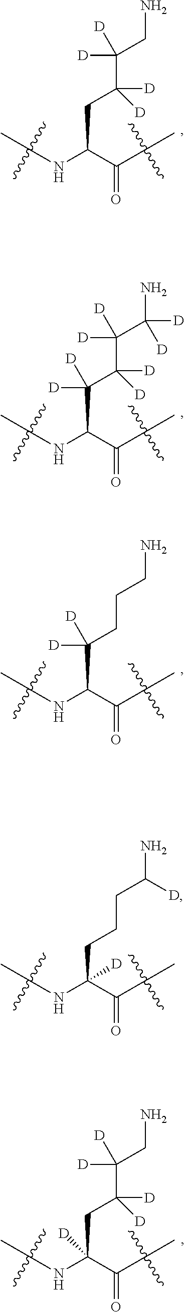

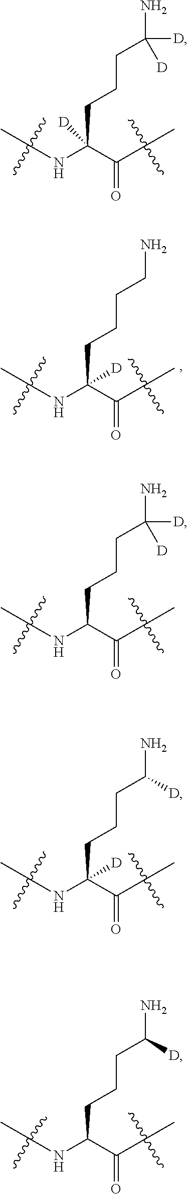

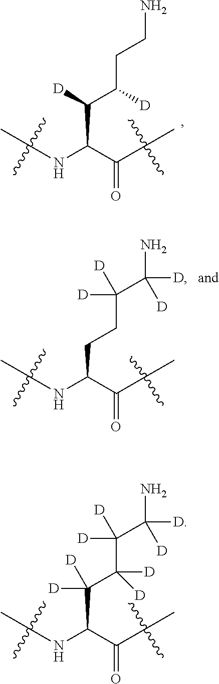

7. The compound of claim 2, selected from the following table:

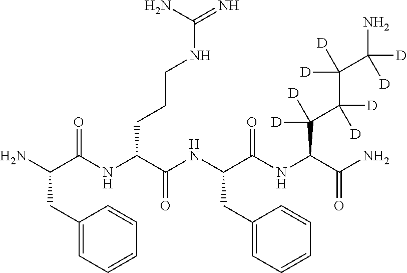 [SMILES: [2H]C([2H])(N)C([2H])([2H])C([2H])([2H])C([2H])([2H])[C@H](NC(=O)[C@H](Cc1ccccc1)NC(=O)[C@@H](CCCNC(=N)N)NC(=O)[C@@H](N)Cc1ccccc1)C(N)=O]

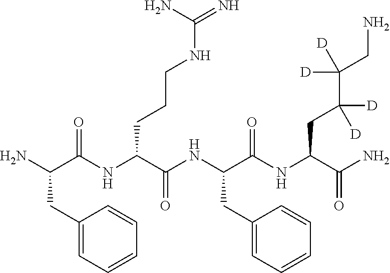 [SMILES: [2H]C([2H])(CN)C([2H])([2H])C[C@H](NC(=O)[C@H](Cc1ccccc1)NC(=O)[C@@H](CCCNC(=N)N)NC(=O)[C@@H](N)Cc1ccccc1)C(N)=O]

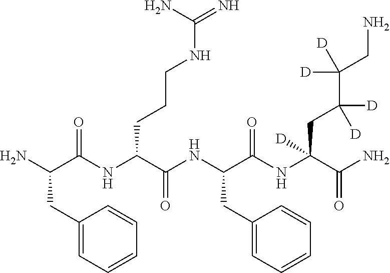 [SMILES: [2H]C([2H])(CN)C([2H])([2H])C[C@]([2H])(NC(=O)[C@H](Cc1ccccc1)NC(=O)[C@@H](CCCNC(=N)N)NC(=O)[C@@H](N)Cc1ccccc1)C(N)=O]

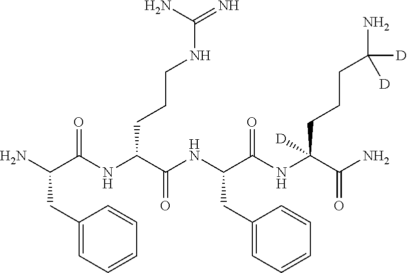 [SMILES: [2H]C([2H])(N)CCC[C@]([2H])(NC(=O)[C@H](Cc1ccccc1)NC(=O)[C@@H](CCCNC(=N)N)NC(=O)[C@@H](N)Cc1ccccc1)C(N)=O]

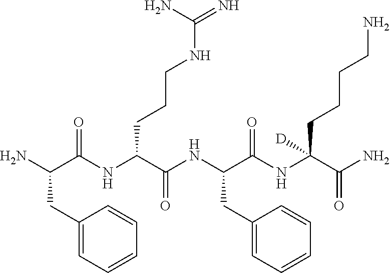 [SMILES: [2H][C@@](CCCCN)(NC(=O)[C@H](Cc1ccccc1)NC(=O)[C@@H](CCCNC(=N)N)NC(=O)[C@@H](N)Cc1ccccc1)C(N)=O]

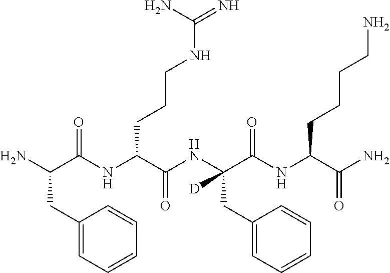 [SMILES: [2H][C@@](Cc1ccccc1)(NC(=O)[C@@H](CCCNC(=N)N)NC(=O)[C@@H](N)Cc1ccccc1)C(=O)N[C@@H](CCCCN)C(N)=O]

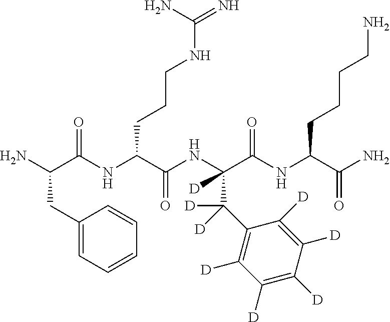 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@H](CCCCN)C(N)=O)c([2H])c1[2H]]

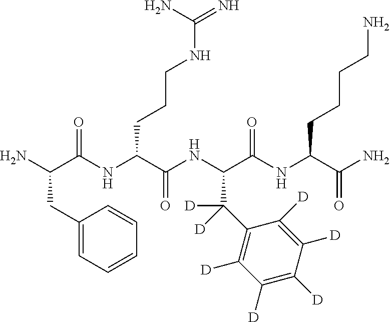 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@@H](CCCCN)C(N)=O)c([2H])c1[2H]]

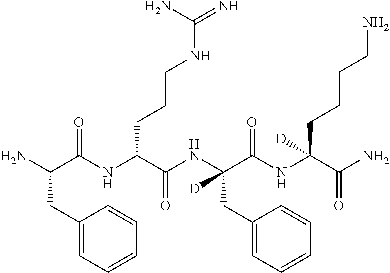 [SMILES: [2H][C@@](CCCCN)(NC(=O)[C@]([2H])(Cc1ccccc1)NC(=O)[C@@H](CCCNC(=N)N)NC(=O)[C@@H](N)Cc1ccccc1)C(N)=O]

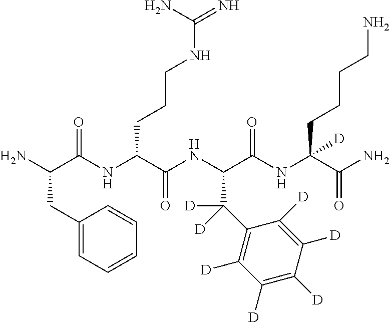 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@@]([2H])(CCCCN)C(N)=O)c([2H])c1[2H]]

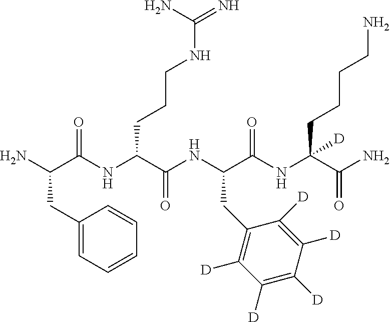 [SMILES: [2H]c1c([2H])c([2H])c(C[C@H](NC(=O)[C@@H](CCCNC(=N)N)NC(=O)[C@@H](N)Cc2ccccc2)C(=O)N[C@@]([2H])(CCCCN)C(N)=O)c([2H])c1[2H]]

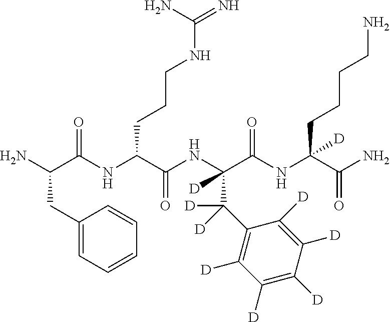 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@]([2H])(CCCCN)C(N)=O)c([2H])c1[2H]]

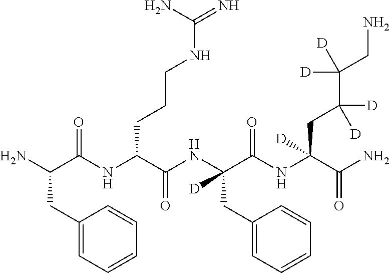 [SMILES: [2H]C([2H])(CN)C([2H])([2H])C[C@]([2H])(NC(=O)[C@]([2H])(Cc1ccccc1)NC(=O)[C@@H](CCCNC(=N)N)NC(=O)[C@@H](N)Cc1ccccc1)C(N)=O]

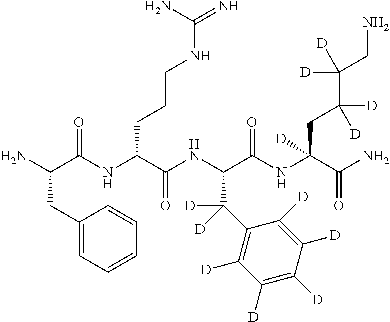 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@@]([2H])(CC([2H])([2H])C([2H])([2H])CN)C(N)=O)c([2H])c1[2H]]

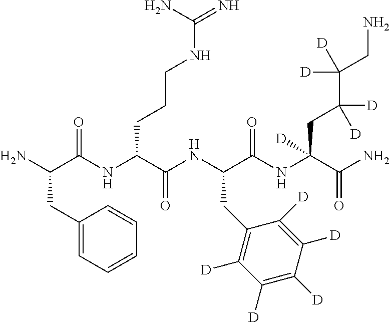 [SMILES: [2H]c1c([2H])c([2H])c(C[C@H](NC(=O)[C@@H](CCCNC(=N)N)NC(=O)[C@@H](N)Cc2ccccc2)C(=O)N[C@@]([2H])(CC([2H])([2H])C([2H])([2H])CN)C(N)=O)c([2H])c1[2H]]

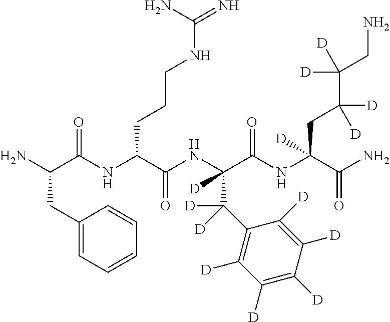 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@]([2H])(CC([2H])([2H])C([2H])([2H])CN)C(N)=O)c([2H])c1[2H]]

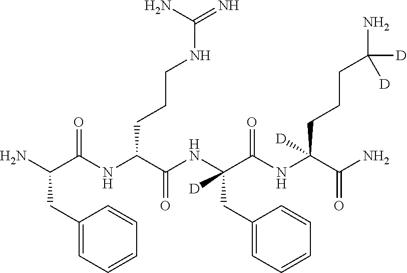 [SMILES: [2H]C([2H])(N)CCC[C@]([2H])(NC(=O)[C@]([2H])(Cc1ccccc1)NC(=O)[C@@H](CCCNC(=N)N)NC(=O)[C@@H](N)Cc1ccccc1)C(N)=O]

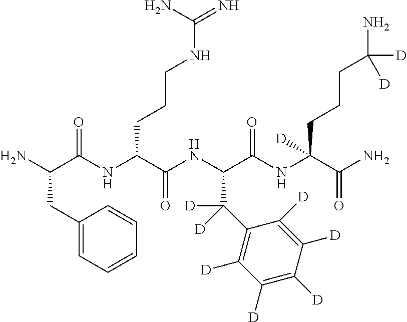 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@@]([2H])(CCCC([2H])([2H])N)C(N)=O)c([2H])c1[2H]]

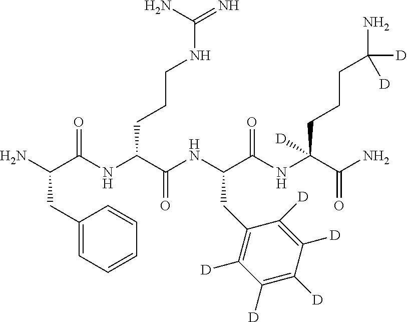 [SMILES: [2H]c1c([2H])c([2H])c(C[C@H](NC(=O)[C@@H](CCCNC(=N)N)NC(=O)[C@@H](N)Cc2ccccc2)C(=O)N[C@@]([2H])(CCCC([2H])([2H])N)C(N)=O)c([2H])c1[2H]]

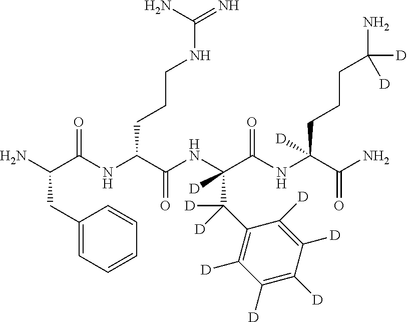 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@]([2H])(CCCC([2H])([2H])N)C(N)=O)c([2H])c1[2H]]

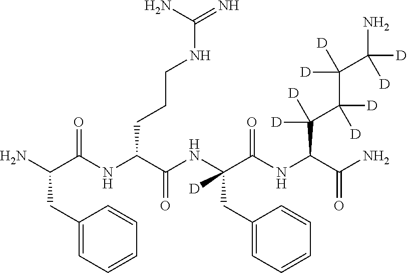 [SMILES: [2H]C([2H])(N)C([2H])([2H])C([2H])([2H])C([2H])([2H])[C@H](NC(=O)[C@]([2H])(Cc1ccccc1)NC(=O)[C@@H](CCCNC(=N)N)NC(=O)[C@@H](N)Cc1ccccc1)C(N)=O]

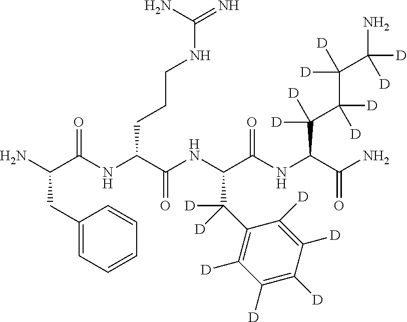 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@H](C(N)=O)C([2H])([2H])C([2H])([2H])C([2H])([2H])C([2H])([2H])N)c([2H])c1[2H]]

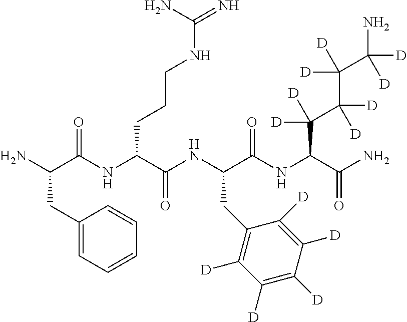 [SMILES: [2H]c1c([2H])c([2H])c(C[C@H](NC(=O)[C@@H](CCCNC(=N)N)NC(=O)[C@@H](N)Cc2ccccc2)C(=O)N[C@H](C(N)=O)C([2H])([2H])C([2H])([2H])C([2H])([2H])C([2H])([2H])N)c([2H])c1[2H]]

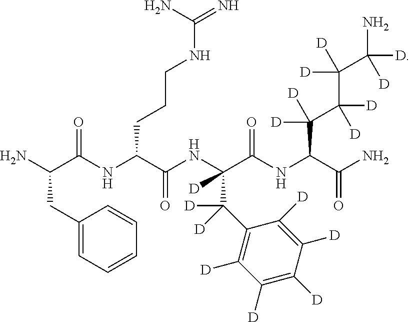

8. The compound of claim 7, selected from the following table:

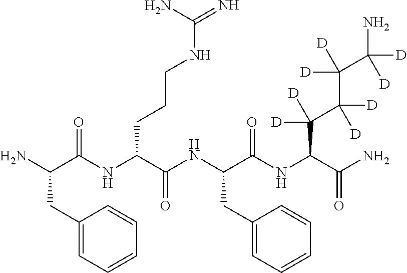 [SMILES: [2H]C([2H])(N)C([2H])([2H])C([2H])([2H])C([2H])([2H])[C@H](NC(=O)[C@H](Cc1ccccc1)NC(=O)[C@@H](CCCNC(=N)N)NC(=O)[C@@H](N)Cc1ccccc1)C(N)=O]

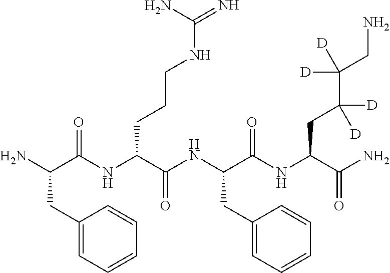 [SMILES: [2H]C([2H])(CN)C([2H])([2H])C[C@H](NC(=O)[C@H](Cc1ccccc1)NC(=O)[C@@H](CCCNC(=N)N)NC(=O)[C@@H](N)Cc1ccccc1)C(N)=O]

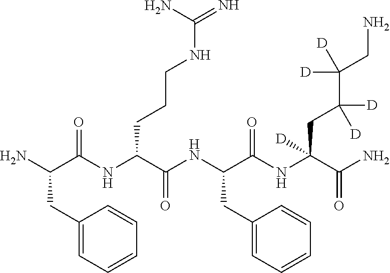 [SMILES: [2H]C([2H])(CN)C([2H])([2H])C[C@]([2H])(NC(=O)[C@H](Cc1ccccc1)NC(=O)[C@@H](CCCNC(=N)N)NC(=O)[C@@H](N)Cc1ccccc1)C(N)=O]

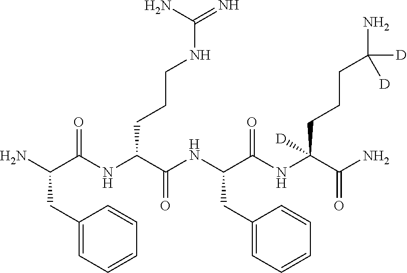 [SMILES: [2H]C([2H])(N)CCC[C@]([2H])(NC(=O)[C@H](Cc1ccccc1)NC(=O)[C@@H](CCCNC(=N)N)NC(=O)[C@@H](N)Cc1ccccc1)C(N)=O]

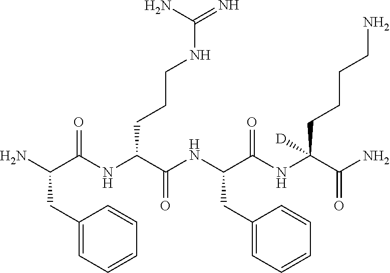 [SMILES: [2H][C@@](CCCCN)(NC(=O)[C@H](Cc1ccccc1)NC(=O)[C@@H](CCCNC(=N)N)NC(=O)[C@@H](N)Cc1ccccc1)C(N)=O]

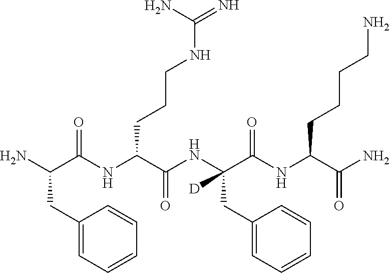 [SMILES: [2H][C@@](Cc1ccccc1)(NC(=O)[C@@H](CCCNC(=N)N)NC(=O)[C@@H](N)Cc1ccccc1)C(=O)N[C@@H](CCCCN)C(N)=O]

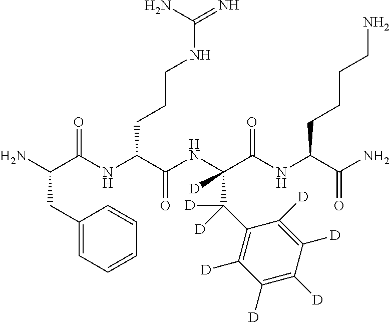 [SMILES: [2H]c1c([2H])c([2H])c(C([2H])([2H])[C@]([2H])(NC(=O)[C@@H](CCCNC(=N)N)NC(=O)[C@@H](N)Cc2ccccc2)C(=O)N[C@@H](CCCCN)C(N)=O)c([2H])c1[2H]]

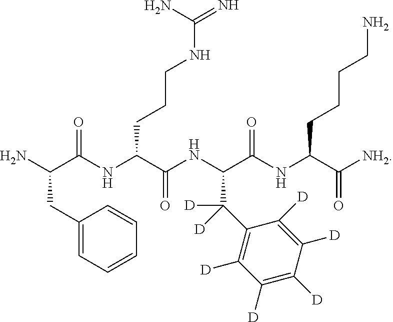 [SMILES: [2H]c1c([2H])c([2H])c(C([2H])([2H])[C@H](NC(=O)[C@@H](CCCNC(=N)N)NC(=O)[C@@H](N)Cc2ccccc2)C(=O)N[C@@H](CCCCN)C(N)=O)c([2H])c1[2H]]

9. A compound of formula (II), or a pharmaceutically acceptable salt thereof:
Aaa⁵-Aaa⁶-Aaa⁷-Aaa⁸-NH₂ (II);
wherein:
Aaa⁵ is a D-arginine residue;
Aaa⁶ is: OH; 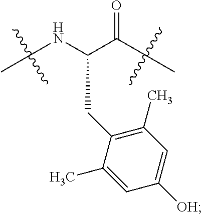 [SMILES: *N[C@@H](Cc1c(C)cc(O)cc1C)C(*)=O]
Aaa⁷ is an L-lysine residue;
Aaa⁸ is an L-phenylalanine residue; and
at least one hydrogen atom is replaced by a deuterium atom.

10. The compound of claim 9, wherein:
Aaa⁵ is a D-arginine residue or an amino acid residue selected from the group consisting of: 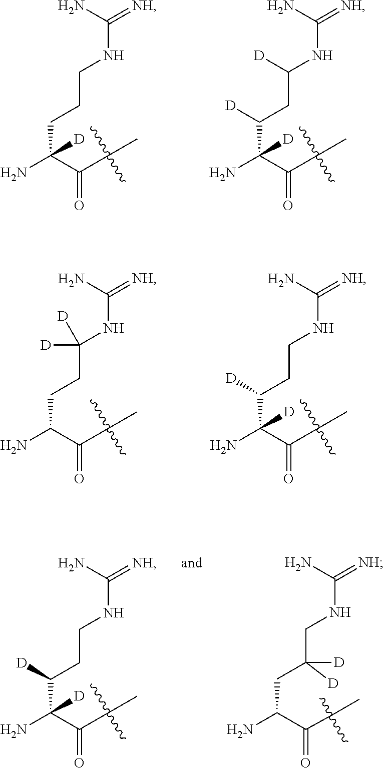
Aaa⁶ is: 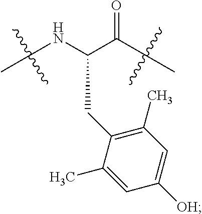 [SMILES: *N[C@@H](Cc1c(C)cc(O)cc1C)C(*)=O]
Aaa⁷ is an L-lysine residue or an amino acid residue selected from the group consisting of: 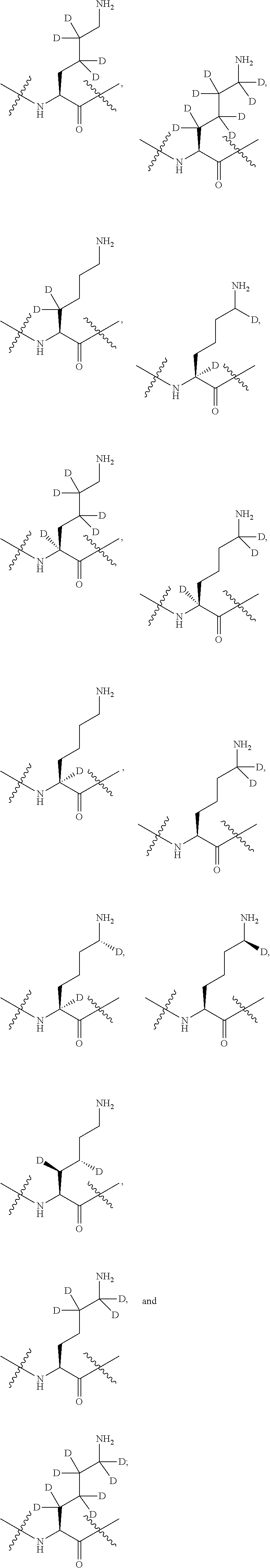
and
Aaa⁸ is an L-phenylalanine residue or an amino acid residue selected from the group consisting of: 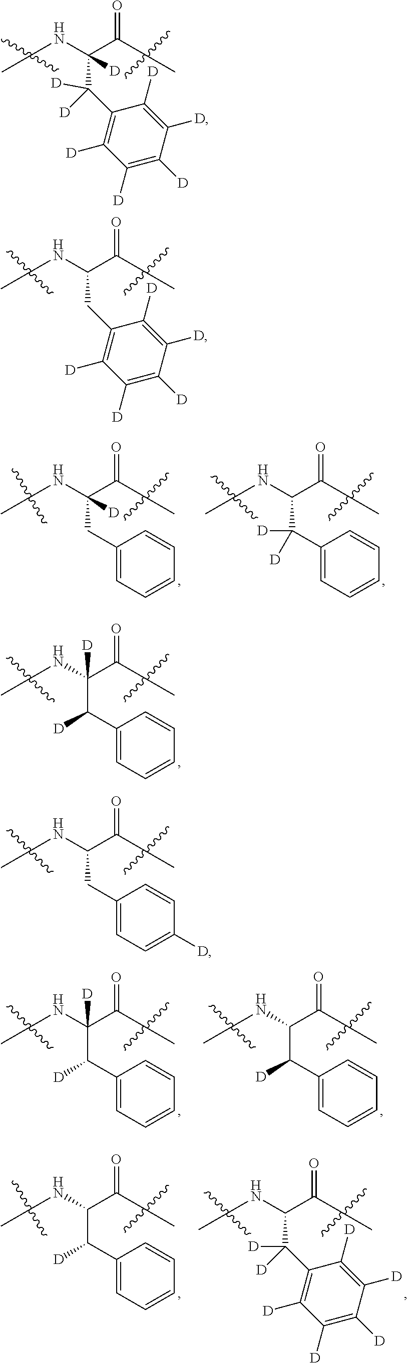 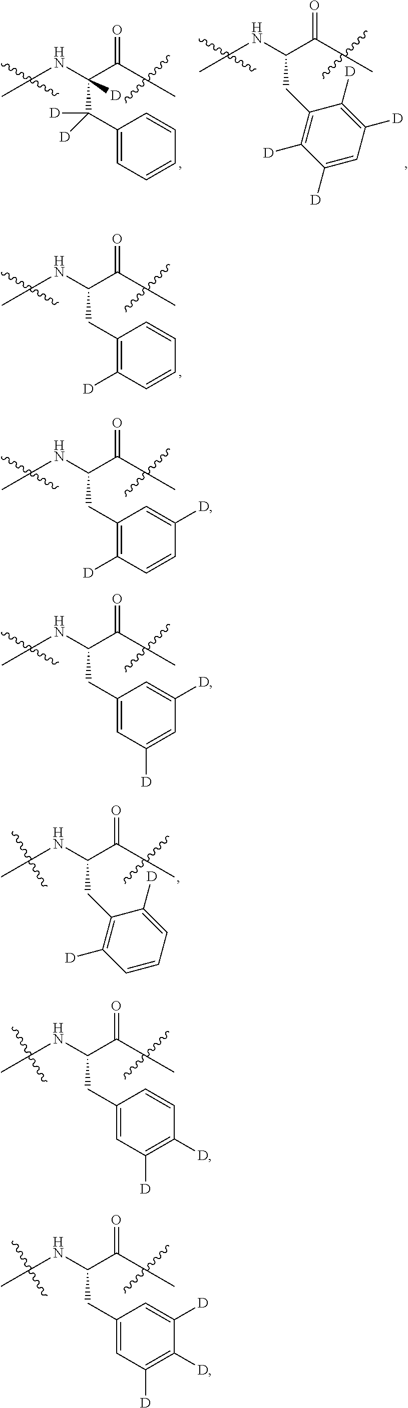 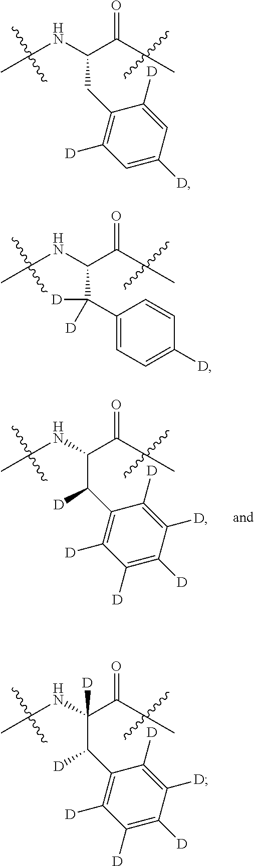
provided that the compound of formula (II) is not D-Arg-DMT-Lys-Phe-NH₂; wherein DMT represents

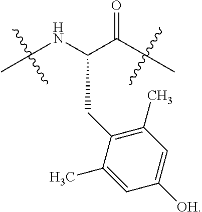 [SMILES: *N[C@@H](Cc1c(C)cc(O)cc1C)C(*)=O]

11. The compound of claim 9 or 10, wherein Aaa⁵ is an amino acid residue selected from the group consisting of:

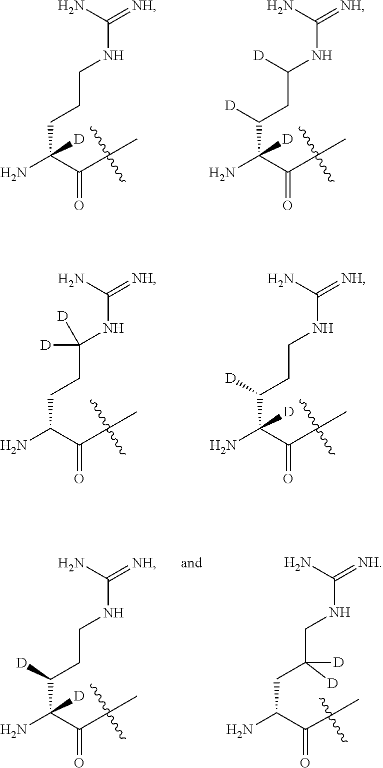

12. The compound of any one of claims 9-11, wherein Aaa⁷ is an amino acid residue selected from the group consisting of:

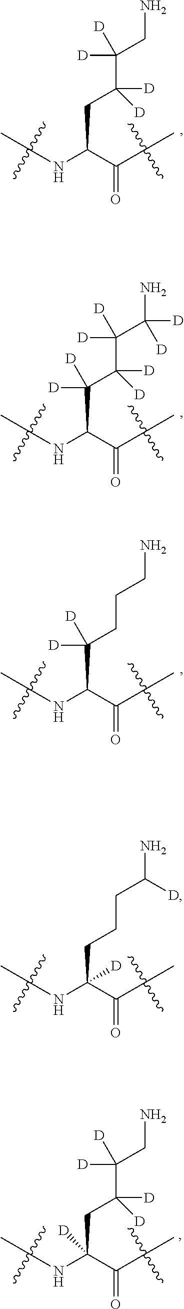

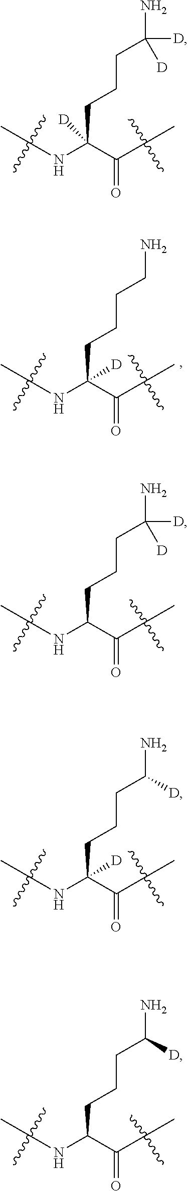

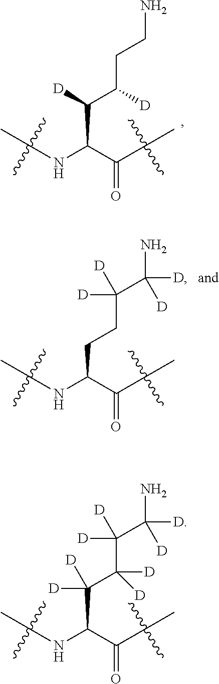

13. The compound of any one of claims 9-12, wherein Aaa⁸ is an amino acid residue selected from the group consisting of:

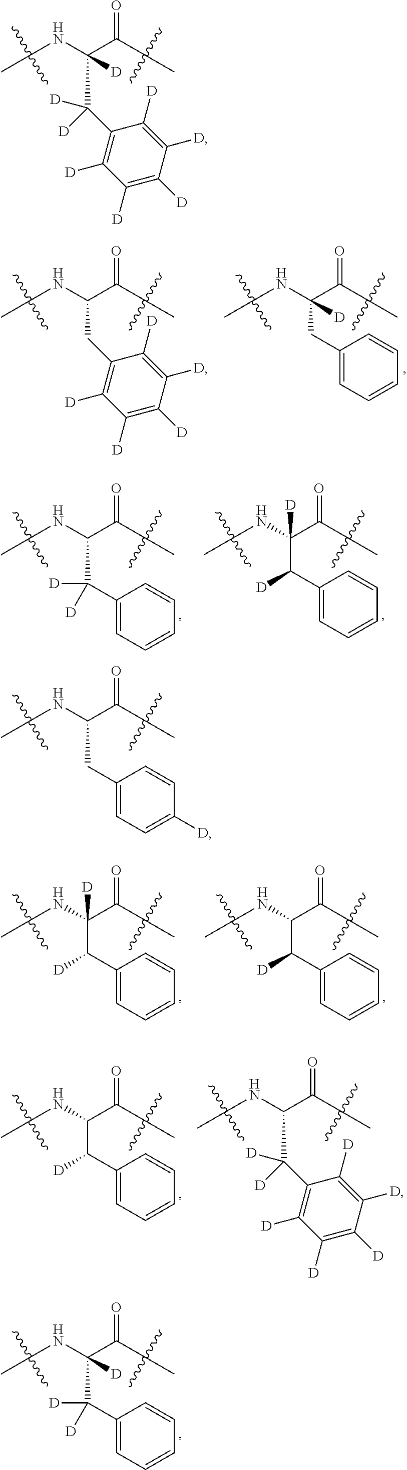

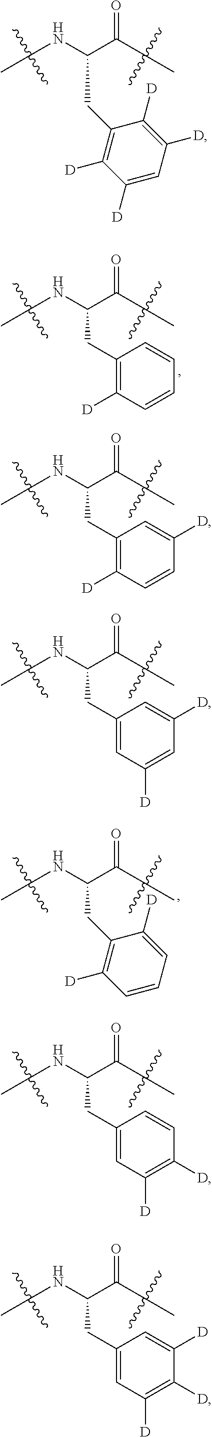

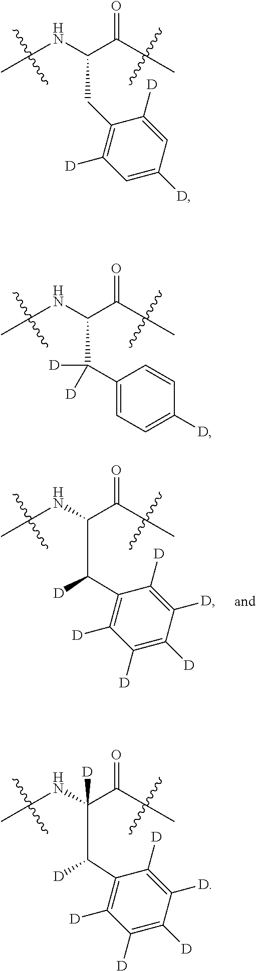

14. The compound of claim 10, selected from the following table:

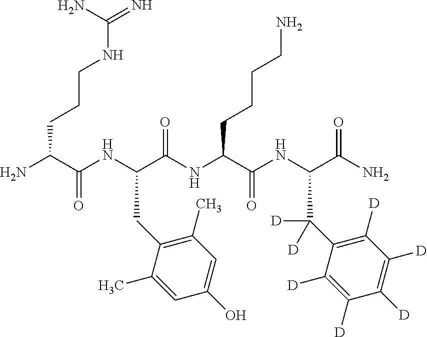 [SMILES: [2H]c1c([2H])c([2H])c(C([2H])([2H])[C@H](NC(=O)[C@H](CCCCN)NC(=O)[C@H](Cc2c(C)cc(O)cc2C)NC(=O)[C@H](N)CCCNC(=N)N)C(N)=O)c([2H])c1[2H]]

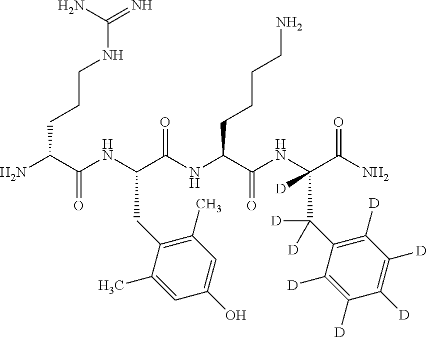 [SMILES: [2H]c1c([2H])c([2H])c(C([2H])([2H])[C@]([2H])(NC(=O)[C@H](CCCCN)NC(=O)[C@H](Cc2c(C)cc(O)cc2C)NC(=O)[C@H](N)CCCNC(=N)N)C(N)=O)c([2H])c1[2H]]

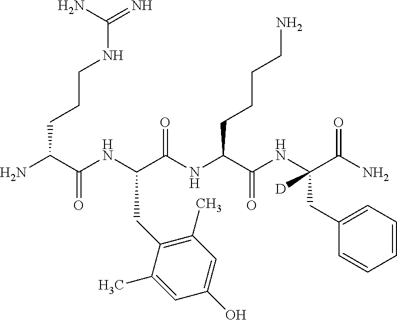 [SMILES: [2H][C@@](Cc1ccccc1)(NC(=O)[C@H](CCCCN)NC(=O)[C@H](Cc1c(C)cc(O)cc1C)NC(=O)[C@H](N)CCCNC(=N)N)C(N)=O]

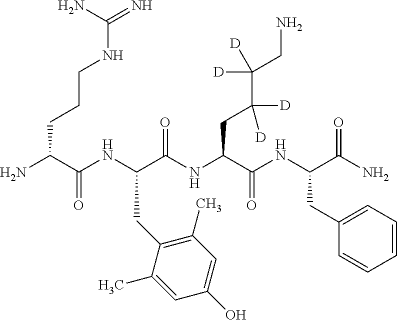 [SMILES: [2H]C([2H])(CN)C([2H])([2H])C[C@H](NC(=O)[C@H](Cc1c(C)cc(O)cc1C)NC(=O)[C@H](N)CCCNC(=N)N)C(=O)N[C@@H](Cc1ccccc1)C(N)=O]

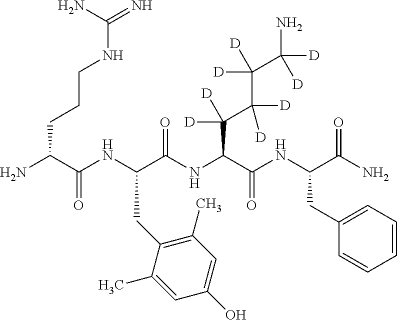 [SMILES: [2H]C([2H])(N)C([2H])([2H])C([2H])([2H])C([2H])([2H])[C@H](NC(=O)[C@H](Cc1c(C)cc(O)cc1C)NC(=O)[C@H](N)CCCNC(=N)N)C(=O)N[C@@H](Cc1ccccc1)C(N)=O]

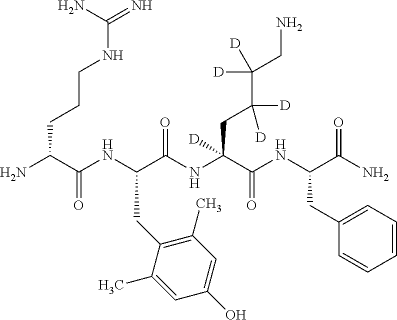 [SMILES: [2H]C([2H])(CN)C([2H])([2H])C[C@]([2H])(NC(=O)[C@H](Cc1c(C)cc(O)cc1C)NC(=O)[C@H](N)CCCNC(=N)N)C(=O)N[C@@H](Cc1ccccc1)C(N)=O]

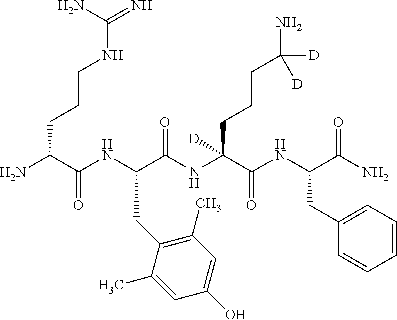 [SMILES: [2H]C([2H])(N)CCC[C@]([2H])(NC(=O)[C@H](Cc1c(C)cc(O)cc1C)NC(=O)[C@H](N)CCCNC(=N)N)C(=O)N[C@@H](Cc1ccccc1)C(N)=O]

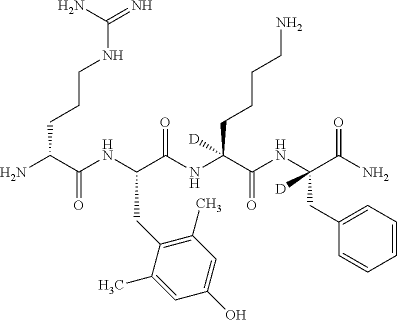 [SMILES: [2H][C@@](Cc1ccccc1)(NC(=O)[C@]([2H])(CCCCN)NC(=O)[C@H](Cc1c(C)cc(O)cc1C)NC(=O)[C@H](N)CCCNC(=N)N)C(N)=O]

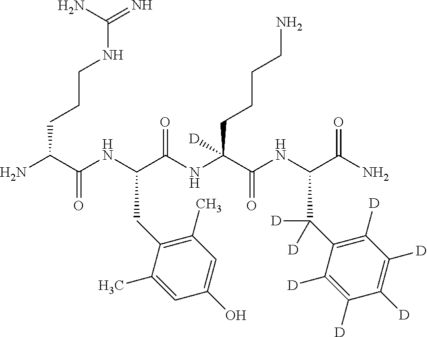 [SMILES: [2H]c1c([2H])c([2H])c(C([2H])([2H])[C@H](NC(=O)[C@]([2H])(CCCCN)NC(=O)[C@H](Cc2c(C)cc(O)cc2C)NC(=O)[C@H](N)CCCNC(=N)N)C(N)=O)c([2H])c1[2H]]

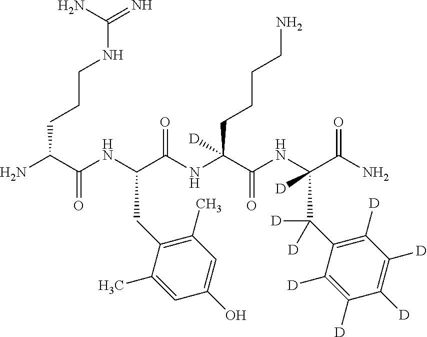 [SMILES: [2H]c1c([2H])c([2H])c(C([2H])([2H])[C@]([2H])(NC(=O)[C@]([2H])(CCCCN)NC(=O)[C@H](Cc2c(C)cc(O)cc2C)NC(=O)[C@H](N)CCCNC(=N)N)C(N)=O)c([2H])c1[2H]]

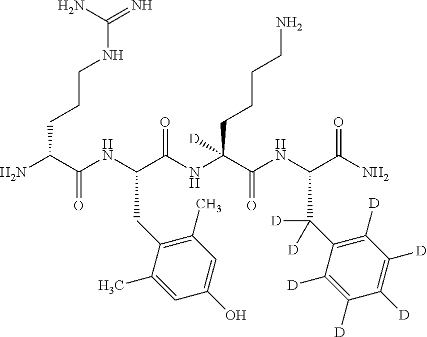 [SMILES: [2H]c1c([2H])c([2H])c(C([2H])([2H])[C@H](NC(=O)[C@]([2H])(CCCCN)NC(=O)[C@H](Cc2c(C)cc(O)cc2C)NC(=O)[C@H](N)CCCNC(=N)N)C(N)=O)c([2H])c1[2H]]

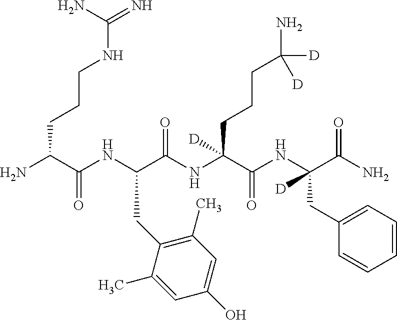 [SMILES: [2H]C([2H])(N)CCC[C@]([2H])(NC(=O)[C@H](Cc1c(C)cc(O)cc1C)NC(=O)[C@H](N)CCCNC(=N)N)C(=O)N[C@@]([2H])(Cc1ccccc1)C(N)=O]

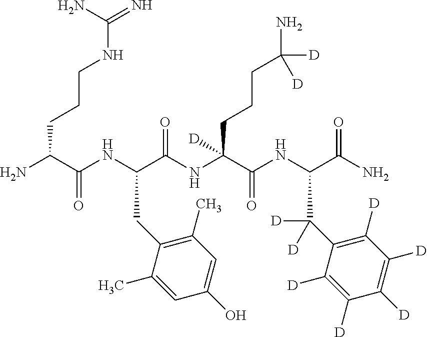 [SMILES: [2H]c1c([2H])c([2H])c(C([2H])([2H])[C@H](NC(=O)[C@]([2H])(CCCC([2H])([2H])N)NC(=O)[C@H](Cc2c(C)cc(O)cc2C)NC(=O)[C@H](N)CCCNC(=N)N)C(N)=O)c([2H])c1[2H]]

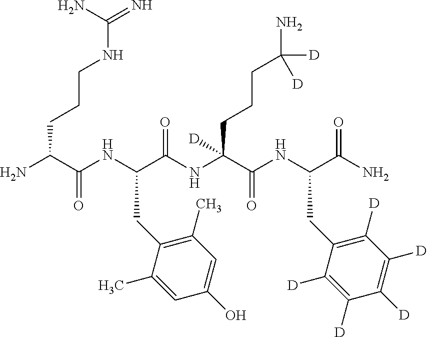 [SMILES: [2H]c1c([2H])c([2H])c(C[C@H](NC(=O)[C@]([2H])(CCCC([2H])([2H])N)NC(=O)[C@H](Cc2c(C)cc(O)cc2C)NC(=O)[C@H](N)CCCNC(=N)N)C(N)=O)c([2H])c1[2H]]

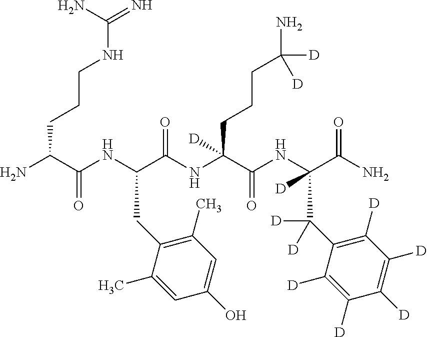 [SMILES: [2H]c1c([2H])c([2H])c(C([2H])([2H])[C@]([2H])(NC(=O)[C@]([2H])(CCCC([2H])([2H])N)NC(=O)[C@H](Cc2c(C)cc(O)cc2C)NC(=O)[C@H](N)CCCNC(=N)N)C(N)=O)c([2H])c1[2H]]

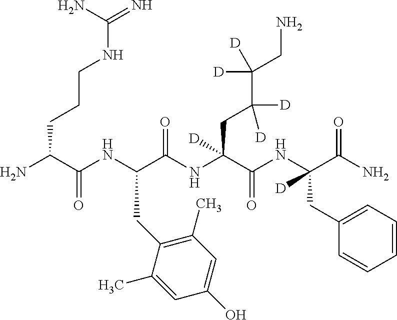 [SMILES: [2H]C([2H])(CN)C([2H])([2H])C[C@]([2H])(NC(=O)[C@H](Cc1c(C)cc(O)cc1C)NC(=O)[C@H](N)CCCNC(=N)N)C(=O)N[C@@]([2H])(Cc1ccccc1)C(N)=O]

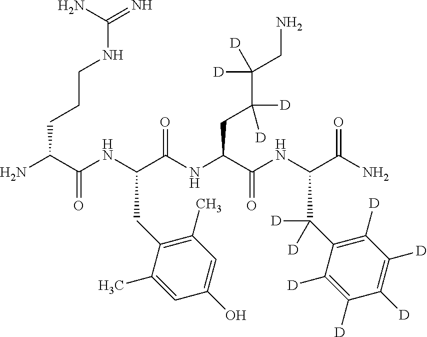 [SMILES: [2H]c1c([2H])c([2H])c(C([2H])([2H])[C@H](NC(=O)[C@H](CC([2H])([2H])C([2H])([2H])CN)NC(=O)[C@H](Cc2c(C)cc(O)cc2C)NC(=O)[C@H](N)CCCNC(=N)N)C(N)=O)c([2H])c1[2H]]

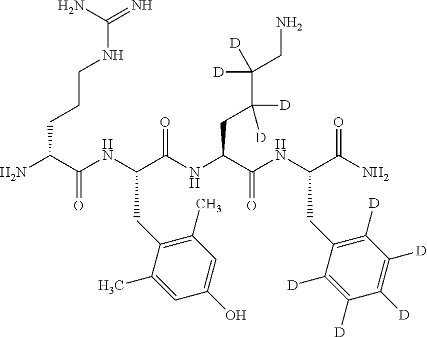 [SMILES: [2H]c1c([2H])c([2H])c(C[C@H](NC(=O)[C@H](CC([2H])([2H])C([2H])([2H])CN)NC(=O)[C@H](Cc2c(C)cc(O)cc2C)NC(=O)[C@H](N)CCCNC(=N)N)C(N)=O)c([2H])c1[2H]]

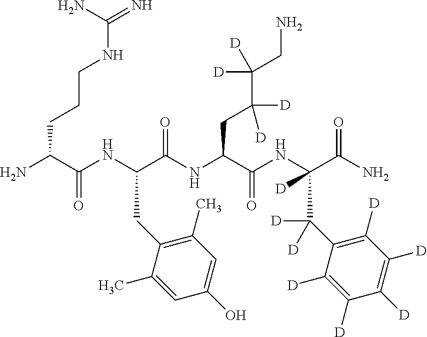 [SMILES: [2H]c1c([2H])c([2H])c(C([2H])([2H])[C@]([2H])(NC(=O)[C@H](CC([2H])([2H])C([2H])([2H])CN)NC(=O)[C@H](Cc2c(C)cc(O)cc2C)NC(=O)[C@H](N)CCCNC(=N)N)C(N)=O)c([2H])c1[2H]]

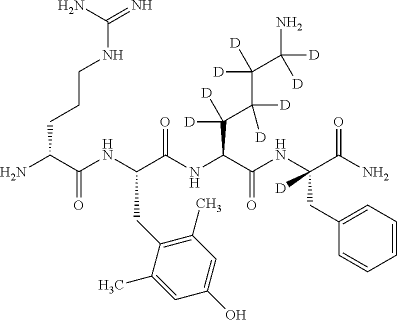 [SMILES: [2H]C([2H])(N)C([2H])([2H])C([2H])([2H])C([2H])([2H])[C@H](NC(=O)[C@H](Cc1c(C)cc(O)cc1C)NC(=O)[C@H](N)CCCNC(=N)N)C(=O)N[C@@]([2H])(Cc1ccccc1)C(N)=O]

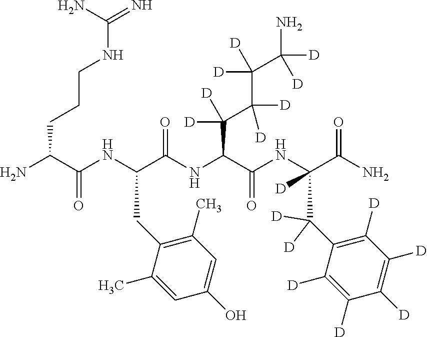 [SMILES: [2H]c1c([2H])c([2H])c(C([2H])([2H])[C@]([2H])(NC(=O)[C@@H](NC(=O)[C@H](Cc2c(C)cc(O)cc2C)NC(=O)[C@H](N)CCCNC(=N)N)C([2H])([2H])C([2H])([2H])C([2H])([2H])C([2H])([2H])N)C(N)=O)c([2H])c1[2H]]

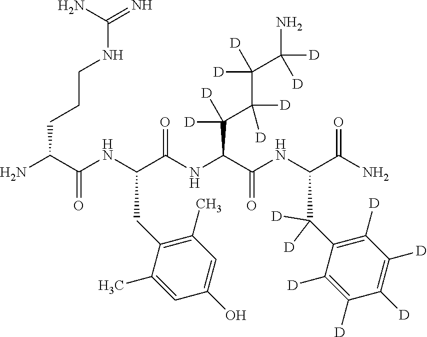 [SMILES: [2H]c1c([2H])c([2H])c(C([2H])([2H])[C@H](NC(=O)[C@@H](NC(=O)[C@H](Cc2c(C)cc(O)cc2C)NC(=O)[C@H](N)CCCNC(=N)N)C([2H])([2H])C([2H])([2H])C([2H])([2H])C([2H])([2H])N)C(N)=O)c([2H])c1[2H]]

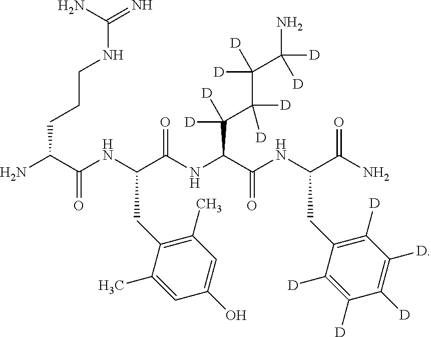

15. The compound of claim 14, selected from the following table:

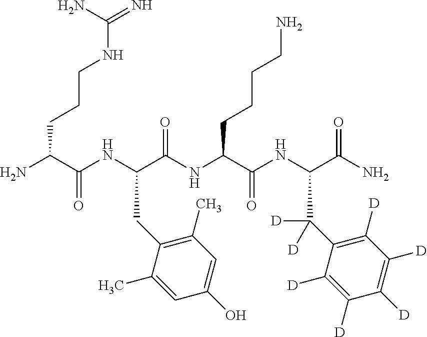 [SMILES: [2H]c1c([2H])c([2H])c(C([2H])([2H])[C@H](NC(=O)[C@H](CCCCN)NC(=O)[C@H](Cc2c(C)cc(O)cc2C)NC(=O)[C@H](N)CCCNC(=N)N)C(N)=O)c([2H])c1[2H]]

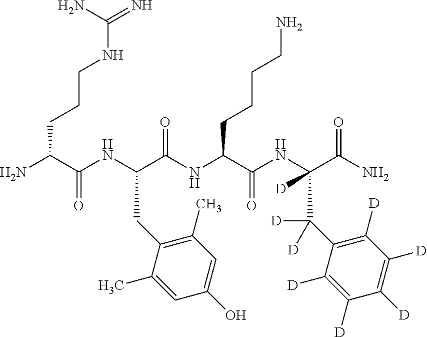 [SMILES: [2H]c1c([2H])c([2H])c(C([2H])([2H])[C@]([2H])(NC(=O)[C@H](CCCCN)NC(=O)[C@H](Cc2c(C)cc(O)cc2C)NC(=O)[C@H](N)CCCNC(=N)N)C(N)=O)c([2H])c1[2H]]

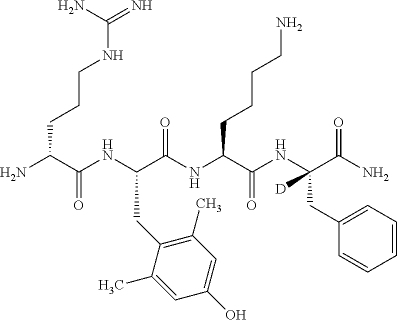 [SMILES: [2H][C@@](Cc1ccccc1)(NC(=O)[C@H](CCCCN)NC(=O)[C@H](Cc1c(C)cc(O)cc1C)NC(=O)[C@H](N)CCCNC(=N)N)C(N)=O]

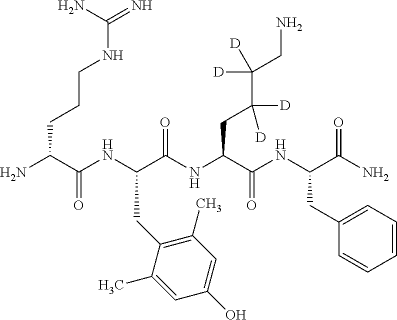 [SMILES: [2H]C([2H])(CN)C([2H])([2H])C[C@H](NC(=O)[C@H](Cc1c(C)cc(O)cc1C)NC(=O)[C@H](N)CCCNC(=N)N)C(=O)N[C@@H](Cc1ccccc1)C(N)=O]

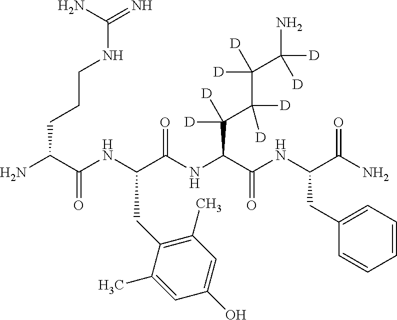 [SMILES: [2H]C([2H])(N)C([2H])([2H])C([2H])([2H])C([2H])([2H])[C@H](NC(=O)[C@H](Cc1c(C)cc(O)cc1C)NC(=O)[C@H](N)CCCNC(=N)N)C(=O)N[C@@H](Cc1ccccc1)C(N)=O]

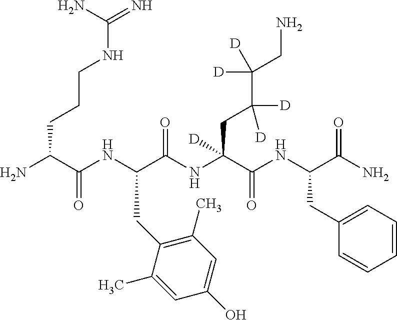 [SMILES: [2H]C([2H])(CN)C([2H])([2H])C[C@]([2H])(NC(=O)[C@H](Cc1c(C)cc(O)cc1C)NC(=O)[C@H](N)CCCNC(=N)N)C(=O)N[C@@H](Cc1ccccc1)C(N)=O]

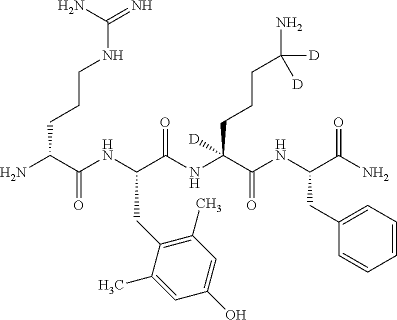 [SMILES: [2H]C([2H])(N)CCC[C@]([2H])(NC(=O)[C@H](Cc1c(C)cc(O)cc1C)NC(=O)[C@H](N)CCCNC(=N)N)C(=O)N[C@@H](Cc1ccccc1)C(N)=O]

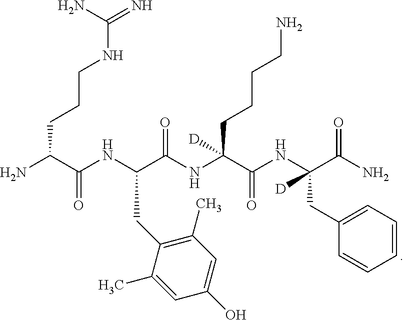 [SMILES: [2H][C@@](Cc1ccccc1)(NC(=O)[C@]([2H])(CCCCN)NC(=O)[C@H](Cc1c(C)cc(O)cc1C)NC(=O)[C@H](N)CCCNC(=N)N)C(N)=O]

16. A pharmaceutical composition, comprising a compound of any one of claims 1-15;
and a pharmaceutically acceptable carrier.

17. A method for treating or preventing ischemia-reperfusion injury in a subject in need thereof comprising administering to the subject a therapeutically effective amount of a compound of any one of claims 1-15.

18. The method of claim 17, wherein the ischemia-reperfusion injury is cardiac ischemia-reperfusion injury.

19. A method for treating or preventing a myocardial infarction in a subject in need thereof comprising administering to the subject a therapeutically effective amount of a compound of any one of claims 1-15.

20. The method of any one of claims 17-19, wherein the compound is administered orally, topically, systemically, intravenously, subcutaneously, intraperitoneally, or intramuscularly.
